(19) United States
(12) Patent Application Publication
(10) Pub. No.: US 2026/0317340 A1
(43) Pub. Date: Oct. 15, 2026

(54) COMPOUNDS, COMPOSITIONS, AND METHODS OF TREATING DISORDERS

(71) Applicant: Chengdu Anticancer Bioscience, Ltd., Chengdu (CN)

(72) Inventors: Jian Huang, Chengdu (CN); Linsheng Zhong, Chengdu (CN); Gang Lv, Chengdu (CN); Jinhua Li, Chengdu (CN); Qiong Shi, Chengdu (CN); Chenglu Yang, Chengdu (CN); Ting Zhang, Chengdu (CN); Jing Zhang, Chengdu (CN)

(21) Appl. No.: 19/485,514

(22) PCT Filed: May 17, 2024
(86) PCT No.: PCT/CN2024/094008

§ 371 Date: Nov. 18, 2025

(30) Foreign Application Priority Data
May 19, 2023 (WO) PCT/CN2023/095252

(51) Int. Cl. C07D 495/04; A61K 31/55; A61P 35/00; C07D 223/16; C07D 223/32; C07D 413/04; C07D 413/14; C07D 417/04

(52) U.S. Cl. CPC C07D 495/04; A61K 31/55; A61P 35/00; C07D 223/16; C07D 223/32; C07D 413/04; C07D 413/14; C07D 417/04

(57) ABSTRACT

The present disclose includes, among other things, compounds that treat or lessen the severity of cancer, pharmaceutical compositions and methods of making and using the same.

### BACKGROUND

Cancer is a term used for diseases in which abnormal cells divide without control and may invade other tissues. Cancer cells may also spread to other parts of the body through the blood and lymph systems.

There are more than 100 different types of cancer, with most cancers named for the organ or type of cell in which they start. For example, cancer that begins in the colon may be referred to as colon cancer; cancer that begins in basal cells of the skin may be referred to as basal cell carcinoma. Common types of cancer include breast cancer and lung cancer.

Cancer types can also be grouped into broader categories. The main categories of cancer include: carcinoma-cancer that begins in the skin or in tissues that line or cover internal organs; sarcoma-cancer that begins in bone, cartilage, fat, muscle, blood vessels, or other connective or supportive tissue; leukemia cancer that starts in blood-forming tissue such as the bone marrow and causes large numbers of abnormal blood cells to be produced and enter the blood; lymphoma and myeloma-cancers that begin in the cells of the immune system; central nervous system cancers-cancers that begin in the tissues of the brain and spinal cord.

Several techniques for treating cancer are known in the art. Such techniques include chemotherapy, radiation therapy, surgery, and transplantation. Each of these techniques, however, have undesirable side effects and varying success rates. Therefore, a need exists to develop new methods for treating cancer and/or diseases associated with cellular proliferation.

### SUMMARY

The present disclosure provides for compounds of formula (I):

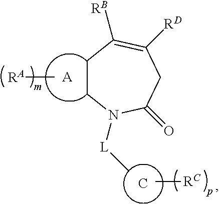

or a pharmaceutically acceptable salt thereof. Additionally, the present disclosure includes, among other things, pharmaceutical compositions, methods of using and methods of making a compound of formula (I).

### DETAILED DESCRIPTION

In some embodiments, the present disclosure includes a compound of Formula (I):

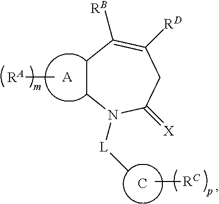

or a pharmaceutically acceptable salt thereof
wherein
X is O═ or S═;
Ring A is selected from the group consisting of optionally substituted phenyl, optionally substituted 5-membered heteroaryl, and optionally substituted 6-membered heteroaryl;
L is selected from the group consisting of a bond, optionally substituted C₁-C₃ alkylene, —CH(D)-, —C(D)₂-, —C(O)—, —C(O)O—, —C(O)NH—, and —S(O)₂—;
Group C is selected from the group consisting of C₁-C₆ aliphatic, —C≡CR₃, optionally substituted phenyl, optionally substituted 5-membered heteroaryl, optionally substituted 6-membered heteroaryl, optionally substituted 6-membered carbocyclyl, and optionally substituted 6-membered heterocyclyl;
each R^{A} is independently selected from the group consisting of halogen, —CN, —C≡CR³, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR¹, —SR¹, —N(R¹)₂, —C(O)OR¹, C(O)N(R¹)₂, —N(H)C(O)R¹, and —N(H)C(O)N(R¹)₂, wherein R^{A} is substituted with 0-4 instances of R¹; or R^{B} is independently selected from the group consisting of hydrogen, halogen, —C≡CR³, optionally substituted 5-6-membered heteroaryl, optionally substituted C₁-C₃ aliphatic, and optionally substituted C₁-C₃ alkoxy, and optionally substituted 3-6-membered carbocyclyl;
each R^{C} is independently selected from the group consisting of halogen, —CN, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR³, —SR³, —N(R³)₂, —C(O)OR³, —C(O)N(R³)₂, —N(H)C(O)R³, and —N(H)C(O)N(R³)₂;
   wherein optionally two instances of R^{C} are taken together with the atoms on which they are attached to form an optionally substituted 6-membered aryl or optionally substituted 6-membered heteroaryl;
R^{D} is selected from the group consisting of optionally substituted 5-membered heteroaryl, —C(O)OR³, C(O)N(R³)₂, —(CH₂)₁₋₃N(R²)₂, —(CH₂)₁₋₃OR², —(CH₂)₁₋₃O(CH₂)₁₋₃R², and —CHO;
each R¹ is independently selected from the group consisting of hydrogen, —C(O)R³, —(CH₂)₁₋₃OR³, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
each R² is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
   wherein optionally two instances of R² are taken together to form an optionally substituted 3-7 membered heterocyclyl ring;
each R³ is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
m is 0, 1, 2, 3, or 4;
p is 0, 1, 2, 3, 4, or 5.

In some embodiments, the present disclosure includes a compound of Formula (I-a), (I-b), or (I-c):

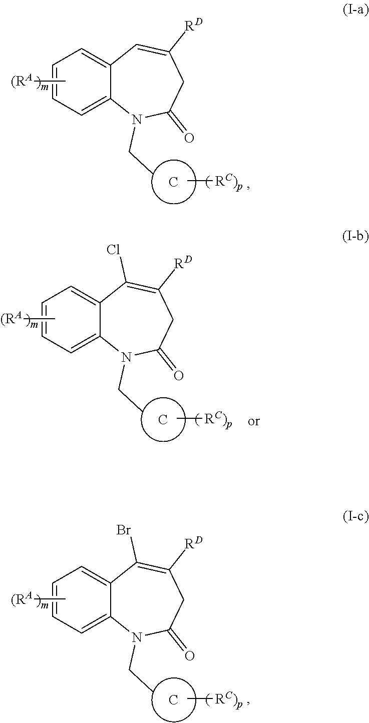

or a pharmaceutically acceptable salt thereof, wherein Group C, R^{A}, R^{C}, R^{D}, m, and p are defined above and described in classes and subclasses herein.

In some embodiments, the present disclosure includes a compound of Formula (I-al), (I-b1) or (I-c1):

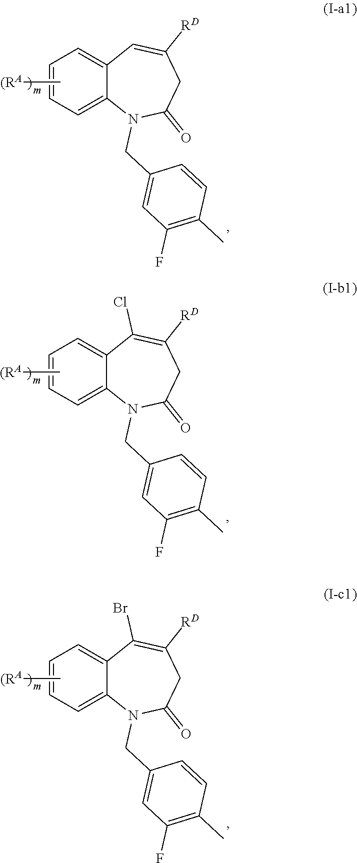

or a pharmaceutically acceptable salt thereof, wherein R^{A}, R^{D}, and m are defined above and described in classes and subclasses herein.

In some embodiments, the present disclosure includes a compound of formula (I-b2):

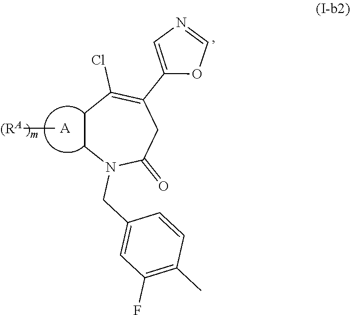

or a pharmaceutically acceptable salt thereof, wherein R^{A} and m are defined above and described in classes and subclasses herein.

In some embodiments, the present disclosure includes a compound of formula (I-c2), (I-c3) or (I-c4)

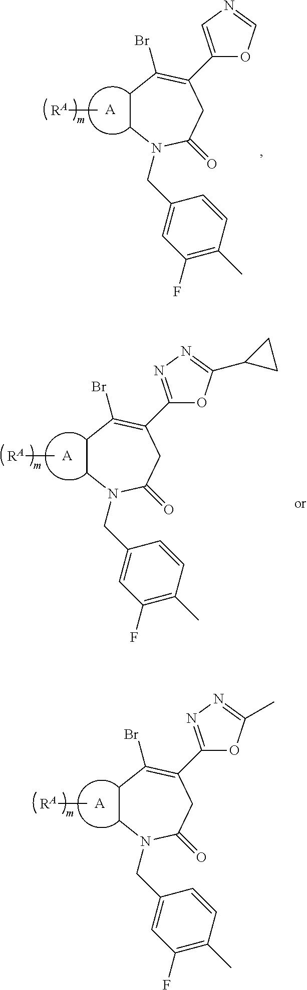

or a pharmaceutically acceptable salt thereof, wherein R^{A} and m are defined above and described in classes and subclasses herein.

In some embodiments, the present disclosure includes a compound of formula (I-e):

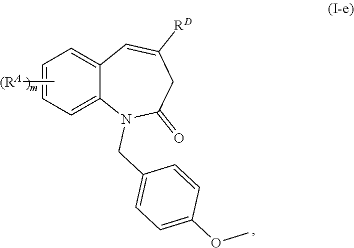

or a pharmaceutically acceptable salt thereof, wherein R^{A}, R^{D}, and m are defined above and described in classes and subclasses herein.

In some embodiments, the present disclosure includes a compound of formula (I-e1):

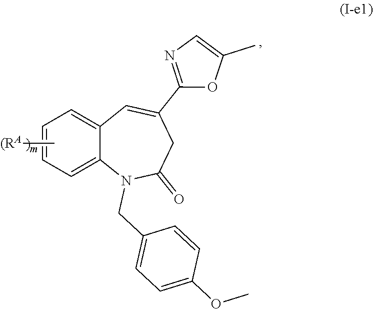

or a pharmaceutically acceptable salt thereof, wherein R^{A}, R^{D}, and m are defined above and described in classes and subclasses herein.

In some embodiments, the present disclosure includes a compound of formula (I-e2):

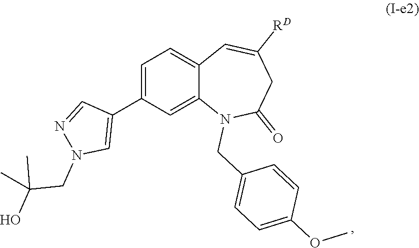

or a pharmaceutically acceptable salt thereof, wherein R^{A}, R^{D}, and m are defined above and described in classes and subclasses herein.

In some embodiments, the present disclosure includes a compound of formula (I-f):

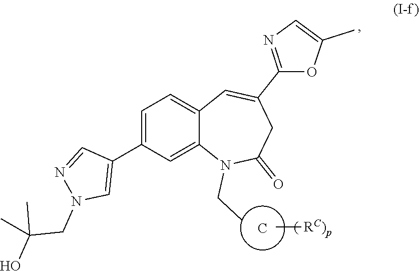

or a pharmaceutically acceptable salt thereof, wherein Ring C, R^{C}, and p are defined above and described in classes and subclasses herein.

### X

In some embodiments, X is O═ or S═. In some embodiments, X is O═. In some embodiments, X is S═.

### Ring A

In some embodiments, Ring A is selected from the group consisting of optionally substituted phenyl, optionally substituted 5-membered heteroaryl and optionally substituted 6-membered heteroaryl. In some embodiments, Ring A is optionally substituted phenyl. In some embodiments, Ring A is optionally substituted 6-membered heteroaryl. In some embodiments, Ring A is an optionally substituted 6-membered heteroaryl selected from pyridine, pyrazine, pyridazine, and pyrimidine. In some embodiments, Ring A is optionally substituted pyridine.

In some embodiments, Ring A is selected from the group consisting of

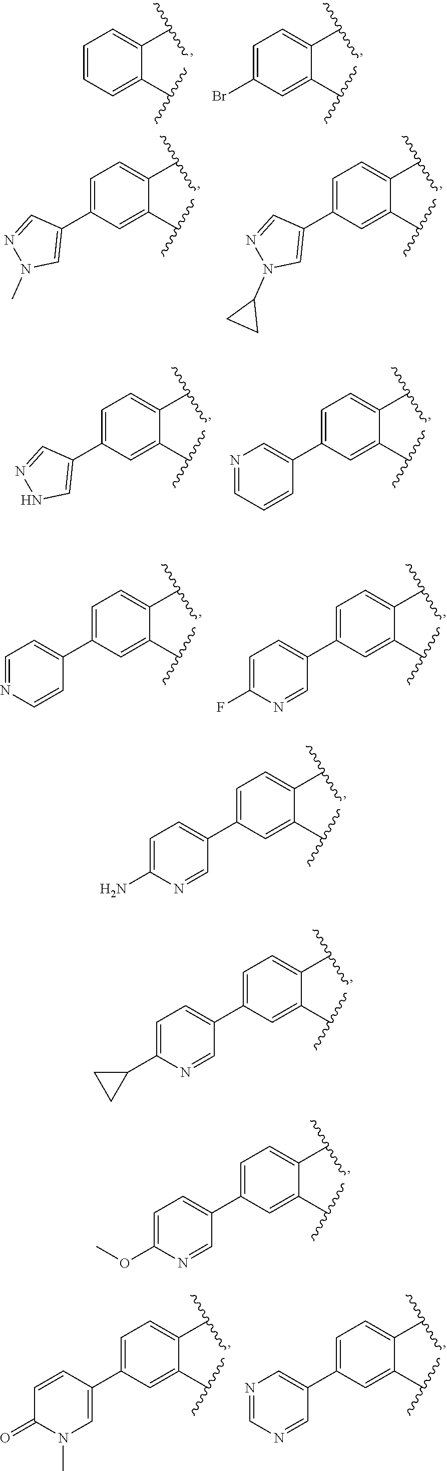

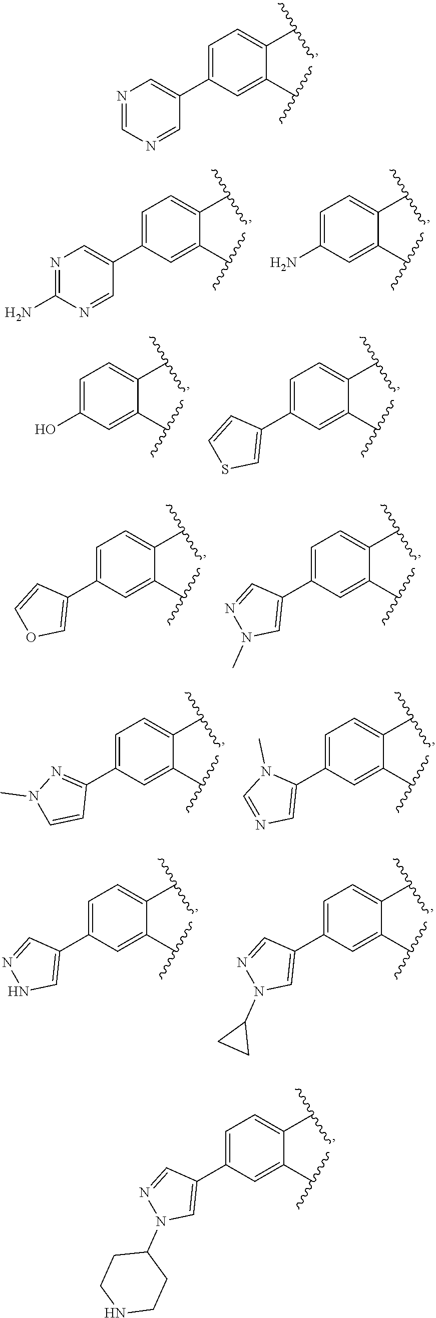

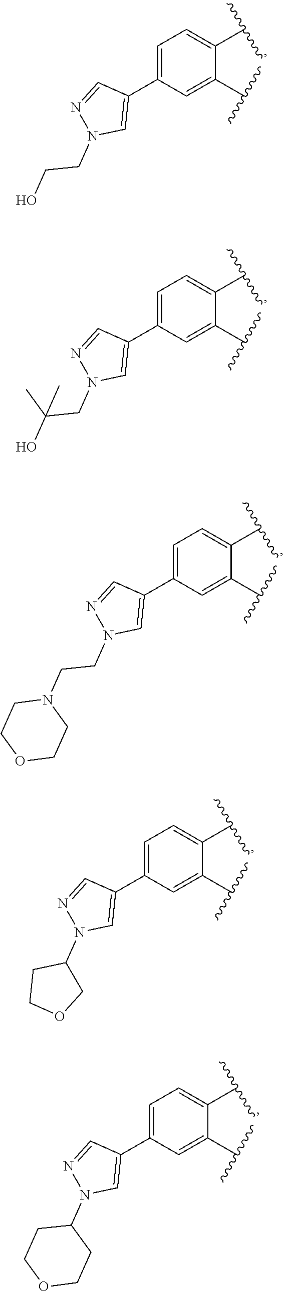 [SMILES: *c1ccc(-c2cnn(CCO)c2)cc1* ; *c1ccc(-c2cnn(CC(C)(C)O)c2)cc1* ; *c1ccc(-c2cnn(CCN3CCOCC3)c2)cc1* ; *c1ccc(-c2cnn(C3CCOC3)c2)cc1* ; *c1ccc(-c2cnn(C3CCOCC3)c2)cc1*]

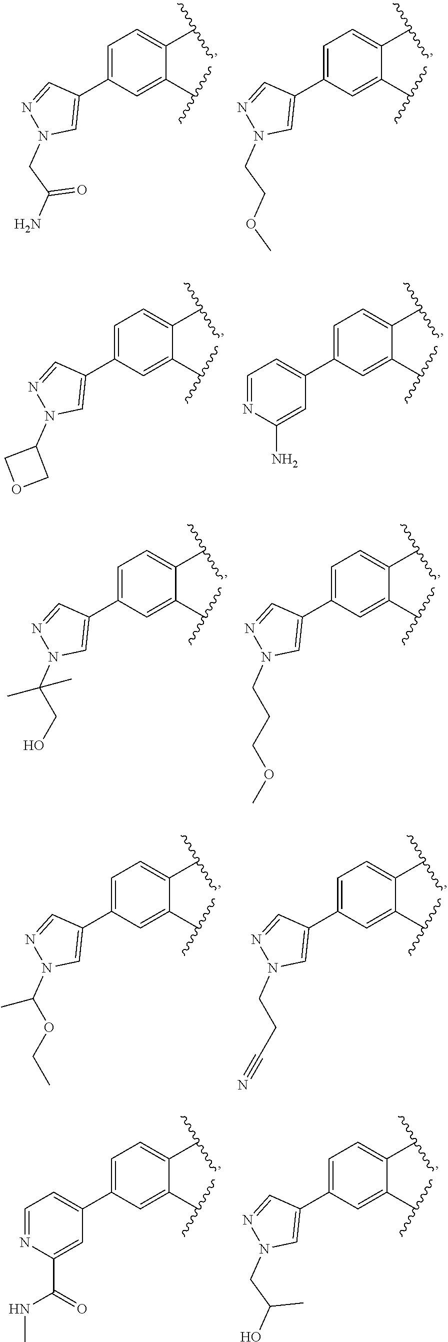

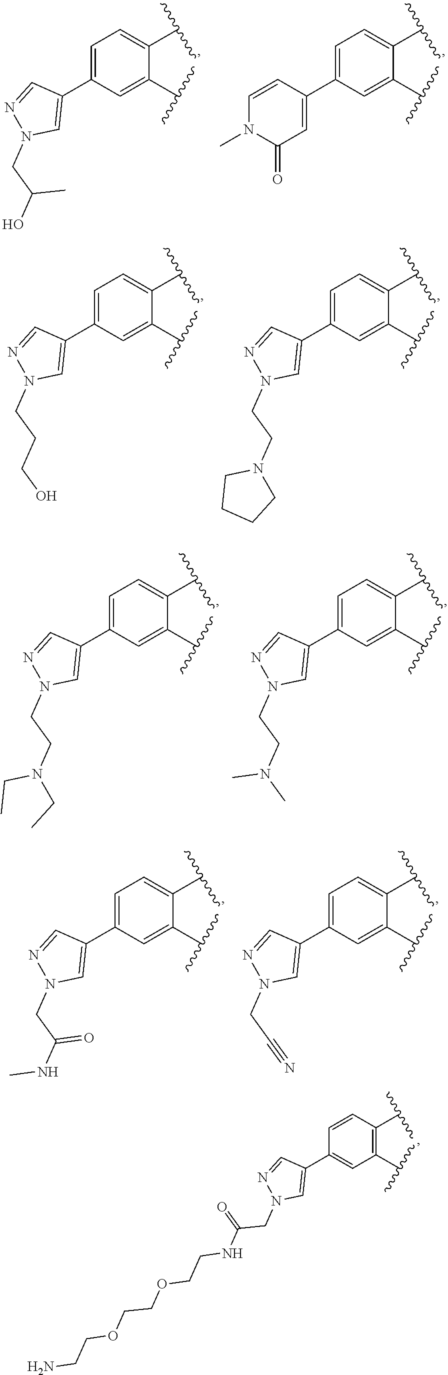

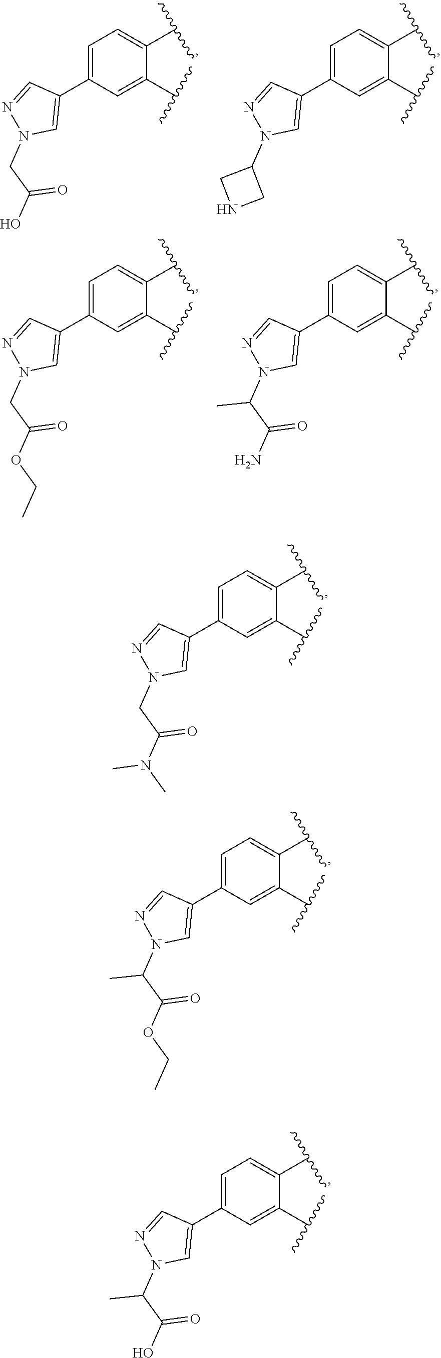

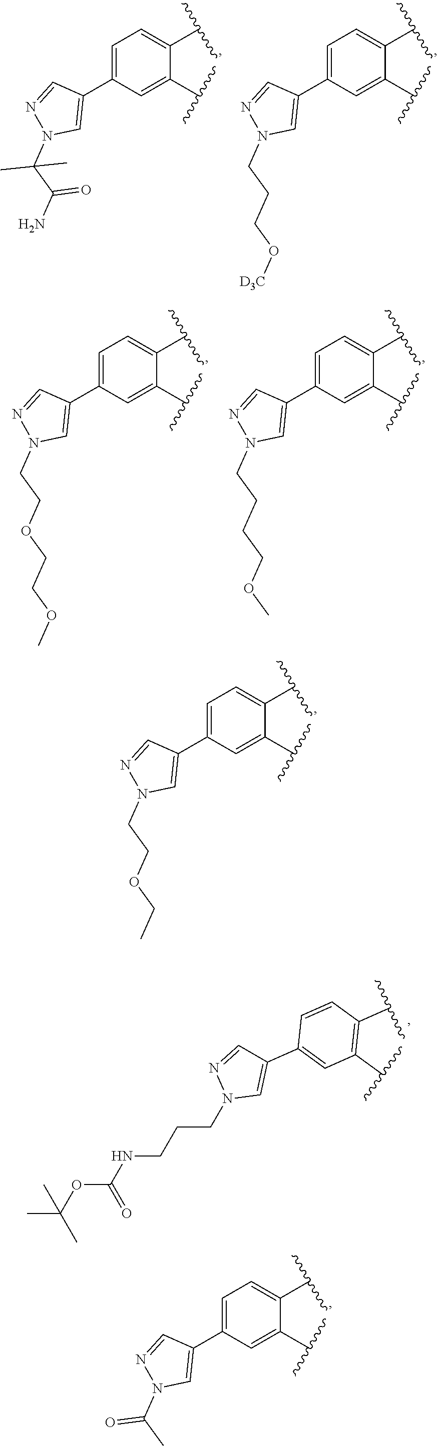

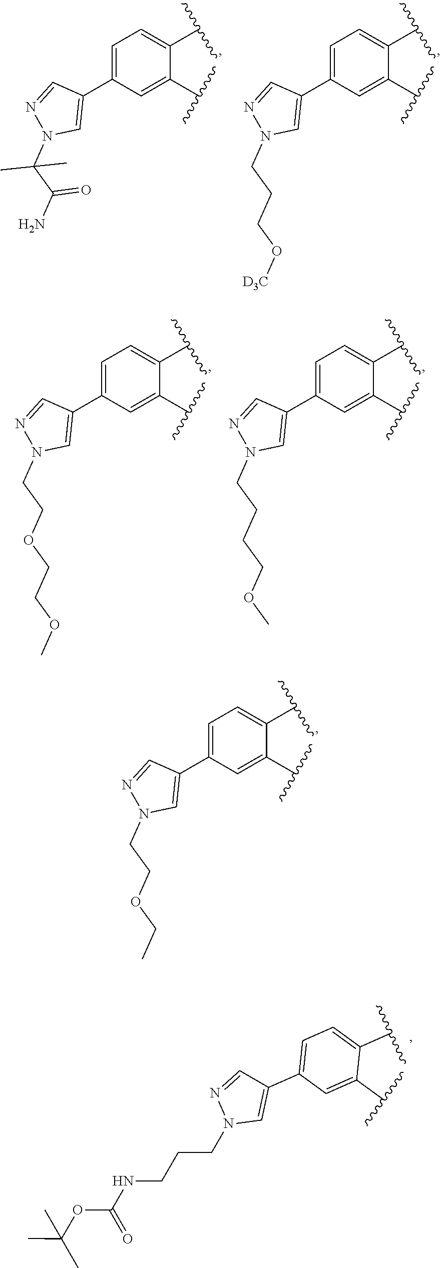 [SMILES: *c1ccc(-c2cnn(C(C)(C)C(N)=O)c2)cc1* ; *c1ccc(-c2cnn(CCCOC([2H])([2H])[2H])c2)cc1* ; *c1ccc(-c2cnn(CCOCCOC)c2)cc1* ; *c1ccc(-c2cnn(CCCCOC)c2)cc1* ; *c1ccc(-c2cnn(CCOCC)c2)cc1* ; *c1ccc(-c2cnn(CCCNC(=O)OC(C)(C)C)c2)cc1*]

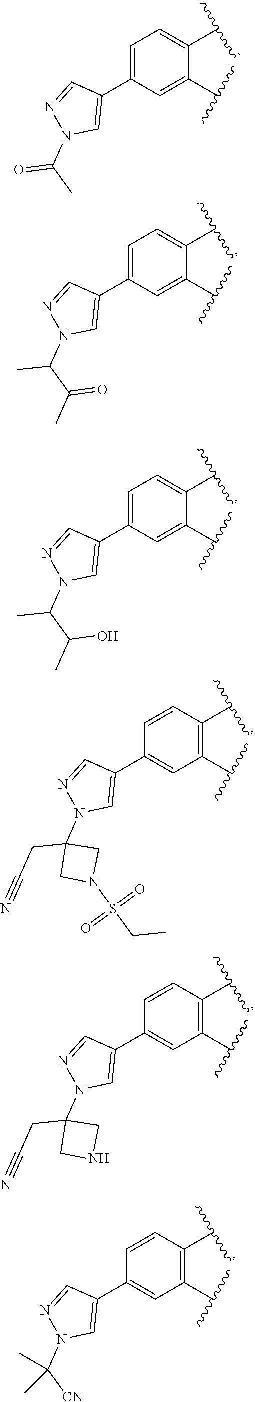 [SMILES: *c1ccc(-c2cnn(C(C)=O)c2)cc1* ; *c1ccc(-c2cnn(C(C)C(C)=O)c2)cc1* ; *c1ccc(-c2cnn(C(C)C(C)O)c2)cc1* ; *c1ccc(-c2cnn(C3(CC#N)CN(S(=O)(=O)CC)C3)c2)cc1* ; *c1ccc(-c2cnn(C3(CC#N)CNC3)c2)cc1* ; *c1ccc(-c2cnn(C(C)(C)C#N)c2)cc1*]

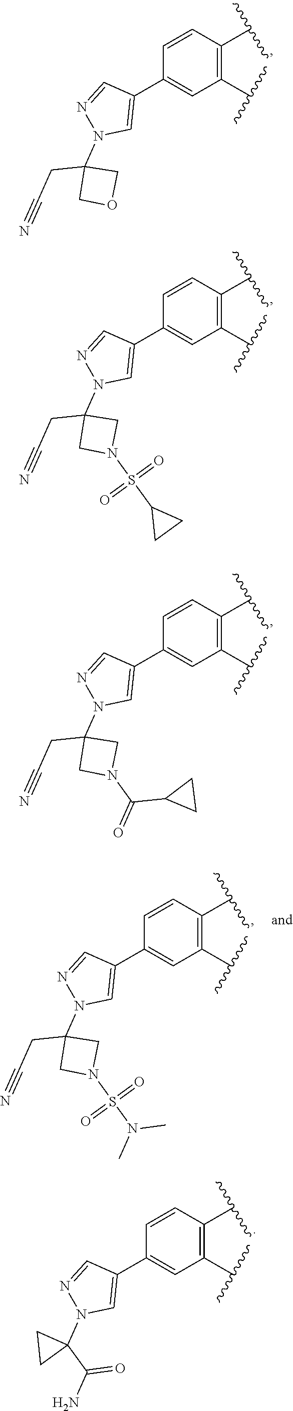 [SMILES: *c1ccc(-c2cnn(C3(CC#N)COC3)c2)cc1* ; *c1ccc(-c2cnn(C3(CC#N)CN(S(=O)(=O)C4CC4)C3)c2)cc1* ; *c1ccc(-c2cnn(C3(CC#N)CN(C(=O)C4CC4)C3)c2)cc1* ; *c1ccc(-c2cnn(C3(CC#N)CN(S(=O)(=O)N(C)C)C3)c2)cc1* ; *c1ccc(-c2cnn(C3(C(N)=O)CC3)c2)cc1*]

In some embodiments, Ring A is

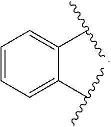 [SMILES: *c1ccccc1*]

In some embodiments, Ring A is

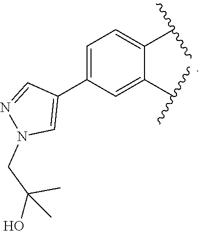 [SMILES: *c1ccc(-c2cnn(CC(C)(C)O)c2)cc1*]

In some embodiments, Ring A is

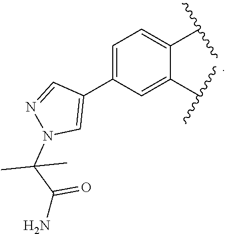 [SMILES: *c1ccc(-c2cnn(C(C)(C)C(N)=O)c2)cc1*]

### Group C

In some embodiments, Group C is selected from the group consisting of C₁-C₆ aliphatic, optionally substituted phenyl, optionally substituted 5-membered heteroaryl, optionally substituted 6-membered heteroaryl, optionally substituted 6-membered carbocyclyl, and optionally substituted 6-membered heterocyclyl. In some embodiments, Group C is selected from the group consisting of optionally substituted phenyl and optionally substituted 6-membered heteroaryl. In some embodiments, Group C is optionally substituted phenyl. In some embodiments, Group C is optionally substituted heteroaryl. In some embodiments, Group C is an optionally substituted 6-membered heteroaryl selected from pyridine, pyrazine, pyridazine, and pyrimidine. In some embodiments, Group C is optionally substituted pyridine. In some embodiments, Group C is optionally substituted phenyl or optionally substituted pyridine. In some embodiments, Group C is optionally substituted phenyl.

### L

In some embodiments, L is selected from the group consisting of a bond, optionally substituted C₁-C₃ alkylene, —C(O)—, —C(O)O—, —C(O)NH—, and —S(O)₂—. In some embodiments, L is selected from the group consisting of a bond, optionally substituted C₁-C₃ alkylene, and —C(O)—. In some embodiments, L is a bond. In some embodiments, L is an optionally substituted C₁-C₃ alkylene chain. In some embodiments, L is an optionally substituted C₁ alkylene chain. In some embodiments, L is an optionally substituted C₂ alkylene chain. In some embodiments, L is an optionally substituted C₃ alkylene chain. In some embodiments, L is an unsubstituted C₁-C₃ alkylene chain. In some embodiments, L is —CH₂—. In some embodiments, L is —C(H)(Me)-. In some embodiments, L is —CH₂CH₂—. In some embodiments, L is —CH₂CH₂CH₂—. In some embodiments, L is —C(O)—. In some embodiments, L is —S(O)₂—.

In some embodiments, L and Group C are taken together to form

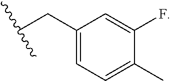 [SMILES: *Cc1ccc(C)c(F)c1]

In some embodiments, L and Group C are taken together to form

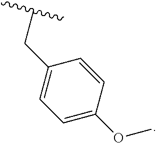 [SMILES: *Cc1ccc(OC)cc1]

In some embodiments, L and Group C are taken together to form

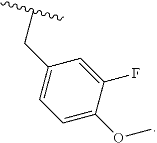 [SMILES: *Cc1ccc(OC)c(F)c1]

In some embodiments, L and Group C are taken together to form

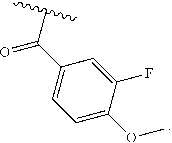 [SMILES: *C(=O)c1ccc(OC)c(F)c1]

In some embodiments, L and Group C are taken together to form

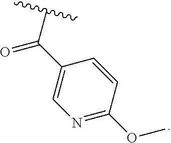 [SMILES: *C(=O)c1ccc(OC)nc1]

In some embodiments, L and Group C are taken together to form a group selected from

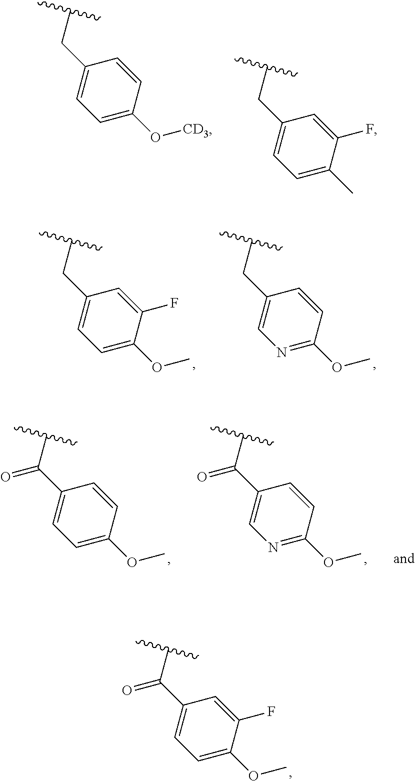

In some embodiments, each R^{A} is independently selected from the group consisting of halogen, —CN, —C≡CR³, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR¹, —SR¹, —N(R¹)₂, —C(O)OR¹, C(O)N(R¹)₂, —N(H)C(O)R¹, and —N(H)C(O)N(R¹)₂, wherein R^{A} is substituted with 0-4 instances of R¹. In some embodiments each R^{A} is independently selected from the group consisting of halogen, —CN, optionally substituted C₁-C₆ aliphatic, —OR¹, —SR¹, —N(R¹)₂, —C(O)OR¹, C(O)N(R¹)₂, —N(H)C(O)R¹, and —N(H)C(O)N(R¹)₂. In some embodiments, R^{A} is selected from the group consisting of halogen, —CN, and —OR¹. In some embodiments R^{A} is selected from the group consisting of F, Cl, Br, CN, —OH, —OMe, OEt, —OCH₂CH₂OMe, —OAc, and —OCH₂CH₂(N-morpholine).

### R^{B}

In some embodiments, R^{B} is independently selected from the group consisting of hydrogen, halogen, —C≡CR³, optionally substituted 5-6-membered heteroaryl, optionally substituted C₁-C₃ aliphatic, and optionally substituted C₁-C₃ alkoxy, and optionally substituted 3-6-membered carbocyclyl. In some embodiments, R^{B} is hydrogen. In some embodiments, R^{B} is halogen. In some embodiments, R^{B} is fluoro. In some embodiments, R^{B} is chloro. In some embodiments, R^{B} is bromo. In some embodiments, R^{B} is C₁-C₃ alkoxy. In some embodiments, R^{B} is methoxy.

### R^{C}

In some embodiments, each R^{C} is independently selected from the group consisting of halogen, —CN, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR³, —SR³, —S(O)₂R³, —N(R³)₂, —C(O)OR³, C(O)N(R³)₂, —N(H)C(O)R³, and —N(H)C(O)N(R³)₂. In some embodiments, each R^{C} is independently selected from the group consisting of halogen, —CN, optionally substituted C₁-C₆ aliphatic, optionally substituted phenyl, —OR³, —S(O)₂R³, and —C(O)OR³. In some embodiments, each R^{C} is independently selected from the group consisting of halogen and optionally substituted C₁-C₆ aliphatic.

In some embodiments, one instance of R^{C} is halogen and one instance is optionally substituted C₁-C₆ aliphatic.

In some embodiments, R^{C} is halogen. In some embodiments, R^{C} is fluorine. In some embodiments, R^{C} is chlorine. In some embodiments, R^{C} is bromine. In some embodiments, R^{C} is iodine.

In some embodiments, R^{C} is —CN. In some embodiments, R^{C} is —S(O)₂Me.

In some embodiments, each R^{C} is independently optionally substituted C₁-C₆ aliphatic. In some embodiments, each R^{C} is independently optionally substituted C₁-C₄ aliphatic. In some embodiments, each R^{C} is independently selected from the group consisting of optionally substituted methyl, optionally substituted ethyl, optionally substituted iso-propyl, optionally substituted n-propyl, optionally substituted n-butyl and optionally substituted tert-butyl. In some embodiments, each R^{C} is independently selected from the group consisting of —CF₃ and —CHF₂.

In some embodiments, R^{C} is —OR³, In some embodiments each R^{C} is independently selected from the group consisting of —OMe, —OCF₃ and —OCHF₂.

In some embodiments, two instances of R^{C} are taken together with the atoms on which they are attached to form an optionally substituted 6-membered aryl or optionally substituted 6-membered heteroaryl. In some embodiments two instances of R^{C} are taken together with Group C to form an optionally substituted naphthyl or optionally substituted 10-membered heteroaryl. In some embodiments two instances of R^{C} are taken together with Group C to form an optionally substituted naphthyl, optionally substituted quinolinyl, or isoquinolinyl. In some embodiments two instances of R^{C} are taken together with Group C to form an optionally substituted naphthyl or optionally substituted quinolinyl. In some embodiments two instances of R^{C} are taken together with Group C to form an optionally substituted naphthyl. In some embodiments two instances of R^{C} are taken together with Group C to form an optionally substituted quinolinyl.

In some embodiments, one instance of R^{C} is C₁₋₃ alkyl and one instance of R^{C} is halogen.

In some embodiments, one instance of R^{C} is methyl and one instance of R^{C} is fluoro.

### R^{D}

In some embodiments, R^{D} is selected from the group consisting of optionally substituted 5-membered heteroaryl, —C(O)OR³, C(O)N(R³)₂, —(CH₂)₁₋₃N(R²)₂, —(CH₂)₁₋₃OR², —(CH₂)₁₋₃O(CH₂)₁₋₃R², and —CHO;

In some embodiments, R^{D} is selected from the group consisting of

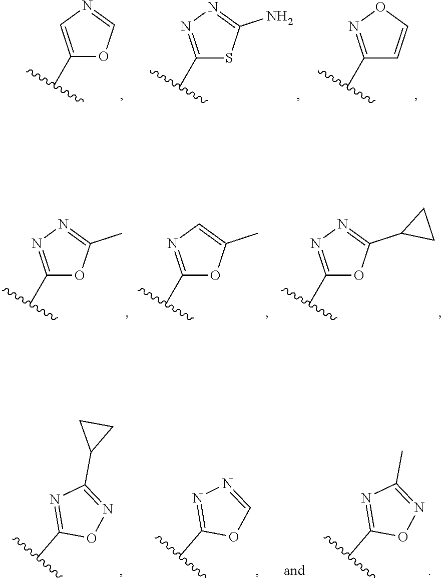

In some embodiments, R^{D} is

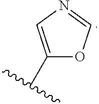 [SMILES: *c1cnco1]

In some embodiments, R^{D} is

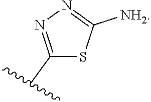 [SMILES: *c1nnc(N)s1]

In some embodiments, R^{D} is

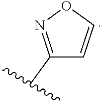 [SMILES: *c1ccon1]

In some embodiments, R^{D} is

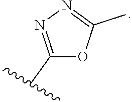 [SMILES: *c1nnc(C)o1]

In some embodiments, R^{D} is

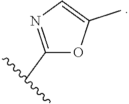 [SMILES: *c1ncc(C)o1]

In some embodiments, R^{D} is

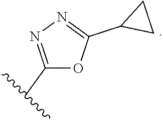 [SMILES: *c1nnc(C2CC2)o1]

In some embodiments, R^{D} is

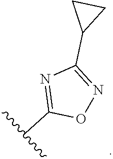 [SMILES: *c1nc(C2CC2)no1]

In some embodiments, R^{D} is

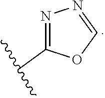 [SMILES: *c1nnco1]

In some embodiments, R^{D} is

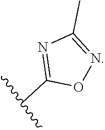 [SMILES: *c1nc(C)no1]

### R¹

In some embodiments, each R¹ is independently selected from the group consisting of hydrogen, —C(O)R³, —(CH₂)₁₋₃OR³, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl. In some embodiments, each R¹ is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl. In some embodiments, each R¹ is independently optionally substituted C₁-C₆ aliphatic. In some embodiments, R¹ is optionally substituted methyl.

### R²

In some embodiments, each R² is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl. In some embodiments, each R² is independently optionally substituted C₁-C₆ aliphatic. In some embodiments, R² is optionally substituted methyl.

### R³

In some embodiments, each R³ is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl. In some embodiments, each R³ is independently optionally substituted C₁-C₆ aliphatic. In some embodiments, each R³ is independently selected from the group consisting of hydrogen, optionally substituted methyl, —CF₃, and —CHF₂.

In some embodiments, the present disclosure includes compounds described in Table 1 or a pharmaceutically acceptable salt thereof

TABLE 1

| Compound | |
|---|---|
| No. | Structure |
| 1 | 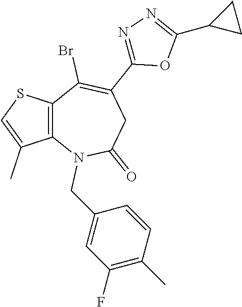 [SMILES: Cc1ccc(CN2C(=O)CC(c3nnc(C4CC4)o3)=C(Br)c3scc(C)c32)cc1F] |
| 2 | 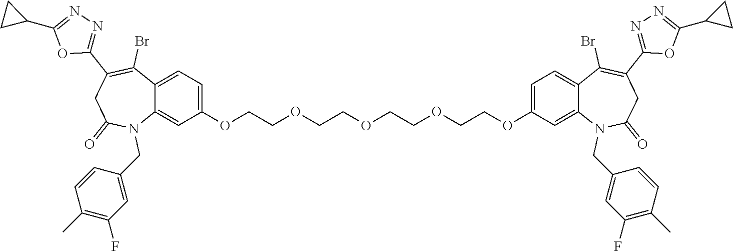 [SMILES: Cc1ccc(CN2C(=O)CC(c3nnc(C4CC4)o3)=C(Br)c3ccc(OCCOCCOCCOCCOc4ccc5c(c4)N(Cc4ccc(C)c(F)c4)C(=O)CC(c4nnc(C6CC6)o4)=C5Br)cc32)cc1F] |
| 3 | 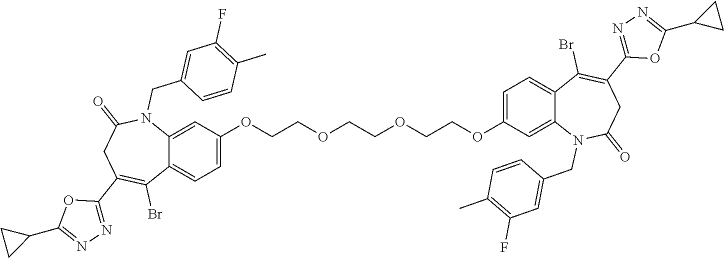 [SMILES: Cc1ccc(CN2C(=O)CC(c3nnc(C4CC4)o3)=C(Br)c3ccc(OCCOCCOCCOc4ccc5c(c4)N(Cc4ccc(C)c(F)c4)C(=O)CC(c4nnc(C6CC6)o4)=C5Br)cc32)cc1F] |
| 4 | 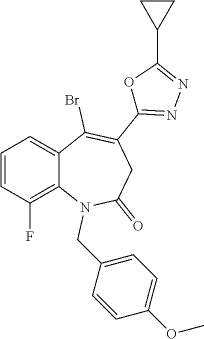 [SMILES: COc1ccc(CN2C(=O)CC(c3nnc(C4CC4)o3)=C(Br)c3cccc(F)c32)cc1] |

| | |
|---|---|
| Compound No. | R = |

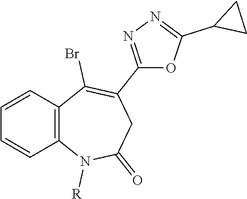

| | |
|---|---|
| 5 | 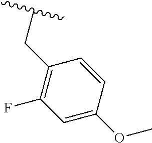 [SMILES: *Cc1ccc(OC)cc1F] |
| 6 | 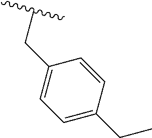 [SMILES: *Cc1ccc(CC)cc1] |

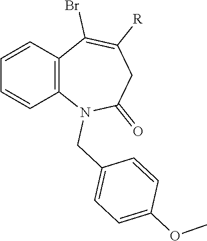

| 7 | 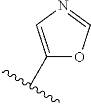 [SMILES: *c1cnco1] |
|---|---|
| 8 | 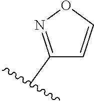 [SMILES: *c1ccon1] |
| 9 | 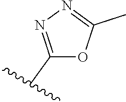 [SMILES: *c1nnc(C)o1] |
| 10 | 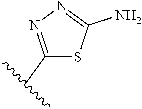 [SMILES: *c1nnc(N)s1] |
| 11 | 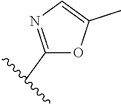 [SMILES: *c1ncc(C)o1] |
| Compound code | Structure |
| 12 | 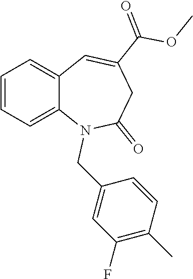 [SMILES: COC(=O)C1=Cc2ccccc2N(Cc2ccc(C)c(F)c2)C(=O)C1] |
| 13 | 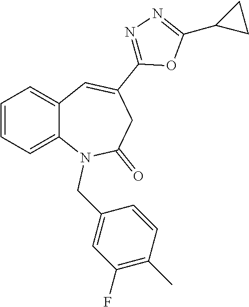 [SMILES: Cc1ccc(CN2C(=O)CC(c3nnc(C4CC4)o3)=Cc3ccccc32)cc1F] |
| 14 | 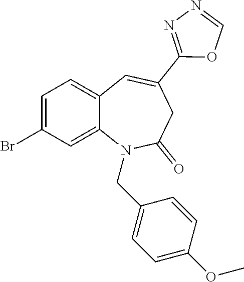 [SMILES: COc1ccc(CN2C(=O)CC(c3nnco3)=Cc3ccc(Br)cc32)cc1] |
| 15 | 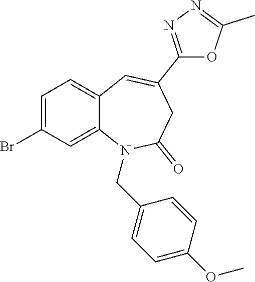 [SMILES: COc1ccc(CN2C(=O)CC(c3nnc(C)o3)=Cc3ccc(Br)cc32)cc1] |
| 16 | 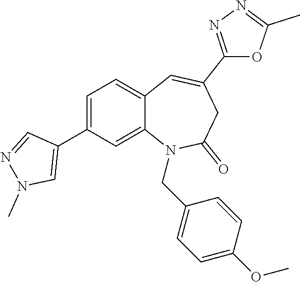 [SMILES: COc1ccc(CN2C(=O)CC(c3nnc(C)o3)=Cc3ccc(-c4cnn(C)c4)cc32)cc1] |
| 17 | 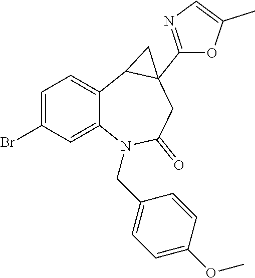 [SMILES: COc1ccc(CN2C(=O)CC3(c4ncc(C)o4)CC3c3ccc(Br)cc32)cc1] |
| Compound No. | Ring A |

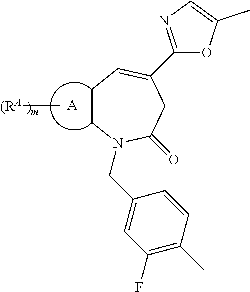

| | |
|---|---|
| 18 | 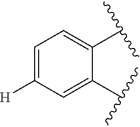 [SMILES: *c1ccccc1*] |
| 19 | 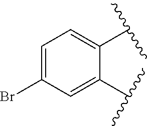 [SMILES: *c1ccc(Br)cc1*] |
| 20 | 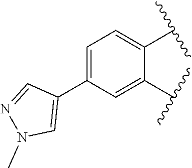 |
| 21 | 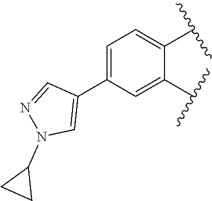 [SMILES: *c1ccc(-c2cnn(C3CC3)c2)cc1*] |
| 22 | 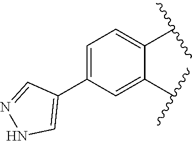 [SMILES: *c1ccc(-c2cn[nH]c2)cc1*] |
| 23 | 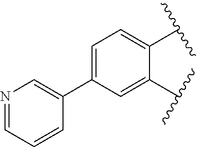 [SMILES: *c1ccc(-c2cccnc2)cc1*] |

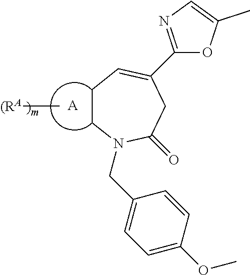

| | |
|---|---|
| 24 | 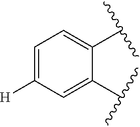 [SMILES: *c1ccccc1*] |
| 25 | 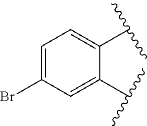 [SMILES: *c1ccc(Br)cc1*] |
| 26 | 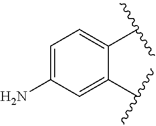 [SMILES: *c1ccc(N)cc1*] |
| 27 | 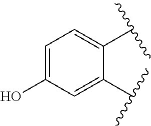 [SMILES: *c1ccc(O)cc1*] |
| 28 | 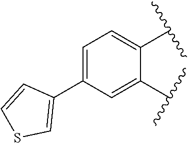 [SMILES: *c1ccc(-c2ccsc2)cc1*] |
| 29 | 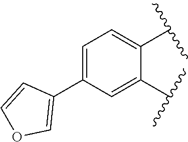 [SMILES: *c1ccc(-c2ccoc2)cc1*] |
| 30 | 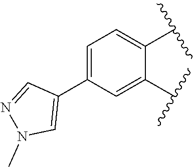 [SMILES: *c1ccc(-c2cnn(C)c2)cc1*] |
| 31 | 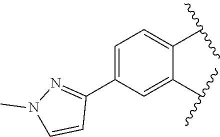 [SMILES: *c1ccc(-c2ccn(C)n2)cc1*] |
| 32 | 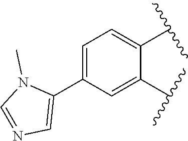 [SMILES: *c1ccc(-c2cncn2C)cc1*] |
| 33 | 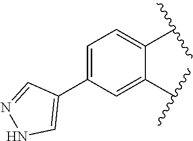 [SMILES: *c1ccc(-c2cn[nH]c2)cc1*] |
| 34 | 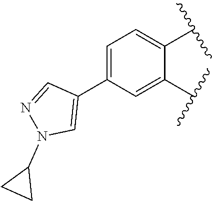 [SMILES: *c1ccc(-c2cnn(C3CC3)c2)cc1*] |
| 35 | 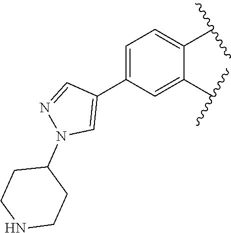 [SMILES: *c1ccc(-c2cnn(C3CCNCC3)c2)cc1*] |
| 36 | 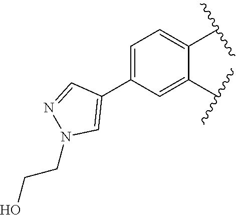 [SMILES: *c1ccc(-c2cnn(CCO)c2)cc1*] |
| 37 | 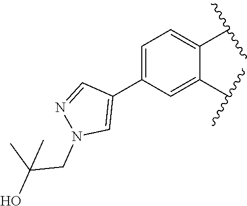 [SMILES: *c1ccc(-c2cnn(CC(C)(C)O)c2)cc1*] |
| 38 | 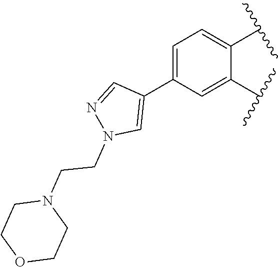 [SMILES: *c1ccc(-c2cnn(CCN3CCOCC3)c2)cc1*] |
| 39 | 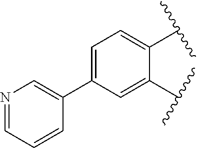 [SMILES: *c1ccc(-c2cccnc2)cc1*] |
| 40 | 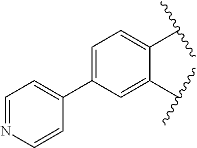 [SMILES: *c1ccc(-c2ccncc2)cc1*] |
| 41 | 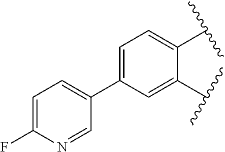 [SMILES: *c1ccc(-c2ccc(F)nc2)cc1*] |
| 42 | 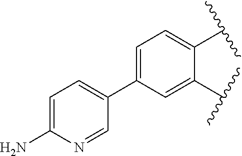 [SMILES: *c1ccc(-c2ccc(N)nc2)cc1*] |
| 43 | 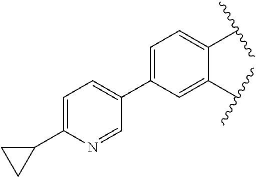 [SMILES: *c1ccc(-c2ccc(C3CC3)nc2)cc1*] |
| 44 | 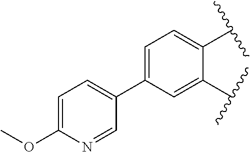 [SMILES: *c1ccc(-c2ccc(OC)nc2)cc1*] |
| 45 | 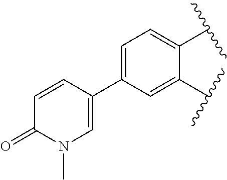 [SMILES: *c1ccc(-c2ccc(=O)n(C)c2)cc1*] |
| 46 | 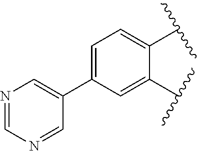 [SMILES: *c1ccc(-c2cncnc2)cc1*] |
| 47 | 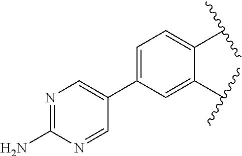 [SMILES: *c1ccc(-c2cnc(N)nc2)cc1*] |
| 48 | 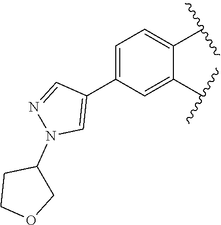 [SMILES: *c1ccc(-c2cnn(C3CCOC3)c2)cc1*] |
| 49 | 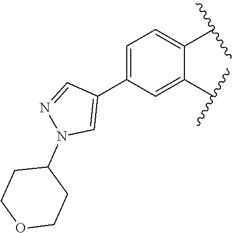 [SMILES: *c1ccc(-c2cnn(C3CCOCC3)c2)cc1*] |
| 50 | 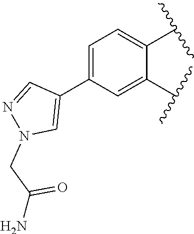 [SMILES: *c1ccc(-c2cnn(CC(N)=O)c2)cc1*] |
| 51 | 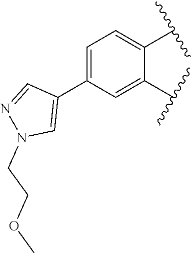 [SMILES: *c1ccc(-c2cnn(CCOC)c2)cc1*] |
| 52 | 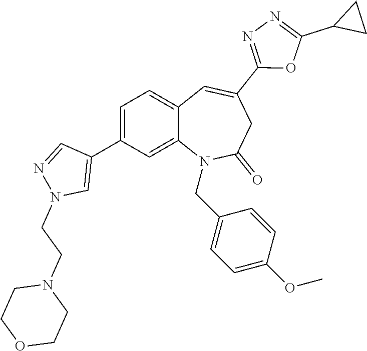 [SMILES: COc1ccc(CN2C(=O)CC(c3nnc(C4CC4)o3)=Cc3ccc(-c4cnn(CCN5CCOCC5)c4)cc32)cc1] |
| 53 | 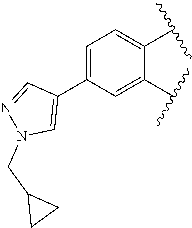 [SMILES: *c1ccc(-c2cnn(CC3CC3)c2)cc1*] |

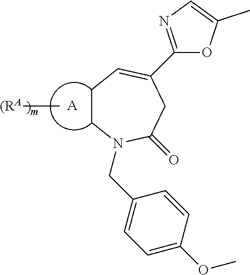

| 54 | 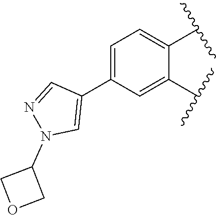 [SMILES: *c1ccc(-c2cnn(C3COC3)c2)cc1*] |
|---|---|
| 55 | 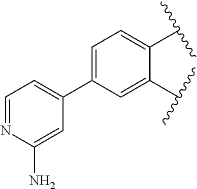 [SMILES: *c1ccc(-c2ccnc(N)c2)cc1*] |
| 56 | 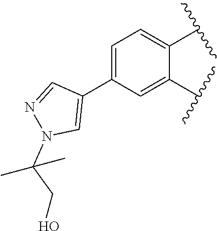 [SMILES: *c1ccc(-c2cnn(C(C)(C)CO)c2)cc1*] |
| 57 | 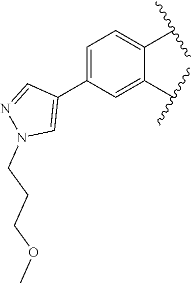 [SMILES: *c1ccc(-c2cnn(CCCOC)c2)cc1*] |
| 58 | 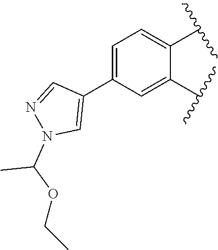 [SMILES: *c1ccc(-c2cnn(C(C)OCC)c2)cc1*] |
| 59 | 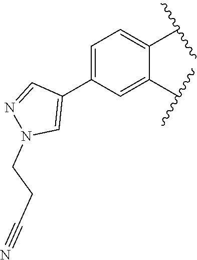 [SMILES: *c1ccc(-c2cnn(CCC#N)c2)cc1*] |
| 60 | 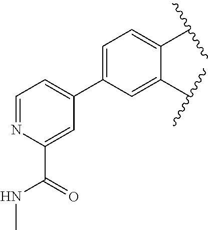 [SMILES: *c1ccc(-c2ccnc(C(=O)NC)c2)cc1*] |
| 61 | 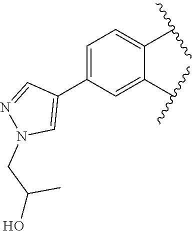 [SMILES: *c1ccc(-c2cnn(CC(C)O)c2)cc1*] |
| 62 | 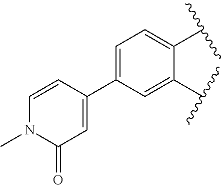 [SMILES: *c1ccc(-c2ccn(C)c(=O)c2)cc1*] |
| 63 | 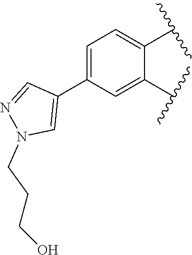 [SMILES: *c1ccc(-c2cnn(CCCO)c2)cc1*] |
| 64 | 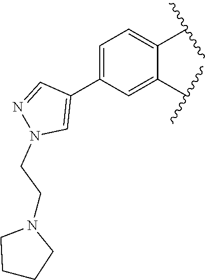 [SMILES: *c1ccc(-c2cnn(CCN3CCCC3)c2)cc1*] |
| 65 | 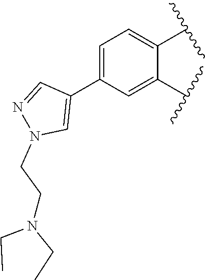 [SMILES: *c1ccc(-c2cnn(CCN(CC)CC)c2)cc1*] |
| 66 | 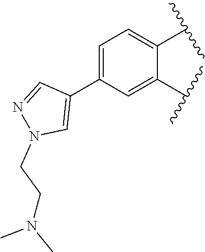 [SMILES: *c1ccc(-c2cnn(CCN(C)C)c2)cc1*] |
| 67 | 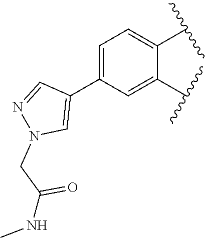 [SMILES: *c1ccc(-c2cnn(CC(=O)NC)c2)cc1*] |
| 68 | 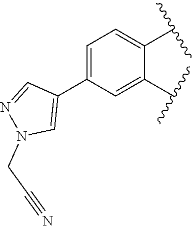 [SMILES: *c1ccc(-c2cnn(CC#N)c2)cc1*] |
| 69 | 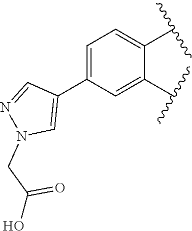 [SMILES: *c1ccc(-c2cnn(CC(=O)O)c2)cc1*] |
| 70 | 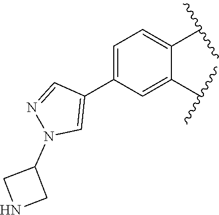 [SMILES: *c1ccc(-c2cnn(C3CNC3)c2)cc1*] |
| 71 | 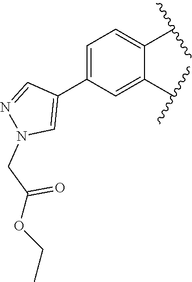 [SMILES: *c1ccc(-c2cnn(CC(=O)OCC)c2)cc1*] |
| 72 | 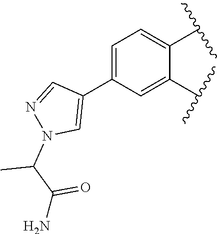 [SMILES: *c1ccc(-c2cnn(C(C)C(N)=O)c2)cc1*] |
| 73 | 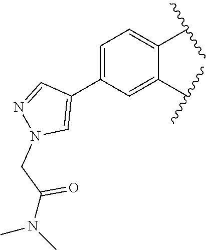 [SMILES: *c1ccc(-c2cnn(CC(=O)N(C)C)c2)cc1*] |
| 74 | 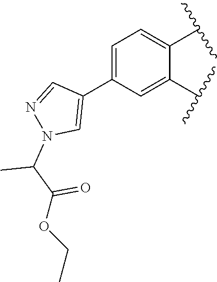 [SMILES: *c1ccc(-c2cnn(C(C)C(=O)OCC)c2)cc1*] |
| 75 | 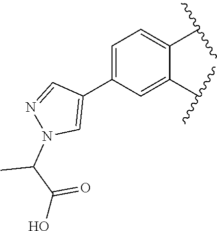 [SMILES: *c1ccc(-c2cnn(C(C)C(=O)O)c2)cc1*] |
| 76 | 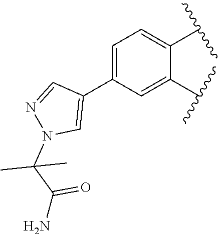 [SMILES: *c1ccc(-c2cnn(C(C)(C)C(N)=O)c2)cc1*] |
| 77 | 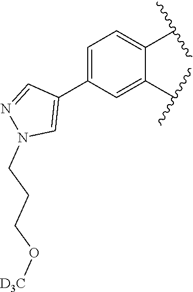 [SMILES: *c1ccc(-c2cnn(CCCOC([2H])([2H])[2H])c2)cc1*] |
| 78 | 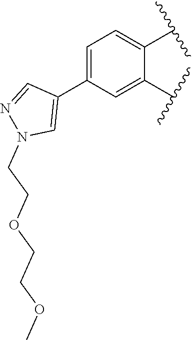 [SMILES: *c1ccc(-c2cnn(CCOCCOC)c2)cc1*] |
| 79 | 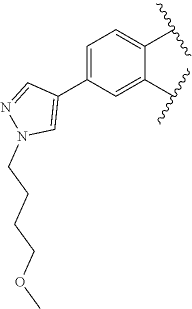 [SMILES: *c1ccc(-c2cnn(CCCCOC)c2)cc1*] |
| 80 | 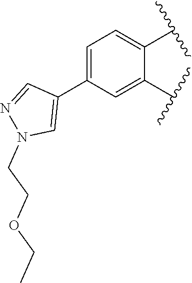 [SMILES: *c1ccc(-c2cnn(CCOCC)c2)cc1*] |
| 81 | 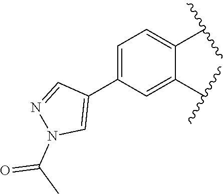 [SMILES: *c1ccc(-c2cnn(C(C)=O)c2)cc1*] |
| 82 | 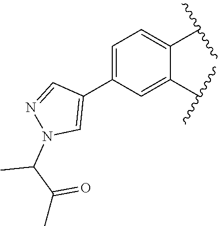 [SMILES: *c1ccc(-c2cnn(C(C)C(C)=O)c2)cc1*] |
| 83 | 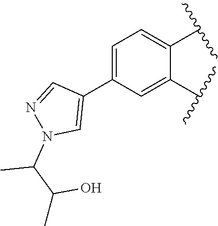 [SMILES: *c1ccc(-c2cnn(C(C)C(C)O)c2)cc1*] |
| 84 | 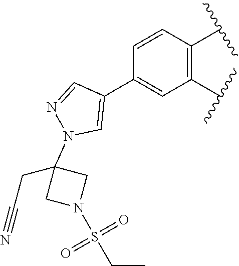 [SMILES: *c1ccc(-c2cnn(C3(CC#N)CN(S(=O)(=O)CC)C3)c2)cc1*] |
| 85 | 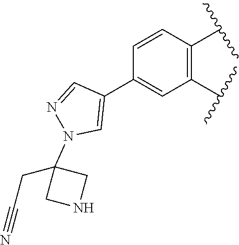 [SMILES: *c1ccc(-c2cnn(C3(CC#N)CNC3)c2)cc1*] |
| 86 | 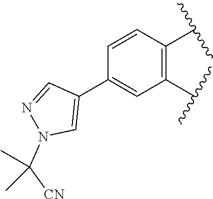 [SMILES: *c1ccc(-c2cnn(C(C)(C)C#N)c2)cc1*] |
| 87 | 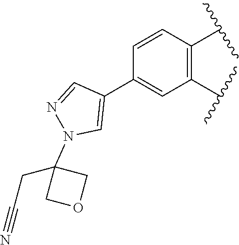 [SMILES: *c1ccc(-c2cnn(C3(CC#N)COC3)c2)cc1*] |
| 88 | 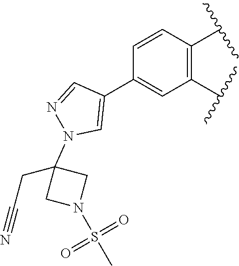 [SMILES: *c1ccc(-c2cnn(C3(CC#N)CN(S(C)(=O)=O)C3)c2)cc1*] |
| 89 | 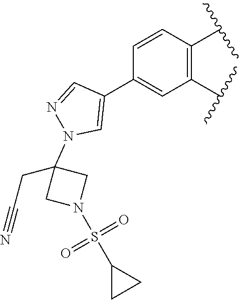 [SMILES: *c1ccc(-c2cnn(C3(CC#N)CN(S(=O)(=O)C4CC4)C3)c2)cc1*] |
| 90 | 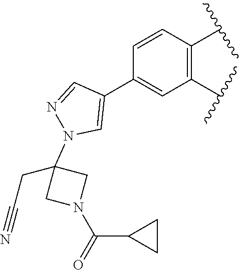 [SMILES: *c1ccc(-c2cnn(C3(CC#N)CN(C(=O)C4CC4)C3)c2)cc1*] |
| 91 | 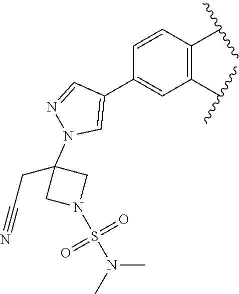 [SMILES: *c1ccc(-c2cnn(C3(CC#N)CN(S(=O)(=O)N(C)C)C3)c2)cc1*] |
| 92 | 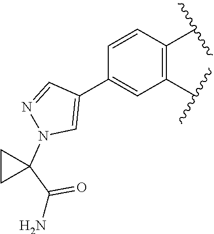 [SMILES: *c1ccc(-c2cnn(C3(C(N)=O)CC3)c2)cc1*] |
| Compound No. | R = |

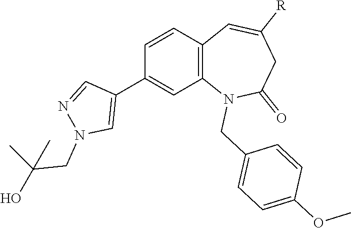

| | |
|---|---|
| 93 | 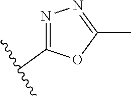 [SMILES: *c1nnc(C)o1] |
| 94 | 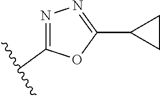 [SMILES: *c1nnc(C2CC2)o1] |
| 95 | 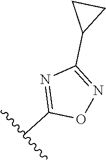 [SMILES: *c1nc(C2CC2)no1] |
| 96 | 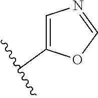 [SMILES: *c1cnco1] |
| 97 | 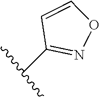 [SMILES: *c1ccon1] |
| 98 | 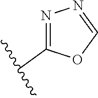 [SMILES: *c1nnco1] |
| 99 | 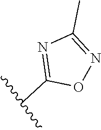 [SMILES: *c1nc(C)no1] |

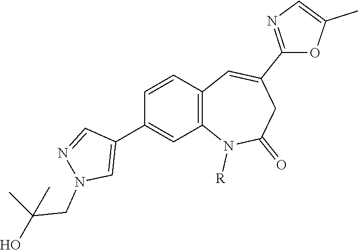

| | |
|---|---|
| 100 | 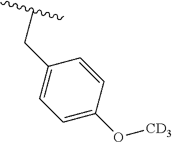 [SMILES: *Cc1ccc(OC([2H])([2H])[2H])cc1] |
| 101 | 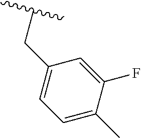 [SMILES: *Cc1ccc(C)c(F)c1] |
| 102 | 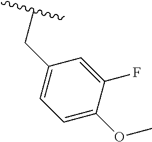 [SMILES: *Cc1ccc(OC)c(F)c1] |
| 103 | 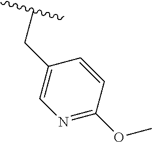 [SMILES: *Cc1ccc(OC)nc1] |

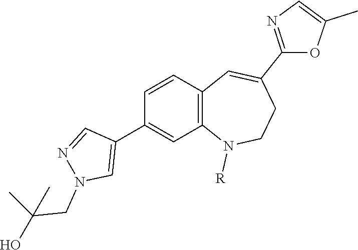

| | |
|---|---|
| 104 | 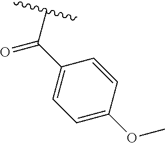 [SMILES: *C(=O)c1ccc(OC)cc1] |
| 105 | 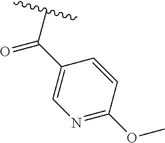 [SMILES: *C(=O)c1ccc(OC)nc1] |
| 106 | 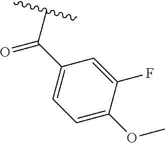 [SMILES: *C(=O)c1ccc(OC)c(F)c1] |
| 107 | 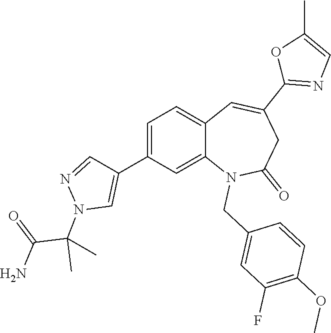 [SMILES: COc1ccc(CN2C(=O)CC(c3ncc(C)o3)=Cc3ccc(-c4cnn(C(C)(C)C(N)=O)c4)cc32)cc1F] |

### Definitions

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “haloaliphatic” refers to an aliphatic group that is substituted with one or more halogen atoms.

The term “alkyl” refers to a straight or branched alkyl group. Exemplary alkyl groups are methyl, ethyl, propyl, isopropyl, butyl, isobutyl, and tert-butyl.

The term “haloalkyl” refers to a straight or branched alkyl group that is substituted with one or more halogen atoms.

The term “halogen” means F, Cl, Br, or I.

The term “aryl” used alone or as part of a larger moiety as in “aralkyl”, “aralkoxy”, or “aryloxyalkyl”, refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14 71 electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the present disclosure may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

The present disclosure includes tautomers of structures of compounds as drawn herein.

Suitable monovalent substituents on a substitutable carbon atom of an “optionally substituted” group are independently halogen; —(CH₂)₀₋₄R^{∘}; —(CH₂)₀₋₄OR^{∘}; O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, —SC(S)SR^{∘}; —(CH₂)₀₋₄SC(O)R^{∘}; —(CH₂)₀₋₄C(O)NR^{∘}₂; C(S)NR^{∘}₂; —C(S)SR^{∘}; —SC(S)SR^{∘}, —(CH₂)₀₋₄OC(O)NR^{∘}₂; C(O)N(OR^{∘})R^{∘}; C(O)C(O)R^{∘}; C(O)CH₂C(O)R^{∘}; C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below. 4-L

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; O(haloR^{•}), —CN, —N₃, —(CH₂)₀₋₂C(O)R^{•}, —(CH₂)₀₋₂C(O)OH, —(CH₂)₀₋₂C(O)OR, —(CH₂)₀₋₂SR^{•}, —(CH₂)₀₋₂SH, (CH₂)₀₋₂NH₂, —(CH₂)₀₋₂NHR^{•}, —(CH₂)₀₋₂NR^{•}₂, NO₂, SiR^{•}₃, OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O, or S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: O(CR*2)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of Ri are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Combinations of substituents and variables envisioned by this disclosure are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof. Examples of such purposes include, but are not limited to, blood transfusion, organ transplantation, biological specimen storage, and biological assays.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a provided compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition.

### I

As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

The term “patient”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound(s) with which it is formulated. Pharmaceutically acceptable carriers, adjuvants or vehicles that may be used in the compositions of the compounds disclosed herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily active metabolite or residue thereof.

The expression “dosage unit form” as used herein refers to a physically discrete unit of agent appropriate for the patient to be treated. It will be understood, however, that total daily usage of compounds and compositions of the present disclosure will be decided by the attending physician within the scope of sound medical judgment. Specific effective dose level for any particular patient or organism will depend upon a variety of factors including disorder being treated and severity of the disorder; activity of specific compound employed; specific composition employed; age, body weight, general health, sex and diet of the patient; time of administration, route of administration, and rate of excretion of a specific compound employed; duration of treatment; drugs used in combination or coincidental with a specific compound employed, and like factors well known in the medical arts.

### Alternative Embodiments

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, 180; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Pharmaceutical Compositions

In some embodiments, the present disclosure provides a composition comprising a compound of Formula (I) and a pharmaceutically acceptable carrier, adjuvant, or vehicle. In some embodiments, the amount of compound in compositions contemplated herein is such that is effective to measurably treat a disease or disorder in a biological sample or in a patient. In certain embodiments, the amount of compound in compositions of this disclosure is such that is effective to measurably treat a disease or disorder in a biological sample or in a patient. In certain embodiments, a composition contemplated by this disclosure is formulated for administration to a patient in need of such composition. In some embodiments, a composition contemplated by this disclosure is formulated for oral administration to a patient.

In some embodiments, compositions of the present disclosure may be administered orally, parenterally, by inhalation spray, topically, rectally, nasally, buccally, vaginally or via an implanted reservoir. In some preferred embodiments, compositions are administered orally, intraperitoneally or intravenously. In some embodiments, sterile injectable forms of the compositions comprising one or more compounds of Formula (I) may be aqueous or oleaginous suspension. In some embodiments, suspensions may be formulated according to techniques known in the art using suitable dispersing or wetting agents and suspending agents. In some embodiments, sterile injectable preparation may also be a sterile injectable solution or suspension in a non-toxic parenterally acceptable diluent or solvent, for example as a solution in 1,3-butanediol. In some embodiments, among the acceptable vehicles and solvents that may be employed are water, Ringer's solution and isotonic sodium chloride solution. In some embodiments, additional examples include, but are not limited to, sterile, fixed oils are conventionally employed as a solvent or suspending medium.

The term “parenteral” as used herein includes subcutaneous, intravenous, intramuscular, intra-articular, intra-synovial, intrasternal, intrathecal, intrahepatic, intralesional and intracranial injection or infusion techniques.

Pharmaceutically acceptable compositions comprising one or more compounds of Formula (I) may be orally administered in any orally acceptable dosage form including, but not limited to, capsules, tablets, aqueous suspensions or solutions. In some embodiments, carriers used include lactose and corn starch. Lubricating agents, such as magnesium stearate, are also typically added. In some embodiments, useful diluents include lactose and dried cornstarch. In some embodiments, when aqueous suspensions are required for oral use, an active ingredient is combined with emulsifying and suspending agents. In some embodiments, certain sweetening, flavoring or coloring agents may also be added.

Alternatively, pharmaceutically acceptable compositions comprising a compound of Formula (I) may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions comprising a compound of Formula (I) may also be administered topically, especially when the target of treatment includes areas or organs readily accessible by topical application, including diseases of the eye, the skin, or the lower intestinal tract. Suitable topical formulations are readily prepared for each of these areas or organs. In some embodiments, pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions comprising a compound of Formula (I) may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, an amount of a compound of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

### Methods of Using Compounds of the Present Disclosure

In some embodiments, the present disclosure provides a method for treating or lessening the severity of a disease or condition associated with cell proliferation in a patient comprising the step of administering to said patient a composition according to the present disclosure.

The term “disease or condition associated with cell proliferation”, as used herein means any disease or other deleterious condition in which cell proliferation is known to play a role. Accordingly, another embodiment of the present disclosure relates to treating or lessening the severity of one or more diseases in which cell proliferation is known to play a role. In some embodiments, a disease or condition associated with cell proliferation is cancer.

In some embodiments, administration of a compound of the present disclosure results in arrest of mitosis or change in DNA content.

In some embodiments, administration of a compound of the present disclosure results in arrest of mitosis. In some embodiments, mitotic arrest is defined as a 10-100% reduction in mitosis. In some embodiments, mitotic arrest is defined as a 20-100% reduction in mitosis. In some embodiments, mitotic arrest is defined as a 30-100% reduction in mitosis. In some embodiments, mitotic arrest is defined as a 40-100% reduction in mitosis. In some embodiments, mitotic arrest is defined as a 50-100% reduction in mitosis. In some embodiments, mitotic arrest is defined as a 60-100% reduction in mitosis. In some embodiments, mitotic arrest is defined as a 70-100% reduction in mitosis. In some embodiments, mitotic arrest is defined as a 80-100% reduction in mitosis. In some embodiments, mitotic arrest is defined as a 90-100% reduction in mitosis. In some embodiments, mitotic arrest is defined as a 100% reduction in mitosis.

In some embodiments, administration of a compound of the present disclosure results in change in DNA content. In some embodiments, change of DNA content is induction of polyploidy.

In some embodiments, compounds and compositions, according to a method of the present disclosure, may be administered using any amount and any route of administration effective for treating or lessening the severity of cancer. The exact amount required will vary from subject to subject, depending on the species, age, and general condition of the subject, severity of the infection, particular agent, its mode of administration, and the like. Compounds of the present disclosure are preferably formulated in dosage unit form for ease of administration and uniformity of dosage.

In some embodiments, cancer is selected from the group consisting of lung cancer and breast cancer. In some embodiments, cancer is lung cancer. In some embodiments, lung cancer is non-small cell lung cancer. In some embodiments, non-small cell lung cancer is lung adenocarcinoma. In some embodiments, cancer is breast cancer. In some embodiments, breast cancer is mammary cancer. In some embodiments, breast cancer is breast adenocarcinoma.

In some embodiments, pharmaceutically acceptable compositions of comprising compounds of the present disclosure can be administered to humans and other animals orally, rectally, parenterally, intracisternally, intravaginally, intraperitoneally, topically (as by powders, ointments, or drops), buccally, as an oral or nasal spray, or the like, depending on the severity of infection being treated. In certain embodiments, compounds of the present disclose may be administered orally or parenterally at dosage levels of about 0.01 mg/kg to about 50 mg/kg and preferably from about 1 mg/kg to about 25 mg/kg, of subject body weight per day, one or more times a day, to obtain desired therapeutic effect.

In some embodiments, one or more additional therapeutic agents, may also be administered in combination with compounds of the present disclosure. In some embodiments, a compound of the present disclosure and one or more additional therapeutic agents may be administered as part of a multiple dosage regime. In some embodiments, a compound of the present disclosure and one or more additional therapeutic agents may be administered may be administered simultaneously, sequentially or within a period of time. In some embodiments, a compound of the present disclosure and one or more additional therapeutic agents may be administered within five hours of one another. In some embodiments, a compound of the present disclosure and one or more additional therapeutic agents may be administered within 24 hours of one another. In some embodiments, a compound of the present disclosure and one or more additional therapeutic agents may be administered within one week of one another.

In some embodiments, a compound of the present disclosure and one or more additional therapeutic agents may be formulated into a single dosage form.

### EXEMPLIFICATION

### 1. Chemistry

### 1.1 General Methods

Unless stated otherwise, all the chemicals required for synthesis were purchased from commercially available suppliers and used without further purification. ¹H NMR spectra was determined with a Bruker Avance III-400 at 400 MHz. LC-MS analysis was performed on a platform equipped with Agilent LC-MS 1260-6110 or Agilent LC-MS 1260-6120, using a Waters X Bridge C18: 50 mm×4.6 mm×3.5 um column. Flash column chromatography was conducted with silica gel (200-300 mesh, Qingdao Haiyang Chemical Co. Ltd., China). Analytical and preparative TLC analysis were performed on GF254 silica gel plates (Yantai Jiangyou Inc., China).

Unless otherwise noted, reagents and all solvents are analytically pure grade and were obtained commercially from vendors such as Chron Chemical or Energy-Chemical.

Abbreviations: TLC: Thin Layer Chromatography, EA: Ethyl Acetate, PE: Petroleum Ether, DMF: N,N-dimethylformamide, THF: Eetrahydrofuran, DCM: Dichloromethane, NaH: Sodium hydride

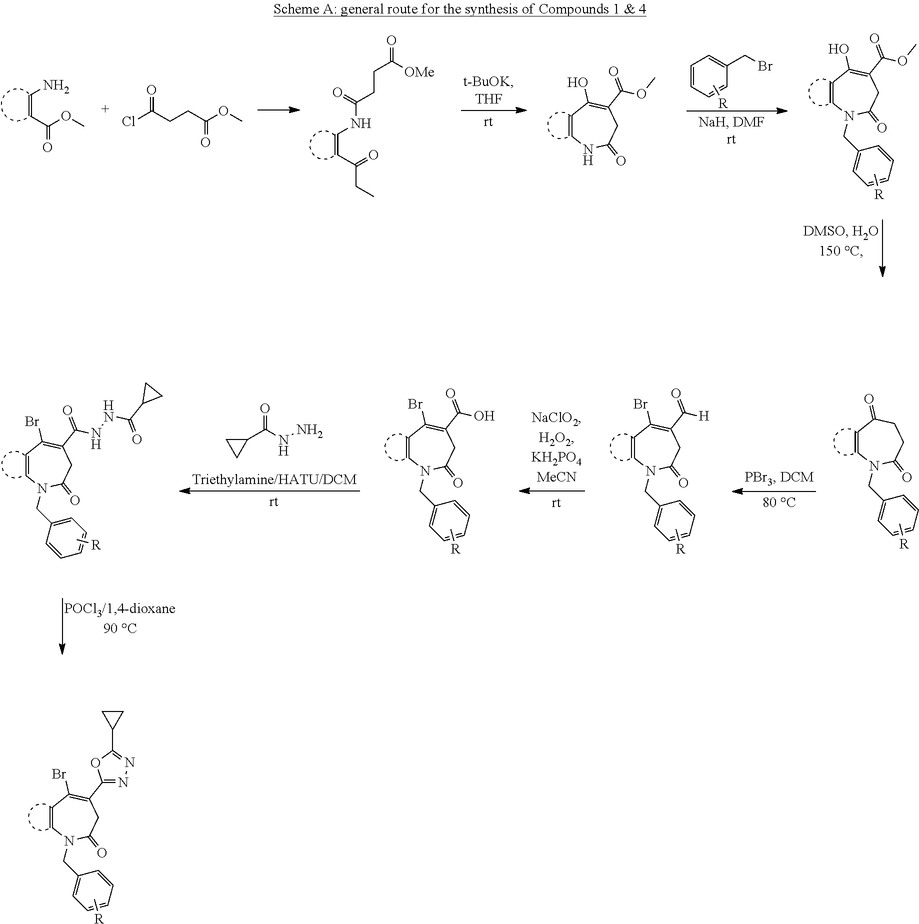

### Example 1. 8-Bromo-7-(5-cyclopropyl-1,3,4-oxadiazol-2-yl)-4-(3-fluoro-4-methylbenzyl)-3-methyl-4,6-dihydro-5H-thieno[3,2-b]azepin-5-one (1)

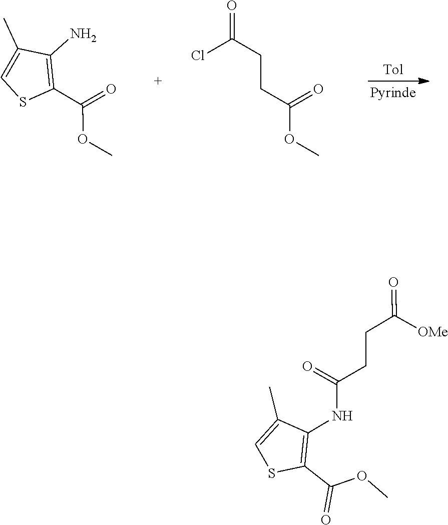

### Methyl 3-(4-methoxy-4-oxobuitanamido)-4-methylthiophene-2-carboxylate

To a solution of methyl 3-amino-4-methylthiophene-2-carboxylate (2.562 g, 15 mmol, 1 equiv) in toluene (40 mL), was added pyridine (1.42 mL, 18 mmol, 1.2 equiv) under Nitrogen and cooled down to −10° C. To this solution was added 3-(carbomethoxy)propionyl chloride (2.25 mL, 18 mmol, 1.2 equiv) dropwise over a period of 10 min, and the reaction mixture was allowed to stir at 80° C. for 2 h. After cooling to rt the white solid was filtered off, the organic layer was extracted three times with water, HCl 0.2 M and 5% Na₂CO₃, respectively, and dried over Na₂SO₄, filtered and concentrated by rotary evaporation to get the crude compound. Then, the resulting concentrate was purified on a silica column to get 3.9 g of the white solid compound with a yield of 75.94%.

TLC Rf=0.4 (PE:EA=2:1)

LCMS (m/z)=286.2 (M+H)

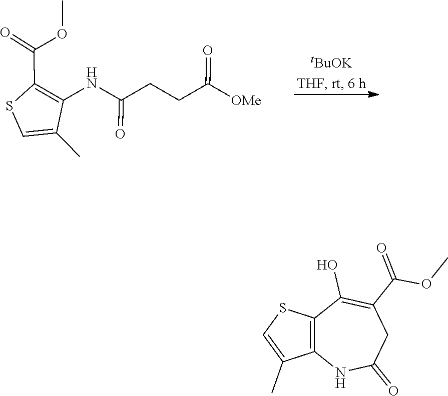 [SMILES: COC(=O)CCC(=O)Nc1c(C)csc1C(=O)OC ; COC(=O)C1=C(O)c2scc(C)c2NC(=O)C1]

### Methyl 8-hydroxy-3-methyl-5-oxo-5,6-dihydro-4H-thieno[3,2-b]azepine-7-carboxylate

To a solution of methyl 3-(4-methoxy-4-oxobutanamido)-4-methylthiophene-2-carboxylate (1 g, 3.5 mmol, 1 equiv) in THF (14 mL) at 5° C. was added a 1 M solution of t-BuOK in THF (10.51 mL, 10.51 mmol, 3 equiv) dropwise over 10 min while maintaining the temperature at 5° C. After 6 h, 5 mL of H₂O followed by 10 mL of 1N HCl were added to bring the solution to pH=4. The resulting mixture was allowed to stir at rt for 40 min. The organic solution was separated and the aqueous solution was extracted with EtOAc (3×100 mL). The organic solutions were combined, dried over anhydrous Na₂SO₄, filtered and concentrated by rotary evaporation to afford crude compound. Then, the resulting concentrate was purified on a silica column to get 667 mg of the white solid compound with a yield of 75.14%, purity=80.510%

TLC R_{f}=0.3 (PE:EA=2:1)

LCMS (m/z)=254.10 (M+H)

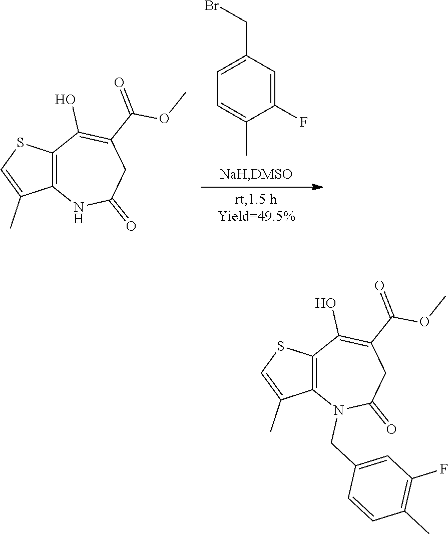

### Methyl 4-(3-fluoro-4-methylbenzyl)-8-hydroxy-3-methyl-5-oxo-5,6-dihydro-4H-thieno[3,2-b]azepine-7-carboxylate

To a solution of methyl 8-hydroxy-3-methyl-5-oxo-5,6-dihydro-4H-thieno[3,2-b]azepine-7-carboxylate (1.13 g, 4.45 mmol, 1 equiv) in DMSO (32 mL) was added NaH 60% dispersion in mineral oil (640 mg, 16 mmol, 3.6 equiv). To this solution was added 4-(bromomethyl)-2-fluoro-1-methylbenzene (813 ul, 5.33 mmol, 1.2 equiv), and the reaction mixture was allowed to stir at rt for 1.5 h. The resulting solution was poured into water (30 mL) and extracted with Ethyl acetate (3×30 mL). The combined organic layers were washed with brine (40 mL), dried over anhydrous Na₂SO₄, and concentrated by rotary evaporation. Then, the resulting concentrate was purified on a silica column to get 827 mg of the white solid compound with a yield of 49.55%, purity=99%.

TLC R_{f}=0.75 (PE:EA=1:1)

LCMS (m/z)=376.50 (M+H).

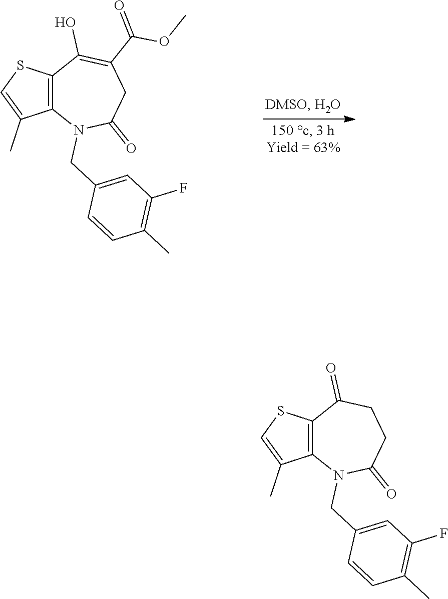 [SMILES: COC(=O)C1=C(O)c2scc(C)c2N(Cc2ccc(C)c(F)c2)C(=O)C1 ; Cc1ccc(CN2C(=O)CCC(=O)c3scc(C)c32)cc1F]

### 4-(3-Fluoro-4-methylbenzyl)-3-methyl-6, 7-dihydro-4H-thieno[3,2-b]azepine-5,8-dione

To the solution of resulting compound methyl 4-(3-fluoro-4-methylbenzyl)-8-hydroxy-3-methyl-5-oxo-5,6-dihydro-4H-thieno[3,2-b]azepine-7-carboxylate (825 mg, 2.2 mmol, 1 equiv) in DMSO (6 mL), was added H₂O (0.46 mL) under nitrogen, and the reaction mixture was allowed to stir at 150° C. for 3 h. The reaction mixture was allowed to cool to rt, ice (60 mL) was added, and the mixture was allowed to stir 12 h. To the flask was added 1N H₂SO₄ (30 mL) at 0° C. and the mixture allowed to stir for 3 h. The resulting precipitate was filtered. Then, the crude product was purified on a silica column to get 440 mg (purity=88%) light yellow oil compound with a yield of 63.02%.

TLC R_{f}=0.6 (PE:EA=1:1);

LCMS (m/z)=318.60 (M+H)

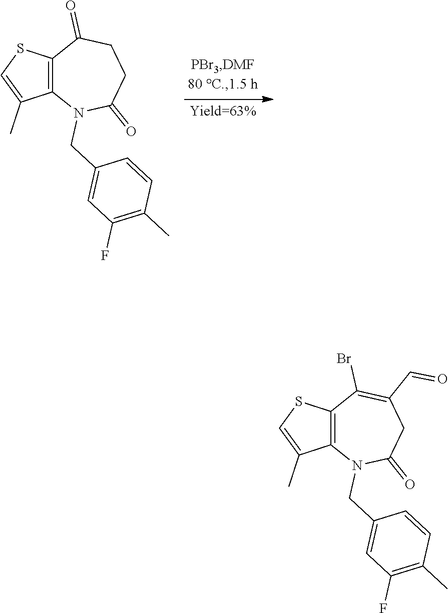 [SMILES: Cc1ccc(CN2C(=O)CCC(=O)c3scc(C)c32)cc1F ; Cc1ccc(CN2C(=O)CC(C=O)=C(Br)c3scc(C)c32)cc1F]

### 8-Bromo-4-(3-fluoro-4-methylbenzyl)-3-methyl-5-oxo-5,6-dihydro-4H-thieno[3,2-b]azepine-7-carbaldehyde

Phosphorus tribromide (171.3 μL, 1.80 mmol, 1.3 equiv) was added to a flask containing N,N-dimethylformamide (14 mL) in an ice bath and stirred for 10 min. The ice bath was replaced with an ambient temperature water bath and stirred for an additional 10-15 min. 4-(3-fluoro-4-methylbenzyl)-3-methyl-6,7-dihydro-4H-thieno[3,2-b]azepine-5,8-dione (440 mg, 1.39 mmol, 1.0 equiv) was added and stirred for 15 min. The reaction mixture was heated to 80° C. and stirred for an additional 1 h. The orange solution was diluted with ice water and neutralised with 20% sodium acetate solution and with EA (3×50 mL). The combined organic extracts were washed with saturated aqueous sodium bicarbonate solution (50 mL), brine (50 mL), and water (3×50 mL). Dried over anhydrous Na₂SO₄, filtered and concentrated by rotary evaporation to get the crude compound. Then, the resulting concentrate was purified on a silica column to get 358 mg (purity=60%) yellow oil compound with a yield of 63.25%.

TLC R_{f}=0.8 (PE:EA=1:1);

LCMS (m/z)=408.23 (M+1)

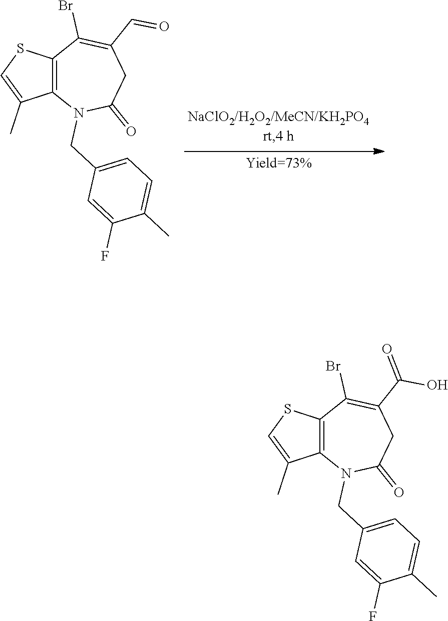 [SMILES: Cc1ccc(CN2C(=O)CC(C=O)=C(Br)c3scc(C)c32)cc1F ; Cc1ccc(CN2C(=O)CC(C(=O)O)=C(Br)c3scc(C)c32)cc1F]

### 8-Bromo-4-(3-fluoro-4-methylbenzyl)-3-methyl-5-oxo-5,6-dihydro-4H-thieno[3,2-b]azepine-7-carboxylic acid

Reactions were carried out by addition of aqueous NaClO₂ (138 mg, 1.53 mmol, 1.76 equiv) to a solution of 8-bromo-4-(3-fluoro-4-methylbenzyl)-3-methyl-5-oxo-5,6-dihydro-4H-thieno[3,2-b]azepine-7-carbaldehyde (355 mg, 0.87 mmol, 1 equiv) and (91 μL, 0.887 mmol, 1.02 equiv) of 30% H₂O₂ in aqueous acetonitrile, at 0° C., buffered with KH₂PO₄ at pH 4~3. The reaction mixture was stirred overnight. After complete reaction, a small amount of the Na₂SO₃ (~0.5 g) was added to destroy the unreacted HOCl and H₂O₂. Acidification with 10% HCl, the mixture was diluted with H₂O (10-20 mL). The compound was extracted with EA (3×30 mL). The organic layer was separated and extracted with water and brine and then dried over anhydrous Na₂SO₄, filtered and concentrated by rotary evaporation to get the crude compound. Then, the resulting concentrate was purified on a silica column to get 270 mg (purity=82%) yellow solid compound with a yield of 73.15%.

TLC R_{f}=0.25 (DCM:MeOH=10:1);

LCMS (m/z)=424.25 (M+H)

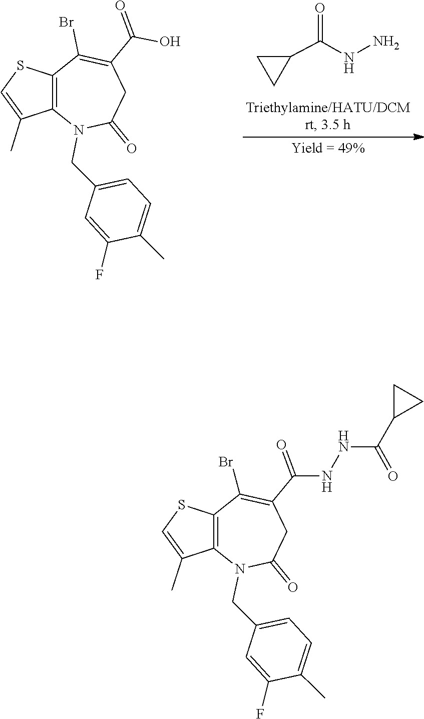 [SMILES: Cc1ccc(CN2C(=O)CC(C(=O)O)=C(Br)c3scc(C)c32)cc1F ; NNC(=O)C1CC1 ; Cc1ccc(CN2C(=O)CC(C(=O)NNC(=O)C3CC3)=C(Br)c3scc(C)c32)cc1F]

### 8-Bromo-N′-(cyclopropanecarbonyl)-4-(3-fluoro-4-methylbenzyl)-3-methyl-5-oxo-5,6-dihydro-4H-thieno[3,2-b]azepine-7-carbohydrazide

8-bromo-4-(3-fluoro-4-methylbenzyl)-3-methyl-5-oxo-5,6-dihydro-4H-thieno[3,2-b]azepine-7-carboxylic acid (270 mg, 0.636 mmol, 1 equiv) in dichloromethane (2.5 mL) was added Cyclopropanecarboxylicacid hydrazide (96 mg, 0.95 mmol, 1.5 equiv), triethylamine (131.5 μL, 0.95 mmol, 1.5 equiv), and HATU (361 mg, 0.95 mmol, 1.5 equiv). The mixture was stirred at room temperature for 3.5h. Then it was concentrated to give a crude, which was purified by silica gel column chromatography to get the product 156 mg (purity=90%) as a yellow solid. Yield: 48.41%.

TLC R_{f}=0.4 (DCM:MeOH=10:1);

LCMS (m/z)=506.70 (M+H)

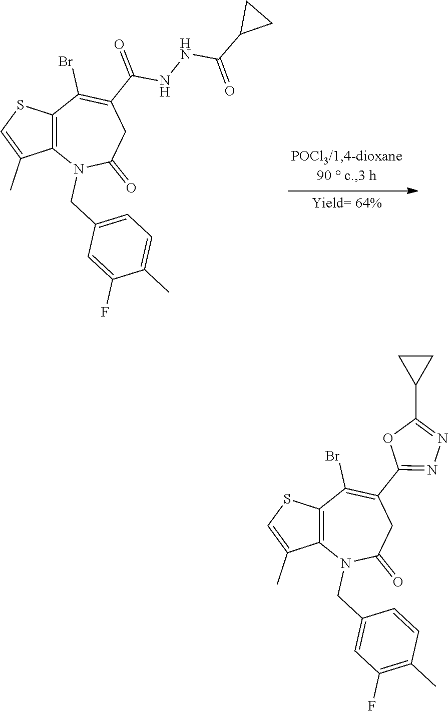 [SMILES: Cc1ccc(CN2C(=O)CC(C(=O)NNC(=O)C3CC3)=C(Br)c3scc(C)c32)cc1F ; Cc1ccc(CN2C(=O)CC(c3nnc(C4CC4)o3)=C(Br)c3scc(C)c32)cc1F]

### 8-Bromo-7-(5-cyclopropyl-1,3,4-oxadiazol-2-yl)-4-(3-fluoro-4-methylbenzyl)-3-methyl-4,6-dihydro-5H-thieno[3,2-b]azepin-5-one (1)

8-bromo-N′-(cyclopropanecarbonyl)-4-(3-fluoro-4-methylbenzyl)-3-methyl-5-oxo-5,6-dihydro-4H-thieno[3,2-b]azepine-7-carbohydrazide (156 mg, 0.308 mmol, 1 equiv) was dissolved in 1,4-dioxane (3.2 mL) and phosphoryl chloride (188 μL, 2.02 mmol, 6.57 equiv) was added and the mixture was heated at 90° C. for 3 h, then cooled to room temperature and diluted with ethyl acetate. The mixture was washed with satd. NaHCO₃ and brine, dried over sodium sulfate, filtered and concentrated in vacuo. The residue was purified by silica gel column chromatography to get the product (98 mg) as a yellow solid. Yield: 65.14%; Purity: 99%.

TLC R_{f}=0.5 (PE:EA=1:1);

LCMS (m/z)=488.48 (M+H)

¹H NMR (400 MHz, DMSO-d₆) δ 7.60 (dd, J=7.3, 1.2 Hz, 1H), 7.10 (t, J=7.9 Hz, 1H), 6.70-6.57 (m, 2H), 5.40 (d, J=15.7 Hz, 1H), 4.52 (d, J=15.7 Hz, 1H), 3.99 (d, J=12.9 Hz, 1H), 3.08 (d, J=12.8 Hz, 1H), 2.34 (tt, J=8.4, 4.8 Hz, 1H), 2.27 (dd, J=4.0, 1.1 Hz, 3H), 2.14 (d, J=2.0 Hz, 3H), 1.27-1.20 (m, 3H), 1.10 (dt, J=4.8, 3.2 Hz, 2H).

### Example 2. 8,8′-((((Oxybis(ethane-2,1-diyl))bis(oxy))bis(ethane-2,1-diyl))bis(oxy))bis(5-bromo-4-(5-cyclopropyl-1,3,4-oxadiazol-2-yl)-1-(3-fluoro-4-methylbenzyl)-1,3-dihydro-2H-benzo[b]azepin-2-one) (2)

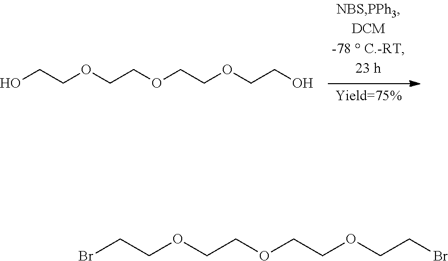 [SMILES: OCCOCCOCCOCCO ; BrCCOCCOCCOCCBr]

### 1-Bromo-2-(2-(2-(2-bromoethoxy)ethoxy)ethoxy)ethane

103 mL DCM was cooled to −78° C. Under stirring NBS (2.75 g, 15.45 mmol, 3 equiv) and triphenylphosphine (4.05 g, 15.45 mmol, 3 equiv) were added. After 10 min, 2,2′-((oxybis(ethane-2,1-diyl))bis(oxy))bis(ethan-1-ol) (1 g, 5.15 mmol, 1 equiv) was added and the solution was stirred overnight and the temperature was slowly increased to room temperature. The DCM was removed in vacuo and the product was taken up on silica gel. The product was then purified by flash chromatography over a short column with 250 mL of PE/EA (4:1). After removal of the solvents in vacuo the product was isolated as a colorless oil compound. Quality=1.23 g (Yield=74.65%).

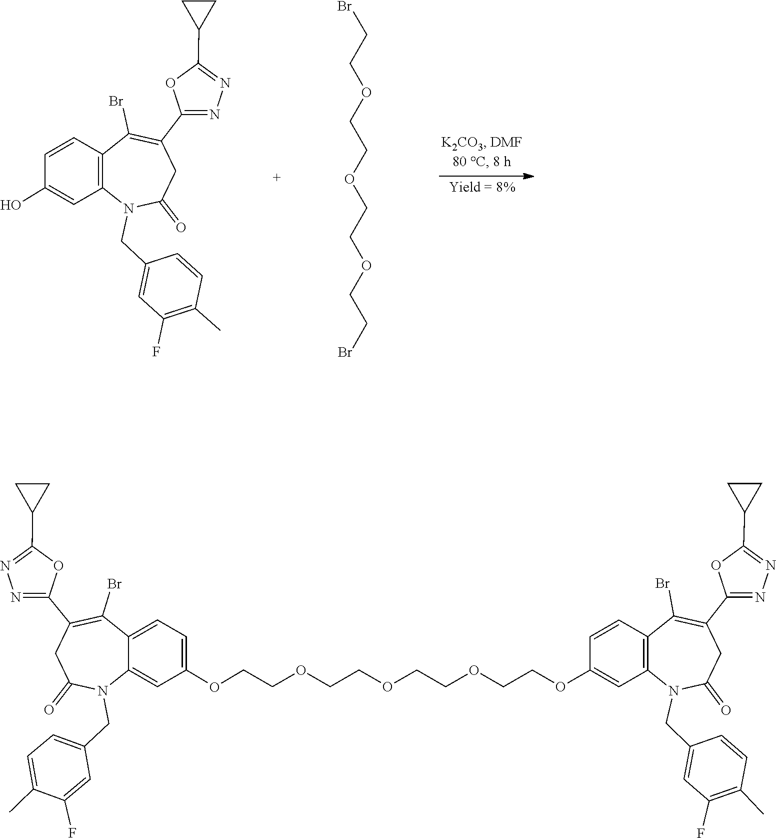 [SMILES: Cc1ccc(CN2C(=O)CC(c3nnc(C4CC4)o3)=C(Br)c3ccc(O)cc32)cc1F ; BrCCOCCOCCOCCBr ; Cc1ccc(CN2C(=O)CC(c3nnc(C4CC4)o3)=C(Br)c3ccc(OCCOCCOCCOCCOc4ccc5c(c4)N(Cc4ccc(C)c(F)c4)C(=O)CC(c4nnc(C6CC6)o4)=C5Br)cc32)cc1F]

To a solution of 5-bromo-4-(5-cyclopropyl-1,3,4-oxadiazol-2-yl)-1-(3-fluoro-4-methylbenzyl)-8-hydroxy-1,3-dihydro-2H-benzo[b]azepin-2-one (100 mg, 0.206 mmol, 1 equiv) in DMF (0.6 mL) was added K₂CO₃ (57 mg, 0.41 mmol, 2 equiv) and 1-bromo-2-(2-(2-(2-bromoethoxy)ethoxy)ethoxy)ethane (36 mg, 0.11 mmol, 0.55 equiv), and the reaction mixture was hated to 85° C. and stirred for 8 h. The solution was quenched with saturated ammonium chloride and extracted with EA (3×30 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a pre-TLC to get 18.5 mg (purity=85%) white solid compound with a yield of 7.95%.

TLC R_{f}=0.1 (PE:EA=1:1);

LCMS (m/z)=1125.25 (M+H)

### Example 3. 8,8′-(((Ethane-1,2-diylbis(oxy))bis(ethane-2,1-diyl))bis(oxy))bis(5-bromo-4-(5-cyclopropyl-1,3,4-oxadiazol-2-yl)-1-(3-fluoro-4-methylbenzyl)-1,3-dihydro-2H-benzo[b]azepin-2-one) (3)

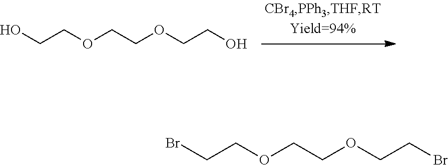 [SMILES: OCCOCCOCCO ; BrCCOCCOCCBr]

### 1,2-Bis(2-bromoethoxy)ethane

2,2′-(ethane-1,2-diylbis(oxy))bis(ethan-1-ol) (1.50 g, 10 mmol, 1 equiv) and CBr₄ (7.30 g, 22 mmol, 2.2 equiv) were dissolved in dry THF. PPh₃ (5.25 g, 20 mmol, 2 equiv) was added slowly under vigorous stirring. The reaction mixture was stirred at rt for 3 h, filtered, and concentrated. The crude material was purified by column chromatography on silica gel (PE/EA, 4:1 v/v) to afford 1,2-bis(2-bromoethoxy)ethane as a colorless oil (Quality=2.6 g, Yield=94.21%).

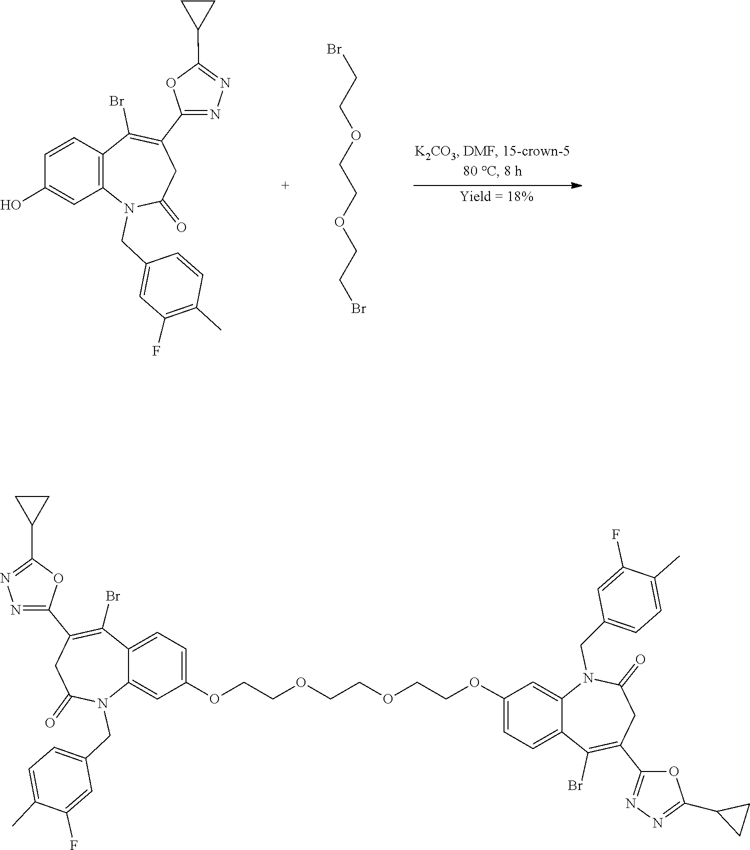 [SMILES: Cc1ccc(CN2C(=O)CC(c3nnc(C4CC4)o3)=C(Br)c3ccc(O)cc32)cc1F ; BrCCOCCOCCBr ; Cc1ccc(CN2C(=O)CC(c3nnc(C4CC4)o3)=C(Br)c3ccc(OCCOCCOCCOc4ccc5c(c4)N(Cc4ccc(C)c(F)c4)C(=O)CC(c4nnc(C6CC6)o4)=C5Br)cc32)cc1F]

### 8,8′-(((Ethane-1,2-diylbis(oxy))bis(ethane-2,1-diyl))bis(oxy))bis(5-bromo-4-(5-cyclopropyl-1,3,4-oxadiazol-2-yl)-1-(3-fluoro-4-methylbenzyl)-1,3-dihydro-2H-benzo[b]azepin-2-one) (3)

To the stirred mixture of K₂CO₃ (22 mg, 0.17 mmol, 1 equiv) and 16-crown-5 (1 drop) in dry DMF (0.5 mL) was added 5-bromo-4-(5-cyclopropyl-1,3,4-oxadiazol-2-yl)-1-(3-fluoro-4-methylbenzyl)-8-hydroxy-1,3-dihydro-2H-benzo[b]azepin-2-one (80 mg, 0.16 mmol, 1 equiv), then the reaction mixture was stirred for 1 h. After that time 1,2-bis(2-bromoethoxy)ethane (22 mg, 0.08 mmol, 0.5 equiv) was added. The solution was stirred at room temperature for 17 h and 8 h in 80° C. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on silica gel to get 34 mg (purity=72%) yellow solid compound with a yield of 19.01%.

TLC R_{f}=0.1 (PE:EA=1:1);

LCMS (m/z)=1081.43 (M+H)

### Example 4. 5-bromo-4-(5-cyclopropyl-1,3,4-oxadiazol-2-yl)-9-fluoro-1-(4-methoxybenzyl)-1,3-dihydro-2H-benzo[b]azepin-2-one (4)

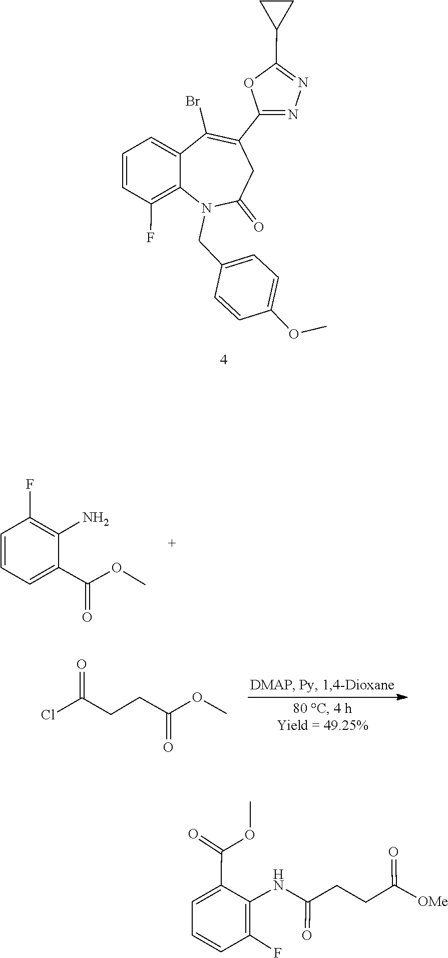

### methyl 3-fluoro-2-(4-methoxy-4-oxobutanamido)benzoate

To a solution of methyl 2-amino-3-fluorobenzoate (4.79 g, 28.37 mmol, 1 equiv) in 1,4-Dioxane (27.89 mL), was added pyridine (2.73 mL, 34 mmol, 1.2 equiv) and DMAP (173 mg, 1.42 mmol, 0.05 equiv) under nitrogen. To this solution was added 3-(carbomethoxy)propionyl chloride (5.53 g, 36.88 mmol, 1.3 equiv) dropwise over a period of 10 min, and the reaction mixture was allowed to stir at 80° C. for 4 h. To the reaction mixture was then added H₂O (40 mL) and the mixture was allowed to stir for 30 min. The organic solution was separated and the aqueous solution was extracted with DCM (3×50 mL). The organic solutions were combined, washed with H₂O (2×50 mL) and brine (1×100 mL), dried over anhydrous Na₂SO₄, filtered and concentrated by rotary evaporation to get the crude compound. Then, the resulting concentrate was purified on a silica column to get 3.958 g (purity=91.46%) of the light yellow solid compound with a yield of 49.25%.

TLC R_{f}=0.4 (PE:EA=2:1)

LCMS (m/z)=284.00 (M+H)

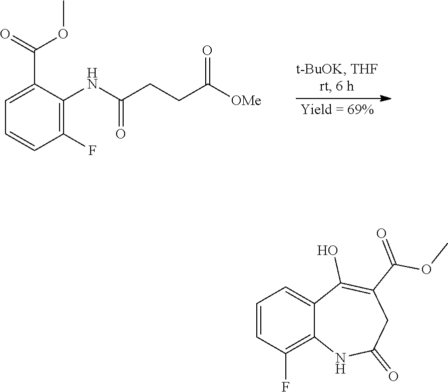 [SMILES: COC(=O)CCC(=O)Nc1c(F)cccc1C(=O)OC ; COC(=O)C1=C(O)c2cccc(F)c2NC(=O)C1]

### methyl 9-fluoro-5-hydroxy-2-oxo-2,3-dihydro-1H-benzo[b]azepine-4-carboxylate

To a solution of methyl 3-fluoro-2-(4-methoxy-4-oxobutanamido)benzoate (3.953 g, 13.97 mmol, 1 equiv) in THF (55 mL) at 5° C. was added a 1 M solution of t-BuOK in THF (41.92 mL, 41.92 mmol, 3 equiv) dropwise over 10 min while maintaining the temperature at 5° C. After 6 h, 20 mL of H₂O followed by 80 mL of 1N HCl were added to bring the solution to pH=4. The resulting mixture was allowed to stir at rt for 40 min. The organic solution was separated, and the aqueous solution was extracted with EtOAc (3×100 mL). The organic solutions were combined, dried over anhydrous Na₂SO₄, filtered and concentrated by rotary evaporation to afford crude compound. Then, the resulting concentrate was purified on a silica column to get 2.419 g of the white solid compound with a yield of 69%. Purity=86.46%

TLC R_{f}=0.6 (PE:EA=2:1)

LCMS (m/z)=252.00 (M+H)

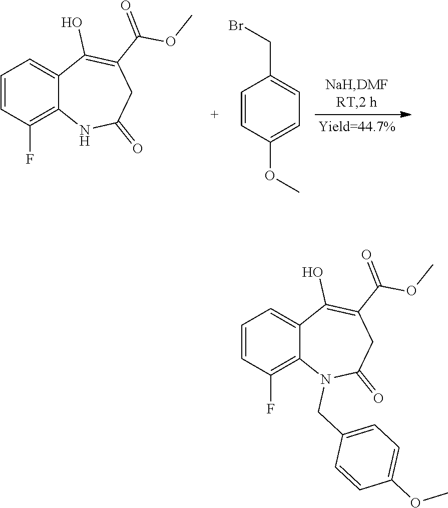

### methyl 9-fluoro-5-hydroxy-l-(4-methoxybenzyl)-2-oxo-2,3-dihydro-1H-benzo[b]azepine-4-carboxylate

To a solution of methyl 9-fluoro-5-hydroxy-2-oxo-2,3-dihydro-1H-benzo[b]azepine-4-carboxylate (5.14 g, 20.46 mmol, 1 equiv) in DMF (166 mL) was added NaH 60% dispersion in mineral oil (2.946 g, 73.65 mmol, 3.6 equiv). To this solution was added 1-(bromomethyl)-4-methoxybenzene (3.615 mL, 24.55 mmol, 1.2 equiv), and the reaction mixture was allowed to stir at rt for 1 h. The resulting solution was poured into water (120 mL) and extracted with Ethyl acetate (3×220 mL). The combined organic layers were washed with brine (1×120 mL), dried over anhydrous Na₂SO₄, and concentrated by rotary evaporation. Then, the resulting concentrate was purified on a silica column to get 3.4 g of the orange oil compound with a yield of 44.7%, purity=95%.

TLC R_{f}=0.55 (PE:EA=10:1);

LCMS (m/z)=472.30 (M+H)

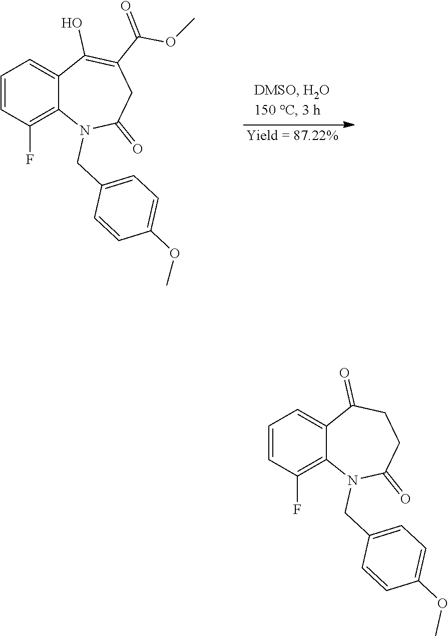 [SMILES: COC(=O)C1=C(O)c2cccc(F)c2N(Cc2ccc(OC)cc2)C(=O)C1 ; COc1ccc(CN2C(=O)CCC(=O)c3cccc(F)c32)cc1]

### 9-fluoro-1-(4-methoxybenzyl)-3,4-dihydro-1H-benzo[b]azepine-2,5-dione

To a solution of methyl 9-fluoro-5-hydroxy-1-(4-methoxybenzyl)-2-oxo-2,3-dihydro-1H-benzo[b]azepine-4-carboxylate (3.4 g, 9.15 mmol, 1 equiv) in DMSO (25 mL), was added H₂O (2 mL) under Nitrogen, and the reaction mixture was allowed to stir at 150° C. for 5 h. The reaction mixture was allowed to cool to rt, ice (11.30 mL) was added, and the mixture was allowed to stir 12 h. To the flask was added 1N HCl (11.30 mL) at 0° C. and the mixture allowed to stir for 3 h. The resulting precipitate was filtered. Then, the crude product was purified on a silica column to get 2.5 g (purity=92.68%) orange oil compound with a yield of 87.22%.

TLC R_{f}=0.2 (PE:EA=1:1)

LCMS (m/z)=314.40 (M+H)

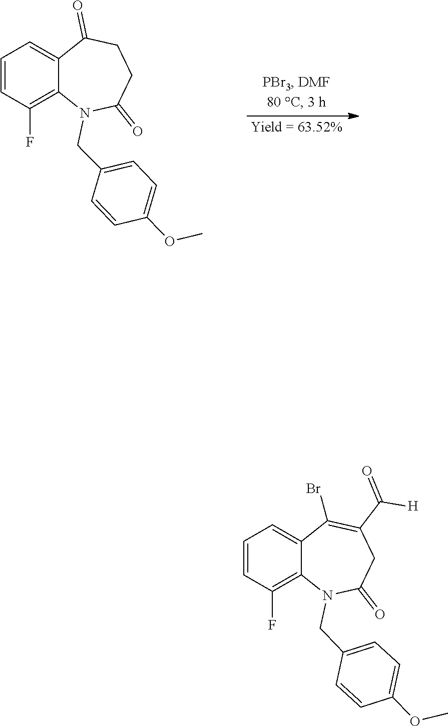 [SMILES: COc1ccc(CN2C(=O)CCC(=O)c3cccc(F)c32)cc1 ; COc1ccc(CN2C(=O)CC(C=O)=C(Br)c3cccc(F)c32)cc1]

### 5-bromo-9-fluoro-1-(4-methoxybenzyl)-2-oxo-2,3-dihydro-1H-benzo[b]azepine-4-carbaldehyde

Tribromophosphane (589 μL, 6.21 mmol, 1.3 equiv) was added to a flask containing N, N-dimethylformamide (48 mL) in an ice bath and stirred for 10 min. The ice bath was replaced with an ambient temperature water bath and stirred for an additional 10-15 min. 9-fluoro-1-(4-methoxybenzyl)-3,4-dihydro-1H-benzo[b]azepine-2,5-dione (1.5 g, 4.78 mmol, 1.0 equiv) was added and stirred for 15 min. The reaction mixture was heated to 80° C. and stirred for an additional 1 h. The orange solution was diluted with ice water and neutralised with 20% sodium acetate solution and extracted with EA (3×10-20 mL). The combined organic extracts were washed with saturated aqueous sodium bicarbonate solution 50 mL and brine 50 mL, dried over anhydrous Na₂SO₄, filtered and concentrated by rotary evaporation to get the crude compound. Then, the resulting concentrate was purified on a silica column to get 900 mg (purity=96%) orange solid compound with a yield of 46%.

TLC R_{f}=0.45 (PE:EA=1:1)

LCMS (m/z)=404.40 (M+H)

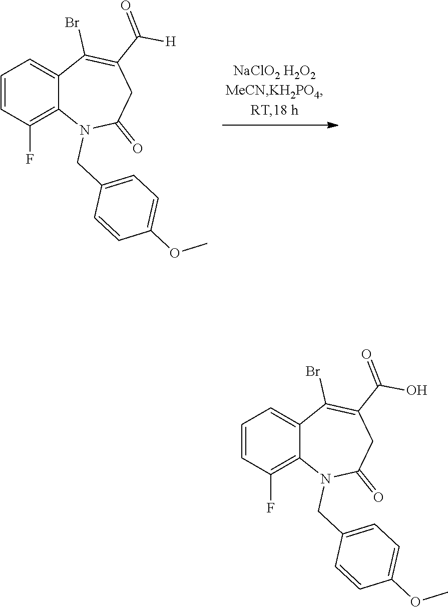 [SMILES: COc1ccc(CN2C(=O)CC(C=O)=C(Br)c3cccc(F)c32)cc1 ; COc1ccc(CN2C(=O)CC(C(=O)O)=C(Br)c3cccc(F)c32)cc1]

### 5-bromo-9-fluoro-1-(4-methoxybenzyl)-2-oxo-2,3-dihydro-1H-benzo[b]azepine-4-carboxylic acid

Reactions were carried out by addition of aqueous NaClO₂ (352 mg, 3.91 mmol, 1.76 equiv) to a solution of 5-bromo-9-fluoro-1-(4-methoxybenzyl)-2-oxo-2,3-dihydro-1H-benzo[b]azepine-4-carbaldehyde (900 mg, 2.22 mmol, 1 equiv) and (231 pl, 2.26 mmol, 1.02 equiv) of 30% H₂O₂ in aqueous acetonitrile, at 0° C., buffered with KH₂PO₄ at pH 4-3. The reaction mixture was stirred overnight. After complete reaction, a small amount of the Na₂SO₃(~0.5 g) was added to destroy the unreacted HOCl and H₂O₂. Acidification with 10% HCl, the mixture was diluted with H₂O (10-20 mL). The compound was extracted with EA (100 mL). The organic layer was separated and extracted with water and brine and then dried over Na₂SO₄ and concentrated to get 950 mg (purity=55%) white solid without purification.

TLC R_{f}=0.2 (DCM:MeOH=10:1)

LCMS (m/z)=420.20 (M+H)

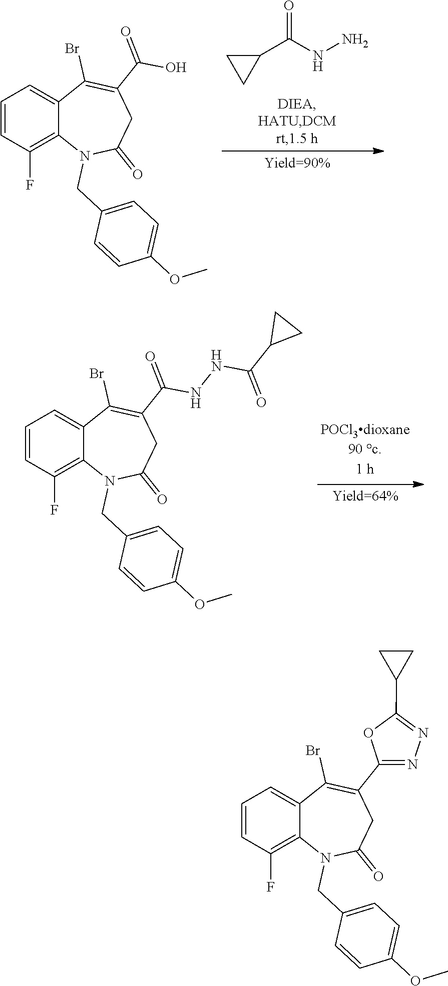 [SMILES: COc1ccc(CN2C(=O)CC(C(=O)O)=C(Br)c3cccc(F)c32)cc1 ; NNC(=O)C1CC1 ; COc1ccc(CN2C(=O)CC(C(=O)NNC(=O)C3CC3)=C(Br)c3cccc(F)c32)cc1 ; COc1ccc(CN2C(=O)CC(c3nnc(C4CC4)o3)=C(Br)c3cccc(F)c32)cc1]

### 5-bromo-4-(5-cyclopropyl-1,3,4-oxadiazol-2-yl)-9-fluoro-1-(4-methoxybenzyl)-1,3-dihydro-2H-benzo[b]azepin-2-one (4)

5-bromo-9-fluoro-1-(4-methoxybenzyl)-2-oxo-2,3-dihydro-1H-benzo[b]azepine-4-carboxylic acid (950 mg, 2.26 mmol, 1 equiv) in dichloromethane (20 mL) was added Cyclopropanecarboxylicacid hydrazide (339 mg, 3.39 mmol, 1.5 equiv), DIEA (590 μL, 3.39 mmol, 1.5 equiv), and HATU (1.718 g, 4.52 mmol, 2 equiv). The mixture was stirred at room temperature for 1.5h. Then it was concentrated to give a crude, which was purified by silica gel column chromatography to get the product 1.03 g (purity=96%) as a white solid. Yield: 90%.

TLC R_{f}=0.4 (DCM:MeOH=10:1)

LCMS (m/z)=502.20 (M+H)

Compound 5-bromo-N′-(cyclopropanecarbonyl)-9-fluoro-1-(4-methoxybenzyl)-2-oxo-2,3-dihydro-1H-benzo[b]azepine-4-carbohydrazide (1.3 g, 2.58 mmol, 1 equiv) was dissolved in dioxane (35 mL), then added phosphorus oxychloride (1.58 mL), and the mixture was stirred at 90° C. for 1 h. After the reaction mixture had cooled to room temperature, ethyl acetate was added and the diluted solution was slowly poured into room temperature water under a water bath. The resulting solution was extracted with portions of ethyl acetate (3×40 mL). The combined organic extracts were dried over anhydrous sodium sulfate and concentrated to give a crude, which was purified by silica gel column chromatography to get the product 800 mg as orange oil. Yield: 64%.

Purity: 97%.

TLC R_{f}=0.5 (PE:EA=1:1);

LCMS (m/z)=484.50 (M+H)

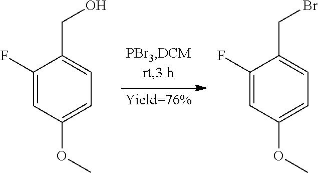

### 1-(Bromomethyl)-2-fluoro-4-methoxybenzene

To a stirred suspension of (2-fluoro-4-methoxyphenyl)methanol (500 mg, 3.20 mmol, 1.00 equiv) in CH2Cl2 (4.9 mL) at 0° C. was added phosphorous tribromide (1.45 g, 5.35 mmol, 1.67 equiv) dropwise. After 15 min, the mixture was warmed to rt and allowed to stir for 3 h, at which point the reaction mixture was quenched with H2O (10 mL). The aqueous phase was extracted with CH2Cl2 (3×10 mL), the combined organic layers were washed with saturated aqueous sodium bicarbonate (10 mL) and brine (10 mL), dried (Na2SO4), filtered and concentrated in vacuo to afford title compound 540 mg yellow oil compound with a yield of 76.99%, purity=38%.

TLC R_{f}=0.9 (PE:EA=1:1);

### Example 5. 5-Bromo-4-(5-cyclopropyl-1,3,4-oxadiazol-2-yl)-1-(2-fluoro-4-methoxybenzyl)-1,3-dihydro-2H-benzo[b]azepin-2-one (5)

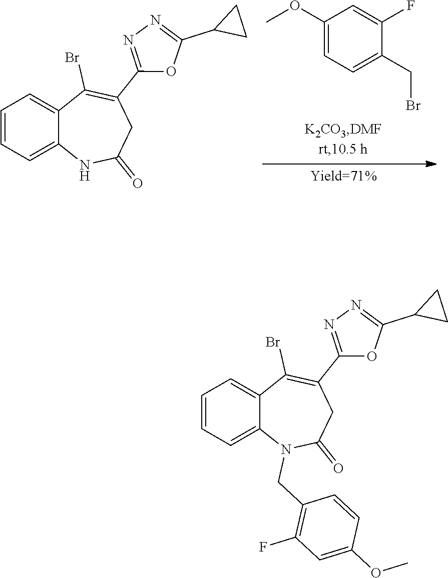

To a stirred solution of 5-bromo-4-(5-cyclopropyl-1,3,4-oxadiazol-2-yl)-1,3-dihydro-2H-benzo[b]azepin-2-one (50 mg, 0.14 mmol, 1 equiv) and K₂CO₃ (59 mg, 0.43 mmol, 3 equiv) in DMF (0.6 mL), was added 1-(bromomethyl)-2-fluoro-4-methoxybenzene (37 mg, 0.17 mmol, 1.2 equiv) at ambient temperature. The reaction mixture was then stirred at rt for 3.5 h. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 48 mg (purity=810%) white solid compound with a yield of 68.62%.

TLC R_{f}=0.55 (PE:EA=1:1);

LCMS (m/z)=484.20 (M+H)

### Example 6. 5-Bromo-4-(5-cyclopropyl-1,3,4-oxadiazol-2-yl)-1-(4-ethylbenzyl)-1,3-dihydro-2H-benzo[b]azepin-2-one (6)

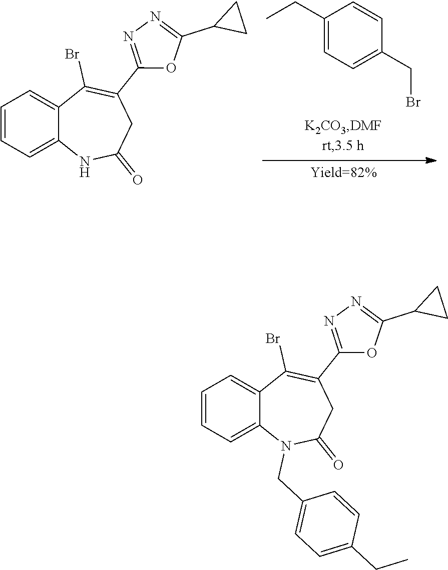

To a stirred solution of 5-bromo-4-(5-cyclopropyl-1,3,4-oxadiazol-2-yl)-1,3-dihydro-2H-benzo[b]azepin-2-one (300 mg, 0.86 mmol, 1 equiv) and K₂CO₃ (359 mg, 2.60 mmol, 3 equiv) in DMF (0.6 mL), was added 1-(bromomethyl)-4-ethylbenzene (207 mg, 1.04 mmol, 1.2 equiv) at ambient temperature. The reaction mixture was then stirred at rt for 3.5 h. The solution was quenched with saturated ammonium chloride and extracted with EA (3×30 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 300 mg (purity=97%) yellow solid compound with a yield of 74.55%.

TLC R_{f}=0.35 (PE:EA=1:1);

LCMS (m/z)=464.20 (M+H)

¹H NMR (400 MHz, DMSO-d₆): δ 7.78 (dd, J=8.0, 1.6 Hz, 1H), 7.56 (dd, J=8.3, 1.3 Hz, 1H), 7.48 (ddd, J=8.4, 7.2, 1.6 Hz, 1H), 7.32 (ddd, J=8.3, 7.2, 1.3 Hz, 1H), 7.08-7.01 (m, 2H), 6.93-6.87 (m, 2H), 5.35 (d, J=16.0 Hz, 1H), 4.90 (d, J=15.9 Hz, 1H), 3.86 (d, J=13.2 Hz, 1H), 3.05 (d, J=13.1 Hz, 1H), 2.50 (q, J=7.7 Hz, 2H), 2.41-2.30 (m, 1H), 1.27-1.20 (m, 2H), 1.17-1.11 (m, 2H), 1.10 (t, J=7.5 Hz, 3H).

### Scheme B: General Route for the Synthesis of Five-Membered Heterocycle Analogs

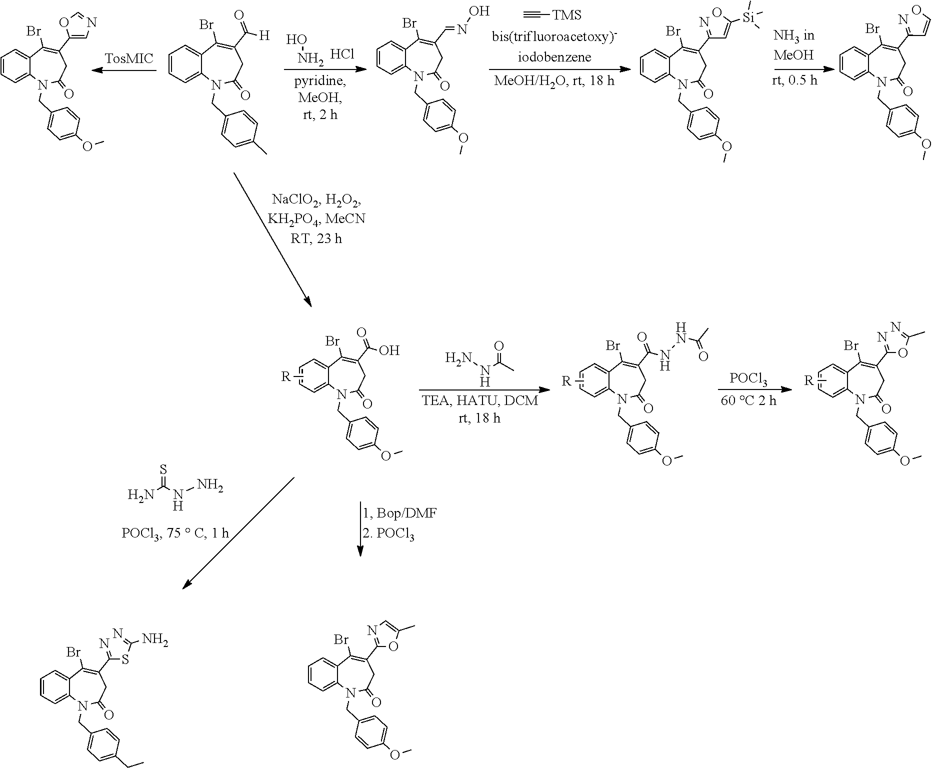

### Example 7. 5-Bromo-1-(4-methoxybenzyl)-4-(oxazol-5-yl)-1,3-dihydro-21-benzo[b]azepin-2-one(7)

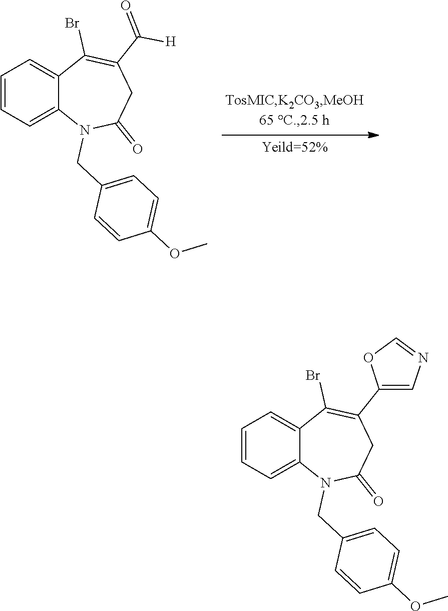 [SMILES: COc1ccc(CN2C(=O)CC(C=O)=C(Br)c3ccccc32)cc1 ; COc1ccc(CN2C(=O)CC(c3cnco3)=C(Br)c3ccccc32)cc1]

The toluenesulfonylmethyl isocyanide(164 mg, 0.84 mmol, 1.2 equiv) was placed in a dry round-bottom flask and dry MeOH (21.7 mL) added under an argon atmosphere. At rt, solid K₂CO₃ (290 mg, 2.1 mmol, 3 equiv) and the 5-bromo-1-(4-methoxybenzyl)-2-oxo-2,3-dihydro-1H-benzo[b]azepine-4-carbaldehyde (270 mg, 0.7 mmol, 1 equiv) were added to the mixture, and the mixture was heated to reflux for 2.5 h. After completion of the reaction on analysis by TLC, which was indicated by the absence of aldehyde starting material, the mixture was quenched with cold water. After the mixture was quenched, all of the solvent was removed under reduced pressure and the product extracted with ethyl acetate (3×30 mL). The combined organic layers were washed with water, brine and dried (Na₂SO₄). The solvent was then removed under reduced pressure and the residue purified by flash chromatography (silica gel) to give the pure white solid (purity=99%, 220 mg, yield=73.90%).

TLC R_{f}=0.45 (PE:EA=1:1);

LCMS (m/z)=425.50 (M+H)

¹H NMR (400 MHz, DMSO-d₆): δ 8.62 (s, 1H), 8.00 (s, 1H), 7.73 (dd, J=8.0, 1.6 Hz, 1H), 7.56 (dd, J=8.3, 1.3 Hz, 1H), 7.43 (ddd, J=8.4, 7.2, 1.6 Hz, 1H), 7.29 (ddd, J=8.3, 7.2, 1.2 Hz, 1H), 6.90-6.86 (m, 2H), 6.78-6.73 (m, 2H), 5.35 (d, J=15.6 Hz, 1H), 4.83 (d, J=15.6 Hz, 1H), 3.66 (s, 3H), 3.67 (d, J=13.1 Hz, 1H), 2.97 (d, J=13.1 Hz, 1H).

### Example 8. 5-Bromo-4-(isoxazol-3-yl)-1-(4-methoxybenzyl)-1,3-dihydro-2H-benzo[b]azepin-2-one (8)

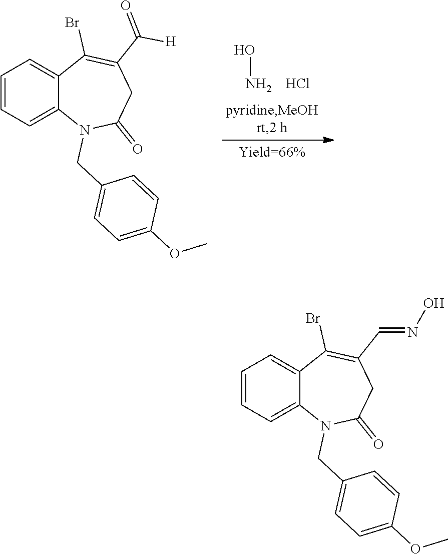

### (E)-5-Bromo-1-(4-methoxybenzyl)-2-oxo-2,3-dihydro-1H-benzo[b]azepine-4-carbaldehyde oxime

A solution of 5-bromo-1-(4-methoxybenzyl)-2-oxo-2,3-dihydro-1H-benzo[b]azepine-4-carbaldehyde (695 mg, 1.8 mmol, 1 equiv) in methanol (45 mL) was treated with hydroxylamine hydrochloride (162 mg, 2.34 mmol, 1.3 equiv) and pyridine (170 mg, 2.16 mmol, 1.2 equiv). The reaction was stirred at room temperature for 2 h and then concentrated. The residue was purified by silica gel chromatography to get 480 mg (purity=85%) yellow solid compound with a yield of 66.46%.

TLC R_{f}=0.45 (PE:EA=1:1);

LCMS (m/z)=401.50 (M+H)

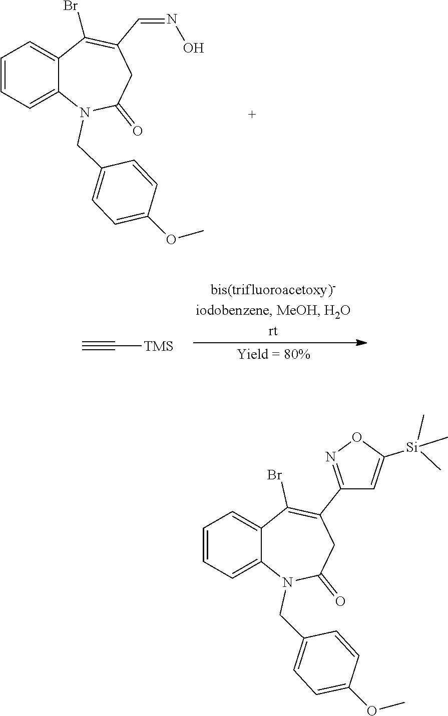

### 5-Bromo-1-(4-methoxybenzyl)-4-(5-(trimethylsilyl)isoxazol-3-yl)-1,3-dihydro-2H-benzo[b]azepin-2-one

To a solution of (E)-5-bromo-1-(4-methoxybenzyl)-2-oxo-2,3-dihydro-1H-benzo[b]azepine-4-carbaldehyde oxime (480 mg, 1.2 mmol, 1 equiv) and trimethylsilyl acetylene (235 mg, 2.40 mmol, 2 equiv) in methanol (78 mL) and water (15.3 mL) was added bis(trifluoroacetoxy)-iodobenzene (567 mg, 1.32 mmol, 1.1 equiv) and the reaction was stirred overnight. The reaction mixture was poured into MTBE, and washed with water and satd. aq. NaHCO₃. The organic layer was concentrated, and the residue was purified by silica gel chromatography to get 479 mg (purity=99%) white solid compound with a yield of 80.24%.

TLC R_{f}=0.8 (PE:EA=1:1);

LCMS (m/z)=497.58 (M+H)

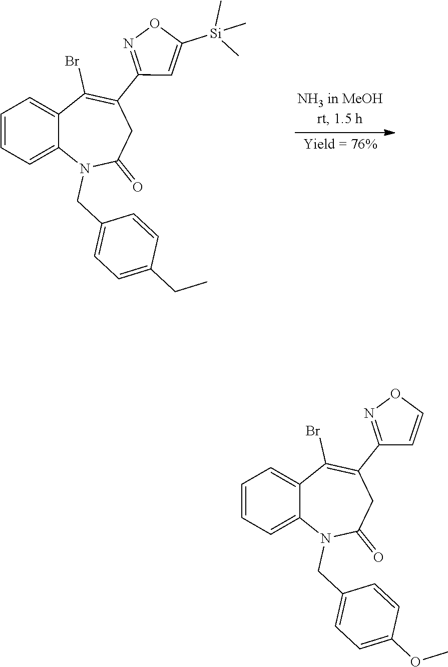 [SMILES: CCc1ccc(CN2C(=O)CC(c3cc([Si](C)(C)C)on3)=C(Br)c3ccccc32)cc1 ; COc1ccc(CN2C(=O)CC(c3ccon3)=C(Br)c3ccccc32)cc1]

### 5-Bromo-4-(isoxazol-3-yl)-1-(4-methoxybenzyl)-1,3-dihydro-2H-benzo[b]azepin-2-one (8)

A solution of 5-bromo-1-(4-methoxybenzyl)-4-(5-(trimethylsilyl)isoxazol-3-yl)-1,3-dihydro-2H-benzo[b]azepin-2-one (429 mg, 0.86 mmol) in 7M ammonia in methanol (104.4 mL, 731 mmol) was stirred at room temperature for 1.5 h. The reaction was concentrated under vacuum and purified by silica gel chromatography to get 278 mg (purity=99%) yellow oil with a yield of 76.01%.

TLC R_{f}=0.6 (PE:EA=1:1);

LCMS (m/z)=425.65 (M+H)

¹H NMR (400 MHz, DMSO-d₆) δ 9.11 (d, J=1.8 Hz, 1H), 7.75 (dd, J=8.0, 1.6 Hz, 1H), 7.59 (d, J=7.9 Hz, 1H), 7.51-7.43 (m, 1H), 7.35-7.27 (m, 1H), 7.11 (d, J=1.8 Hz, 1H), 6.93 (d, J=8.6 Hz, 2H), 6.78 (d, J=8.6 Hz, 2H), 5.40 (d, J=15.6 Hz, 1H), 4.84 (d, J=15.6 Hz, 1H), 3.67 (s, 3H), 3.62 (d, J=12.9 Hz, 1H), 3.07 (d, J=12.9 Hz, 1H).

### Example 9. 5-Bromo-1-(4-methoxybenzyl)-4-(5-methyl-1,3,4-oxadiazol-2-yl)-1,3-dihydro-2H-benzo[b]azepin-2-one (9)

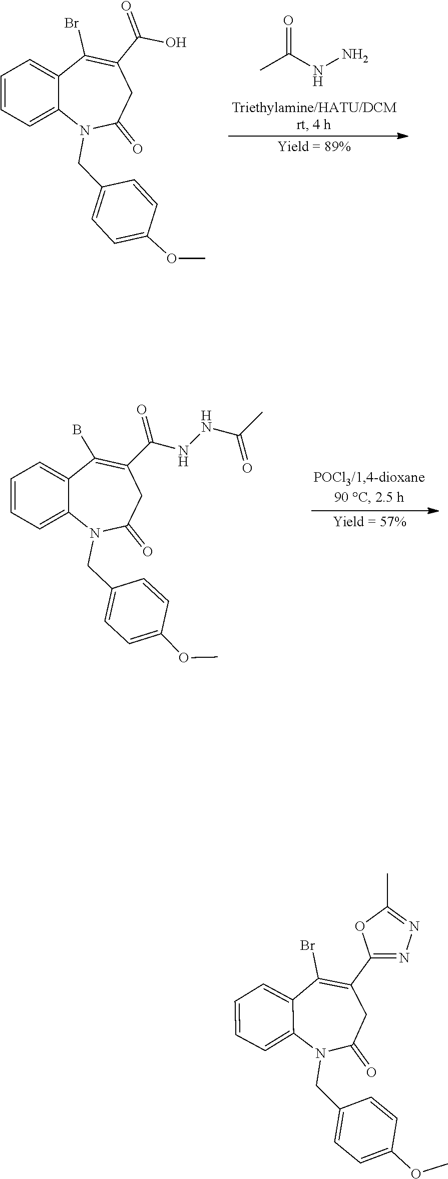

5-bromo-1-(4-methoxybenzyl)-2-oxo-2,3-dihydro-1H-benzo[b]azepine-4-carboxylic acid (480 mg, 1.19 mmol, 1 equiv) in dichloromethane (4 mL) was added acetohydrazide (132 mg, 1.79 mmol, 1.5 equiv), triethylamine (295 μL, 1.79 mmol, 1.5 equiv), and HATU (680 mg, 1.79 mmol, 1.5 equiv). The mixture was stirred at room temperature for 4 h. Then it was concentrated to give a crude, which was purified by silica gel column chromatography (PE/EA, 1:4) to get the product 490 mg (purity=90%) as a white solid. Yield: 89.59%.

TLC R_{f}=0.5 (DCM:MeOH=10:1);

LCMS (m/z)=458.60 (M+H)

N′-acetyl-5-bromo-1-(4-methoxybenzyl)-2-oxo-2,3-dihydro-1H-benzo[b]azepine-4-carbohydrazide (490 mg, 1.07 mmol, 1 equiv) was dissolved in 1,4-dioxane (58.8 mL) and phosphoryl chloride (642 μL, 7.02 mmol, 6.57 equiv) was added and the mixture was heated at 90° C. for 2.5 h, then cooled to room temperature and diluted with ethyl acetate. The mixture was washed with satd. NaHCO₃ and brine, dried over sodium sulfate, filtered and concentrated in vacuo. The residue was purified by silica gel column chromatography to get the product (270 mg) as a yellow solid. Yield: 57.36%; Purity: 92%.

TLC R_{f}=0.3 (PE:EA=1:1);

LCMS (m/z)=440.52 (M+H)

¹H NMR (400 MHz, DMSO-d₆): δ 7.78 (dd, J=8.1, 1.5 Hz, 1H), 7.58 (dd, J=8.4, 1.2 Hz, 1H), 7.48 (ddd, J=8.4, 7.2, 1.6 Hz, 1H), 7.34-7.29 (m, 1H), 6.94-6.89 (m, 2H), 6.78-6.74 (m, 2H), 5.37 (d, J=15.6 Hz, 1H), 4.84 (d, J=15.6 Hz, 1H), 3.84 (d, J=13.2 Hz, 1H), 3.66 (s, 3H), 3.06 (d, J=13.2 Hz, 1H), 2.61 (s, 3H).

### Example 10. 4-(5-Amino-1,3,4-thiadiazol-2-yl)-5-bromo-1-(4-methoxybenzyl)-1,3-dihydro-2H-benzo[b]azepin-2-one (10)

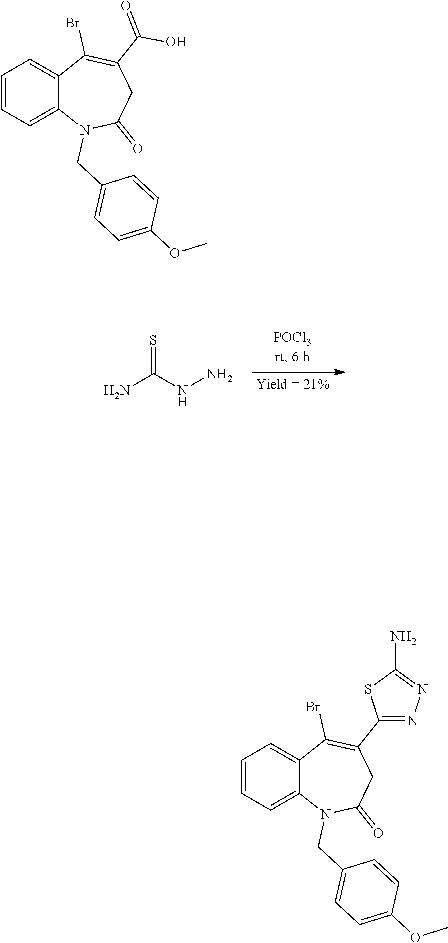 [SMILES: COc1ccc(CN2C(=O)CC(C(=O)O)=C(Br)c3ccccc32)cc1 ; NNC(N)=S ; COc1ccc(CN2C(=O)CC(c3nnc(N)s3)=C(Br)c3ccccc32)cc1]

A stirring mixture of 5-bromo-1-(4-methoxybenzyl)-2-oxo-2,3-dihydro-1H-benzo[b]azepine-4-carboxylic acid (350 mg, 0.87 mmol, 1 equiv), N-aminothiourea (158 mg, 1.74 mmol, 2 equiv) and POCl₃ (2.1 mL) was stirred at rt for 6 h. Then, water (30 ml) was added. The mixture was basified to pH=8 by drop wise addition of 50% NaOH solution under stirring, and extracted with EA (3×20 mL). The organic solutions were combined, washed with H₂O (2×20 mL) and brine (20 mL), dried over anhydrous Na₂SO₄, filtered and concentrated by rotary evaporation to get the crude compound. Then, the resulting concentrate was purified on a silica column to get 84 mg (purity=94%) yellow solid compound with a yield of 21.11%.

TLC R_{f}=0.35 (DCM:MeOH=10:1)

LCMS (m/z)=457.50 (M+H)

¹H NMR (400 MHz, DMSO-d₆) δ 7.74 (dd, J=8.0, 1.6 Hz, 1H), 7.67 (s, 2H), 7.56 (d, J=7.8 Hz, 1H), 7.44 (td, J=8.4, 7.8, 1.6 Hz, 1H), 7.33-7.25 (m, 1H), 6.88 (d, J=8.7 Hz, 2H), 6.75 (d, J=8.7 Hz, 2H), 5.36 (d, J=15.6 Hz, 1H), 4.83 (d, J=15.7 Hz, 1H), 4.42 (d, J=12.6 Hz, 1H), 3.67 (s, 3H), 2.87 (d, J=12.6 Hz, 1H).

### Example 11. 5-Bromo-1-(4-methoxybenzyl)-4-(5-methyloxazol-2-yl)-1,3-dihydro-2H-benzo[b]azepin-2-one (11)

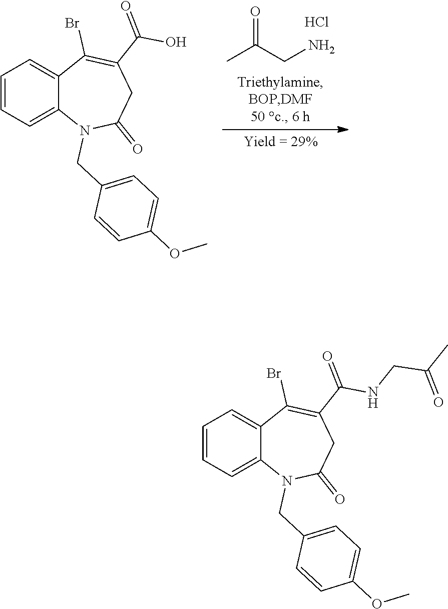

### 5-Bromo-1-(4-methoxybenzyl)-2-oxo-N-(2-oxopropyl)-2,3-dihydro-1H-benzo[b]azepine-4-carboxamide

A mixture of 5-bromo-1-(4-methoxybenzyl)-2-oxo-2,3-dihydro-1H-benzo[b]azepine-4-carboxylic acid (1.4 g, 3.48 mmol, 1 equiv), 1-aminoacetone HCl salt (476 mg, 4.35 mmol, 1.25 equiv), BOP reagent (1.92 g, 4.35 mmol, 1.25 equiv), and triethylamine (1.185 mL, 8.53 mmol, 2.45 equiv) in DMF (10.2 mL) was stirred at 50° C. for 6 h. The reaction mixture was partitioned between ethyl acetate (10 mL) and water (5 mL). The ethyl acetate layer was washed with brine, dried (Na₂SO₄), and concentrated under reduced pressure. Then, the resulting concentrate was purified on a silica column to get 460 mg (purity=86%) yellow oil compound with a yield of 28.90%.

TLC R_{f}=0.7 (DCM:MeOH=10:1)

LCMS (m/z)=457.50 (M+H)

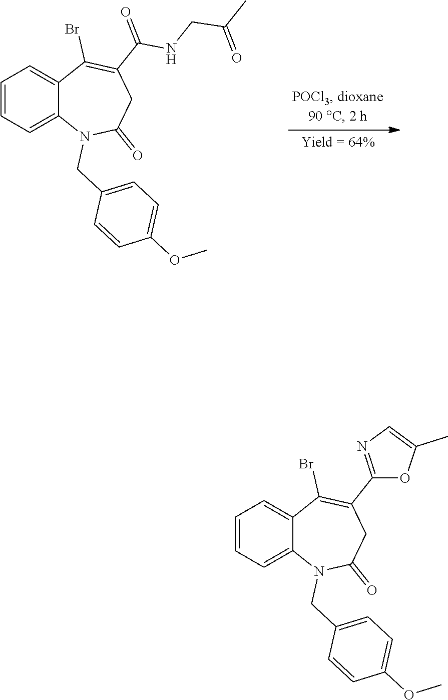 [SMILES: COc1ccc(CN2C(=O)CC(C(=O)NCC(C)=O)=C(Br)c3ccccc32)cc1 ; COc1ccc(CN2C(=O)CC(c3ncc(C)o3)=C(Br)c3ccccc32)cc1]

### 5-Bromo-1-(4-methoxybenzyl)-4-(5-methyloxazol-2-yl)-1,3-dihydro-2H-benzo[b]azepin-2-one (11)

5-bromo-1-(4-methoxybenzyl)-2-oxo-N-(2-oxopropyl)-2,3-dihydro-1H-benzo[b]azepine-4-carboxamide (460 mg, 1.01 mmol, 1 equiv) was dissolved in 1,4-dioxane (10.1 mL) and phosphoryl chloride (616 μL, 6.61 mmol, 6.57 equiv) was added and the mixture was heated at 90° C. for 2 h, then cooled to room temperature and diluted with ethyl acetate. The mixture was washed with satd. NaHCO₃ and brine, dried over sodium sulfate, filtered and concentrated in vacuo. The residue was purified by silica gel column chromatography to get the product (283 mg) as a yellow oil. Yield: 64.04%; Purity: 97%.

TLC R_{f}=0.75 (PE:EA=1:1);

LCMS (m/z)=441.06 (M+H)

¹H NMR (400 MHz, DMSO-d₆) δ 7.78 (dd, J=8.0, 1.5 Hz, 1H), 7.55 (dd, J=8.3, 0.9 Hz, 1H), 7.44 (td, J=8.4, 7.8, 1.5 Hz, 1H), 7.33-7.26 (m, 1H), 7.15 (d, J=1.2 Hz, 1H), 6.92 (d, J=8.7 Hz, 2H), 6.80-6.73 (m, 2H), 5.34 (d, J=15.7 Hz, 1H), 4.85 (d, J=15.7 Hz, 1H), 3.99 (d, J=12.9 Hz, 1H), 3.67 (s, 3H), 2.94 (d, J=12.9 Hz, 1H), 2.41 (d, J=1.1 Hz, 3H)

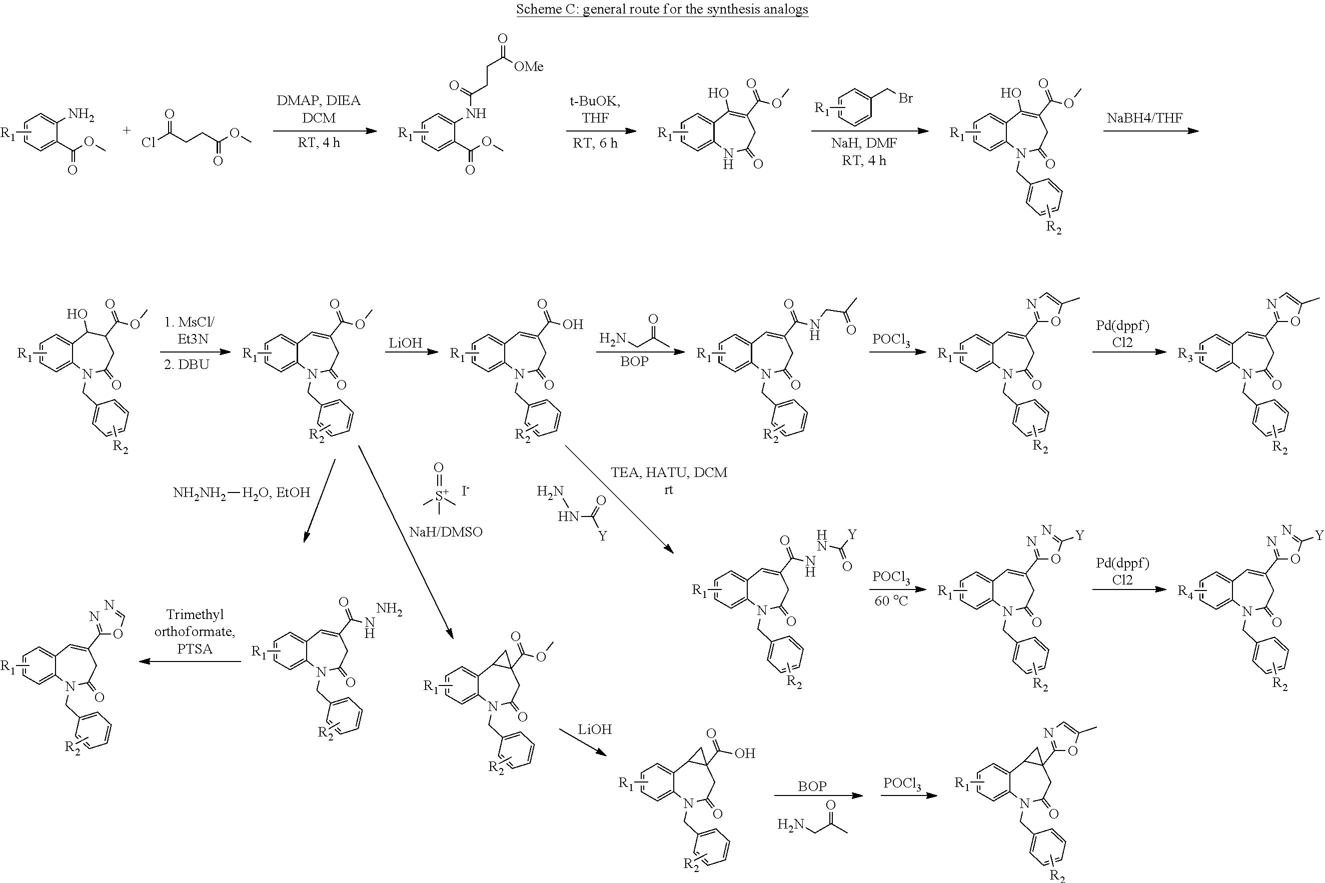

### Example 12. methyl 1-(3-fluoro-4-methylbenzyl)-2-oxo-2,3-dihydro-1H-benzo[b]azepine-4-carboxylate (12)

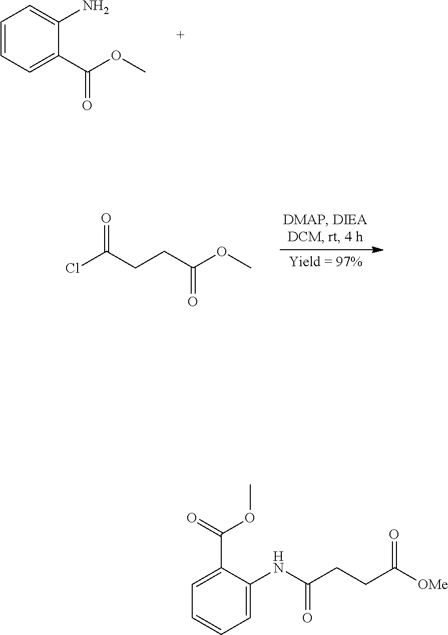

### Methyl 2-(3-methoxy-3-oxopropanamido) benzoate

To a solution of methyl 2-aminobenzoate (2.27 g, 15 mmol, 1 equiv) in DCM (45 mL), was added DIEA (2.79 g, 19.5 mmol, 1.3 equiv) and DMAP (9 mg, 0.075 mmol, 0.5% equiv) under nitrogen. To this solution was added 3-(carbomethoxy) propionyl chloride (2.94 g, 19.5 mmol, 1.3 equiv) dropwise over a period of 10 min, and the reaction mixture was allowed to stir at rt for 4 h. To the reaction mixture was then added H₂O (40 mL) and the mixture was allowed to stir for 30 min. The organic solution was separated, and the aqueous solution was extracted with EA (3×100 mL). The organic solutions were combined, washed with H₂O (2×50 mL) and brine (1×100 mL), dried over anhydrous Na₂SO₄, filtered and concentrated by rotary evaporation to get the crude compound. Then, the resulting concentrate was purified on a silica column to get 3.89 g (purity=98%) of the light-yellow oil compound with a yield of 97.76%.

TLC R_{f}=0.5 (PE:EA=2:1);

LCMS (m/z)=266.2 (M+H);

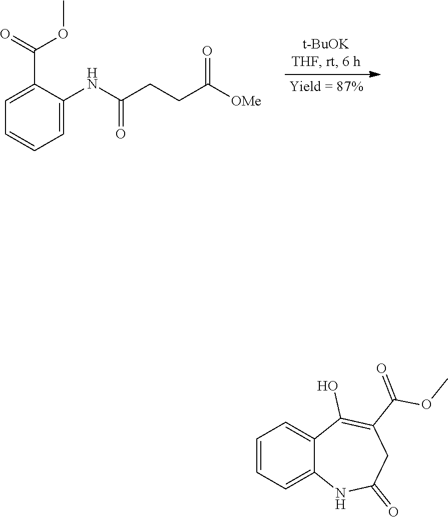 [SMILES: COC(=O)CCC(=O)Nc1ccccc1C(=O)OC ; COC(=O)C1=C(O)c2ccccc2NC(=O)C1]

### Methyl 5-hydroxy-2-oxo-2,3-dihydro-1H-benzo[b]azepine-4-carboxylate

To a solution of methyl 2-(3-methoxy-3-oxopropanamido)benzoate (4.95 g, 18.66 mmol, 1 equiv) in THF (74 mL) at 5° C. was added a 1 M solution of t-BuOK in THF (55.98 mL, 55.98 mmol, 3 equiv) dropwise over 10 min while maintaining the temperature at 5° C. After 6 h, 200 mL of H₂O followed by 1N HCl were added to bring the solution to pH=4. The resulting mixture was allowed to stir at rt for 40 min. The organic solution was separated, and the aqueous solution was extracted with EtOAc (3×100 mL). The organic solutions were combined, dried over anhydrous Na₂SO₄, filtered and concentrated by rotary evaporation to afford crude compound. Then, the resulting concentrate was purified on a silica column to get 3.79 g of the white solid compound with a yield of 87.08%. Purity=95%

TLC R_{f}=0.3 (PE:EA=2:1);

LCMS (m/z)=234.3 (M+H)

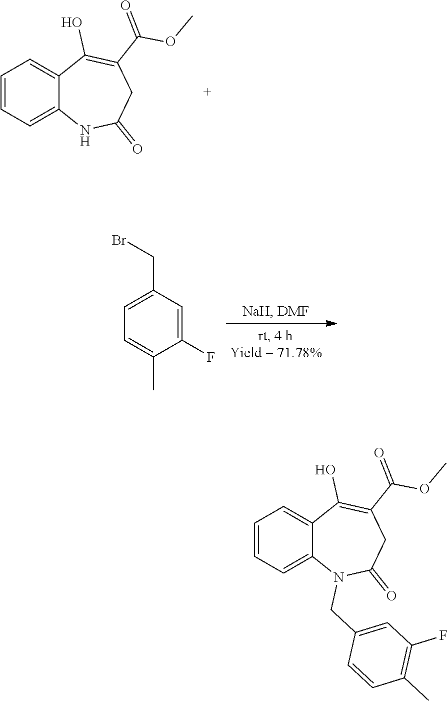

### methyl 1-(3-fluoro-4-methylbenzyl)-5-hydroxy-2-oxo-2,3-dihydro-H-benzo[b]azepine-4-carboxylate

To a solution of methyl 5-hydroxy-2-oxo-2,3-dihydro-1H-benzo[b]azepine-4-carboxylate (3.20 g, 13.72 mmol, 1.00 equiv) in DMF (90 mL) was added NaH 60% dispersion in mineral oil (1.65 g, 41.16 mmol, 3.00 equiv). To this solution was added 4-(bromomethyl)-2-fluoro-1-methylbenzene (3.34 g, 16.46 mmol, 1.20 equiv), and the reaction mixture was allowed to stir at room temperature for 4 h. The resulting solution was poured into water (60 mL) and extracted with EA (3×100 mL). The combined organic layers were washed with brine (1×100 mL), dried over anhydrous Na₂SO₄, and concentrated in vacuo. The crude was purified by silica gel column chromatography to get target product (3.50 g, 71.78%) as a white solid compound.

TLC R_{f}=0.5 (PE:EA=2:1)

LCMS (m/z)=356.2 (M+H).

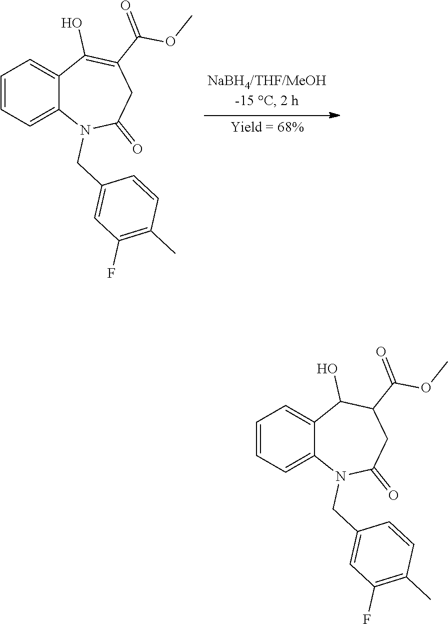 [SMILES: COC(=O)C1=C(O)c2ccccc2N(Cc2ccc(C)c(F)c2)C(=O)C1 ; COC(=O)C1CC(=O)N(Cc2ccc(C)c(F)c2)c2ccccc2C1O]

### methyl 1-(3-fluoro-4-methylbenzyl)-5-hydroxy-2-oxo-2,3,4,5-tetrahydro-1H-benzo[b]azepine-4-carboxylate

To a mixture of methyl 1-(3-fluoro-4-methylbenzyl)-5-hydroxy-2-oxo-2,3-dihydro-1H-benzo[b]azepine-4-carboxylate (710 mg, 2 mmol, 1 equiv) in THF (20 ml) was added NaBH₄ (181 mg, 4.8 mmol, 2.4 equiv) and MeOH (2 ml) at −40° C. The mixture was stirred at −15° C. for 2 h.

To the reaction mixture was added NH₄Cl aqueous solution (20 ml) and water at −15° C., and the mixture was extracted with EtOAc. The organic layer was washed with water and brine, dried over MgSO₄, and concentrated in vacuo. The residue was purified by silica gel column chromatography to get the product (490 mg) as a white solid. Yield: 68.55%; Purity: 99%.

TLC R_{f}=0.5 (PE:EA=1:1);

LCMS (m/z)=358.70 (M+H)

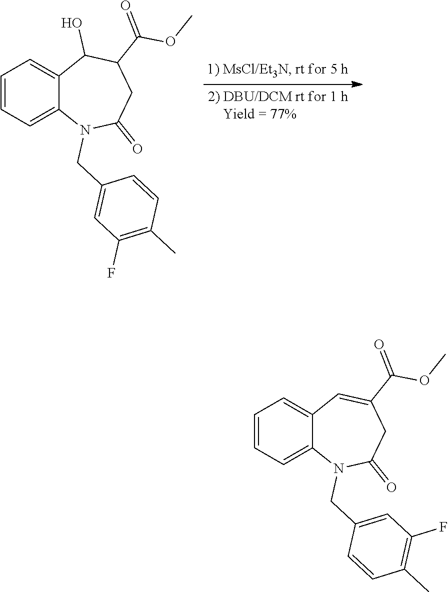 [SMILES: COC(=O)C1CC(=O)N(Cc2ccc(C)c(F)c2)c2ccccc2C1O ; COC(=O)C1=Cc2ccccc2N(Cc2ccc(C)c(F)c2)C(=O)C1]

### methyl 1-(3-fluoro-4-methylbenzyl)-2-oxo-2,3-dihyd-o-1H-benzo[b]azepine-4-carboxylate (12)

Methanesulfonyl chloride (200 μL, 2.74 mmol, 2 equiv) was added to a solution of methyl 1-(3-fluoro-4-methylbenzyl)-5-hydroxy-2-oxo-2,3,4,5-tetrahydro-1H-benzo[b]azepine-4-carboxylate (490 mg, 1.37 mmol, 1 equiv) and triethylamine (577 μL, 4.11 mmol, 3 equiv) in CH₂C₁₂ (5 mL) under ice cooling. The reaction mixture was stirred at room temperature for 5h. Then DBU (838 μL, 5.48 mmol, 4 equiv) was added dropwise under ice cooling. The reaction mixture was stirred at r.t. for 1 h and then poured into water, 1 M HCl was added (to pH=3) and the mixture was extracted with EA (2×50 mL). The combined organic layers were washed with water (50 mL) and brine (50 mL), dried (Na₂SO₄) and evaporated in vacuo to give the crude product, which was purified by silica gel column chromatography to get the product (360 mg) as oil. Yield: 77.37%; Purity: 98%.

TLC R_{f}=0.5 (PE:EA=2:1);

LCMS (m/z)=340.60 (M+H)

### Example 13. 4-(5-cyclopropyl-1,3,4-oxadiazol-2-yl)-1-(3-fluoro-4-methylbenzyl)-1,3-dihydro-2H-benzo[b]azepin-2-one (13)

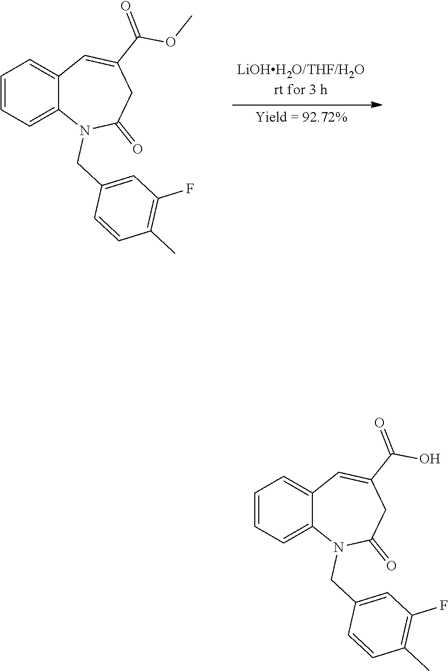 [SMILES: COC(=O)C1=Cc2ccccc2N(Cc2ccc(C)c(F)c2)C(=O)C1 ; Cc1ccc(CN2C(=O)CC(C(=O)O)=Cc3ccccc32)cc1F]

### 1-(3-fluoro-4-methylbenzyl)-2-oxo-2,3-dihydro-1H-benzo[b]azepine-4-carboxylic acid

To a stirred solution methyl 1-(3-fluoro-4-methylbenzyl)-2-oxo-2,3-dihydro-1H-benzo[b]azepine-4-carboxylate (360 mg, 1.06 mmol, 1 equiv) in 1:1:1 mixture solvent (5.1 mL), was added LiOH·H₂O (222 mg, 5.3 mmol, 5 equiv) at ambient temperature. The reaction mixture was then stirred at rt for 3 h. The solution was quenched with 1N HCl and extracted with EA (3×30 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 320 mg (purity=99%) white solid compound with a yield of 92.72%.

TLC R_{f}=0.25 (DCM:MeOH=10:1);

LCMS (m/z)=326.60 (M+H)

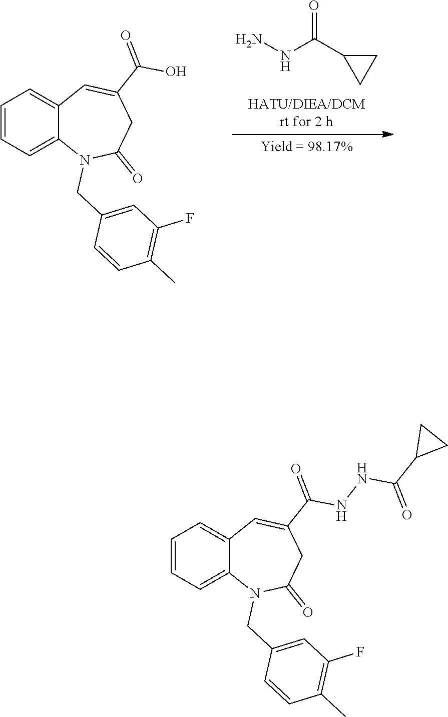 [SMILES: Cc1ccc(CN2C(=O)CC(C(=O)O)=Cc3ccccc32)cc1F ; NNC(=O)C1CC1 ; Cc1ccc(CN2C(=O)CC(C(=O)NNC(=O)C3CC3)=Cc3ccccc32)cc1F]

### N′-(cyclopropanecarbonyl)-1-(3-fluoro-4-methylbenzyl)-2-oxo-2,3-dihydro-1H-benzo[b]azepine-4-carbohydrazide

1-(3-fluoro-4-methylbenzyl)-2-oxo-2,3-dihydro-1H-benzo[b]azepine-4-carboxylic acid (325 mg, 1 mmol, 1 equiv) in dichloromethane (5 mL) was added Cyclopropanecarboxylicacid hydrazide (150 mg, 1.5 mmol, 1.5 equiv), triethylamine (216 μL, 1.5 mmol, 1.5 equiv), and HATU (760 mg, 2 mmol, 2 equiv). The mixture was stirred at room temperature for 2 h. Then it was concentrated to give a crude, which was purified by silica gel column chromatography to get the product 400 mg (purity=99%) as a yellow solid. Yield: 98.17%.

TLC R_{f}=0.35 (PE:EA=1:2);

LCMS (m/z)=408.60 (M+H)

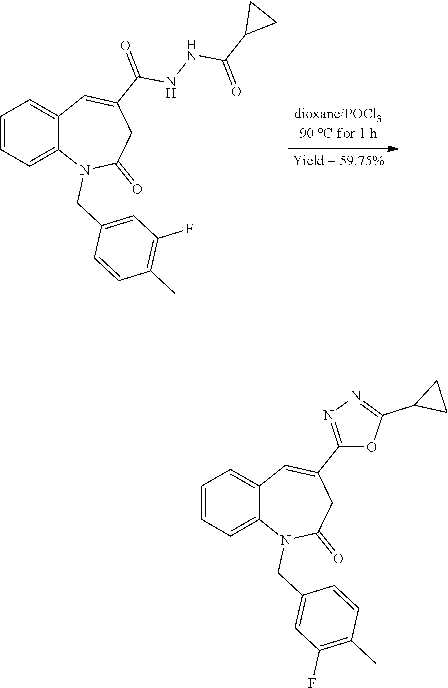 [SMILES: Cc1ccc(CN2C(=O)CC(C(=O)NNC(=O)C3CC3)=Cc3ccccc32)cc1F ; Cc1ccc(CN2C(=O)CC(c3nnc(C4CC4)o3)=Cc3ccccc32)cc1F]

### 4-(5-cyclopropyl-1,3,4-oxadiazol-2-yl)-1-(3-fluoro-4-methylbenzyl)-1,3-dihydro-2H-benzo[b]azepin-2-one (13)

N′-(cyclopropanecarbonyl)-1-(3-fluoro-4-methylbenzyl)-2-oxo-2,3-dihydro-1H-benzo[b]azepine-4-carbohydrazide (450 mg, 1.1 mmol, 1 equiv) was dissolved in 1,4-dioxane (11 mL) and phosphoryl chloride (475 μL, 7.26 mmol, 6.57 equiv) was added and the mixture was heated at 90° C. for 1 h, then cooled to room temperature and diluted with ethyl acetate. The mixture was washed with satd. NaHCO₃ and brine, dried over sodium sulfate, filtered and concentrated in vacuo. The residue was purified by silica gel column chromatography to get the product (257 mg) as a yellow solid. Yield: 59.75%; Purity: 99%.

TLC R_{f}=0.35 (PE:EA=2:1);

LCMS (m/z)=390.70 (M+H)

¹H NMR (400 MHz, Chloroform-d) δ 7.56 (s, 1H), 7.41-7.32 (m, 3H), 7.20 (dt, J=7.6, 4.2 Hz, 1H), 7.04 (td, J=7.8, 0.8 Hz, 1H), 6.81-6.69 (m, 2H), 5.06 (s, 2H), 4.15-2.95 (br, 2H), 2.26-2.16 (m, 4H, CH₃ and CH overlapped), 1.28-1.17 (m, 4H).

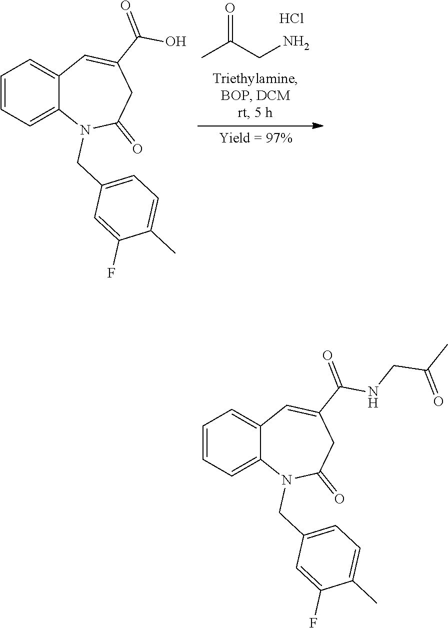

### 1-(3-fluoro-4-methylbenzyl)-2-oxo-N-(2-oxopropyl)-2,3-dihydro-1H-benzo[b]azepine-4-carboxamide

A mixture of 1-(3-fluoro-4-methylbenzyl)-2-oxo-2,3-dihydro-1H-benzo[b]azepine-4-carboxylic acid (149 mg, 0.46 mmol, 1 equiv), 1-aminoacetone HCl salt (75 mg, 0.69 mmol, 1.5 equiv), BOP reagent (304 mg, 0.69 mmol, 1.5 equiv), and triethylamine (200 uL, 1.14 mmol, 2.5 equiv) in DCM (2.3 mL) was stirred for 3 h. The reaction mixture was purified on a silica column to get 170 mg (purity=99%) white solid compound with a yield of 97%.

TLC R_{f}=0.6 (DCM:MeOH=10:1)

LCMS (m/z)=381.50 (M+H)

### Example 14. 8-bromo-1-(4-methoxybenzyl)-4-(1,3,4-oxadiazol-2-yl)-1,3-dihydro-2H-benzo[b]azepin-2-one (14)

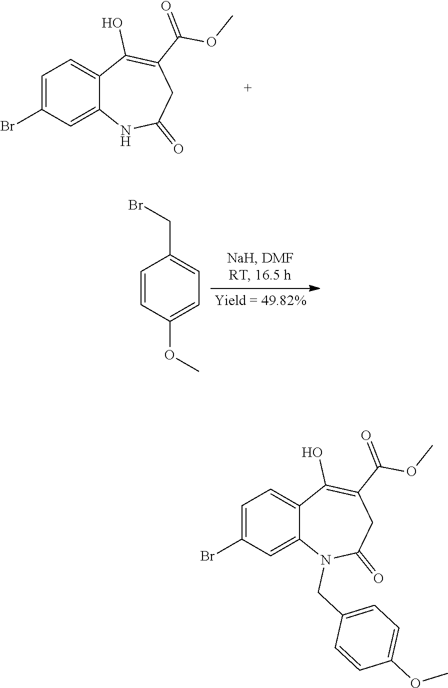

### methyl 8-bromo-5-hydroxy-1-(4-methoxybenzyl)-2-oxo-2,3-dihydro-1H-benzo[b]azepine-4-carboxylate

To a solution of methyl 8-bromo-5-hydroxy-2-oxo-2,3-dihydro-1H-benzo[b]azepine-4-carboxylate (10 g, 32.04 mmol, 1 equiv) in DMF (560 mL) was added NaH 60% dispersion in mineral oil (4.61 g, 115.34 mmol, 3.6 equiv). To this solution was added 1-(bromomethyl)-4-methoxybenzene (5.58 mL, 38.45 mmol, 1.2 equiv), and the reaction mixture was allowed to stir at rt for 16.5 h. The resulting solution was poured into water (120 mL) and extracted with Ethyl acetate (3×300 mL). The combined organic layers were washed with brine (1×120 mL), dried over anhydrous Na₂SO₄, and concentrated by rotary evaporation. Then, the resulting concentrate was purified on a silica column to get 6.9 g of the white solid compound with a yield of 49.82%, purity=98%.

TLC R_{f}=0.5 (PE:EA=4:1);

LCMS (m/z)=432.25 (M+H)

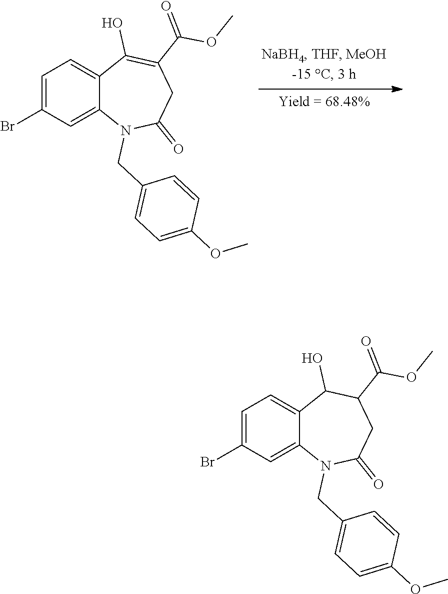 [SMILES: COC(=O)C1=C(O)c2ccc(Br)cc2N(Cc2ccc(OC)cc2)C(=O)C1 ; COC(=O)C1CC(=O)N(Cc2ccc(OC)cc2)c2cc(Br)ccc2C1O]

### methyl 8-bromo-5-hydroxy-1-(4-methoxybenzyl)-2-oxo-2,3,4,5-tetrahydro-1H-benzo[b]azepine-4-carboxylate

To a mixture of methyl 8-bromo-5-hydroxy-1-(4-methoxybenzyl)-2-oxo-2,3-dihydro-1H-benzo[b]azepine-4-carboxylate (1 g, 2.31 mmol, 1 equiv) in THE (23 ml) was added NaBH₄ (210 mg, 5.55 mmol, 2.4 equiv) and MeOH (2.3 ml) at −40° C. The mixture was stirred at −15° C. for 3 h. To the reaction mixture was added NH₄Cl aqueous solution (20 ml) and water at −15° C., and the mixture was extracted with EtOAc. The organic layer was washed with water and brine, dried over MgSO₄, and concentrated in vacuo. The residue was purified by silica gel column chromatography to get the product (688 mg) as a white solid. Yield: 68.48%; Purity: 99%.

TLC R_{f}=0.1 (PE:EA=4:1);

LCMS (m/z)=433.80 (M+H)

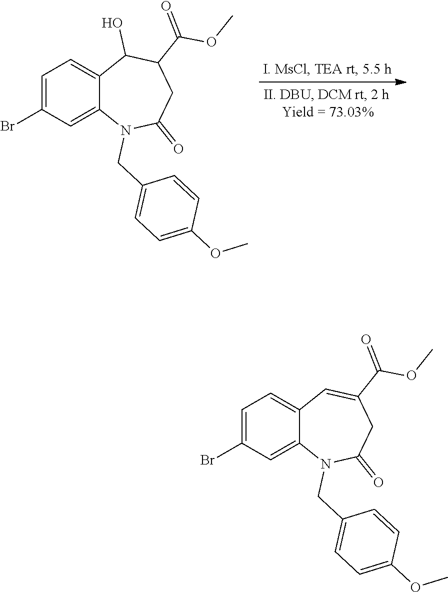 [SMILES: COC(=O)C1CC(=O)N(Cc2ccc(OC)cc2)c2cc(Br)ccc2C1O ; COC(=O)C1=Cc2ccc(Br)cc2N(Cc2ccc(OC)cc2)C(=O)C1]

### methyl 8-bromo-1-(4-methoxybenzyl)-2-oxo-2,3-dihydro-1H-benzo[b]azepine-4-carboxylate

Methanesulfonyl chloride (1.44 mL, 18.42 mmol, 2 equiv) was added to a solution of methyl 8-bromo-5-hydroxy-1-(4-methoxybenzyl)-2-oxo-2,3,4,5-tetrahydro-1H-benzo[b]azepine-4-carboxylate (4 g, 9.21 mmol, 1 equiv) and triethylamine (3.84 mL, 27.63 mmol, 3 equiv) in CH₂Cl₂ (33.6 mL) under ice cooling. The reaction mixture was stirred 5.5 h at r.t. Then DBU (5.52 mL, 36.84 mmol, 4 equiv) was added dropwise under ice cooling. The reaction mixture was stirred at r.t for 1 h and then poured into water, 1 M HCl was added (pH 3) and the mixture was extracted with EA (2×300 mL). The combined organic layers were washed with water (100 mL) and brine (100 mL), dried (Na₂SO₄) and evaporated in vacuo to give the crude product, which was purified by silica gel column chromatography to get the product (2.8 g) as white solid. Yield: 73.03%; Purity: 99%.

TLC R_{f}=0.6 (PE:EA=4:1);

LCMS (m/z)=416.00 (M+H)

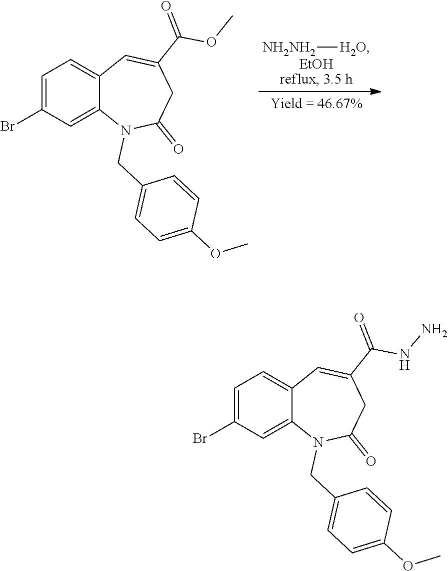

### 8-bromo-1-(4-methoxybenzyl)-2-oxo-2,3-dihydro-1H-benzo[b]azepine-4-carbohydrazide

A mixture of methyl 8-bromo-1-(4-methoxybenzyl)-2-oxo-2,3-dihydro-1H-benzo[b]azepine-4-carboxylate (300 mg, 0.72 mmol, 1 equiv) and hydrazine hydrate (960 uL) in EtOH (4.8 mL) was refluxed for 3.5 h. After cooling to room temperature, the solution was extracted with EA (3×80 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 140 mg (purity=63%) white solid compound with a yield of 46.67%.

TLC R_{f}=0.15 (PE:EA=1:4);

LCMS (m/z)=415.90 (M+H)

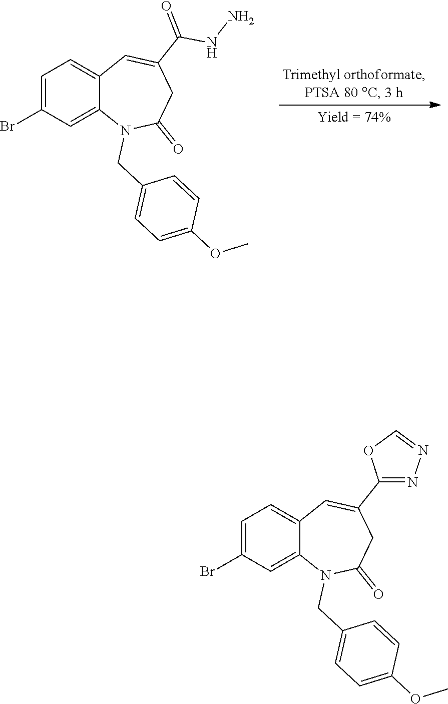 [SMILES: COc1ccc(CN2C(=O)CC(C(=O)NN)=Cc3ccc(Br)cc32)cc1 ; COc1ccc(CN2C(=O)CC(c3nnco3)=Cc3ccc(Br)cc32)cc1]

### 8-bromo-1-(4-methoxybenzyl)-4-(1,3,4-oxadiazol-2-yl)-1,3-dihydro-2H-benzo[b]azepin-2-one (14)

A mixture of 8-bromo-1-(4-methoxybenzyl)-2-oxo-2,3-dihydro-1H-benzo[b]azepine-4-carbohydrazide (140 mg, 0.33 mmol, 1 equiv), trimethyl orthoformate (9.4 mL), and PTSA (75 mg, 0.43 mmol, 1.3 equiv) was heated at 80° C. for 3 h. After cooling to room temperature, the solution was extracted with EA (3×50 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 107 mg (purity=93%) white solid compound with a yield of 74.64%.

TLC R_{f}=0.4 (PE:EA=1:1);

LCMS (m/z)=426.40 (M+H)

### Example 15. 8-bromo-1-(4-methoxybenzyl)-4-(5-methyl-1,3,4-oxadiazol-2-yl)-1,3-dihydro-2H-benzo[b]azepin-2-one (15)

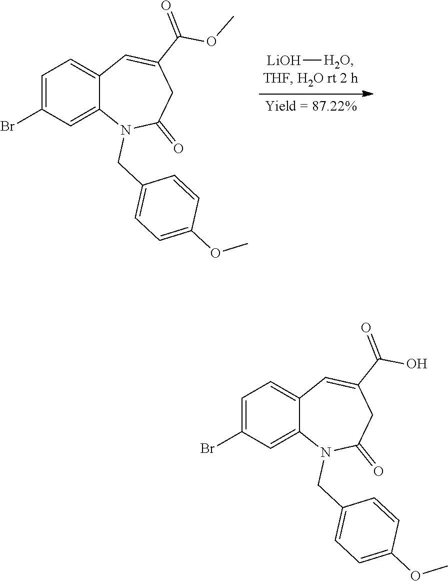

### 8-bromo-1-(4-methoxybenzyl)-2-oxo-2,3-dihydro-1H-benzo[b]azepine-4-carboxylic acid

To a stirred solution methyl 8-bromo-1-(4-methoxybenzyl)-2-oxo-2,3-dihydro-1H-benzo[b]azepine-4-carboxylate (2.8 g, 6.73 mmol, 1 equiv) in 1:1:1 mixture solvent MeOH/THF/H₂O (32 mL), was added LiOH·H₂O (1.41 g, 33.63 mmol, 5 equiv) at ambient temperature. The reaction mixture was then stirred at rt for 2 h. The solution was quenched with 1N HCl and extracted with EA (3×100 mL). The organic solutions were combined, washed with H₂O (2×100 mL) and brine (1×100 mL), dried over anhydrous Na₂SO₄, filtered and concentrated by rotary evaporation to get the crude compound. Then, the resulting concentrate was purified on a silica column to get 2.36 g (purity=97%) white solid compound with a yield of 87.22%.

TLC R_{f}=0.3 (DCM:MeOH=10:1);

LCMS (m/z)=401.90 (M+H)

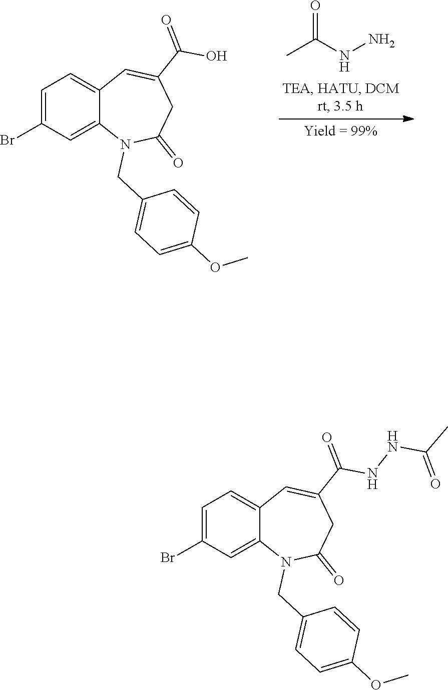

### N′-acetyl-8-bromo-1-(4-methoxybenzyl)-2-oxo-2,3-dihydro-1H-benzo[b]azepine-4-carbohydrazide

8-bromo-1-(4-methoxybenzyl)-2-oxo-2,3-dihydro-1H-benzo[b]azepine-4-carboxylic acid (1 g, 2.49 mmol, 1 equiv) in dichloromethane (10 mL) was added acetohydrazide (276 mg, 3.73 mmol, 1.5 equiv), triethylamine (516 μL, 3.73 mmol, 1.5 equiv), and HATU (1.42 g, 3.73 mmol, 1.5 equiv). The mixture was stirred at room temperature for 3.5 h. Then it was concentrated to give a crude, which was purified by silica gel column chromatography (DCM/MeOH, 10:1) to get the product 1.13 g (purity=99%) as a white solid. Yield=99.18%.

TLC R_{f}=0.2 (DCM:MeOH=10:1);

LCMS (m/z)=458.42 (M+H)

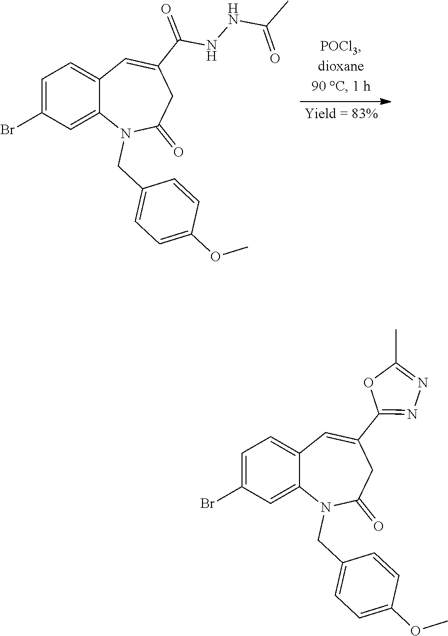 [SMILES: COc1ccc(CN2C(=O)CC(C(=O)NNC(C)=O)=Cc3ccc(Br)cc32)cc1 ; COc1ccc(CN2C(=O)CC(c3nnc(C)o3)=Cc3ccc(Br)cc32)cc1]

### 8-bromo-1-(4-methoxybenzyl)-4-(5-methyl-1,3,4-oxadiazol-2-yl)-1,3-dihydro-2H-benzo[b]azepin-2-one (15)

N′-acetyl-8-bromo-1-(4-methoxybenzyl)-2-oxo-2,3-dihydro-1H-benzo[b]azepine-4-carbohydrazide (1.13 g, 2.47 mmol, 1 equiv) was dissolved in 1,4-dioxane (22.6 mL) and phosphoryl chloride(1.5 mL, 16.20 mmol, 6.57 equiv) was added and the mixture was heated at 90° C. for 1 h, then cooled to room temperature and diluted with ethyl acetate. The mixture was washed with sated. NaHCO₃ and brine, dried over sodium sulfate, filtered and concentrated in vacuum. The residue was purified by silica gel column chromatography to get the product (910 mg) as a yellow solid. Yield: 83.83%; Purity: 98%.

TLC R_{f}=0.75 (DCM:MeOH=10:1);

LCMS (m/z)=440.58 (M+H)

¹H NMR (400 MHz, Chloroform-d) δ 7.56 (d, J=1.9 Hz, 1H), 7.52 (s, 1H), 7.35-7.29 (m, 1H), 7.20 (d, J=8.4 Hz, 1H), 7.00 (d, J=8.7 Hz, 2H), 6.80-6.72 (m, 2H), 5.05 (s, 2H), 4.41-3.06 (br, 5H, CH₃ and CH₂ overlapped, 3.75 (s, 3H)), 2.60 (s, 3H).

### Example 16. 1-(4-methoxybenzyl)-4-(5-methyl-1,3,4-oxadiazol-2-yl)-8-(1-methyl-1H-pyrazol-4-yl)-1,3-dihydro-2H-benzo[b]azepin-2-one (16)

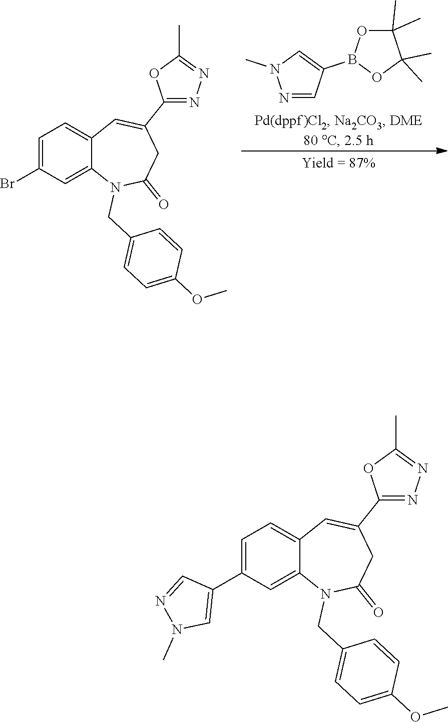

8-bromo-1-(4-methoxybenzyl)-4-(5-methyl-1,3,4-oxadiazol-2-yl)-1,3-dihydro-2H-benzo[b]azepin-2-one (810 mg, 1.84 mmol, 1 equiv) and 1-methyl-3-(4,4,5,5-tetramethyl-1,3,2-dioxaborolan-2-yl)-1H-pyrazole (765 mg, 3.68 mmol, 2 equiv) and Pd(dppf)Cl₂ (336 mg, 0.459 mmol, 0.25 equiv) were placed in a round-bottom flask, followed by DME (32.4 mL) and 2N Na₂CO₃ aqueous solution (16.2 mL). The solution was degassed and heated at 80° C. for 2.5 h. The solution was quenched with saturated ammonium chloride and extracted with EA (3×80 mL). The organic solutions were combined, washed with H₂O (2×20 mL) and brine (1×80 mL), dried over anhydrous Na₂SO₄, filtered and concentrated by rotary evaporation to get the crude compound. Then, the resulting concentrate was purified on a silica column to get 710 mg (purity=99%) white solid compound with a yield of 87.42%.

TLC R_{f}=0.1 (PE:EA=1:1);

LCMS (m/z)=442.50 (M+H)

### Example 17. 6-bromo-4-(4-methoxybenzyl)-1a-(5-methyloxazol-2-yl)-1a,2,4,8b-tetrahydrobenzo[b]cyclopropa[d]azepin-3(1H)-one (17)

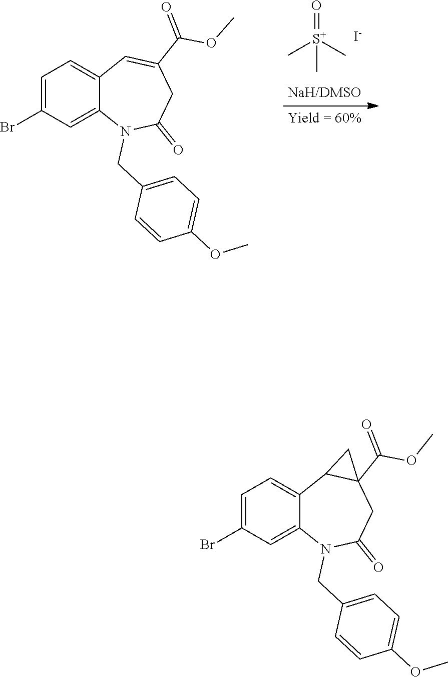

### methyl 6-bromo-4-(4-methoxybenzyl)-3-oxo-2,3,4,8b-tetrahydrobenzo[b]cyclopropa[d]azepine-1a(1H)-carboxylate

Trimethylsulfoxonium iodide (660 mg, 3.0 mmol, 3 equiv) was added to a suspension of NaH (123 mg in 60% oil dispersion, 3.1 mmol, 3.1 equiv) in DMSO (20 mL). The reaction mixture was stirred at rt for 15 min, after which a solution of methyl 8-bromo-1-(4-methoxybenzyl)-2-oxo-2,3-dihydro-1H-benzo[b]azepine-4-carboxylate (416 mg, 1.0 mmol, 1 equiv) in DMSO (10 mL) was added. The mixture was stirred at rt for 3 h. The mixture was quenched by the addition of H2O, upon which an off-white precipitate formed that was collected by filtration. Then, the resulting concentrate was purified on a silica column to get 260 mg (purity=97%) white solid compound with a yield of 60%.

TLC R_{f}=0.5 (PE:EA=1:1);

LCMS (m/z)=430.10 (M+H)

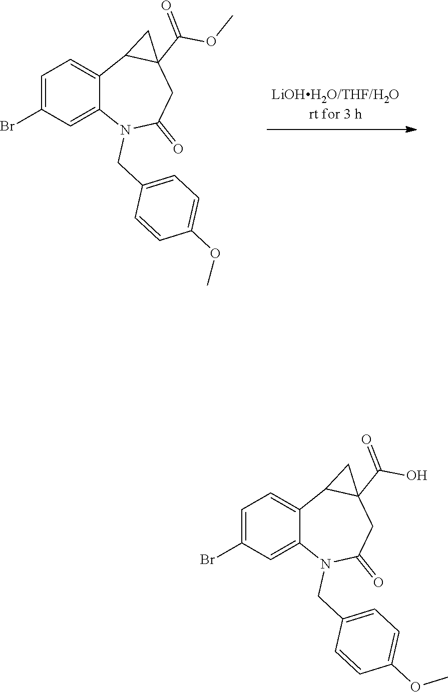 [SMILES: COC(=O)C12CC(=O)N(Cc3ccc(OC)cc3)c3cc(Br)ccc3C1C2 ; COc1ccc(CN2C(=O)CC3(C(=O)O)CC3c3ccc(Br)cc32)cc1]

### 6-bromo-4-(4-methoxybenzyl)-3-oxo-2,3,4,8b-tetrahydrobenzo[b]cyclopropa[d]azepine-1a(1H)-carboxylic acid

To a stirred solution of methyl 6-bromo-4-(3-fluoro-4-methoxybenzyl)-3-oxo-2,3,4,8b-tetrahydrobenzo[b]cyclopropa[d]azepine-1a(1H)-carboxylate (258 mg, 0.6 mmol, 1 equiv) in 1:1:1 mixture solvent MeOH/THF/H₂O (3 mL), was added LiOH·H₂O (125 mg, 3 mmol, 5 equiv) at ambient temperature. The reaction mixture was then stirred at rt for 3 h. The solution was quenched with 1N HCl and extracted with EA (3×100 mL). The organic solutions were combined, washed with H₂O (2×100 mL) and brine (1×200 mL), dried over anhydrous Na₂SO₄, filtered and concentrated by rotary evaporation to get the crude compound without purification 270 mg (purity=99%) white solid compound.

TLC R_{f}=0.2 (DCM:MeOH=10:1);

LCMS (m/z)=416.30 (M+H)

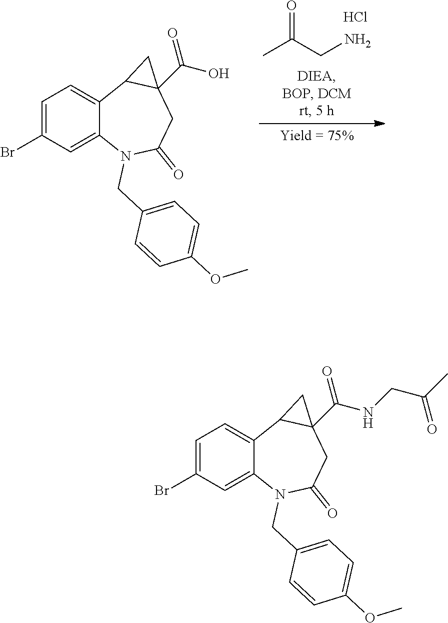

### 6-bromo-4-(4-methoxybenzyl)-3-oxo-N-(2-oxopropyl)-2,3,4,8b-tetrahydrobenzo[b]cyclopropa[d]azepine-1a(1H)-carboxamide

A mixture of 6-bromo-4-(4-methoxybenzyl)-3-oxo-2,3,4,8b-tetrahydrobenzo[b]cyclopropa[d]azepine-1a(1H)-carboxylic acid (270 mg, 0.648 mmol, 1 equiv), 1-aminoacetone HCl salt (106 mg, 0.97 mmol, 1.5 equiv), BOP reagent (430 mg, 0.97 mmol, 1.5 equiv), and DIEA (268 uL, 1.62 mmol, 2.5 equiv) in DCM (3 mL) was stirred for 3 h. The reaction mixture was purified on a silica column to get 230 mg (purity=99%) white solid compound with a yield of 75.23%.

TLC R_{f}=0.25 (PE:EA=1:2)

LCMS (m/z)=471.40 (M+H)

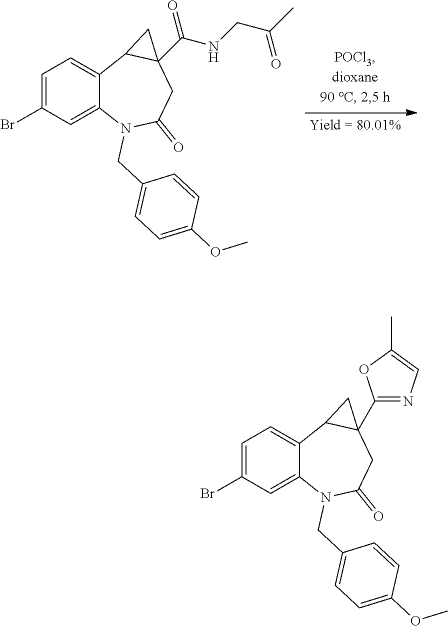 [SMILES: COc1ccc(CN2C(=O)CC3(C(=O)NCC(C)=O)CC3c3ccc(Br)cc32)cc1 ; COc1ccc(CN2C(=O)CC3(c4ncc(C)o4)CC3c3ccc(Br)cc32)cc1]

### 6-bromo-4-(4-methoxybenzyl)-la-(5-methyloxazol-2-yl)-1a,2,4,8b-tetrahydrobenzo[b]cyclopropa[d]azepin-3(1H)-one (17)

6-bromo-4-(4-methoxybenzyl)-3-oxo-N-(2-oxopropyl)-2,3,4,8b-tetrahydrobenzo[b]cyclopropa[d]azepine-1a(1H)-carboxamide (230 mg, 0.48 mmol, 1 equiv) was dissolved in 1,4-dioxane (5 mL) and phosphoryl chloride(298 uL, 3.2 mmol, 6.57 equiv) was added and the mixture was heated at 90° C. for 2.5 h, then cooled to room temperature and diluted with ethyl acetate. The mixture was washed with sated. NaHCO₃ and brine, dried over sodium sulfate, filtered and concentrated in vacuum. The residue was purified by silica gel column chromatography to get the product (177 mg) as a yellow solid. Yield: 80.01%; Purity: 97%.

TLC R_{f}=0.5 (PE:EA=1:1);

LCMS (m/z)=453.30 (M+H)

### Example 18. 1-(3-fluoro-4-methylbenzyl)-4-(5-methyloxazol-2-yl)-1,3-dihydro-2H-benzo[b]azepin-2-one (18)

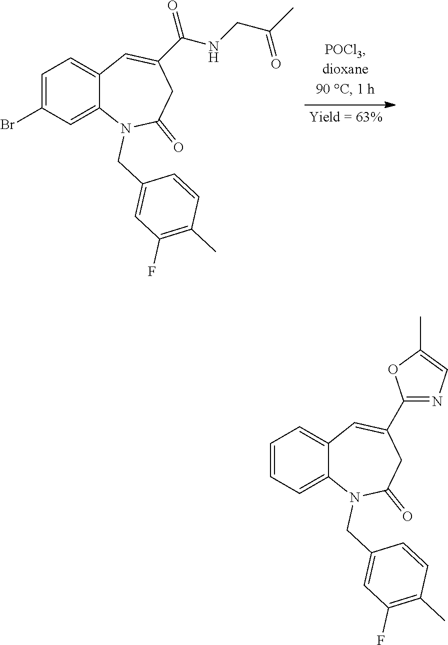 [SMILES: CC(=O)CNC(=O)C1=Cc2ccc(Br)cc2N(Cc2ccc(C)c(F)c2)C(=O)C1 ; Cc1cnc(C2=Cc3ccccc3N(Cc3ccc(C)c(F)c3)C(=O)C2)o1]

1-(3-fluoro-4-methylbenzyl)-2-oxo-N-(2-oxopropyl)-2,3-dihydro-1H-benzo[b]azepine-4-carboxamide (170 mg, 0.45 mmol, 1 equiv) was dissolved in 1,4-dioxane (4 mL) and phosphoryl chloride(274 uL, 2.94 mmol, 6.57 equiv) was added and the mixture was heated at 90° C. for 1 h, then cooled to room temperature and diluted with ethyl acetate. The mixture was washed with sated. NaHCO₃ and brine, dried over sodium sulfate, filtered and concentrated in vacuum. The residue was purified by silica gel column chromatography to get the product (130 mg) as a yellow solid. Yield: 80%; Purity: 99%.

TLC R_{f}=0.8 (PE:EA=1:1);

LCMS (m/z)=363.30 (M+H)

¹H NMR (400 MHz, Chloroform-d) δ 7.55 (s, 1H), 7.36 (dd, J=7.7, 1.6 Hz, 1H), 7.34-7.28 (m, 2H), 7.18 (ddd, J=7.7, 6.7, 1.8 Hz, 1H), 7.08-7.00 (m, 1H), 6.86 (q, J=1.2 Hz, 1H), 6.81-6.72 (m, 2H), 5.06 (s, 2H), 4.13-2.79 (br, 2H), 2.39 (d, J=1.3 Hz, 3H), 2.19 (d, J=1.9 Hz, 3H).

### Example 19. 8-bromo-1-(3-fluoro-4-methylbenzyl)-4-(5-methyloxazol-2-yl)-1,3-dihydro-2H-benzo[b]azepin-2-one (19)

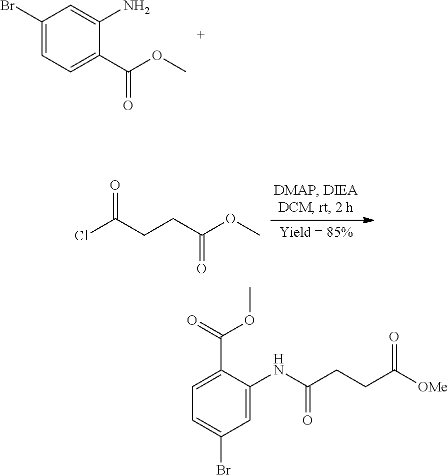

### methyl 4-bromo-2-(4-methoxy-4-oxobutanamido)benzoate

To a solution of methyl 2-amino-4-bromobenzoate (25 g, 108.67 mmol, 1 equiv) in DCM (326 mL), was added DIEA (28.4 mL, 163 mmol, 1.5 equiv) and DMAP (610 mg, 5.4 mmol, 0.5% equiv) under nitrogen. To this solution was added 3-(carbomethoxy) propionyl chloride (17.3 mL, 141.27 mmol, 1.3 equiv) dropwise over a period of 10 min, and the reaction mixture was allowed to stir at r.t. for 2 h. To the reaction mixture was then added H₂O (40 mL) and the mixture was allowed to stir for 30 min. The organic solution was separated, and the aqueous solution was extracted with DCM (3×100 mL). The organic solutions were combined, washed with H₂O (2×50 mL) and brine (1×100 mL), dried over anhydrous Na₂SO₄, filtered and concentrated by rotary evaporation to get the crude compound. Then, the resulting concentrate was purified on a silica column to get 31.9 g (purity=96%) of the light-yellow solid compound with a yield of 85.30%.

TLC R_{f}=0.8 (PE:EA=1:1);

LCMS (m/z)=343.90 (M+H);

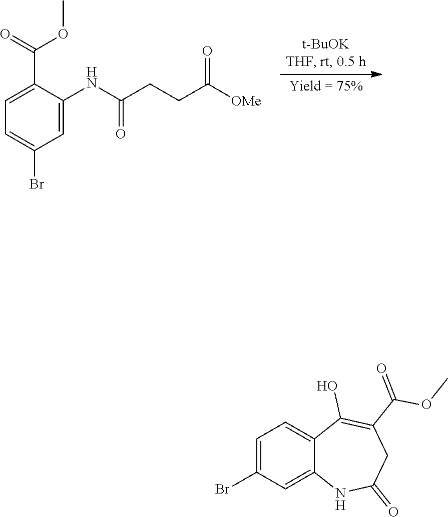 [SMILES: COC(=O)CCC(=O)Nc1cc(Br)ccc1C(=O)OC ; COC(=O)C1=C(O)c2ccc(Br)cc2NC(=O)C1]

### methyl 8-bromo-5-hydroxy-2-oxo-2,3-dihydro-1H-benzo[b]azepine-4-carboxylate

To a solution of methyl 4-bromo-2-(4-methoxy-4-oxobutanamido)benzoate (31.70 g, 92.1 mmol, 1 equiv) in THF (364 mL) at 5° C. was added a 1 M solution of t-BuOK in THF (276.3 mL, 276.3 mmol, 3 equiv) dropwise over 10 min while maintaining the temperature at 5° C. After 0.5 h, 200 mL of H₂O followed by 1N HCl were added to bring the solution to pH=4. The resulting mixture was allowed to stir at r.t. for 40 min. The organic solution was separated, and the aqueous solution was extracted with EtOAc (3×300 mL). The organic solutions were combined, dried over anhydrous Na₂SO₄, filtered and concentrated by rotary evaporation to afford crude compound. Then, the resulting concentrate was purified by Beating and purification (solvent EA, 50 mL) to get 21.6 g of the white solid compound with a yield of 75.14%. Purity=99%

TLC R_{f}=0.3 (PE:EA=4:1);

LCMS (m/z)=311.60 (M+H)

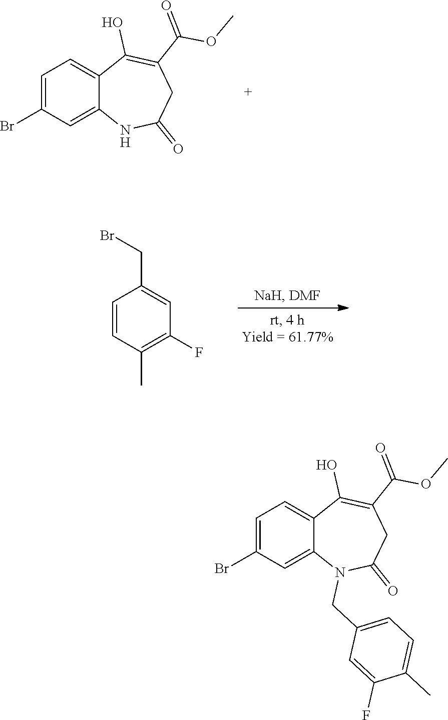

### methyl 8-bromo-1-(3-fluoro-4-methylbenzyl)-5-hydroxy-2-oxo-2,3-dihydro-1H-benzo[b]azepine-4-carboxylate

To a solution of methyl 9-bromo-5-hydroxy-2-oxo-2,3-dihydro-1H-benzo[b]azepine-4-carboxylate (6.4 g, 20.50 mmol, 1 equiv) in DMF (120 mL) was added NaH 60% dispersion in mineral oil (2.46 g, 61.51 mmol, 3 equiv). To this solution was added 4-(bromomethyl)-2-fluoro-1-methylbenzene (5.41 g, 26.66 mmol, 1.3 equiv), and the reaction mixture was allowed to stir at r.t for 4 h. The resulting solution was poured into water (120 mL) and extracted with Ethyl acetate (3×120 mL). The combined organic layers were washed with brine (1×120 mL), dried over anhydrous Na₂SO₄, and concentrated by rotary evaporation. Then, the resulting concentrate was 9U purified on a silica column to get 5.5 g of the white solid compound with a yield of 61.77%, purity=99%.

TLC R_{f}=0.75 (PE:EA=2:1)

LCMS (m/z)=434.27 (M+H)

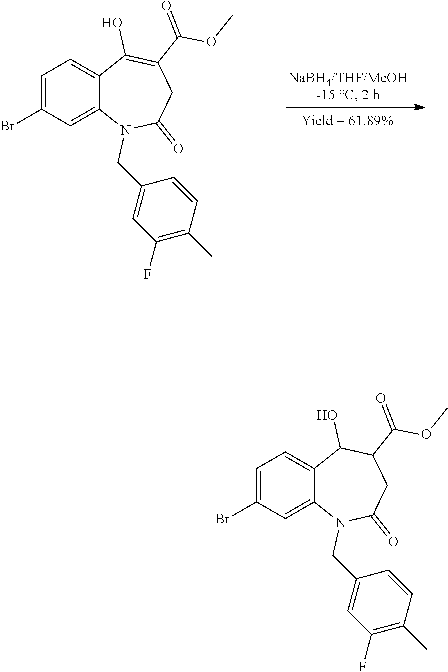 [SMILES: COC(=O)C1=C(O)c2ccc(Br)cc2N(Cc2ccc(C)c(F)c2)C(=O)C1 ; COC(=O)C1CC(=O)N(Cc2ccc(C)c(F)c2)c2cc(Br)ccc2C1O]

### methyl 8-bromo-1-(3-fluoro-4-methylbenzyl)-5-hydroxy-2-oxo-2,3,4,5-tetrahydro-1H-benzo[b]azepine-4-carboxylate

To a mixture of methyl 8-bromo-1-(3-fluoro-4-methylbenzyl)-5-hydroxy-2-oxo-2,3-dihydro-1H-benzo[b]azepine-4-carboxylate (2.17 g, 5 mmol, 1 equiv) in THF (50 ml) was added NaBH₄ (453 mg, 12 mmol, 2.4 equiv) and MeOH (5 ml) at −40° C. The mixture was stirred at −15° C. for 2 h. To the reaction mixture was added NH₄Cl aqueous solution (50 ml) and water at −15° C., and the mixture was extracted with EtOAc. The organic layer was washed with water and brine, dried over MgSO₄, and concentrated in vacuo. The residue was purified by silica gel column chromatography to get the product (1.35 g) as a white solid. Yield: 61.89%; Purity: 99%.

TLC R_{f}=0.55 (PE:EA=1:1);

LCMS (m/z)=437.80 (M+H)

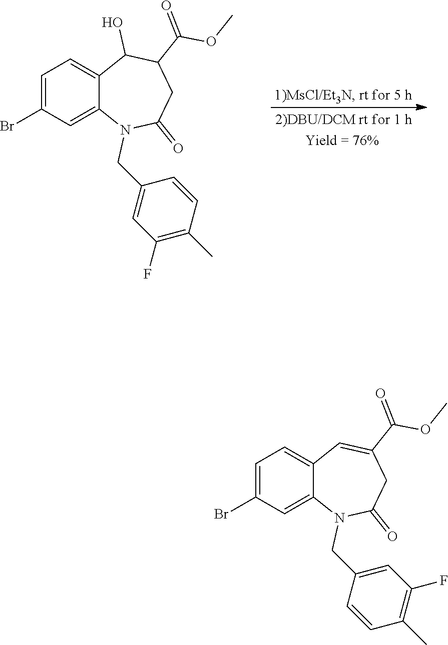 [SMILES: COC(=O)C1CC(=O)N(Cc2ccc(C)c(F)c2)c2cc(Br)ccc2C1O ; COC(=O)C1=Cc2ccc(Br)cc2N(Cc2ccc(C)c(F)c2)C(=O)C1]

### methyl 8-bromo-1-(3-fluoro-4-methylbenzyl)-2-oxo-2,3-dihydro-1H-benzo[b]azepine-4-carboxylate

Methanesulfonyl chloride (700 μL, 8 mmol, 2 equiv) was added to a solution of methyl 8-bromo-1-(3-fluoro-4-methylbenzyl)-5-hydroxy-2-oxo-2,3,4,5-tetrahydro-1H-benzo[b]azepine-4-carboxylate (1.75 g, 4 mmol, 1 equiv) and triethylamine (1.6 mL, 12 mmol, 3 equiv) in CH₂Cl₂ (14 mL) under ice cooling. The reaction mixture was stirred 5 h at r.t. Then DBU (2.4 mL, 16 mmol, 4 equiv) was added dropwise under ice cooling. The reaction mixture was stirred at rt for 1 h and then poured into water, 1 M HCl was added (pH=3) and the mixture was extracted with EA (2×300 mL). The combined organic layers were washed with water (300 mL) and brine (300 mL), dried (Na₂SO₄) and evaporated in vacuo to give the crude product, which was purified by silica gel column chromatography to get the product (1.28 g) as oil. Yield: 76.51%; Purity: 98%.

TLC R_{f}=0.5 (DMC:MeOH=20:1);

LCMS (m/z)=418.20 (M+H)

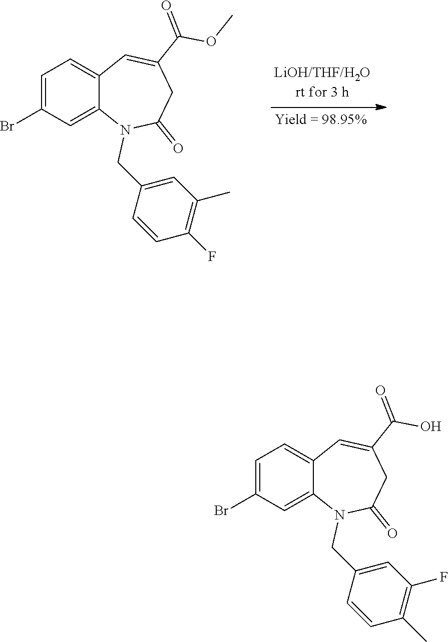 [SMILES: COC(=O)C1=Cc2ccc(Br)cc2N(Cc2ccc(F)c(C)c2)C(=O)C1 ; Cc1ccc(CN2C(=O)CC(C(=O)O)=Cc3ccc(Br)cc32)cc1F]

### 8-bromo-1-(3-fluoro-4-methylbenzyl)-2-oxo-2,3-dihydro-1H-benzo[b]azepine-4-caboxylic acid

To a stirred solution methyl 8-bromo-1-(3-fluoro-4-methylbenzyl)-2-oxo-2,3-dihydro-1H-benzo[b]azepine-4-carboxylate (1.25 g, 3 mmol, 1 equiv) in 1:1:1 mixture solvent MeOH/THF/H₂O (15 mL), was added LiOH·H₂O (629 mg, 15 mmol, 5 equiv) at ambient temperature. The reaction mixture was then stirred at rt for 3 h. The solution was quenched with 1N HCl and extracted with EA (3×50 mL). The organic solutions were combined, washed with H₂O (2×50 mL) and brine (100 mL), dried over anhydrous Na₂SO₄, filtered and concentrated by rotary evaporation to get the crude compound. Then, the resulting concentrate was purified on a silica column to get 1.2 g (purity=99%) white solid compound with a yield of 98.95%.

TLC R_{f}=0.2 (DCM:MeOH=10:1);

LCMS (m/z)=404.40 (M+H)

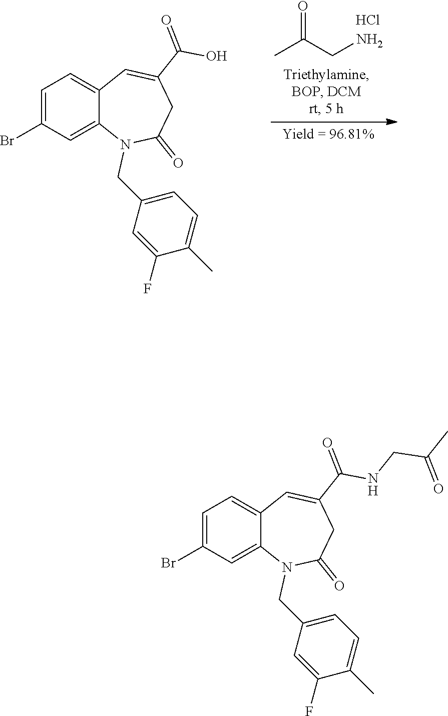

### 8-bromo-1-(3-fluoro-4-methylbenzyl)-2-oxo-N-(2-oxopropyl)-2,3-dihydro-1H-benzo[b]azepine-4-carboxamide

A mixture of 8-bromo-1-(3-fluoro-4-methylbenzyl)-2-oxo-2,3-dihydro-1H-benzo[b]azepine-4-carboxylic acid (780 mg, 1.93 mmol, 1 equiv), 1-aminoacetone HCl salt (317 mg, 2.89 mmol, 1.5 equiv), BOP reagent (1.28 g, 2.89 mmol, 1.5 equiv), and triethylamine (828 μL, 4.82 mmol, 2.5 equiv) in DCM (9.5 mL) was stirred for 3 h. The reaction mixture was purified on a silica column to get 858 mg (purity=99%) white solid compound with a yield of 96.81%.

TLC R_{f}=0.4 (PE:EA=1:2)

LCMS (m/z)=459.48 (M+H)

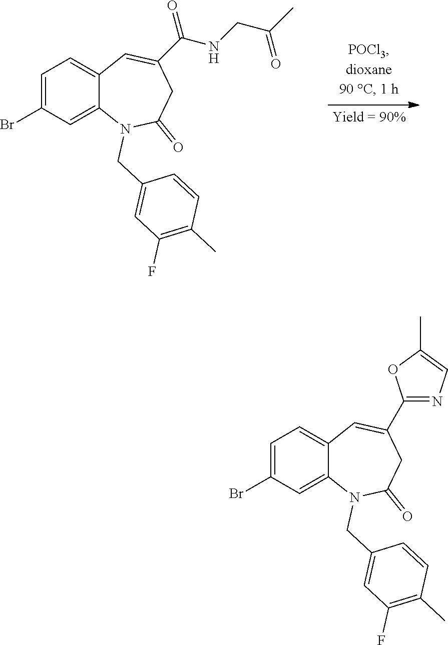 [SMILES: CC(=O)CNC(=O)C1=Cc2ccc(Br)cc2N(Cc2ccc(C)c(F)c2)C(=O)C1 ; Cc1cnc(C2=Cc3ccc(Br)cc3N(Cc3ccc(C)c(F)c3)C(=O)C2)o1]

### 8-bromo-1-(3-fluoro-4-methylbenzyl)-4-(5-methyloxazol-2-yl)-1,3-dihydro-2H-benzo[b]azepin-2-one (19)

8-bromo-1-(3-fluoro-4-methylbenzyl)-2-oxo-N-(2-oxopropyl)-2,3-dihydro-1H-benzo[b]azepine-4-carboxamide (780 mg, 1.7 mmol, 1 equiv) was dissolved in 1,4-dioxane (17 mL) and phosphoryl chloride(1 mL, 11 mmol, 6.57 equiv) was added and the mixture was heated at 90° C. for 1 h, then cooled to room temperature and diluted with ethyl acetate. The mixture was washed with sated. NaHCO₃ and brine, dried over sodium sulfate, filtered and concentrated in vacuum. The residue was purified by silica gel column chromatography to get the product (680 mg) as a yellow solid. Yield: 90.64%; Purity: 99%.

TLC R_{f}=0.8 (PE:EA=1:1);

LCMS (m/z)=441.45 (M+H)

¹H NMR (400 MHz, Chloroform-d) δ 7.50 (s, 1H), 7.48 (d, J=1.9 Hz, 1H), 7.30 (dd, J=8.3, 1.8 Hz, 1H), 7.22 (d, J=8.4 Hz, 1H), 7.05 (t, J=7.8 Hz, 1H), 6.87 (d, J=1.3 Hz, 1H), 6.79-6.70 (m, 2H), 5.05 (s, 2H), 4.22-2.91 (br, 2H), 2.39 (d, J=1.2 Hz, 3H), 2.20 (d, J=1.8 Hz, 3H).

### Example 20. 1-(3-fluoro-4-methylbenzyl)-8-(1-methyl-1H-pyrazol-4-yl)-4-(5-methyloxazol-2-yl)-1,3-dihydro-2H-benzo[b]azepin-2-one (20)

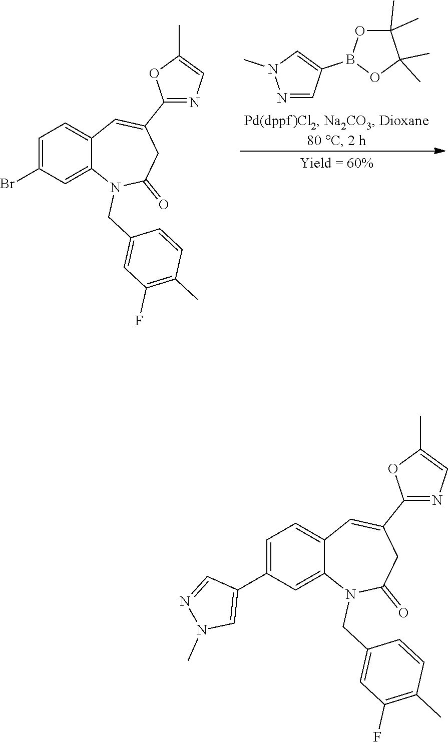

8-bromo-1-(3-fluoro-4-methylbenzyl)-4-(5-methyloxazol-2-yl)-1,3-dihydro-2H-benzo[b]azepin-2-one (132 mg, 0.3 mmol, 1 equiv) and 1-methyl-3-(4,4,5,5-tetramethyl-1,3,2-dioxaborolan-2-yl)-1H-pyrazole (124 mg, 0.6 mmol, 2 equiv) and Pd(dppf)Cl₂ (55 mg, 0.075 mmol, 0.25 equiv) were placed in a round-bottom flask, followed by 1,4-dioxane (15 mL) and 2N Na₂CO₃ aqueous solution (7.5 mL). The solution was degassed and heated at 80° C. for 2 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 80 mg (purity=98%) yellow solid compound with a yield of 60.26%.

TLC R_{f}=0.1 (PE:EA=3:1);

LCMS (m/z)=443.02 (M+H)

### Example 21. 8-(1-Cyclopropyl-1H-pyrazol-4-yl)-1-(3-fluoro-4-methylbenzyl)-4-(5-methyloxazol-2-yl)-1,3-dihydro-2H-benzo[b]azepin-2-one (21)

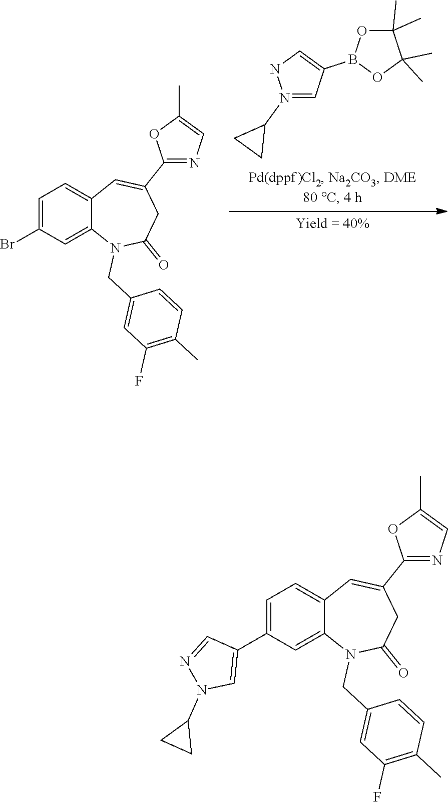

8-bromo-1-(3-fluoro-4-methylbenzyl)-4-(5-methyloxazol-2-yl)-1,3-dihydro-2H-benzo[b]azepin-2-one (100 mg, 0.22 mmol, 1 equiv) and 1-cyclopropyl-4-(4,4,5,5-tetramethyl-1,3,2-dioxaborolan-2-yl)-1H-pyrazole (93 uL, 0.45 mmol, 2 equiv) and Pd(dppf)Cl₂ (41.4 mg, 0.056 mmol, 0.25 equiv) were placed in a round-bottom flask, followed by DME (4.2 mL) and 2N Na₂CO₃ aqueous solution (2.1 mL). The solution was degassed and heated at 80° C. for 4 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 43 mg (purity=88%) yellow oil compound with a yield of 40.50%.

TLC R_{f}=0.4 (PE:EA=1:1);

LCMS (m/z)=469.40 (M+H)

### Example 22. 1-(3-fluoro-4-methylbenzyl)-4-(5-methyloxazol-2-yl)-8-(1H-pyrazol-4-yl)-1,3-dihydro-2H-benzo[b]azepin-2-one (22)

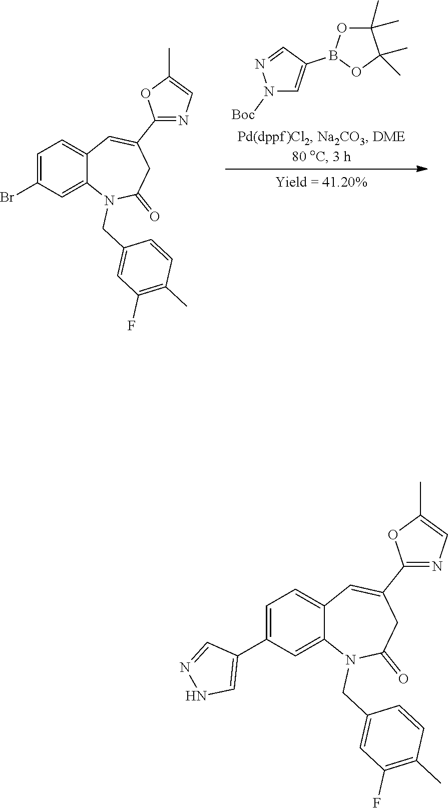 [SMILES: Cc1cnc(C2=Cc3ccc(Br)cc3N(Cc3ccc(C)c(F)c3)C(=O)C2)o1 ; CC(C)(C)OC(=O)n1cc(B2OC(C)(C)C(C)(C)O2)cn1 ; Cc1cnc(C2=Cc3ccc(-c4cn[nH]c4)cc3N(Cc3ccc(C)c(F)c3)C(=O)C2)o1]

8-bromo-1-(3-fluoro-4-methylbenzyl)-4-(5-methyloxazol-2-yl)-1,3-dihydro-2H-benzo[b]azepin-2-one (100 mg, 0.22 mmol, 1 equiv) and tert-butyl 4-(4,4,5,5-tetramethyl-1,3,2-dioxaborolan-2-yl)-1H-pyrazole-1-carboxylate (133 mg, 0.45 mmol, 2 equiv) and Pd(dppf)Cl₂ (41.4 mg, 0.056 mmol, 0.25 equiv) were placed in a round-bottom flask, followed by DME (4.2 mL) and 2N Na₂CO₃ aqueous solution (2.1 mL). The solution was degassed and heated at 80° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 40 mg (purity=99%) yellow solid compound with a yield of 41.20%.

TLC R_{f}=0.25 (PE:EA=1:1);

LCMS (m/z)=429.30 (M+H)

### Example 23. 1-(3-fluoro-4-methylbenzyl)-4-(5-methyloxazol-2-yl)-8-(pyridin-3-yl)-1,3-dihydro-2H-benzo[b]azepin-2-one (23)

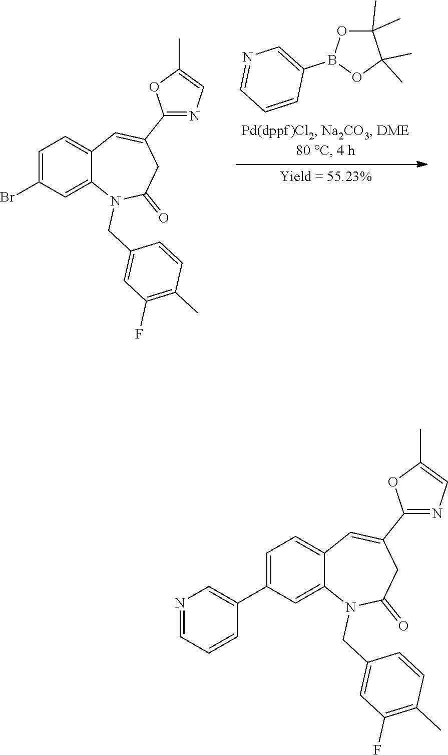

8-bromo-1-(3-fluoro-4-methylbenzyl)-4-(5-methyloxazol-2-yl)-1,3-dihydro-2H-benzo[b]azepin-2-one (100 mg, 0.22 mmol, 1 equiv) and 3-(4,4,5,5-tetramethyl-1,3,2-dioxaborolan-2-yl)pyridine (92 mg, 0.45 mmol, 2 equiv) and Pd(dppf)Cl₂ (41.4 mg, 0.056 mmol, 0.25 equiv) were placed in a round-bottom flask, followed by DME (4.2 mL) and 2N Na₂CO₃ aqueous solution (2.1 mL). The solution was degassed and heated at 80° C. for 4 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 55 mg (purity=99%) yellow oil compound with a yield of 55.23%.

TLC R_{f}=0.2 (PE:EA=1:1);

LCMS (m/z)=440.10 (M+H)

### Example 24. 1-(4-methoxybenzyl)-4-(5-methyloxazol-2-yl)-1,3-dihydro-2H-benzo[b]azepin-2-one (24)

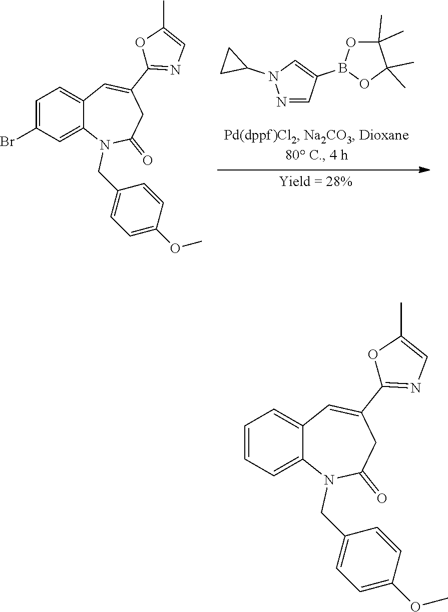

8-bromo-1-(4-methoxybenzyl)-4-(5-methyloxazol-2-yl)-1,3-dihydro-2H-benzo[b]azepin-2-one (200 mg, 0.46 mmol, 1 equiv) and 1-methyl-3-(4,4,5,5-tetramethyl-1,3,2-dioxaborolan-2-yl)-1H-pyrazole (184 uL, 0.91 mmol, 2 equiv) and Pd(dppf)Cl2 (83 mg, 0.12 mmol, 0.25 equiv) were placed in a round-bottom flask, followed by 1,4-dioxane (8.4 mL) and 2N Na₂CO₃ aqueous solution (4.2 mL). The solution was degassed and heated at 80° C. for 4 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 60 mg (purity=90%) yellow solid compound with a yield of 28%.

TLC R_{f}=0.2 (PE:EA=1:1);

LCMS (m/z)=361.10 (M+H)

### Example 25. 8-bromo-1-(4-methoxybenzyl)-4-(5-methyloxazol-2-yl)-1,3-dihydro-2H-benzo[b]azepin-2-one (25)

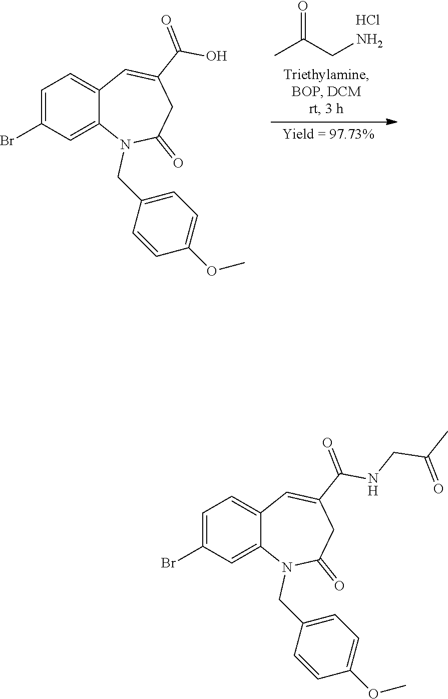

### 8-bromo-1-(4-methoxybenzyl)-2-oxo-N-(2-oxopropyl)-2,3-dihydro-1H-benzo[b]azepine-4-carboxamide

A mixture of 8-bromo-1-(4-methoxybenzyl)-2-oxo-2,3-dihydro-1H-benzo[b]azepine-4-carboxylic acid (1.26 g, 3.13 mmol, 1 equiv), 1-aminoacetone HCl salt (514 mg, 4.7 mmol, 1.5 equiv), BOP reagent (2.08 g, 4.7 mmol, 1.5 equiv), and triethylamine (1.37 mL, 7.83 mmol, 2.5 equiv) in DCM (15.7 mL) was stirred for 3 h. The reaction mixture was purified on a silica column to get 1.4 g (purity=99%) white solid compound with a yield of 97.73%.

TLC R_{f}=0.6 (DCM:MeOH=10:1)

LCMS (m/z)=457.40 (M+H)

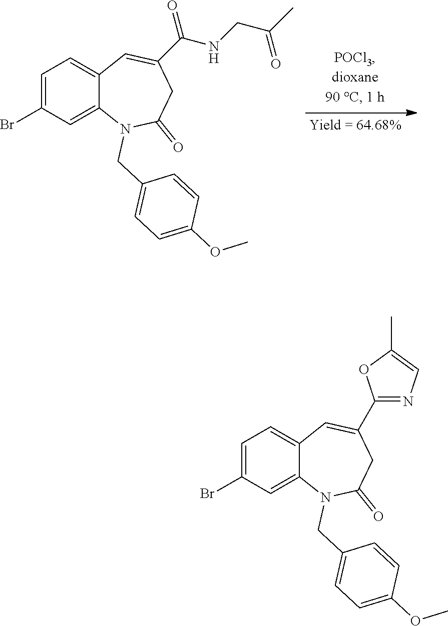 [SMILES: COc1ccc(CN2C(=O)CC(C(=O)NCC(C)=O)=Cc3ccc(Br)cc32)cc1 ; COc1ccc(CN2C(=O)CC(c3ncc(C)o3)=Cc3ccc(Br)cc32)cc1]

### 8-bromo-1-(4-methoxybenzyl)-4-(5-methyloxazol-2-yl)-1,3-dihydro-2H-benzo[b]azepin-2-one (25)

8-bromo-1-(4-methoxybenzyl)-2-oxo-N-(2-oxopropyl)-2,3-dihydro-1H-benzo[b]azepine-4-carboxamide (1.4 g, 3.05 mmol, 1 equiv) was dissolved in 1,4-dioxane (28 mL) and phosphoryl chloride(1.9 mL, 20.03 mmol, 6.57 equiv) was added and the mixture was heated at 90° C. for 1 h, then cooled to room temperature and diluted with ethyl acetate. The mixture was washed with sated. NaHCO₃ and brine, dried over sodium sulfate, filtered and concentrated in vacuum. The residue was purified by silica gel column chromatography to get the product (870 mg) as a yellow solid. Yield: 64.68%; Purity: 99%.

TLC R_{f}=0.8 (PE:EA=1:1);

LCMS (m/z)=439.25 (M+H)

### Example 26. 8-amino-1-(4-methoxybenzyl)-4-(5-methyloxazol-2-yl)-1,3-dihydro-2H-benzo[b]azepin-2-one (26)

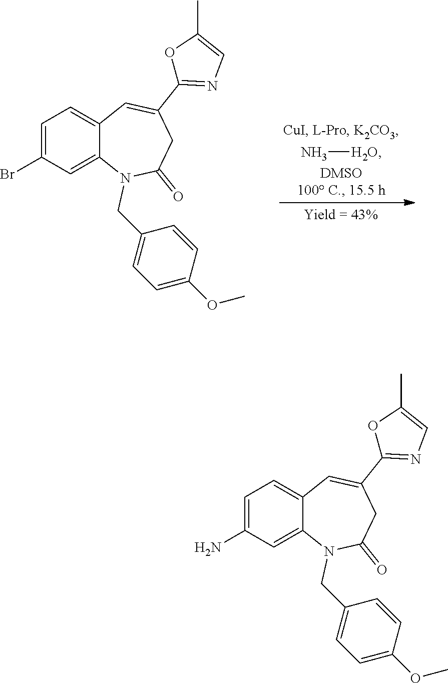

8-bromo-1-(4-methoxybenzyl)-4-(5-methyloxazol-2-yl)-1,3-dihydro-2H-benzo[b]azepin-2-one (100 mg, 0.22 mmol, 1 equiv) and CuI (8 mg, 0.045 mmol, 0.2 equiv) and L-Proline (10 mg, 0.091 mmol, 0.4 equiv) and K₂CO₃ (94 mg, 0.68 mmol, 3 equiv) were placed in a round-bottom flask, followed by DMSO (0.92 mL) and Ammonium hydroxide (0.23 mL). The solution was degassed and heated at 100° C. for 15.5 h. The solution was quenched with saturated ammonium chloride and extracted with EA (3×30 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 37 mg (purity=96%) yellow solid compound with a yield of 43.30%.

TLC R_{f}=0.15 (PE:EA=1:1);

LCMS (m/z)=376.40 (M+H)

### Example 27. 8-hydroxy-1-(4-methoxybenzyl)-4-(5-methyloxazol-2-yl)-1,3-dihydro-2H-benzo[b]azepin-2-one (27)

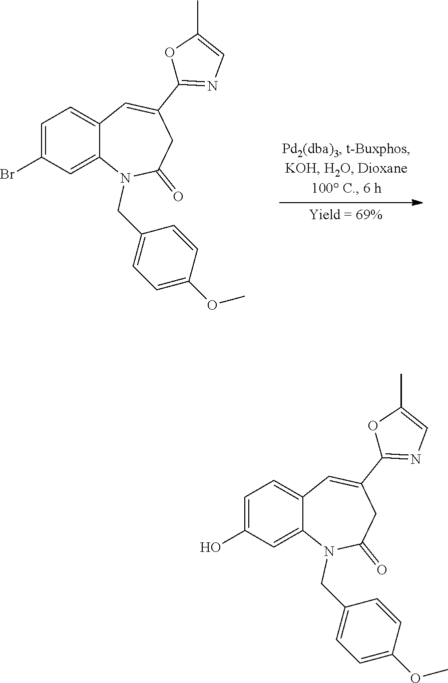 [SMILES: COc1ccc(CN2C(=O)CC(c3ncc(C)o3)=Cc3ccc(Br)cc32)cc1 ; COc1ccc(CN2C(=O)CC(c3ncc(C)o3)=Cc3ccc(O)cc32)cc1]

A mixture of 8-bromo-1-(4-methoxybenzyl)-4-(5-methyloxazol-2-yl)-1,3-dihydro-2H-benzo[b]azepin-2-one (88 mg, 0.2 mmol, 1 equiv), Pd₂(dba)₃ (15.5 mg, 0.017 mmol, 0.085 equiv), t-Buxphos (16.19 mg, 0.0382 mmol, 0.191 equiv), potassium hydroxide (28.78 mg, 0.514 mmol, 2.57 equiv), 1,4-dioxane (5 mL), and water (0.5 mL)was heated under nitrogen at 100° C. for 6 h. The reaction mixture was cooled to room temperature, filtered through Celite, and evaporated in vacuo. Then, the resulting concentrate was purified on a silica column to get 52 mg (purity=93.9%) yellow solid compound with a yield of 69%.

TLC R_{f}=0.5 (DCM:MeOH=10:1);

LCMS (m/z)=376.90 (M+H)

### Example 28. 1-(4-methoxybenzyl)-4-(5-methyloxazol-2-yl)-8-(thiophen-3-yl)-1,3-dihydro-2H-benzo[b]azepin-2-one (28)

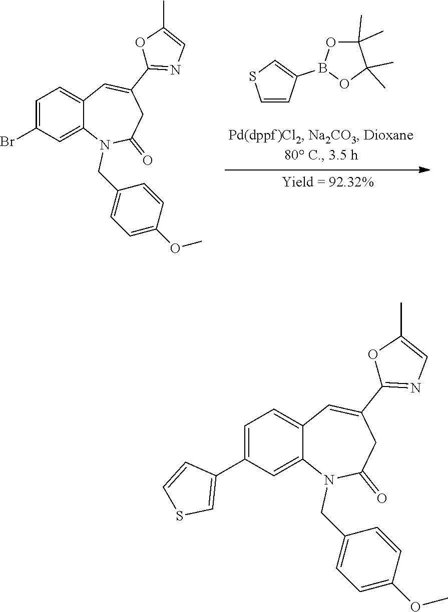 [SMILES: COc1ccc(CN2C(=O)CC(c3ncc(C)o3)=Cc3ccc(Br)cc32)cc1 ; CC1(C)OB(c2ccsc2)OC1(C)C ; COc1ccc(CN2C(=O)CC(c3ncc(C)o3)=Cc3ccc(-c4ccsc4)cc32)cc1]

8-bromo-1-(4-methoxybenzyl)-4-(5-methyloxazol-2-yl)-1,3-dihydro-2H-benzo[b]azepin-2-one (100 mg, 0.22 mmol, 1 equiv) and 4,4,5,5-tetramethyl-2-(thiophen-3-yl)-1,3,2-dioxaborolane (95 mg, 0.45 mmol, 2 equiv) and Pd(dppf)Cl₂ (41.6 mg, 0.056 mol, 0.25 equiv) were placed in a round-bottom flask, followed by 1,4-dioxane (4.2 mL) and 2N Na₂CO₃ aqueous solution (2.1 mL). The solution was degassed and heated at 80° C. for 3.5 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 93 mg (purity=84%) yellow solid compound with a yield of 92.32%.

TLC R_{f}=0.55 (PE:EA=1:1);

LCMS (m/z)=443.30 (M+H)

### Example 29. 8-(furan-3-yl)-1-(4-methoxybenzyl)-4-(5-methyloxazol-2-yl)-1,3-dihydro-2H-benzo[b]azepin-2-one (29)

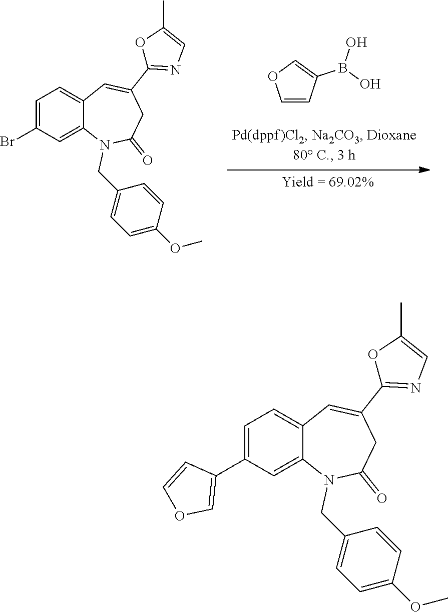 [SMILES: COc1ccc(CN2C(=O)CC(c3ncc(C)o3)=Cc3ccc(Br)cc32)cc1 ; OB(O)c1ccoc1 ; COc1ccc(CN2C(=O)CC(c3ncc(C)o3)=Cc3ccc(-c4ccoc4)cc32)cc1]

8-bromo-1-(4-methoxybenzyl)-4-(5-methyloxazol-2-yl)-1,3-dihydro-2H-benzo[b]azepin-2-one (100 mg, 0.22 mmol, 1 equiv) and furan-3-ylboronic acid (88 mg, 0.45 mmol, 2 equiv) and Pd(dppf)Cl₂ (41.6 mg, 0.057 mol, 0.25 equiv) were placed in a round-bottom flask, followed by 1,4-dioxane (4.2 mL) and 2N Na₂CO₃ aqueous solution (2.1 mL). The solution was degassed and heated at 80° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 67 mg (purity=93%) white solid compound with a yield of 69.02%.

TLC R_{f}=0.2 (PE:EA=1:1);

LCMS (m/z)=427.30 (M+H)

¹H NMR (400 MHz, Chloroform-d) δ 7.67 (t, J=1.2 Hz, 1H), 7.52 (s, 1H), 7.47 (t, J=1.7 Hz, 1H), 7.46 (d, J=1.7 Hz, 1H), 7.34 (d, J=8.1 Hz, 1H), 7.28 (dd, J=8.2, 1.7 Hz, 1H), 7.09 (d, J=8.7 Hz, 2H), 6.86 (d, J=1.4 Hz, 1H), 6.79 (d, J=8.7 Hz, 2H), 6.57 (dd, J=1.9, 0.9 Hz, 1H), 5.07 (s, 2H), 4.57-2.74 (br, 5H, CH₃ and CH₂ overlapped, 3.76 (s, 3H)), 2.39 (d, J=1.2 Hz, 3H).

### Example 30. 1-(4-methoxybenzyl)-8-(1-methyl-1H-pyrazol-4-yl)-4-(5-methyloxazol-2-yl)-1,3-dihydro-2H-benzo[b]azepin-2-one (Compound 30)

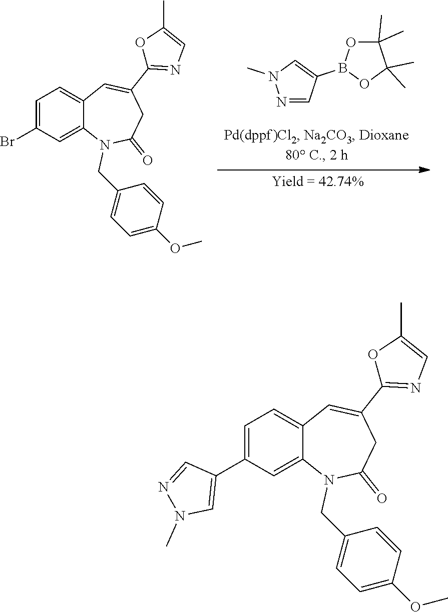

8-bromo-1-(4-methoxybenzyl)-4-(5-methyloxazol-2-yl)-1,3-dihydro-2H-benzo[b]azepin-2-one (70 mg, 0.16 mmol, 1 equiv) and 1-methyl-3-(4,4,5,5-tetramethyl-1,3,2-dioxaborolan-2-yl)-1H-pyrazole (66 mg, 0.31 mmol, 2 equiv) and Pd(dppf)Cl₂ (29 mg, 0.04 mmol, 0.25 equiv) were placed in a round-bottom flask, followed by 1,4-dioxane (3 mL) and 2N Na₂CO₃ aqueous solution (1.5 mL). The solution was degassed and heated at 80° C. for 2 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 30 mg (purity=98%) yellow solid compound with a yield of 42.74%.

TLC R_{f}=0.15 (PE:EA=1:1);

LCMS (m/z)=441.52 (M+H)

### Example 31. 1-(4-methoxybenzyl)-8-(1-methyl-1H-pyrazol-3-yl)-4-(5-methyloxazol-2-yl)-1,3-dihydro-2H-benzo[b]azepin-2-one (31)

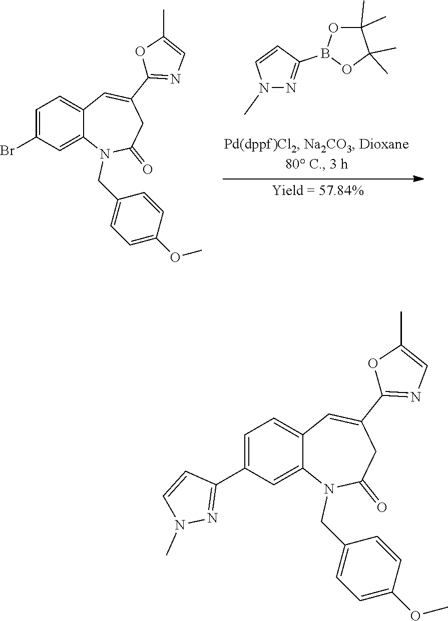

8-bromo-1-(4-methoxybenzyl)-4-(5-methyloxazol-2-yl)-1,3-dihydro-2H-benzo[b]azepin-2-one (150 mg, 0.34 mmol, 1 equiv) and 1-methyl-3-(4,4,5,5-tetramethyl-1,3,2-dioxaborolan-2-yl)-1H-pyrazole (142 mg, 0.68 mmol, 2 equiv) and Pd(dppf)Cl₂ (62 mg, 0.09 mmol, 0.25 equiv) were placed in a round-bottom flask, followed by 1,4-dioxane (7.8 mL) and 2N Na₂CO₃ aqueous solution (3.9 mL). The solution was degassed and heated at 80° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 87 mg (purity=83%) white solid compound with a yield of 57.84%.

TLC R_{f}=0.25 (DCM:MeOH=10:1);

LCMS (m/z)=441.40 (M+H)

¹H NMR (400 MHz, Chloroform-d) δ 7.66 (s, 1H), 7.53 (s, 1H), 7.50 (s, 1H), 7.44 (d, J=1.7 Hz, 1H), 7.32 (d, J=8.1 Hz, 1H), 7.24 (d, J=1.6 Hz, 1H), 7.08 (d, J=8.6 Hz, 2H), 6.85 (d, J=1.3 Hz, 1H), 6.82-6.74 (m, 2H), 5.07 (s, 2H), 4.52-2.73 (br, 8H, CH₃ and CH₂ overlapped, 3.94 (s, 3H), 3.76 (s, 3H)), 2.38 (d, J=1.1 Hz, 3H).

### Example 32. 1-(4-methoxybenzyl)-8-(1-methyl-1H-imidazol-5-yl)-4-(5-methyloxazol-2-yl)-1,3-dihydro-2H-benzo[b]azepin-2-one (32)

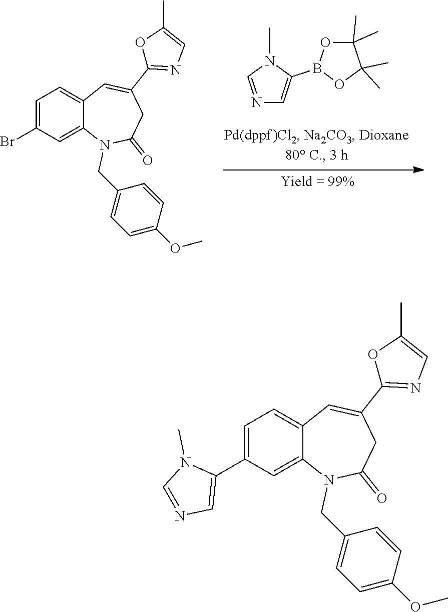 [SMILES: COc1ccc(CN2C(=O)CC(c3ncc(C)o3)=Cc3ccc(Br)cc32)cc1 ; Cn1cncc1B1OC(C)(C)C(C)(C)O1 ; COc1ccc(CN2C(=O)CC(c3ncc(C)o3)=Cc3ccc(-c4cncn4C)cc32)cc1]

8-bromo-1-(4-methoxybenzyl)-4-(5-methyloxazol-2-yl)-1,3-dihydro-2H-benzo[b]azepin-2-one (150 mg, 0.34 mmol, 1 equiv) and 1-methyl-5-(4,4,5,5-tetramethyl-1,3,2-dioxaborolan-2-yl)-1H-imidazole (142 mg, 0.68 mmol, 2 equiv) and Pd(dppf)Cl₂ (62 mg, 0.09 mmol, 0.25 equiv) were placed in a round-bottom flask, followed by 1,4-dioxane (7.8 mL) and 2N Na₂CO₃ aqueous solution (3.9 mL). The solution was degassed and heated at 80° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 150 mg (purity=94%) yellow solid compound with a yield of 99%.

TLC R_{f}=0.4 (DCM:MeOH=10:1);

LCMS (m/z)=441.60 (M+H)

### Example 33. 1-(4-methoxybenzyl)-4-(5-methyloxazol-2-yl)-8-(1H-pyrazol-4-yl)-1,3-dihydro-2H-benzo[b]azepin-2-one (33)

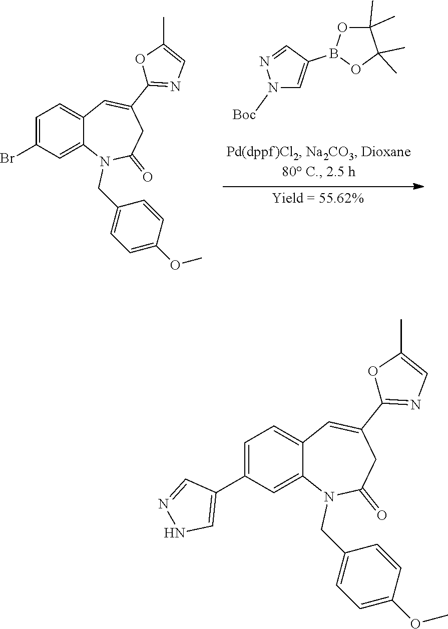 [SMILES: COc1ccc(CN2C(=O)CC(c3ncc(C)o3)=Cc3ccc(Br)cc32)cc1 ; CC(C)(C)OC(=O)n1cc(B2OC(C)(C)C(C)(C)O2)cn1 ; COc1ccc(CN2C(=O)CC(c3ncc(C)o3)=Cc3ccc(-c4cn[nH]c4)cc32)cc1]

8-bromo-1-(4-methoxybenzyl)-4-(5-methyloxazol-2-yl)-1,3-dihydro-2H-benzo[b]azepin-2-one (100 mg, 0.23 mmol, 1 equiv) and tert-butyl 4-(4,4,5,5-tetramethyl-1,3,2-dioxaborolan-2-yl)-1H-pyrazole-1-carboxylate (133 mg, 0.45 mmol, 2 equiv) and Pd(dppf)Cl₂ (41.3 mg, 0.057 mmol, 0.25 equiv) were placed in a round-bottom flask, followed by 1,4-dioxane (4.2 mL) and 2N Na₂CO₃ aqueous solution (2.1 mL). The solution was degassed and heated at 80° C. for 2.5 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 54 mg (purity=99%) yellow solid compound with a yield of 55.62%.

TLC R_{f}=0.1 (PE:EA=1:1);

LCMS (m/z)=427.40 (M+H)

### Example 34. 8-(1-cyclopropyl-1H-pyrazol-4-yl)-1-(4-methoxybenzyl)-4-(5-methyloxazol-2-yl)-1,3-dihydro-2H-benzo[b]azepin-2-one (34)

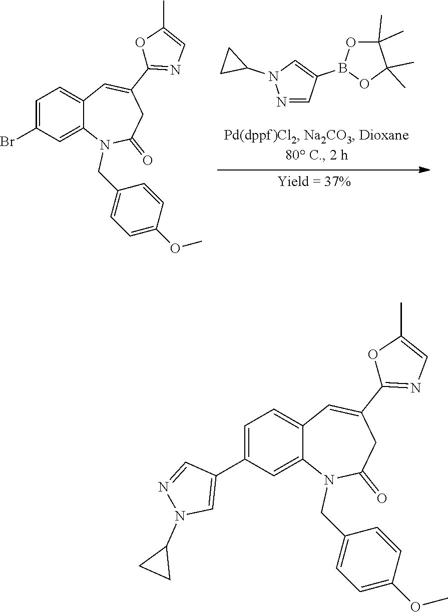

8-bromo-1-(4-methoxybenzyl)-4-(5-methyloxazol-2-yl)-1,3-dihydro-2H-benzo[b]azepin-2-one (100 mg, 0.23 mmol, 1 equiv) and 1-methyl-3-(4,4,5,5-tetramethyl-1,3,2-dioxaborolan-2-yl)-1H-pyrazole (93 uL, 0.46 mmol, 2 equiv) and Pd(dppf)Cl₂ (41.3 mg, 0.057 mmol, 0.25 equiv) were placed in a round-bottom flask, followed by 1,4-dioxane (4.2 mL) and 2N Na₂CO₃ aqueous solution (2.1 mL). The solution was degassed and heated at 80° C. for 2 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 40 mg (purity=89%) yellow solid compound with a yield of 37.67%.

TLC R_{f}=0.15 (PE:EA=1:1);

LCMS (m/z)=467.40 (M+H)

### Example 35. 1-(4-methoxybenzyl)-4-(5-methyloxazol-2-yl)-8-(1-(piperidin-4-yl)-1H-pyrazol-4-yl)-1,3-dihydro-2H-benzo[b]azepin-2-one (35)

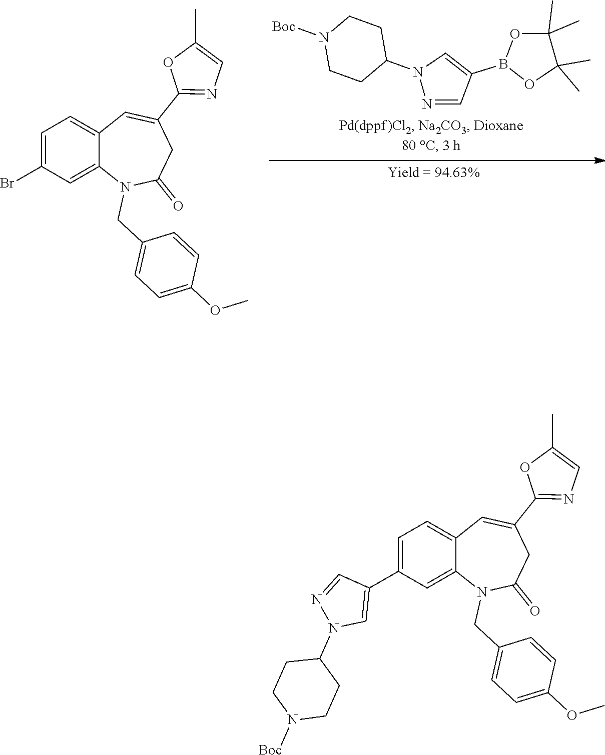 [SMILES: COc1ccc(CN2C(=O)CC(c3ncc(C)o3)=Cc3ccc(Br)cc32)cc1 ; CC(C)(C)OC(=O)N1CCC(n2cc(B3OC(C)(C)C(C)(C)O3)cn2)CC1 ; COc1ccc(CN2C(=O)CC(c3ncc(C)o3)=Cc3ccc(-c4cnn(C5CCN(C(=O)OC(C)(C)C)CC5)c4)cc32)cc1]

### tert-butyl 4-(4-(1-(4-methoxybenzyl)-4-(5-methyloxazol-2-yl)-2-oxo-2,3-dihydro-1H-benzo[b]azepin-8-yl)-1H-pyrazol-1-yl)piperidine-1-carboxylate

8-bromo-1-(4-methoxybenzyl)-4-(5-methyloxazol-2-yl)-1,3-dihydro-2H-benzo[b]azepin-2-one (150 mg, 0.34 mmol, 1 equiv) and tert-butyl 4-(4-(4,4,5,5-tetramethyl-1,3,2-dioxaborolan-2-yl)-1H-pyrazol-1-yl)piperidine-1-carboxylate (257 mg, 0.68 mmol, 2 equiv) and Pd(dppf)Cl₂ (62 mg, 0.09 mmol, 0.25 equiv) were placed in a round-bottom flask, followed by 1,4-dioxane (7.8 mL) and 2N Na₂CO₃ aqueous solution (3.9 mL). The solution was degassed and heated at 80° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 197 mg (purity=92%) black solid compound with a yield of 94.63%.

TLC R_{f}=0.15 (DCM:EA=2:1);

LCMS (m/z)=610.80 (M+H)

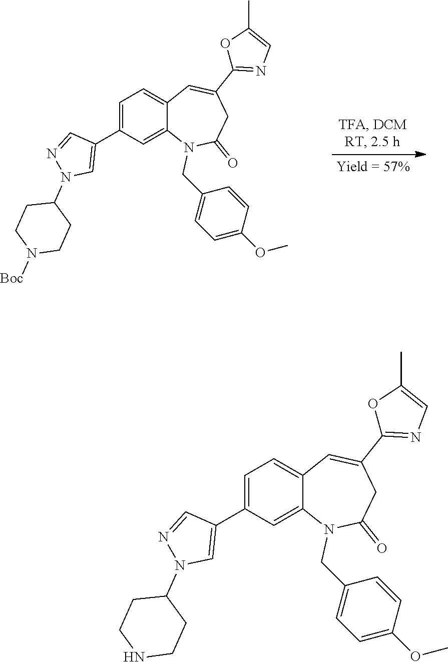 [SMILES: COc1ccc(CN2C(=O)CC(c3ncc(C)o3)=Cc3ccc(-c4cnn(C5CCN(C(=O)OC(C)(C)C)CC5)c4)cc32)cc1 ; COc1ccc(CN2C(=O)CC(c3ncc(C)o3)=Cc3ccc(-c4cnn(C5CCNCC5)c4)cc32)cc1]

### 1-(4-methoxybenzyl)-4-(5-methyloxazol-2-yl)-8-(1-(piperidin-4-yl)-1H-pyrazol-4-yl)-1,3-dihydro-2H-benzo[b]azepin-2-one (35)

tert-butyl 4-(4-(1-(4-methoxybenzyl)-4-(5-methyloxazol-2-yl)-2-oxo-2,3-dihydro-1H-benzo[b]azepin-8-yl)-1H-pyrazol-1-yl)piperidine-1-carboxylate (197 mg, 0.32 mmol, 1 equiv) were placed in a round-bottom flask, followed by DCM (3 mL) and TFA (1.5 mL). The solution stirred at room temperature for 3 h. The solution was quenched with saturated ammonium chloride and extracted with DCM (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 93 mg (purity=99%) white solid compound with a yield of 57%.

TLC R_{f}=0.1 (DCM:MeOH=10:1);

LCMS (m/z)=510.5 (M+H)

### Example 36. 8-(1-(2-hydroxyethyl)-1H-pyrazol-4-yl)-1-(4-methoxybenzyl)-4-(5-methyloxazol-2-yl)-1,3-dihydro-2H-benzo[b]azepin-2-one (36)

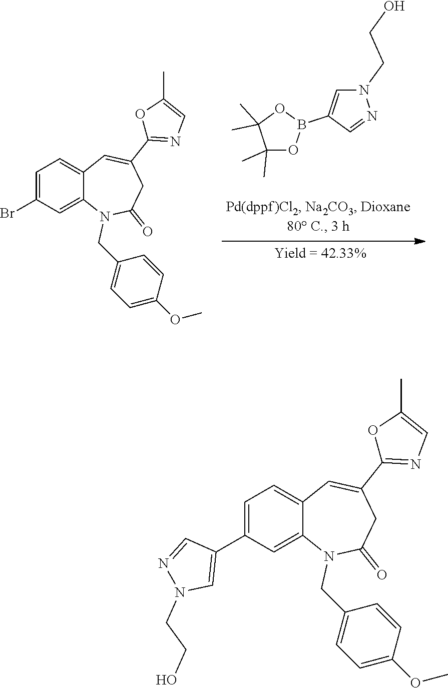 [SMILES: COc1ccc(CN2C(=O)CC(c3ncc(C)o3)=Cc3ccc(Br)cc32)cc1 ; CC1(C)OB(c2cnn(CCO)c2)OC1(C)C ; COc1ccc(CN2C(=O)CC(c3ncc(C)o3)=Cc3ccc(-c4cnn(CCO)c4)cc32)cc1]

8-bromo-1-(4-methoxybenzyl)-4-(5-methyloxazol-2-yl)-1,3-dihydro-2H-benzo[b]azepin-2-one (150 mg, 0.34 mmol, 1 equiv) and 2-(4-(4,4,5,5-tetramethyl-1,3,2-dioxaborolan-2-yl)-1H-pyrazol-1-yl)ethan-1-ol (162 mg, 0.68 mmol, 2 equiv) and Pd(dppf)Cl₂ (62 mg, 0.09 mmol, 0.25 equiv) were placed in a round-bottom flask, followed by 1,4-dioxane (7.8 mL) and 2N Na₂CO₃ aqueous solution (3.9 mL). The solution was degassed and heated at 80° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 68 mg (purity=99%) white solid compound with a yield of 42.33%.

TLC R_{f}=0.5 (DCM:MeOH=10:1);

LCMS (m/z)=471.50 (M+H)

### Example 37. 8-(1-(2-hydroxy-2-methylpropyl)-1H-pyrazol-4-yl)-1-(4-methoxybenzyl)-4-(5-methyloxazol-2-yl)-1,3-dihydro-2H-benzo[b]azepin-2-one (37)

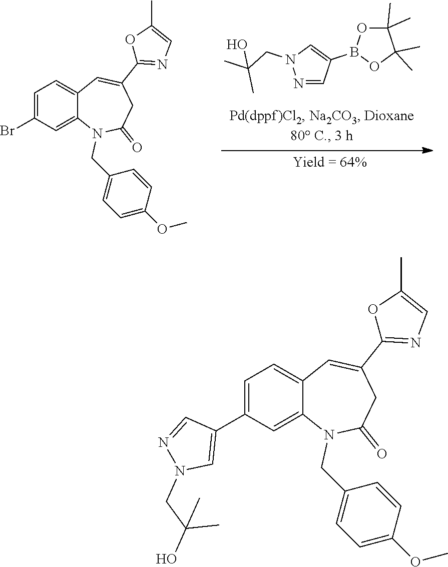

8-bromo-1-(4-methoxybenzyl)-4-(5-methyloxazol-2-yl)-1,3-dihydro-2H-benzo[b]azepin-2-one (100 mg, 0.23 mmol, 1 equiv) and 2-methyl-1-(4-(4,4,5,5-tetramethyl-1,3,2-dioxaborolan-2-yl)-1H-pyrazol-1-yl)propan-2-ol (121 mg, 0.45 mmol, 2 equiv) and Pd(dppf)Cl₂ (41.3 mg, 0.057 mol, 0.25 equiv) were placed in a round-bottom flask, followed by 1,4-dioxane (4.2 mL) and 2N Na₂CO₃ aqueous solution (2.1 mL). The solution was degassed and heated at 80° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 73 mg (purity=99%) yellow solid compound with a yield of 64.32%.

TLC R_{f}=0.1 (PE:EA=1:1);

LCMS (m/z)=499.70 (M+H)

### Example 38. 1-(4-methoxybenzyl)-4-(5-methyloxazol-2-yl)-8-(1-(2-morpholinoethyl)-1H-pyrazol-4-yl)-1,3-dihydro-2H-benzo[b]azepin-2-one (38)

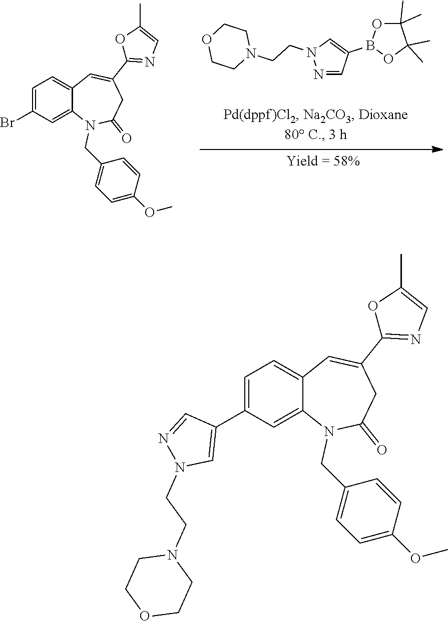

8-bromo-1-(4-methoxybenzyl)-4-(5-methyloxazol-2-yl)-1,3-dihydro-2H-benzo[b]azepin-2-one (150 mg, 0.34 mmol, 1 equiv) and 4-(2-(4-(4,4,5,5-tetramethyl-1,3,2-dioxaborolan-2-yl)-1H-pyrazol-1-yl)ethyl)morpholine (209 mg, 0.68 mmol, 2 equiv) and Pd(dppf)Cl₂ (62 mg, 0.09 mmol, 0.25 equiv) were placed in a round-bottom flask, followed by 1,4-dioxane (7.8 mL) and 2N Na₂CO₃ aqueous solution (3.9 mL). The solution was degassed and heated at 80° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 107 mg (purity=93%) black solid compound with a yield of 58.07%.

TLC R_{f}=0.6 (DCM:MeOH=10:1);

LCMS (m/z)=540.40 (M+H)

### Example 39. 1-(4-methoxybenzyl)-4-(5-methyloxazol-2-yl)-8-(pyridin-3-yl)-1,3-dihydro-2H-benzo[b]azepin-2-one (39)

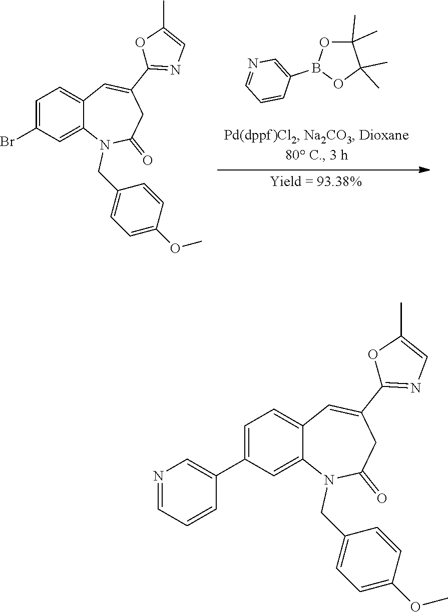 [SMILES: COc1ccc(CN2C(=O)CC(c3ncc(C)o3)=Cc3ccc(Br)cc32)cc1 ; CC1(C)OB(c2cccnc2)OC1(C)C ; COc1ccc(CN2C(=O)CC(c3ncc(C)o3)=Cc3ccc(-c4cccnc4)cc32)cc1]

8-bromo-1-(4-methoxybenzyl)-4-(5-methyloxazol-2-yl)-1,3-dihydro-2H-benzo[b]azepin-2-one (100 mg, 0.23 mmol, 1 equiv) and 3-(4,4,5,5-tetramethyl-1,3,2-dioxaborolan-2-yl)pyridine (93 mg, 0.45 mmol, 2 equiv) and Pd(dppf)Cl₂ (41.3 mg, 0.057 mmol, 0.25 equiv) were placed in a round-bottom flask, followed by 1,4-dioxane (4.2 mL) and 2N Na₂CO₃ aqueous solution (2.1 mL). The solution was degassed and heated at 80° C. for 2 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 93 mg (purity=99%) yellow solid compound with a yield of 93.38%.

TLC R_{f}=0.1 (PE:EA=1:1);

LCMS (m/z)=438.40 (M+H)

### Example 40. 1-(4-methoxybenzyl)-4-(5-methyloxazol-2-yl)-8-(pyridin-4-yl)-1,3-dihydro-2H-benzo[b]azepin-2-one (40)

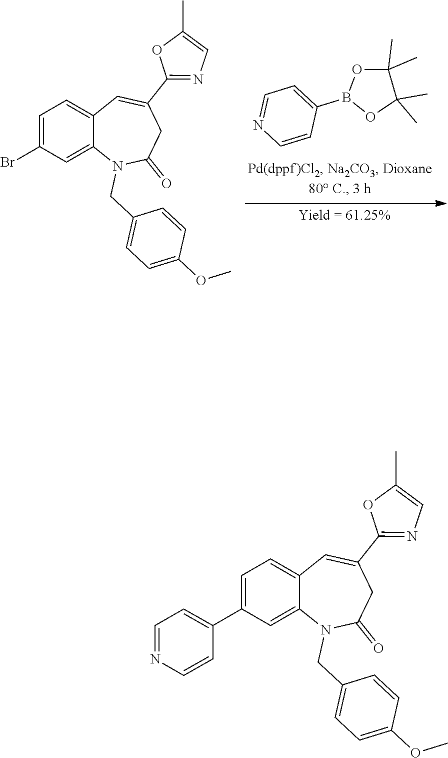 [SMILES: COc1ccc(CN2C(=O)CC(c3ncc(C)o3)=Cc3ccc(Br)cc32)cc1 ; CC1(C)OB(c2ccncc2)OC1(C)C ; COc1ccc(CN2C(=O)CC(c3ncc(C)o3)=Cc3ccc(-c4ccncc4)cc32)cc1]

8-bromo-1-(4-methoxybenzyl)-4-(5-methyloxazol-2-yl)-1,3-dihydro-2H-benzo[b]azepin-2-one (100 mg, 0.23 mmol, 1 equiv) and 4-(4,4,5,5-tetramethyl-1,3,2-dioxaborolan-2-yl)pyridine (93 mg, 0.45 mmol, 2 equiv) and Pd(dppf)Cl₂ (41.3 mg, 0.057 mol, 0.25 equiv) were placed in a round-bottom flask, followed by 1,4-dioxane (4.2 mL) and 2N Na₂CO₃ aqueous solution (2.1 mL). The solution was degassed and heated at 80° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 61 mg (purity=98%) yellow solid compound with a yield of 61.25%.

TLC R_{f}=0.1 (PE:EA=1:1);

LCMS (m/z)=438.10 (M+H)

### Example 41. 8-(6-fluoropyridin-3-yl)-1-(4-methoxybenzyl)-4-(5-methyloxazol-2-yl)-1,3-dihydro-2H-benzo[b]azepin-2-one (41)

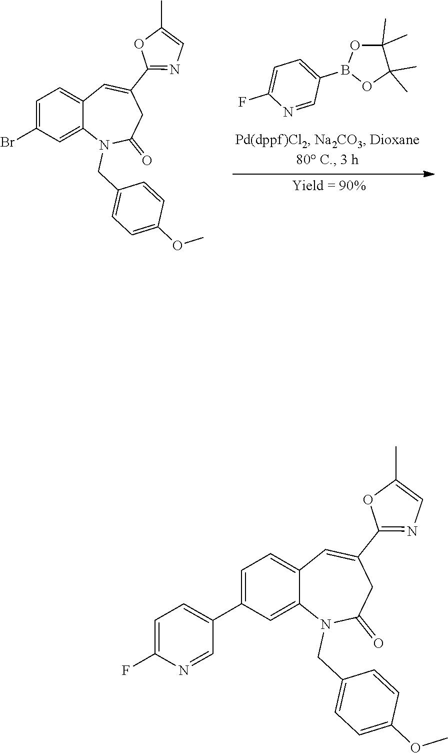 [SMILES: COc1ccc(CN2C(=O)CC(c3ncc(C)o3)=Cc3ccc(Br)cc32)cc1 ; CC1(C)OB(c2ccc(F)nc2)OC1(C)C ; COc1ccc(CN2C(=O)CC(c3ncc(C)o3)=Cc3ccc(-c4ccc(F)nc4)cc32)cc1]

8-bromo-1-(4-methoxybenzyl)-4-(5-methyloxazol-2-yl)-1,3-dihydro-2H-benzo[b]azepin-2-one (100 mg, 0.23 mmol, 1 equiv) and 2-fluoro-5-(4,4,5,5-tetramethyl-1,3,2-dioxaborolan-2-yl)pyridine (101 mg, 0.45 mmol, 2 equiv) and Pd(dppf)Cl₂ (41.3 mg, 0.057 mol, 0.25 equiv) were placed in a round-bottom flask, followed by 1,4-dioxane (4.2 mL) and 2N Na₂CO₃ aqueous solution (2.1 mL). The solution was degassed and heated at 80° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 94 mg (purity=96%) yellow solid compound with a yield of 90.66%.

TLC R_{f}=0.5 (PE:EA=1:1);

LCMS (m/z)=456.30 (M+H)

### Example 42. 8-(6-aminopyridin-3-yl)-1-(4-methoxybenzyl)-4-(5-methyloxazol-2-yl)-1,3-dihydro-2H-benzo[b]azepin-2-one (42)

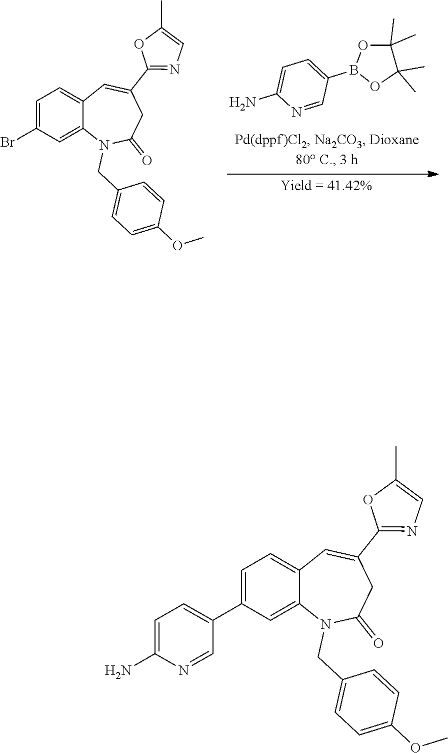 [SMILES: COc1ccc(CN2C(=O)CC(c3ncc(C)o3)=Cc3ccc(Br)cc32)cc1 ; CC1(C)OB(c2ccc(N)nc2)OC1(C)C ; COc1ccc(CN2C(=O)CC(c3ncc(C)o3)=Cc3ccc(-c4ccc(N)nc4)cc32)cc1]

8-bromo-1-(4-methoxybenzyl)-4-(5-methyloxazol-2-yl)-1,3-dihydro-2H-benzo[b]azepin-2-one (150 mg, 0.34 mmol, 1 equiv) and 5-(4,4,5,5-tetramethyl-1,3,2-dioxaborolan-2-yl)pyridin-2-amine (150 mg, 0.68 mmol, 2 equiv) and Pd(dppf)Cl₂ (62 mg, 0.09 mmol, 0.25 equiv) were placed in a round-bottom flask, followed by 1,4-dioxane (7.8 mL) and 2N Na₂CO₃ aqueous solution (3.9 mL). The solution was degassed and heated at 80° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 64 mg (purity=97%) brownness solid compound with a yield of 41.42%.

TLC R_{f}=0.25 (DCM:MeOH=10:1);

LCMS (m/z)=453.00 (M+H)

### Example 43. 8-(6-cyclopropylpyridin-3-yl)-1-(4-methoxybenzyl)-4-(5-methyloxazol-2-yl)-1,3-dihydro-2H-benzo[b]azepin-2-one (43)

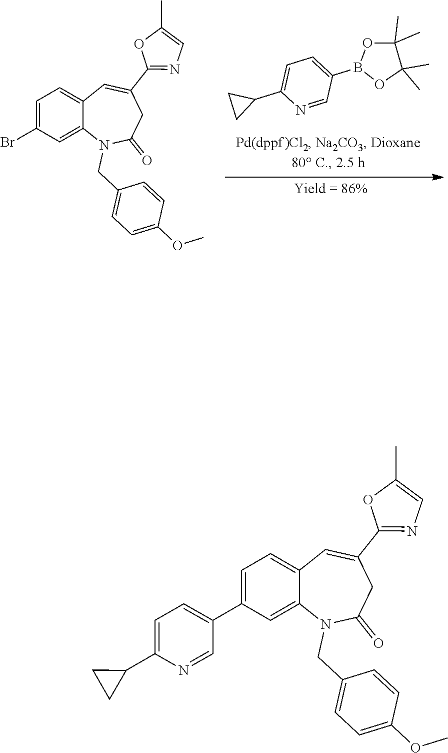 [SMILES: COc1ccc(CN2C(=O)CC(c3ncc(C)o3)=Cc3ccc(Br)cc32)cc1 ; CC1(C)OB(c2ccc(C3CC3)nc2)OC1(C)C ; COc1ccc(CN2C(=O)CC(c3ncc(C)o3)=Cc3ccc(-c4ccc(C5CC5)nc4)cc32)cc1]

8-bromo-1-(4-methoxybenzyl)-4-(5-methyloxazol-2-yl)-1,3-dihydro-2H-benzo[b]azepin-2-one (100 mg, 0.22 mmol, 1 equiv) and 2-cyclopropyl-5-(4,4,5,5-tetramethyl-1,3,2-dioxaborolan-2-yl)pyridine (111 mg, 0.45 mmol, 2 equiv) and Pd(dppf)Cl₂ (41.3 mg, 0.057 mol, 0.25 equiv) were placed in a round-bottom flask, followed by 1,4-dioxane (4.2 mL) and 2N Na₂CO₃ aqueous solution (2.1 mL). The solution was degassed and heated at 80° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 94 mg (purity=97%) white solid compound with a yield of 86%.

TLC R_{f}=0.5 (PE:EA=1:1);

LCMS (m/z)=478.30 (M+H)

### Example 44. 1-(4-methoxybenzyl)-8-(6-methoxypyridin-3-yl)-4-(5-methyloxazol-2-yl)-1,3-dihydro-2H-benzo[b]azepin-2-one (44)

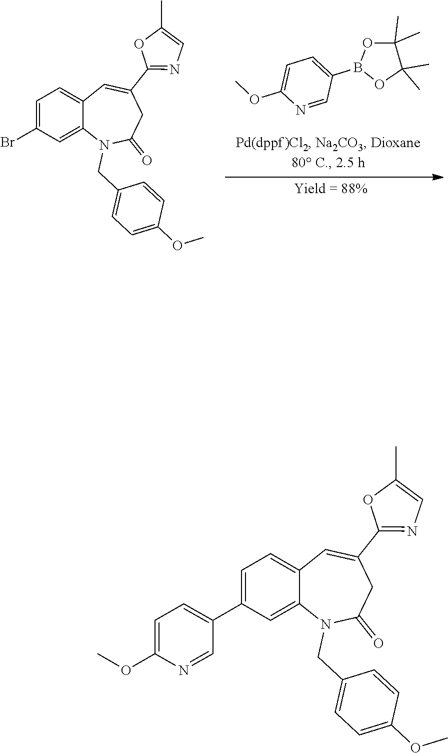

8-bromo-1-(4-methoxybenzyl)-4-(5-methyloxazol-2-yl)-1,3-dihydro-2H-benzo[b]azepin-2-one (100 mg, 0.22 mmol, 1 equiv) and 2-methoxy-5-(4,4,5,5-tetramethyl-1,3,2-dioxaborolan-2-yl)pyridine (108 mg, 0.46 mmol, 2 equiv) and Pd(dppf)Cl₂ (41.3 mg, 0.057 mol, 0.25 equiv) were placed in a round-bottom flask, followed by 1,4-dioxane (4.2 mL) and 2N Na₂CO₃ aqueous solution (2.1 mL). The solution was degassed and heated at 80° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 95 mg (purity=96%) yellow solid compound with a yield of 88%.

TLC R_{f}=0.5 (PE:EA=1:1);

LCMS (m/z)=468.30 (M+H)

### Example 45. 1-(4-methoxybenzyl)-8-(1-methyl-6-oxo-1,6-dihydropyridin-3-yl)-4-(5-methyloxazol-2-yl)-1,3-dihydro-2H-benzo[b]azepin-2-one (45)

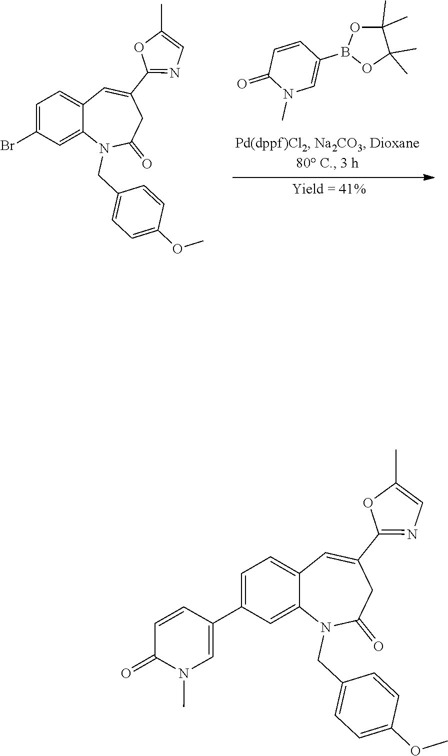 [SMILES: COc1ccc(CN2C(=O)CC(c3ncc(C)o3)=Cc3ccc(Br)cc32)cc1 ; Cn1cc(B2OC(C)(C)C(C)(C)O2)ccc1=O ; COc1ccc(CN2C(=O)CC(c3ncc(C)o3)=Cc3ccc(-c4ccc(=O)n(C)c4)cc32)cc1]

8-bromo-1-(4-methoxybenzyl)-4-(5-methyloxazol-2-yl)-1,3-dihydro-2H-benzo[b]azepin-2-one (150 mg, 0.34 mmol, 1 equiv) and 1-methyl-5-(4,4,5,5-tetramethyl-1,3,2-dioxaborolan-2-yl)pyridin-2(1H)-one (160 mg, 0.68 mmol, 2 equiv) and Pd(dppf)Cl2 (62 mg, 0.09 mmol, 0.25 equiv) were placed in a round-bottom flask, followed by 1,4-dioxane (7.8 mL) and 2N Na₂CO₃ aqueous solution (3.9 mL). The solution was degassed and heated at 80° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 65 mg (purity=94%) yellow solid compound with a yield of 41%.

TLC R_{f}=0.2 (DCM:MeOH=10:1);

LCMS (m/z)=468.30 (M+H)

### Example 46. 1-(4-methoxybenzyl)-4-(5-methyloxazol-2-yl)-8-(pyrimidin-5-yl)-1,3-dihydro-2H-benzo[b]azepin-2-one (46)

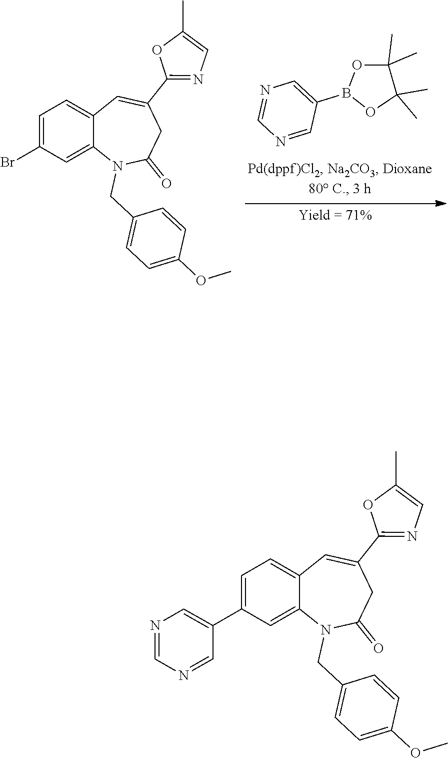 [SMILES: COc1ccc(CN2C(=O)CC(c3ncc(C)o3)=Cc3ccc(Br)cc32)cc1 ; CC1(C)OB(c2cncnc2)OC1(C)C ; COc1ccc(CN2C(=O)CC(c3ncc(C)o3)=Cc3ccc(-c4cncnc4)cc32)cc1]

8-bromo-1-(4-methoxybenzyl)-4-(5-methyloxazol-2-yl)-1,3-dihydro-2H-benzo[b]azepin-2-one (100 mg, 0.22 mmol, 1 equiv) and 5-(4,4,5,5-tetramethyl-1,3,2-dioxaborolan-2-yl)pyrimidine (94 mg, 0.46 mmol, 2 equiv) and Pd(dppf)Cl₂ (41.3 mg, 0.057 mol, 0.25 equiv) were placed in a round-bottom flask, followed by 1,4-dioxane (4.2 mL) and 2N Na₂CO₃ aqueous solution (2.1 mL). The solution was degassed and heated at 80° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 72 mg (purity=94%) black solid compound with a yield of 710%.

TLC R_{f}=0.1 (PE:EA=1:1);

LCMS (m/z)=439.10 (M+H)

### Example 47. 8-(2-aminopyrimidin-5-yl)-1-(4-methoxybenzyl)-4-(5-methyloxazol-2-yl)-1,3-dihydro-2H-benzo[b]azepin-2-one (47)

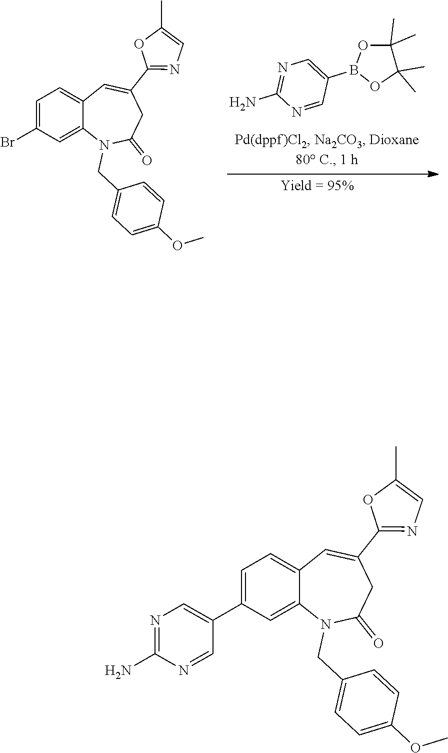 [SMILES: COc1ccc(CN2C(=O)CC(c3ncc(C)o3)=Cc3ccc(Br)cc32)cc1 ; CC1(C)OB(c2cnc(N)nc2)OC1(C)C ; COc1ccc(CN2C(=O)CC(c3ncc(C)o3)=Cc3ccc(-c4cnc(N)nc4)cc32)cc1]

8-bromo-1-(4-methoxybenzyl)-4-(5-methyloxazol-2-yl)-1,3-dihydro-2H-benzo[b]azepin-2-one (150 mg, 0.34 mmol, 1 equiv) and 5-(4,4,5,5-tetramethyl-1,3,2-dioxaborolan-2-yl)pyrimidin-2-amine (150 mg, 0.68 mmol, 2 equiv) and Pd(dppf)Cl₂ (62 mg, 0.09 mmol, 0.25 equiv) were placed in a round-bottom flask, followed by 1,4-dioxane (7.8 mL) and 2N Na₂CO₃ aqueous solution (3.9 mL). The solution was degassed and heated at 80° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 110 mg (purity=92%) yellow solid compound with a yield of 71%.

TLC R_{f}=0.45 (DCM:MeOH=10:1);

LCMS (m/z)=454.30 (M+H)

### Example 48. 1-(4-methoxybenzyl)-4-(5-methyloxazol-2-yl)-8-(1-(tetrahydrofuran-3-yl)-1H-pyrazol-4-yl)-1,3-dihydro-2H-benzo[b]azepin-2-one (48)

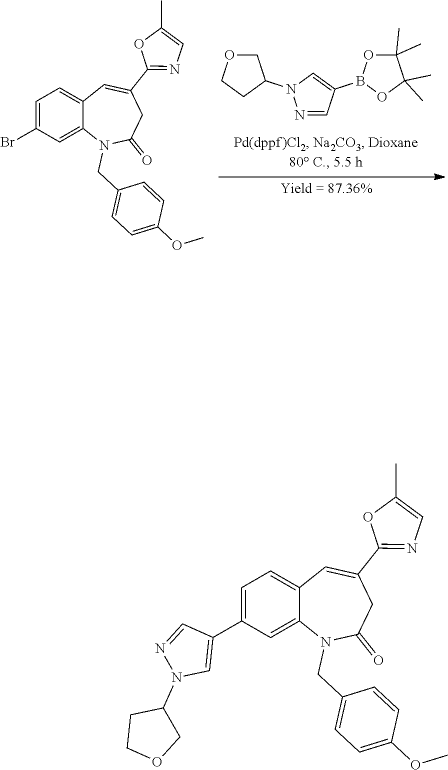 [SMILES: COc1ccc(CN2C(=O)CC(c3ncc(C)o3)=Cc3ccc(Br)cc32)cc1 ; CC1(C)OB(c2cnn(C3CCOC3)c2)OC1(C)C ; COc1ccc(CN2C(=O)CC(c3ncc(C)o3)=Cc3ccc(-c4cnn(C5CCOC5)c4)cc32)cc1]

8-bromo-1-(4-methoxybenzyl)-4-(5-methyloxazol-2-yl)-1,3-dihydro-2H-benzo[b]azepin-2-one (100 mg, 0.228 mmol, 1 equiv) and 1-(tetrahydrofuran-3-yl)-4-(4,4,5,5-tetramethyl-1,3,2-dioxaborolan-2-yl)-1H-pyrazole (120 mg, 0.455 mmol, 2 equiv) and Pd(dppf)Cl₂ (42 mg, 0.057 mmol, 0.25 equiv) were placed in a round-bottom flask, followed by 1,4-dioxane (4.2 mL) and 2N Na₂CO₃ aqueous solution (2.1 mL). The solution was degassed and heated at 80° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 93 mg (purity=99%) yellow solid compound with a yield of 87.36%.

TLC R_{f}=0.05 (PE:EA=1:1);

LCMS (m/z)=497.60 (M+H)

### Example 49. 1-(4-methoxybenzyl)-4-(5-methyloxazol-2-yl)-8-(1-(tetrahydro-2H-pyran-4-yl)-1H-pyrazol-4-yl)-1,3-dihydro-2H-benzo[b]azepin-2-one (49)

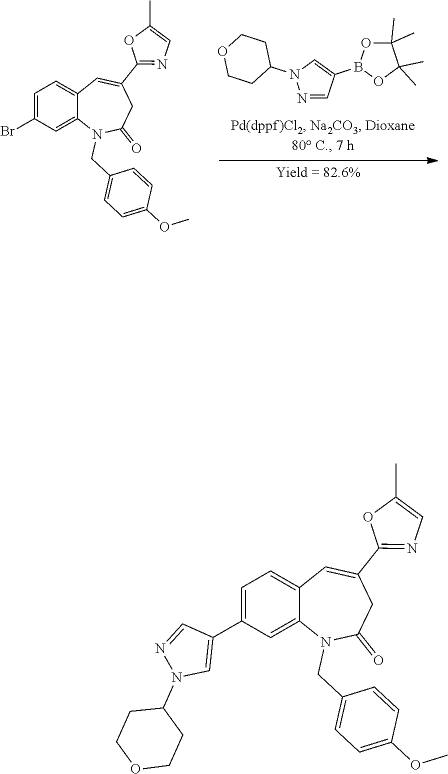 [SMILES: COc1ccc(CN2C(=O)CC(c3ncc(C)o3)=Cc3ccc(Br)cc32)cc1 ; CC1(C)OB(c2cnn(C3CCOCC3)c2)OC1(C)C ; COc1ccc(CN2C(=O)CC(c3ncc(C)o3)=Cc3ccc(-c4cnn(C5CCOCC5)c4)cc32)cc1]

8-bromo-1-(4-methoxybenzyl)-4-(5-methyloxazol-2-yl)-1,3-dihydro-2H-benzo[b]azepin-2-one (100 mg, 0.228 mmol, 1 equiv) and 1-(tetrahydro-2H-pyran-4-yl)-4-(4,4,5,5-tetramethyl-1,3,2-dioxaborolan-2-yl)-1H-pyrazole (127 mg, 0.455 mmol, 2 equiv) and Pd(dppf)Cl₂ (42 mg, 0.057 mmol, 0.25 equiv) were placed in a round-bottom flask, followed by 1,4-dioxane (4.2 mL) and 2N Na₂CO₃ aqueous solution (2.1 mL). The solution was degassed and heated at 80° C. for 7 h.

The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 96 mg (purity=98%) yellow solid compound with a yield of 82.6%.

TLC R_{f}=0.05 (PE:EA=1:1);

LCMS (m/z)=511.20 (M+H)

### Example 50. 2-(4-(1-(4-methoxybenzyl)-4-(5-methyloxazol-2-yl)-2-oxo-2,3-dihydro-1H-benzo[b]azepin-8-yl)-1H-pyrazol-1-yl)acetamide (50)

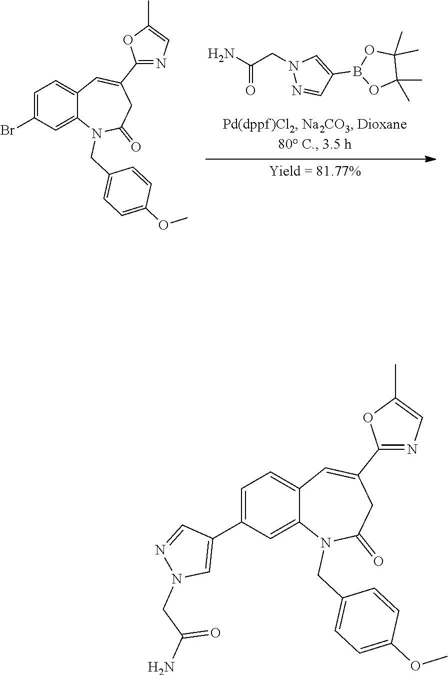 [SMILES: COc1ccc(CN2C(=O)CC(c3ncc(C)o3)=Cc3ccc(Br)cc32)cc1 ; CC1(C)OB(c2cnn(CC(N)=O)c2)OC1(C)C ; COc1ccc(CN2C(=O)CC(c3ncc(C)o3)=Cc3ccc(-c4cnn(CC(N)=O)c4)cc32)cc1]

8-bromo-1-(4-methoxybenzyl)-4-(5-methyloxazol-2-yl)-1,3-dihydro-2H-benzo[b]azepin-2-one (100 mg, 0.228 mmol, 1 equiv) and 2-(4-(4,4,5,5-tetramethyl-1,3,2-dioxaborolan-2-yl)-1H-pyrazol-1-yl)acetamide (114 mg, 0.455 mmol, 2 equiv) and Pd(dppf)Cl₂ (42 mg, 0.057 mmol, 0.25 equiv) were placed in a round-bottom flask, followed by 1,4-dioxane (4.2 mL) and 2N Na₂CO₃ aqueous solution (2.1 mL). The solution was degassed and heated at 80° C. for 3.5 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 90 mg (purity=98%) yellow solid compound with a yield of 81.77%.

TLC R_{f}=0.15 (EA=100%);

LCMS (m/z)=484.40 (M+H)

### Example 51. 1-(4-methoxybenzyl)-8-(1-(2-methoxyethyl)-1H-pyrazol-4-yl)-4-(5-methyloxazol-2-yl)-1,3-dihydro-2H-benzo[b]azepin-2-one (51)

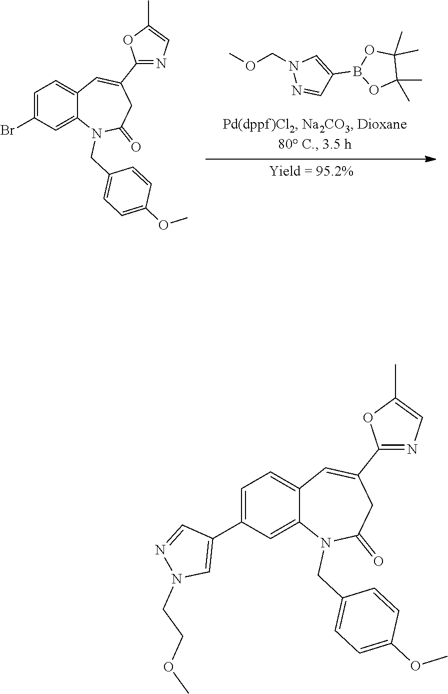

8-bromo-1-(4-methoxybenzyl)-4-(5-methyloxazol-2-yl)-1,3-dihydro-2H-benzo[b]azepin-2-one (100 mg, 0.228 mmol, 1 equiv) and 1-(2-methoxyethyl)-4-(4,4,5,5-tetramethyl-1,3,2-dioxaborolan-2-yl)-1H-pyrazole (115 mg, 0.455 mmol, 2 equiv) and Pd(dppf)Cl2 (42 mg, 0.057 mmol, 0.25 equiv) were placed in a round-bottom flask, followed by 1,4-dioxane (4.2 mL) and 2N Na₂CO₃ aqueous solution (2.1 mL). The solution was degassed and heated at 80° C. for 3.5 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 105 mg (purity=97%) yellow oil compound with a yield of 95.2%.

TLC R_{f}=0.05 (EA=100%);

LCMS (m/z)=485.50 (M+H)

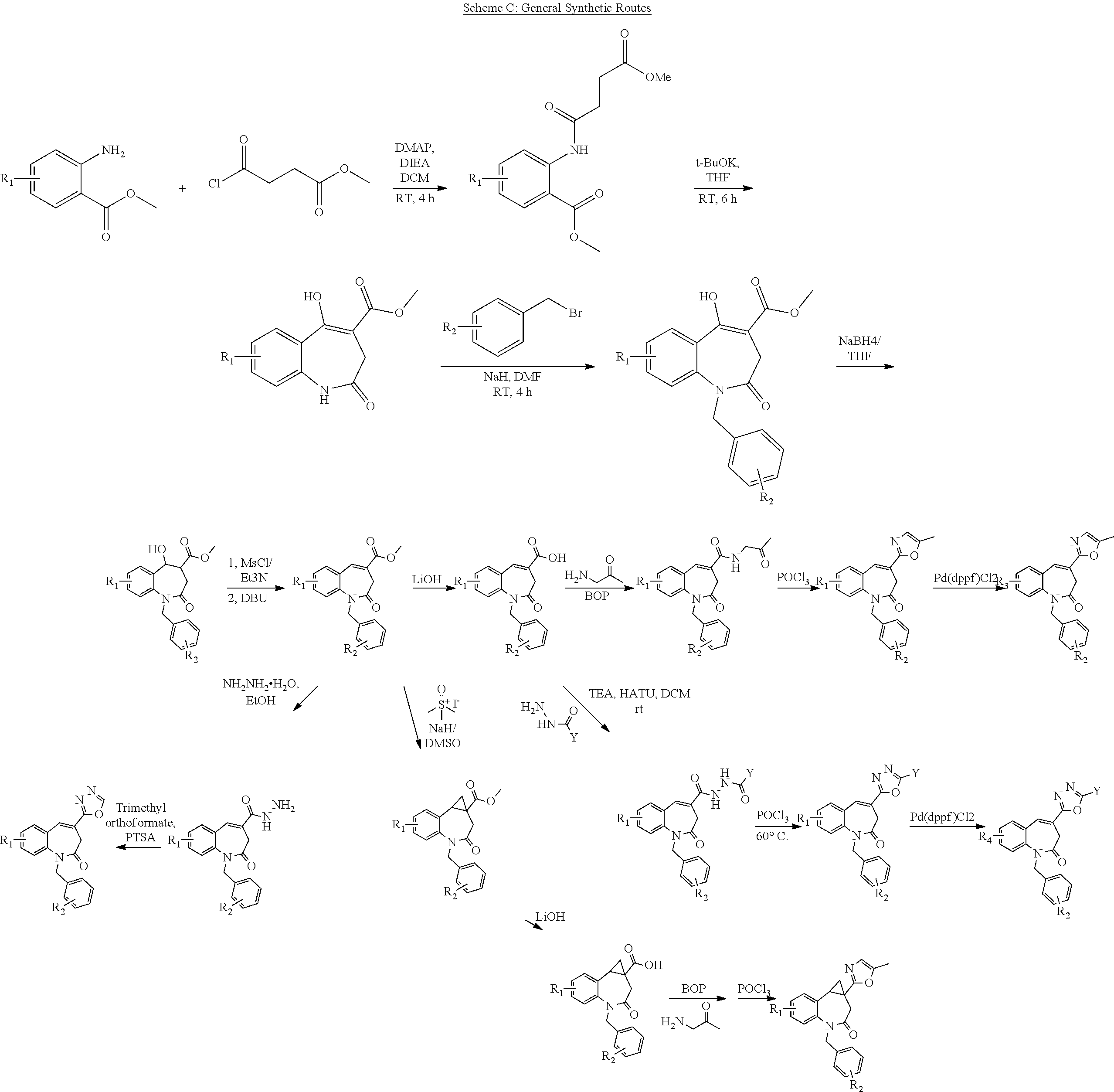

### Example 52. 4-(5-cyclopropyl-1,3,4-oxadiazol-2-yl)-1-(4-methoxybenzyl)-8-(1-(2-morpholinoethyl)-1H-pyrazol-4-yl)-1,3-dihydro-2H-benzo[b]azepin-2-one (52)

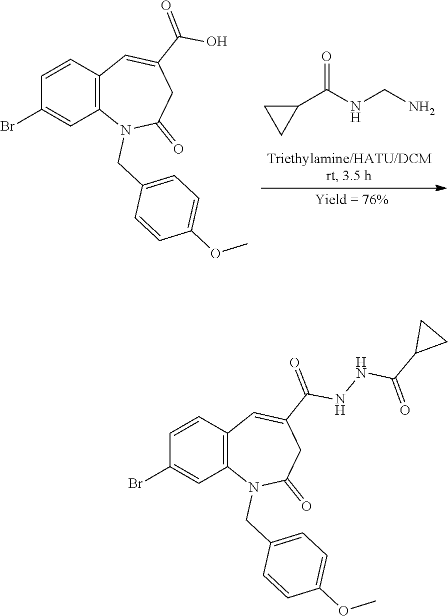 [SMILES: COc1ccc(CN2C(=O)CC(C(=O)O)=Cc3ccc(Br)cc32)cc1 ; NCNC(=O)C1CC1 ; COc1ccc(CN2C(=O)CC(C(=O)NNC(=O)C3CC3)=Cc3ccc(Br)cc32)cc1]

### bromo-N′-(cyclopropanecarbonyl)-1-(4-methoxybenzyl)-2-oxo-2,3-dihydro-1H-benzo[b]azepine-4-carbohydrazide

8-bromo-1-(4-methoxybenzyl)-2-oxo-2,3-dihydro-1H-benzo[b]azepine-4-carboxylic acid (524 mg, 1.3 mmol, 1 equiv) in dichloromethane (8 mL) was added Cyclopropanecarboxylicacid hydrazide (195 mg, 1.95 mmol, 1.5 equiv), triethylamine (339 μL, 1.95 mmol, 1.5 equiv), and HATU (988 mg, 2.6 mmol, 1.5 equiv). The mixture was stirred at room temperature for 3.5h. Then it was concentrated to give a crude, which was purified by silica gel column chromatography to get the product 480 mg (purity=100%) as a white solid. Yield: 76%.

TLC R_{f}=0.2 (PE:EA=1:2);

LCMS (m/z)=484.40 (M+H)

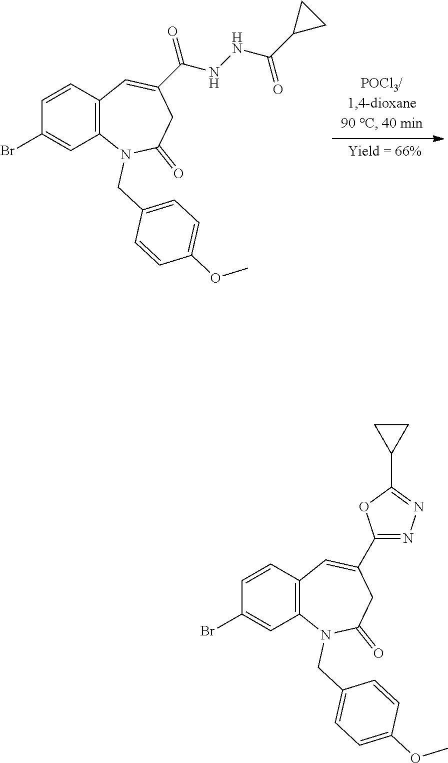 [SMILES: COc1ccc(CN2C(=O)CC(C(=O)NNC(=O)C3CC3)=Cc3ccc(Br)cc32)cc1 ; COc1ccc(CN2C(=O)CC(c3nnc(C4CC4)o3)=Cc3ccc(Br)cc32)cc1]

### 8-bromo-4-(5-cyclopropyl-1,3,4-oxadiazol-2-yl)-1-(4-methoxybenzyl)-1,3-dihydro-2H-benzo[b]azepin-2-one

8-bromo-N′-(cyclopropanecarbonyl)-1-(4-methoxybenzyl)-2-oxo-2,3-dihydro-1H-benzo[b]azepine-4-carbohydrazide (484 mg, 1 mmol, 1 equiv) was dissolved in 1,4-dioxane (15 mL) and phosphoryl chloride (612 μL, 6.57 mmol, 6.57 equiv) was added and the mixture was heated at 90° C. for 40 min, then cooled to room temperature and diluted with ethyl acetate. The mixture was washed with satd. NaHCO₃ and brine, dried over sodium sulfate, filtered and concentrated in vacuo. The residue was purified by silica gel column chromatography to get the product (310 mg) as a white solid. Yield: 66%; Purity: 98%.

TLC R_{f}=0.45 (PE:EA=1:1);

LCMS (m/z)=465.90 (M+H)

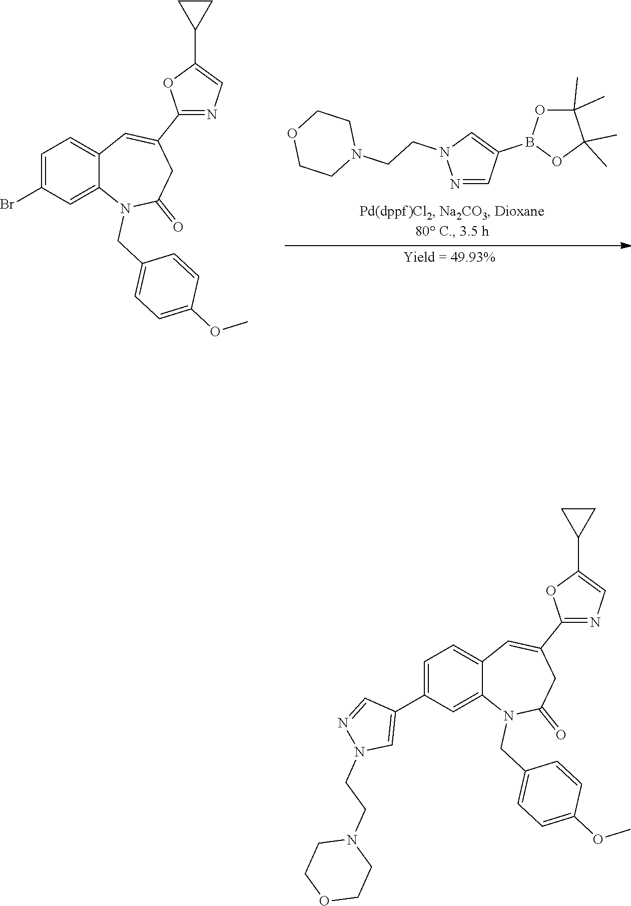 [SMILES: COc1ccc(CN2C(=O)CC(c3ncc(C4CC4)o3)=Cc3ccc(Br)cc32)cc1 ; CC1(C)OB(c2cnn(CCN3CCOCC3)c2)OC1(C)C ; COc1ccc(CN2C(=O)CC(c3ncc(C4CC4)o3)=Cc3ccc(-c4cnn(CCN5CCOCC5)c4)cc32)cc1]

### 4-(5-cyclopropyl-1,3,4-oxadiazol-2-yl)-1-(4-methoxybenzyl)-8-(1-(2-morpholinoethyl)-1H-pyrazol-4-yl)-1,3-dihydro-2H-benzo[b]azepin-2-one (52)

8-bromo-4-(5-cyclopropyl-1,3,4-oxadiazol-2-yl)-1-(4-methoxybenzyl)-1,3-dihydro-2H-benzo[b]azepin-2-one (150 mg, 0.32 mmol, 1 equiv) and 4-(2-(4-(4,4,5,5-tetramethyl-1,3,2-dioxaborolan-2-yl)-1H-pyrazol-1-yl)ethyl)morpholine (198 mg, 0.64 mmol, 2 equiv) and Pd(dppf)Cl₂ (59 mg, 0.08 mmol, 0.25 equiv) were placed in a round-bottom flask, followed by 1,4-dioxane (5.8 mL) and 2N Na₂CO₃ aqueous solution (2.9 mL). The solution was degassed and heated at 80° C. for 3.5 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 91 mg (purity=100%) yellow oil compound with a yield of 49.93%.

TLC R_{f}=0.05 (EA=100%);

LCMS (m/z)=566.90 (M+H)

### Example 53. 8-(1-(cyclopropylmethyl)-1H-pyrazol-4-yl)-1-(4-methoxybenzyl)-4-(5-methyloxazol-2-yl)-1,3-dihydro-2H-benzo[b]azepin-2-one (Compound 53)

8-bromo-4-(5-cyclopropyl-1,3,4-oxadiazol-2-yl)-1-(4-methoxybenzyl)-1,3-dihydro-2H-benzo[b]azepin-2-one (200 mg, 0.455 mmol, 1 equiv) and 1-(cyclopropylmethyl)-4-(4,4,5,5-tetramethyl-1,3,2-dioxaborolan-2-yl)-1H-pyrazole (226 mg, 0.91 mmol, 2 equiv) and Pd(dppf)Cl₂ (82 mg, 0.114 mmol, 0.25 equiv) were placed in a round-bottom flask, followed by 1,4-dioxane (8.4 mL) and 2N Na₂CO₃ aqueous solution (4.2 mL). The solution was degassed and heated at 80° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 192 mg (purity=93%) yellow oil compound with a yield of 87.76%.

TLC R_{f}=0.15 (PE:EA=1:1);

LCMS (m/z)=481.40 (M+H)

### Example 54. 1-(4-Methoxybenzyl)-4-(5-methyloxazol-2-yl)-8-(1-(oxetan-3-yl)-1H-pyrazol-4-yl)-1,3-dihydro-2H-benzo[b]azepin-2-one (54)

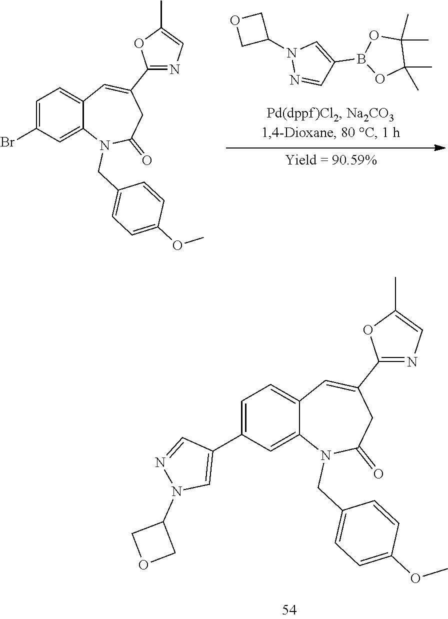 [SMILES: COc1ccc(CN2C(=O)CC(c3ncc(C)o3)=Cc3ccc(Br)cc32)cc1 ; CC1(C)OB(c2cnn(C3COC3)c2)OC1(C)C ; COc1ccc(CN2C(=O)CC(c3ncc(C)o3)=Cc3ccc(-c4cnn(C5COC5)c4)cc32)cc1]

8-bromo-1-(4-methoxybenzyl)-4-(5-methyloxazol-2-yl)-1,3-dihydro-2H-benzo[b]azepin-2-one (200 mg, 0.455 mmol, 1 equiv) and 1-(oxetan-3-yl)-4-(4,4,5,5-tetramethyl-1,3,2-dioxaborolan-2-yl)-1H-pyrazole (228 mg, 0.91 mmol, 2 equiv) and Pd(dppf)Cl₂ (83 mg, 0.114 mmol, 0.25 equiv) were placed in a round-bottom flask, followed by 1,4-dioxane (8.4 mL) and 2N Na₂CO₃ aqueous solution (4.2 mL). The solution was degassed and heated at 80° C. for 1 h. The solution was quenched with saturated ammonium chloride and extracted with ethyl acetate (3×10 mL). The combined organic layers were washed with H₂O (2×10 mL) and brine solution solution (1×20 mL). The combined organic layers were dried over anhydrous Na₂SO₄, filtered, and concentrated by rotary evaporation to get the crude compound. Then, the resulting concentrate was purified on a silica column to get 199 mg (purity=94%) yellow solid compound with a yield of 90.59%.

TLC R_{f}=0.25 (DCM:EA=2:1);

LCMS (m/z)=483.30 (M+H)

### Example 55. 8-(2-Aminopyridin-4-yl)-1-(4-methoxybenzyl)-4-(5-methyloxazol-2-yl)-1,3-dihydro-2H-benzo[b]azepin-2-one (55)

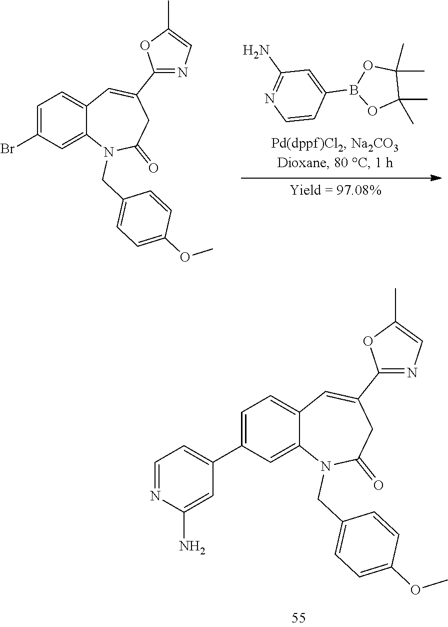 [SMILES: COc1ccc(CN2C(=O)CC(c3ncc(C)o3)=Cc3ccc(Br)cc32)cc1 ; CC1(C)OB(c2ccnc(N)c2)OC1(C)C ; COc1ccc(CN2C(=O)CC(c3ncc(C)o3)=Cc3ccc(-c4ccnc(N)c4)cc32)cc1]

8-bromo-1-(4-methoxybenzyl)-4-(5-methyloxazol-2-yl)-1,3-dihydro-2H-benzo[b]azepin-2-one (200 mg, 0.455 mmol, 1 equiv) and 4-(4,4,5,5-tetramethyl-1,3,2-dioxaborolan-2-yl)pyridin-2-amine (200 mg, 0.91 mmol, 2 equiv) and Pd(dppf)Cl2 (83 mg, 0.114 mmol, 0.25 equiv) were placed in a round-bottom flask, followed by 1,4-dioxane (8.4 mL) and 2N Na₂CO₃ aqueous solution (4.2 mL). The solution was degassed and heated at 80° C. for 1 h. The solution was quenched with saturated ammonium chloride and extracted with EA (3×10 mL). The organic solutions were combined, washed with H₂O (2×10 mL) and brine solution (1×20 mL), dried over anhydrous Na₂SO₄, filtered and concentrated by rotary evaporation to get the crude compound. Then, the resulting concentrate was purified on a silica column to get 200 mg (purity=98%) yellow solid compound with a yield of 97.08%.

TLC R_{f}=0.05 (DCM:EA=2:1);

LCMS (m/z)=453.30 (M+H)

### Example 56. 8-(1-(1-hydroxy-2-methylpropan-2-yl)-1H-pyrazol-4-yl)-1-(4-methoxybenzyl)-4-(5-methyloxazol-2-yl)-1,3-dihydro-2H-benzo[b]azepin-2-one: (56)

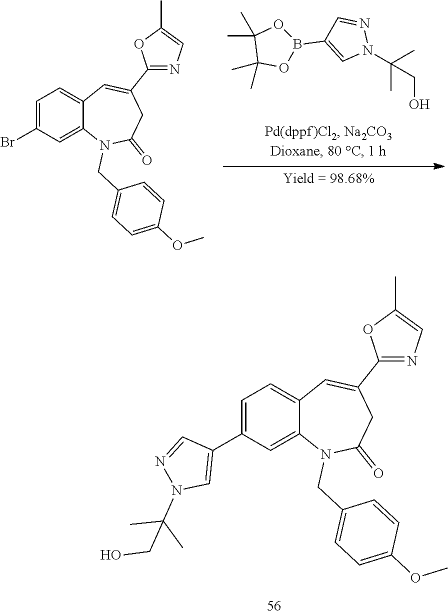 [SMILES: COc1ccc(CN2C(=O)CC(c3ncc(C)o3)=Cc3ccc(Br)cc32)cc1 ; CC(C)(CO)n1cc(B2OC(C)(C)C(C)(C)O2)cn1 ; COc1ccc(CN2C(=O)CC(c3ncc(C)o3)=Cc3ccc(-c4cnn(C(C)(C)CO)c4)cc32)cc1]

8-bromo-1-(4-methoxybenzyl)-4-(5-methyloxazol-2-yl)-1,3-dihydro-2H-benzo[b]azepin-2-one (200 mg, 0.455 mmol, 1 equiv) and 2-methyl-2-(4-(4,4,5,5-tetramethyl-1,3,2-dioxaborolan-2-yl)-1H-pyrazol-1-yl)propan-1-ol (242 mg, 0.91 mmol, 2 equiv) and Pd(dppf)Cl₂ (83 mg, 0.114 mmol, 0.25 equiv) were placed in a round-bottom flask, followed by 1,4-dioxane (8.4 mL) and 2N Na₂CO₃ aqueous solution (4.2 mL). The solution was degassed and heated at 80° C. for 1 h. The solution was quenched with saturated ammonium chloride and extracted with EA (3×10 mL). The organic solutions were combined, washed with H₂O (2×10 mL) and brine solution (1×20 mL), dried over anhydrous Na₂SO₄, filtered and concentrated by rotary evaporation to get the crude compound. Then, the resulting concentrate was purified on a silica column to get 224 mg (purity=97%) yellow solid compound with a yield of 99.11%.

TLC R_{f}=0.2 (DCM:EA=2:1);

LCMS (m/z)=499.50 (M+H)

¹H NMR (400 MHz, Chloroform-d) δ 7.67 (s, 1H), 7.66 (s, 1H), 7.49 (s, 1H), 7.44 (d, J=1.6 Hz, 1H), 7.31 (d, J=8.1 Hz, 1H), 7.26 (dd, J=8.0, 1.7 Hz, 1H), 7.13-7.06 (m, 2H), 6.84 (d, J=1.3 Hz, 1H), 6.82-6.75 (m, 2H), 5.05 (s, 2H), 4.01-2.91 (br., 2H), 3.82 (s, 2H), 3.75 (s, 3H), 3.61 (s, 1H), 2.37 (d, J=1.2 Hz, 3H), 1.58 (s, 6H).

### Example 57. 1-(4-Methoxybenzyl)-8-(1-(3-methoxypropyl)-1H-pyrazol-4-yl)-4-(5-methyloxazol-2-yl)-1,3-dihydro-2H-benzo[b]azepin-2-one (57)

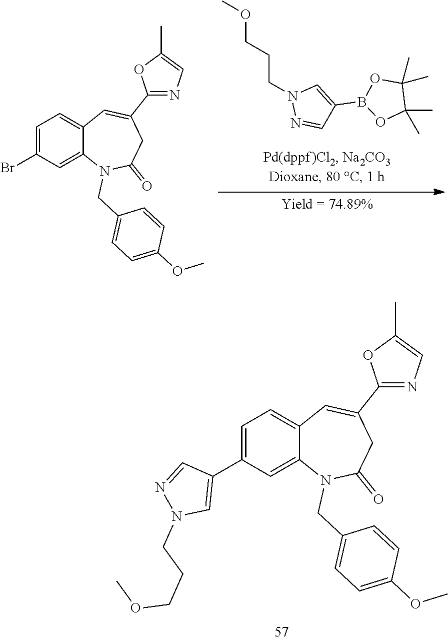

8-bromo-1-(4-methoxybenzyl)-4-(5-methyloxazol-2-yl)-1,3-dihydro-2H-benzo[b]azepin-2-one (200 mg, 0.455 mmol, 1 equiv) and 1-(3-methoxypropyl)-4-(4,4,5,5-tetramethyl-1,3,2-dioxaborolan-2-yl)-1H-pyrazole (245 mg, 0.91 mmol, 2 equiv) and Pd(dppf)Cl2 (83 mg, 0.114 mmol, 0.25 equiv) were placed in a round-bottom flask, followed by 1,4-dioxane (8.4 mL) and 2N Na₂CO₃ aqueous solution (4.2 mL). The solution was degassed and heated at 80° C. for 1 h. The solution was quenched with saturated ammonium chloride and extracted with EA (3×10 mL). The organic solutions were combined, washed with H₂O (2×10 mL) and brine solution (1×20 mL), dried over anhydrous Na₂SO₄, filtered and concentrated by rotary evaporation to get the crude compound. Then, the resulting concentrate was purified on a silica column to get 170 mg (purity=98%) yellow solid compound with a yield of 75.22%.

TLC R_{f}=0.3 (DCM:EA=2:1);

LCMS (m/z)=499.40 (M+H)

¹H NMR (400 MHz, Chloroform-d) δ 7.67 (s, 1H), 7.56 (s, 1H), 7.50 (s, 1H), 7.45 (s, 1H), 7.31 (d, J=8.0 Hz, 1H), 7.26 (d, J=7.8 Hz, 1H), 7.08 (d, J=8.2 Hz, 2H), 6.84 (s, 1H), 6.78 (d, J=8.1 Hz, 2H), 5.06 (s, 2H), 4.24 (t, J=6.7 Hz, 2H), 3.75 (s, 3H), 3.33 (merged peaks, 5H), 2.38 (s, 3H), 2.13 (p, J=6.3 Hz, 2H).

### Example 58. 8-(1-(1-Ethoxyethyl)-1H-pyrazol-4-yl)-1-(4-methoxybenzyl)-4-(5-methyloxazol-2-yl)-1,3-dihydro-2H-benzo[b]azepin-2-one (58)

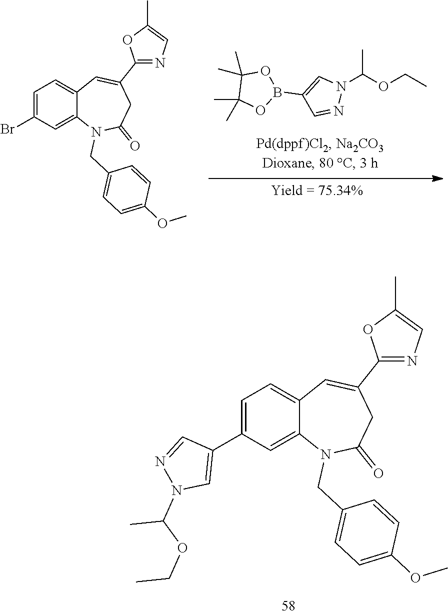 [SMILES: COc1ccc(CN2C(=O)CC(c3ncc(C)o3)=Cc3ccc(Br)cc32)cc1 ; CCOC(C)n1cc(B2OC(C)(C)C(C)(C)O2)cn1 ; CCOC(C)n1cc(-c2ccc3c(c2)N(Cc2ccc(OC)cc2)C(=O)CC(c2ncc(C)o2)=C3)cn1]

8-bromo-1-(4-methoxybenzyl)-4-(5-methyloxazol-2-yl)-1,3-dihydro-2H-benzo[b]azepin-2-one (200 mg, 0.455 mmol, 1 equiv) and 1-(1-ethoxyethyl)-4-(4,4,5,5-tetramethyl-1,3,2-dioxaborolan-2-yl)-1H-pyrazole (242 mg, 0.91 mmol, 2 equiv) and Pd(dppf)Cl₂ (83 mg, 0.114 mmol, 0.25 equiv) were placed in a round-bottom flask, followed by 1,4-dioxane (8.4 mL) and 2N Na₂CO₃ aqueous solution (4.2 mL). The solution was degassed and heated at 80° C. for 1 h. The solution was quenched with saturated ammonium chloride and extracted with EA (3×10 mL). The organic solutions were combined, washed with H₂O (2×10 mL) and brine solution (1×20 mL), dried over anhydrous Na₂SO₄, filtered and concentrated by rotary evaporation to get the crude compound. Then, the resulting concentrate was purified on a silica column to get 171 mg (purity=94%) yellow oil compound with a yield of 75.34%.

TLC R_{f}=0.6 (DCM:EA=2:1);

LCMS (m/z)=499.40 (M+H)

¹H NMR (400 MHz, Chloroform-d) δ 7.75 (s, 1H), 7.67 (s, 1H), 7.50 (s, 1H), 7.47 (d, J=1.5 Hz, 1H), 7.33 (d, J=8.1 Hz, 1H), 7.29 (dd, J=8.0, 1.5 Hz, 1H), 7.09 (d, J=8.6 Hz, 2H), 6.84 (d, J=1.3 Hz, 1H), 6.78 (d, J=8.6 Hz, 2H), 5.52 (q, J=6.0 Hz, 1H), 5.07 (s, 2H), 3.75 (s, 3H), 3.49 (dd, J=9.5, 7.0 Hz, 1H), 3.40-3.34 (m, 1H), 2.37 (s, 3H), 2.06 (s, 2H), 1.69 (d, J=6.0 Hz, 3H), 1.16 (t, J=7.0 Hz, 3H).

### Example 59. 3-(4-(1-(4-Methoxybenzyl)-4-(5-methyloxazol-2-yl)-2-oxo-2,3-dihydro-1H-benzo[b]azepin-8-yl)-1H-pyrazol-1-yl)propanenitrile (59)

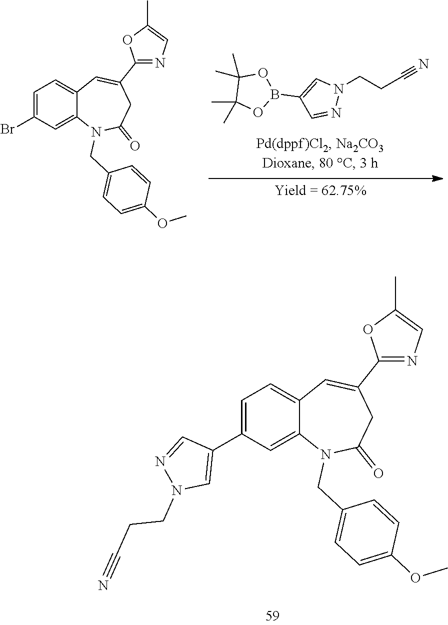 [SMILES: COc1ccc(CN2C(=O)CC(c3ncc(C)o3)=Cc3ccc(Br)cc32)cc1 ; CC1(C)OB(c2cnn(CCC#N)c2)OC1(C)C ; COc1ccc(CN2C(=O)CC(c3ncc(C)o3)=Cc3ccc(-c4cnn(CCC#N)c4)cc32)cc1]

bromo-4-(5-cyclopropyl-1,3,4-oxadiazol-2-yl)-1-(4-methoxybenzyl)-1,3-dihydro-2H-benzo[b]azepin-2-one (200 mg, 0.455 mmol, 1 equiv) and 3-(4-(4,4,5,5-tetramethyl-1,3,2-dioxaborolan-2-yl)-1H-pyrazol-1-yl)propanenitrile (225 mg, 0.91 mmol, 2 equiv) and Pd(dppf)Cl₂ (83 mg, 0.114 mmol, 0.25 equiv) were placed in a round-bottom flask, followed by 1,4-dioxane (8.4 mL) and 2N Na₂CO₃ aqueous solution (4.2 mL). The solution was degassed and heated at 80° C. for 1 h. The solution was quenched with saturated ammonium chloride and extracted with EA (3×10 mL). The organic solutions were combined, washed with H₂O (2×10 mL) and brine solution (1×20 mL), dried over anhydrous Na₂SO₄, filtered and concentrated by rotary evaporation to get the crude compound. Then, the resulting concentrate was purified on a silica column to get 137 mg (purity=98%) yellow solid compound with a yield of 62.75%.

TLC R_{f}=0.25 (DCM:EA=2:1);

LCMS (m/z)=480.30 (M+H)

¹H NMR (400 MHz, DMSO-d₆) δ 8.34 (s, 1H), 8.03 (s, 1H), 7.81 (d, J=1.6 Hz, 1H), 7.50 (s, 1H), 7.47 (d, J=8.2 Hz, 1H), 7.40 (dd, J=8.1, 1.6 Hz, 1H), 7.01 (d, J=1.4 Hz, 1H), 6.91 (d, J=8.7 Hz, 2H), 6.74 (d, J=8.7 Hz, 2H), 5.19 (s, 2H), 4.42 (t, J=6.3 Hz, 2H), 3.64 (s, 3H), 3.11 (t, J=6.3 Hz, 2H), 2.50 (merged peaks with DMSO, 2H), 2.38 (d, J=1.2 Hz, 3H).

### Example 60. 4-(1-(4-Methoxybenzyl)-4-(5-methyloxazol-2-yl)-2-oxo-2,3-dihydro-1H-benzo[b]azepin-8-yl)-N-methylpicolinamide (60)

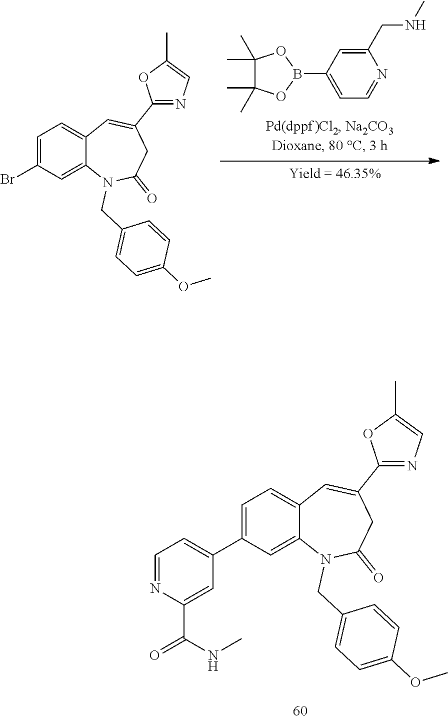

bromo-4-(5-cyclopropyl-1,3,4-oxadiazol-2-yl)-1-(4-methoxybenzyl)-1,3-dihydro-2H-benzo[b]azepin-2-one (230 mg, 0.524 mmol, 1 equiv) and N-methyl-4-(4,4,5,5-tetramethyl-1,3,2-dioxaborolan-2-yl)picolinamide (274 mg, 1.05 mmol, 2 equiv) and Pd(dppf)Cl₂ (96 mg, 0.13 mmol, 0.25 equiv) were placed in a round-bottom flask, followed by 1,4-dioxane (8.4 mL) and 2N Na₂CO₃ aqueous solution (4.2 mL). The solution was degassed and heated at 80° C. for 1 h. The solution was quenched with saturated ammonium chloride and extracted with EA (3×10 mL). The organic solutions were combined, washed with H₂O (2×10 mL) and brine solution (1×20 mL), dried over anhydrous Na₂SO₄, filtered and concentrated by rotary evaporation to get the crude compound. Then, the resulting concentrate was purified on a silica column to get 120 mg (purity=99%) yellow solid compound with a yield of 46.35%.

TLC R_{f}=0.4 (DCM:EA=2:1);

LCMS (m/z)=495.30 (M+H)

¹H NMR (400 MHz, DMSO-d₆) δ 8.82 (q, J=4.9 Hz, 1H), 8.70 (d, J=5.1 Hz, 1H), 8.34-8.23 (m, 1H), 8.02 (s, 1H), 7.91 (d, J=3.2 Hz, 1H), 7.65 (s, 2H), 7.59 (s, 1H), 7.04 (d, J=1.3 Hz, 1H), 6.95 (d, J=8.7 Hz, 2H), 6.76 (d, J=8.7 Hz, 2H), 5.22 (s, 2H), 4.17-3.01 (br., 2H), 3.64 (s, 3H), 2.86 (d, J=4.8 Hz, 3H), 2.39 (s, 3H).

### Example 61. 8-(1-(2-hydroxypropyl)-1H-pyrazol-4-yl)-1-(4-methoxybenzyl)-4-(5-methyloxazol-2-yl)-1,3-dihydro-2H-benzo[b]azepin-2-one (61)

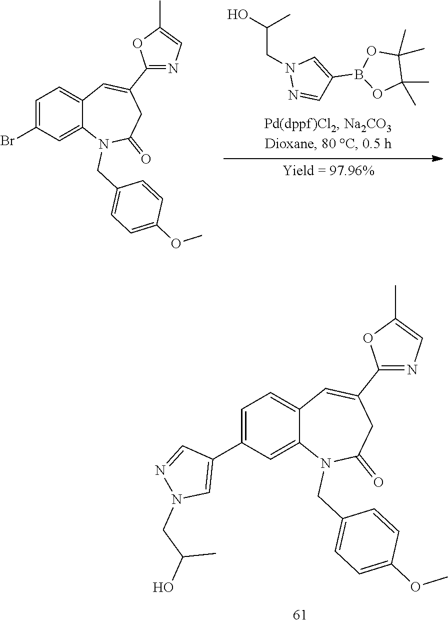 [SMILES: COc1ccc(CN2C(=O)CC(c3ncc(C)o3)=Cc3ccc(Br)cc32)cc1 ; CC(O)Cn1cc(B2OC(C)(C)C(C)(C)O2)cn1 ; COc1ccc(CN2C(=O)CC(c3ncc(C)o3)=Cc3ccc(-c4cnn(CC(C)O)c4)cc32)cc1]

8-bromo-1-(4-methoxybenzyl)-4-(5-methyloxazol-2-yl)-1,3-dihydro-2H-benzo[b]azepin-2-one (87 mg, 0.198 mmol, 1 equiv) and 1-(4-(4,4,5,5-tetramethyl-1,3,2-dioxaborolan-2-yl)-1H-pyrazol-1-yl)propan-2-ol (100 mg, 0.396 mmol, 2 equiv) and Pd(dppf)Cl2 (36 mg, 0.495 mmol, 0.25 equiv) were placed in a round-bottom flask, followed by 1,4-dioxane (3.6 mL) and 2N Na₂CO₃ aqueous solution (1.8 mL). The solution was degassed and heated at 80° C. for 0.5 h. The solution was quenched with saturated ammonium chloride and extracted with EA (3×10 mL). The organic solutions were combined, washed with H₂O (2×10 mL) and brine solution (1×20 mL), dried over anhydrous Na₂SO₄, filtered and concentrated by rotary evaporation to get the crude compound. Then, the resulting concentrate was purified on a silica column to get 94 mg (purity=97%) yellow solid compound with a yield of 98.94%.

TLC R_{f}=0.15 (DCM:EA=2:1);

LCMS (m/z)=485.2 (M+H)

¹H NMR (400 MHz, Chloroform-d) δ 7.68 (s, 1H), 7.61 (s, 1H), 7.49 (s, 1H), 7.44 (s, 1H), 7.31 (d, J=8.1 Hz, 1H), 7.25 (d, J=6.9 Hz, 1H), 7.07 (d, J=8.6 Hz, 2H), 6.84 (s, 1H), 6.77 (d, J=8.6 Hz, 2H), 5.06 (s, 2H), 4.24 (t, J=8.2 Hz, 1H), 4.18 (dd, J=13.7, 2.5 Hz, 1H), 4.02 (dd, J=13.7, 7.8 Hz, 1H), 3.75 (s, 3H), 3.34 (s, 2H), 2.37 (s, 3H), 1.25 (d, J=6.2 Hz, 3H).

### Example 62. 1-(4-Methoxybenzyl)-8-(1-methyl-2-oxo-1,2-dihydropyridin-4-yl)-4-(5-methyloxazol-2-yl)-1,3-dihydro-2H-benzo[b]azepin-2-one (62)

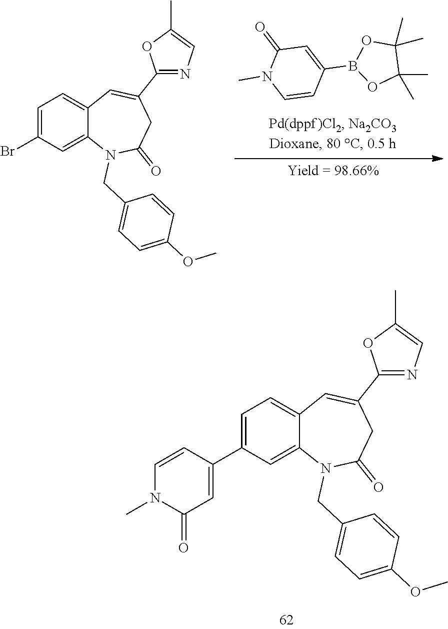

8-bromo-1-(4-methoxybenzyl)-4-(5-methyloxazol-2-yl)-1,3-dihydro-2H-benzo[b]azepin-2-one (200 mg, 0.455 mmol, 1 equiv) and 1-methyl-4-(4,4,5,5-tetramethyl-1,3,2-dioxaborolan-2-yl)pyridin-2(1H)-one (214 mg, 0.910 mmol, 2 equiv) and Pd(dppf)Cl₂ (83 mg, 0.114 mmol, 0.25 equiv) were placed in a round-bottom flask, followed by 1,4-dioxane (8.4 mL) and 2N Na₂CO₃ aqueous solution (4.2 mL). The solution was degassed and heated at 80° C. for 0.5 h. The solution was quenched with saturated ammonium chloride and extracted with EA (3×10 mL). The organic solutions were combined, washed with H₂O (2×10 mL) and brine solution (1×20 mL), dried over anhydrous Na₂SO₄, filtered and concentrated by rotary evaporation to get the crude compound. Then, the resulting concentrate was purified on a silica column to get 210 mg (purity=96%) yellow solid compound with a yield of 98.66%.

TLC R_{f}=0.05 (DCM:EA=2:1);

LCMS (m/z)=468.1 (M+H)

¹H NMR (400 MHz, Chloroform-d) δ 7.53 (s, 1H), 7.52 (s, 1H), 7.39 (d, J=8.1 Hz, 1H), 7.33 (dt, J=8.1, 2.1 Hz, 2H), 7.02 (s, 1H), 7.00 (s, 1H), 6.86 (d, J=1.4 Hz, 1H), 6.81-6.67 (m, 3H), 6.26 (d, J=6.8 Hz, 1H), 5.10 (s, 2H), 4.12-3.03 (br., 2H), 3.73 (s, 3H), 3.57 (s, 3H), 2.38 (d, J=1.1 Hz, 3H).

### Example 63. 1-(4-Methoxybenzyl)-8-(1-methyl-2-oxo-1,2-dihydropyridin-4-yl)-4-(5-methyl oxazol-2-yl)-1,3-dihydro-2H-benzo[b]azepin-2-one (63)

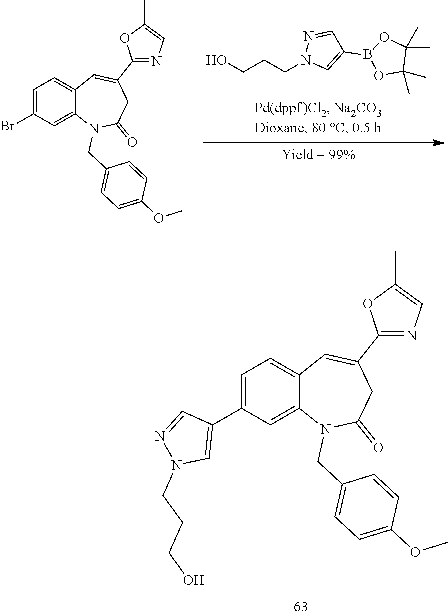 [SMILES: COc1ccc(CN2C(=O)CC(c3ncc(C)o3)=Cc3ccc(Br)cc32)cc1 ; CC1(C)OB(c2cnn(CCCO)c2)OC1(C)C ; COc1ccc(CN2C(=O)CC(c3ncc(C)o3)=Cc3ccc(-c4cnn(CCCO)c4)cc32)cc1]

8-bromo-1-(4-methoxybenzyl)-4-(5-methyloxazol-2-yl)-1,3-dihydro-2H-benzo[b]azepin-2-one (87 mg, 0.198 mmol, 1 equiv) and 3-(4-(4,4,5,5-tetramethyl-1,3,2-dioxaborolan-2-yl)-1H-pyrazol-1-yl)propan-1-ol (100 mg, 0.396 mmol, 2 equiv) and Pd(dppf)Cl₂ (50 mg, 0.362 mmol, 0.25 equiv) were placed in a round-bottom flask, followed by 1,4-dioxane (3.6 mL) and 2N Na₂CO₃ aqueous solution (1.8 mL). The solution was degassed and heated at 80° C. for 0.5 h. The solution was quenched with saturated ammonium chloride and extracted with EA (3×10 mL). The organic solutions were combined, washed with H₂O (2×10 mL) and brine solution (1×20 mL), dried over anhydrous Na₂SO₄, filtered and concentrated by rotary evaporation to get the crude compound. Then, the resulting concentrate was purified on a silica column to get 95 mg (purity=98%) yellow oil compound with a yield of 99%.

TLC R_{f}=0.05 (DCM:EA=2:1);

LCMS (m/z)=485.5 (M+H)

¹H NMR (400 MHz, Chloroform-d) δ 7.67 (s, 1H), 7.58 (s, 1H), 7.49 (s, 1H), 7.43 (s, 1H), 7.30 (d, J=8.1 Hz, 1H), 7.24 (d, J=8.3 Hz, 1H), 7.08 (d, J=8.6 Hz, 2H), 6.84 (d, J=1.3 Hz, 1H), 6.78 (d, J=8.7 Hz, 2H), 5.06 (s, 2H), 4.31 (t, J=6.4 Hz, 2H), 4.12-3.09 (br., 2H), 3.75 (s, 3H), 3.65 (t, J=5.6 Hz, 2H), 2.37 (s, 3H), 2.08 (p, J=5.8 Hz, 2H).

### Example 64. 1-(4-methoxybenzyl)-4-(5-methyloxazol-2-yl)-8-(1-(2-(pyrrolidin-1-yl)ethyl)-1H-pyrazol-4-yl)-1,3-dihydro-2H-benzo[b]azepin-2-one (64)

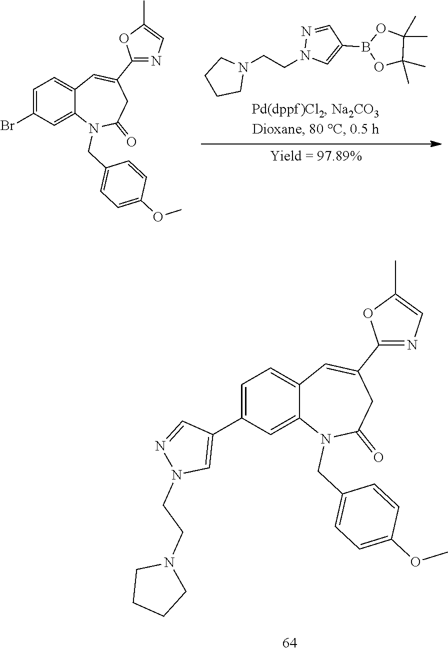 [SMILES: COc1ccc(CN2C(=O)CC(c3ncc(C)o3)=Cc3ccc(Br)cc32)cc1 ; CC1(C)OB(c2cnn(CCN3CCCC3)c2)OC1(C)C ; COc1ccc(CN2C(=O)CC(c3ncc(C)o3)=Cc3ccc(-c4cnn(CCN5CCCC5)c4)cc32)cc1]

8-bromo-1-(4-methoxybenzyl)-4-(5-methyloxazol-2-yl)-1,3-dihydro-2H-benzo[b]azepin-2-one (189 mg, 0.43 mmol, 1 equiv) and 1-(2-(pyrrolidin-1-yl)ethyl)-4-(4,4,5,5-tetramethyl-1,3,2-dioxaborolan-2-yl)-1H-pyrazole (250 mg, 0.860 mmol, 2 equiv) and Pd(dppf)Cl2 (79 mg, 0.108 mmol, 0.25 equiv) were placed in a round-bottom flask, followed by 1,4-dioxane (8 mL) and 2N Na₂CO₃ aqueous solution (4 mL). The solution was degassed and heated at 80° C. for 0.5 h. The solution was quenched with saturated ammonium chloride and extracted with EA (3×10 mL). The organic solutions were combined, washed with H₂O (2×10 mL) and brine solution (1×20 mL), dried over anhydrous Na₂SO₄, filtered and concentrated by rotary evaporation to get the crude compound. Then, the resulting concentrate was purified on a silica column to get 207 mg (purity=97%) brown oil compound with a yield of 92%.

TLC R_{f}=0.3 (DCM:MeOH=10:1);

LCMS (m/z)=524.60 (M+H)

¹H NMR (400 MHz, Chloroform-d) δ 7.71 (s, 1H), 7.66 (s, 1H), 7.48 (s, 1H), 7.45 (s, 1H), 7.30 (d, J=8.1 Hz, 1H), 7.25 (d, J=6.4 Hz, 1H), 7.06 (d, J=8.7 Hz, 2H), 6.83 (s, 1H), 6.76 (d, J=8.7 Hz, 2H), 5.06 (s, 2H), 4.37 (t, J=6.6 Hz, 2H), 3.73 (s, 3H), 4.12-3.14 (br., 2H), 3.11 (t, J=6.6 Hz, 2H), 2.65 (s, 4H), 2.36 (s, 3H), 1.82 (s, 4H).

### Example 65. 1-(4-methoxybenzyl)-4-(5-methyloxazol-2-yl)-8-(1-(2-(pyrrolidin-1-yl)ethyl)-1H-pyrazol-4-yl)-1,3-dihydro-2H-benzo[b]azepin-2-one (65)

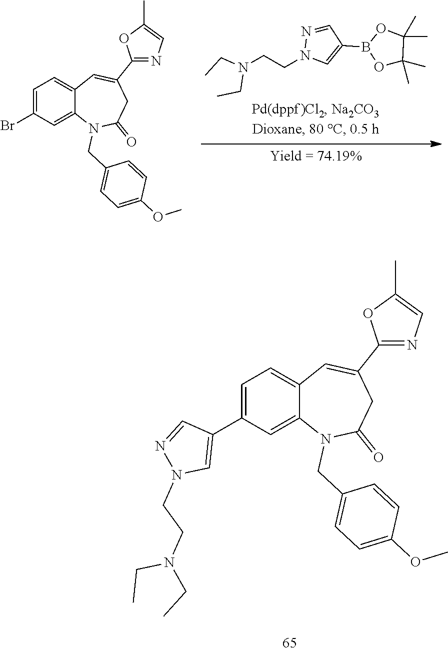 [SMILES: COc1ccc(CN2C(=O)CC(c3ncc(C)o3)=Cc3ccc(Br)cc32)cc1 ; CCN(CC)CCn1cc(B2OC(C)(C)C(C)(C)O2)cn1 ; CCN(CC)CCn1cc(-c2ccc3c(c2)N(Cc2ccc(OC)cc2)C(=O)CC(c2ncc(C)o2)=C3)cn1]

8-bromo-1-(4-methoxybenzyl)-4-(5-methyloxazol-2-yl)-1,3-dihydro-2H-benzo[b]azepin-2-one (187 mg, 0.426 mmol, 1 equiv) and 1-(2-(pyrrolidin-1-yl)ethyl)-4-(4,4,5,5-tetramethyl-1,3,2-dioxaborolan-2-yl)-1H-pyrazole (250 mg, 0.851 mmol, 2 equiv) and Pd(dppf)Cl2 (78 mg, 0.106 mmol, 0.25 equiv) were placed in a round-bottom flask, followed by 1,4-dioxane (8 mL) and 2N Na₂CO₃ aqueous solution (4 mL). The solution was degassed and heated at 80° C. for 0.5 h. The solution was quenched with saturated ammonium chloride and extracted with EA (3×10 mL). The organic solutions were combined, washed with H₂O (2×10 mL) and brine solution (1×20 mL), dried over anhydrous Na₂SO₄, filtered and concentrated by rotary evaporation to get the crude compound. Then, the resulting concentrate was purified on a silica column to get 166 mg (purity=99%) yellow oil compound with a yield of 74.19%.

TLC R_{f}=0.35 (DCM:MeOH=10:1);

LCMS (m/z)=526.60 (M+H)

¹H NMR (400 MHz, Chloroform-d) δ 7.67 (s, 1H), 7.66 (s, 1H), 7.50 (s, 1H), 7.44 (d, J=1.6 Hz, 1H), 7.31 (d, J=8.1 Hz, 1H), 7.28-7.22 (m, 1H), 7.08 (d, J=8.7 Hz, 2H), 6.84 (d, J=1.3 Hz, 1H), 6.77 (d, J=8.7 Hz, 2H), 5.07 (s, 2H), 4.23 (t, J=6.5 Hz, 2H), 4.16-3.18 (br., 2H), 3.74 (s, 3H), 2.93 (t, J=6.5 Hz, 2H), 2.59 (q, J=7.1 Hz, 4H), 2.38 (d, J=1.2 Hz, 3H), 1.02 (t, J=7.0 Hz, 6H).

### Example 66. 8-(1-(2-(Dimethylamino)ethyl)-1H-pyrazol-4-yl)-1-(4-methoxybenzyl)-4-(5-methyloxazol-2-yl)-1,3-dihydro-2H-benzo[b]azepin-2-one (66)

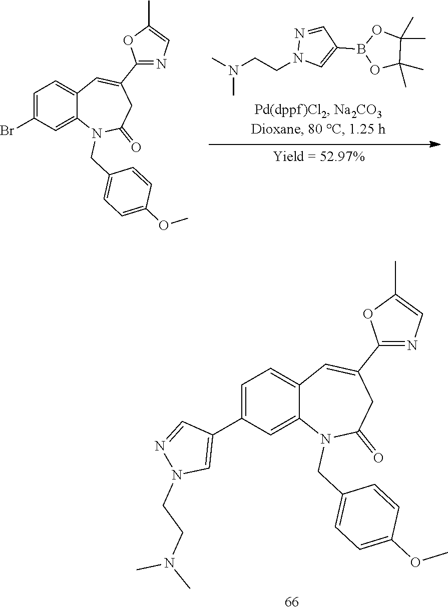

8-bromo-1-(4-methoxybenzyl)-4-(5-methyloxazol-2-yl)-1,3-dihydro-2H-benzo[b]azepin-2-one (200 mg, 0.455 mmol, 1 equiv) and N,N-dimethyl-2-(4-(4,4,5,5-tetramethyl-1,3,2-dioxaborolan-2-yl)-1H-pyrazol-1-yl)ethan-1-amine (241 mg, 0.91 mmol, 2 equiv) and Pd(dppf)Cl₂ (83 mg, 0.114 mmol, 0.25 equiv) were placed in a round-bottom flask, followed by 1,4-dioxane (8.4 mL) and 2N Na₂CO₃ aqueous solution (4.2 mL). The solution was degassed and heated at 80° C. for 1.25 h. The solution was quenched with saturated ammonium chloride and extracted with EA (3×10 mL). The organic solutions were combined, washed with H₂O (2×10 mL) and brine solution (1×20 mL), dried over anhydrous Na₂SO₄, filtered and concentrated by rotary evaporation to get the crude compound. Then, the resulting concentrate was purified on a silica column to get 120 mg (purity=94%) yellow solid compound with a yield of 52.97%.

TLC R_{f}=0.35 (DCM:MeOH=10:1);

LCMS (m/z)=498.50 (M+H)

¹H NMR (400 MHz, Chloroform-d) δ 7.68 (s, 1H), 7.66 (s, 1H), 7.49 (s, 1H), 7.44 (d, J=1.6 Hz, 1H), 7.30 (d, J=8.1 Hz, 1H), 7.26 (d, J=8.1 Hz, 1H), 7.07 (d, J=8.7 Hz, 2H), 6.83 (d, J=1.4 Hz, 1H), 6.77 (d, J=8.7 Hz, 2H), 5.06 (s, 2H), 4.30 (t, J=6.5 Hz, 2H), 4.06-3.17 (br., 2H), 3.74 (s, 3H), 2.88 (t, J=6.4 Hz, 2H), 2.37 (d, J=1.2 Hz, 3H), 2.33 (s, 6H).

### Example 67. 2-(4-(1-(4-Methoxybenzyl)-4-(5-methyloxazol-2-yl)-2-oxo-2,3-dihydro-1H-benzo[b]azepin-8-yl)-1H-pyrazol-1-yl)-N-methylacetamide (67)

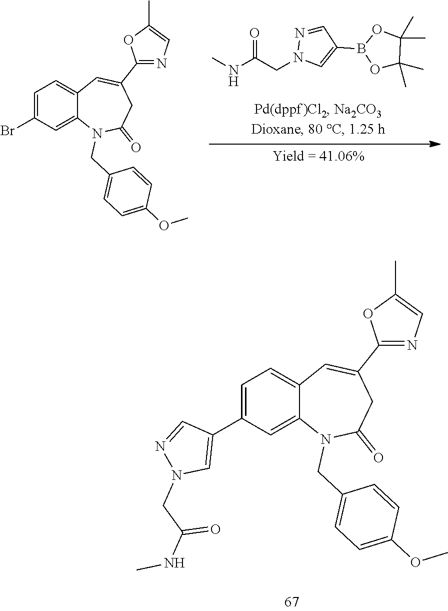

8-bromo-1-(4-methoxybenzyl)-4-(5-methyloxazol-2-yl)-1,3-dihydro-2H-benzo[b]azepin-2-one (200 mg, 0.455 mmol, 1 equiv) and N,N-dimethyl-2-(4-(4,4,5,5-tetramethyl-1,3,2-dioxaborolan-2-yl)-1H-pyrazol-1-yl)ethan-1-amine (241 mg, 0.911 mmol, 2 equiv) and Pd(dppf)Cl₂ (83 mg, 0.114 mmol, 0.25 equiv) were placed in a round-bottom flask, followed by 1,4-dioxane (8.4 mL) and 2N Na₂CO₃ aqueous solution (4.2 mL). The solution was degassed and heated at 80° C. for 1.25 h. The solution was quenched with saturated ammonium chloride and extracted with EA (3×10 mL). The organic solutions were combined, washed with H₂O (2×10 mL) and brine solution (1×20 mL), dried over anhydrous Na₂SO₄, filtered and concentrated by rotary evaporation to get the crude compound. Then, the resulting concentrate was purified on a silica column to get 93 mg (purity=97%) yellow solid compound with a yield of 41.06%.

TLC R_{f}=0.7 (DCM:MeOH=10:1);

LCMS (m/z)=498.40 (M+H)

¹H NMR (400 MHz, DMSO-d₆) δ 8.23 (s, 1H), 8.05 (q, J=4.6 Hz, 1H), 7.95 (s, 1H), 7.81 (d, J=1.7 Hz, 1H), 7.49 (s, 1H), 7.46 (d, J=8.2 Hz, 1H), 7.40 (dd, J=8.0, 1.6 Hz, 1H), 7.01 (d, J=1.3 Hz, 1H), 6.90 (d, J=8.6 Hz, 2H), 6.74 (d, J=8.7 Hz, 2H), 5.18 (s, 2H), 4.79 (s, 2H), 4.13-3.13 (br., 2H), 3.64 (s, 3H), 2.63 (d, J=4.6 Hz, 3H), 2.38 (d, J=1.2 Hz, 3H).

### Example 68. 2-(4-(1-(4-methoxybenzyl)-4-(5-methyloxazol-2-yl)-2-oxo-2,3-dihydro-1H-benzo[b]azepin-8-yl)-1H-pyrazol-1-yl)acetonitrile (68)

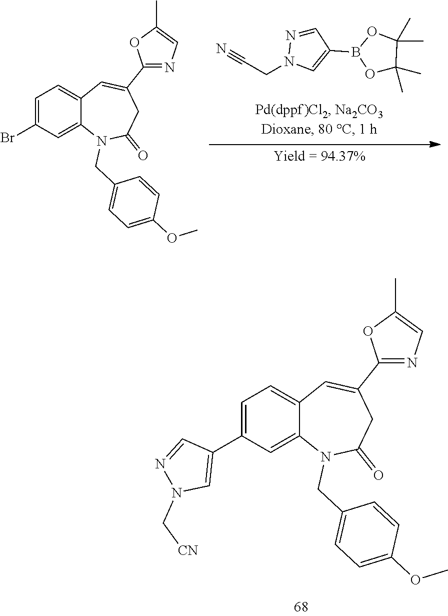 [SMILES: COc1ccc(CN2C(=O)CC(c3ncc(C)o3)=Cc3ccc(Br)cc32)cc1 ; CC1(C)OB(c2cnn(CC#N)c2)OC1(C)C ; COc1ccc(CN2C(=O)CC(c3ncc(C)o3)=Cc3ccc(-c4cnn(CC#N)c4)cc32)cc1]

8-bromo-1-(4-methoxybenzyl)-4-(5-methyloxazol-2-yl)-1,3-dihydro-2H-benzo[b]azepin-2-one (200 mg, 0.455 mmol, 1 equiv) and 2-(4-(4,4,5,5-tetramethyl-1,3,2-dioxaborolan-2-yl)-1H-pyrazol-1-yl)acetonitrile (212 mg, 0.911 mmol, 2 equiv) and Pd(dppf)Cl2 (83 mg, 0.114 mmol, 0.25 equiv) were placed in a round-bottom flask, followed by 1,4-dioxane (8.4 mL) and 2N Na₂CO₃ aqueous solution (4.2 mL). The solution was degassed and heated at 80° C. for 1 h. The solution was quenched with saturated ammonium chloride and extracted with EA (3×10 mL). The organic solutions were combined, washed with H₂O (2×10 mL) and brine solution (1×20 mL), dried over anhydrous Na₂SO₄, filtered and concentrated by rotary evaporation to get the crude compound. Then, the resulting concentrate was purified on a silica column to get 200 mg (purity=98%) yellow solid compound with a yield of 94.37%.

TLC R_{f}=0.5 (DCM:EA=2:1);

LCMS (m/z)=466.30 (M+H)

¹H NMR (400 MHz, Chloroform-d) δ 7.73 (d, J=7.4 Hz, 2H), 7.51 (s, 1H), 7.45 (s, 1H), 7.34 (d, J=7.3 Hz, 1H), 7.26 (d, merged peak with CDCl3, J=7.3 Hz, 1H), 7.08 (d, J=7.9 Hz, 2H), 6.85 (s, 1H), 6.79 (d, J=7.6 Hz, 2H), 5.11 (s, 2H), 5.06 (s, 2H), 4.13-2.93 (br., 2H), 3.75 (s, 3H), 2.38 (s, 3H).

### Example 69. 2-(4-(1-(4-Methoxybenzyl)-4-(5-methyloxazol-2-yl)-2-oxo-2,3-dihydro-1H-benzo[b]azepin-8-yl)-1H-pyrazol-1-yl)acetic acid (69)

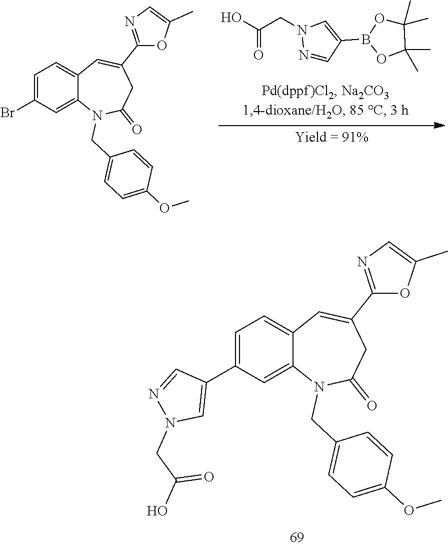 [SMILES: COc1ccc(CN2C(=O)CC(c3ncc(C)o3)=Cc3ccc(Br)cc32)cc1 ; CC1(C)OB(c2cnn(CC(=O)O)c2)OC1(C)C ; COc1ccc(CN2C(=O)CC(c3ncc(C)o3)=Cc3ccc(-c4cnn(CC(=O)O)c4)cc32)cc1]

8-bromo-1-(4-methoxybenzyl)-4-(5-methyloxazol-2-yl)-1,3-dihydro-2H-benzo[b]azepin-2-one (100 mg, 0.235 mmol, 1 equiv) and 2-(4-(4,4,5,5-tetramethyl-1,3,2-dioxaborolan-2-yl)-1H-pyrazol-1-yl)acetic acid (92.4 mg, 0.352 mmol, 2 equiv) and were placed in a round-bottom flask, followed by 1,4-dioxane/H₂O (8:2, 5 mL) and Na₂CO₃ (62.3 mg, 0.352 mmol, 2 equiv) the solution was degassed for 5 min then add Pd(dppf)Cl2 (34.4 mg, 0.047 mol, 0.2 equiv) and heated at 80° C. for 3 h. After TLC complies the reaction mass was cooled to RT, quenched with saturated ammonium chloride and extracted with EA (3×15 mL). The organic solutions were combined, washed with H₂O (2×10 mL) and brine solution (1×10 mL), dried over anhydrous Na₂SO₄, filtered and concentrated by rotary evaporation to get the crude compound. The crude was purified by silica column to get pure compound (100 mg, purity=98.3%, yield 91%) as an off white solid.

TLC R_{f}=0.4 (EA)

LCMS (m/z)=485.30 (M+H)

### Example 70. 8-(1-(Azetidin-3-yl)-1H-pyrazol-4-yl)-1-(4-methoxybenzyl)-4-(5-methyloxazol-2-yl)-1,3-dihydro-2H-benzo[b]azepin-2-one (70)

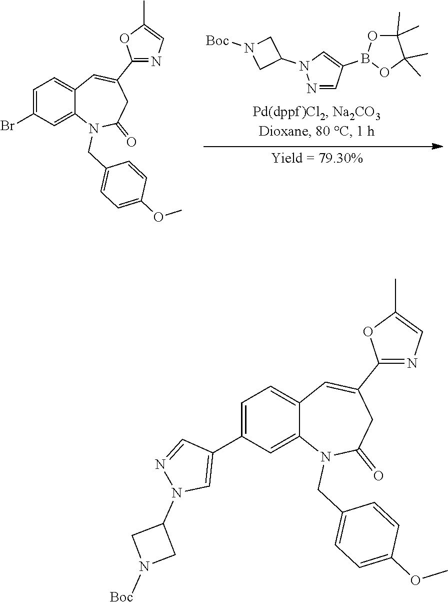 [SMILES: COc1ccc(CN2C(=O)CC(c3ncc(C)o3)=Cc3ccc(Br)cc32)cc1 ; CC(C)(C)OC(=O)N1CC(n2cc(B3OC(C)(C)C(C)(C)O3)cn2)C1 ; COc1ccc(CN2C(=O)CC(c3ncc(C)o3)=Cc3ccc(-c4cnn(C5CN(C(=O)OC(C)(C)C)C5)c4)cc32)cc1]

### 2-(4-(1-(4-methoxybenzyl)-4-(5-methyloxazol-2-yl)-2-oxo-2,3-dihydro-1H-benzo[b]azepin-8-yl)-1H-pyrazol-1-yl)acetonitrile

8-bromo-1-(4-methoxybenzyl)-4-(5-methyloxazol-2-yl)-1,3-dihydro-2H-benzo[b]azepin-2-one (200 mg, 0.455 mmol, 1 equiv) and 2-(4-(4,4,5,5-tetramethyl-1,3,2-dioxaborolan-2-yl)-1H-pyrazol-1-yl)acetonitrile (318 mg, 0.911 mmol, 2 equiv) and Pd(dppf)Cl2 (83 mg, 0.114 mmol, 0.25 equiv) were placed in a round-bottom flask, followed by 1,4-dioxane (8.4 mL) and 2N Na₂CO₃ aqueous solution (4.2 mL). The solution was degassed and heated at 80° C. for 1 h. The solution was quenched with saturated ammonium chloride and extracted with EA (3×10 mL). The organic solutions were combined, washed with H₂O (2×10 mL) and brine solution (1×20 mL), dried over anhydrous Na₂SO₄, filtered and concentrated by rotary evaporation to get the crude compound. Then, the resulting concentrate was purified on a silica column to get 210 mg (purity=94%) yellow solid compound with a yield of 79.30%.

TLC R_{f}=0.5 (DCM:EA=2:1);

LCMS (m/z)=582.50 (M+H)

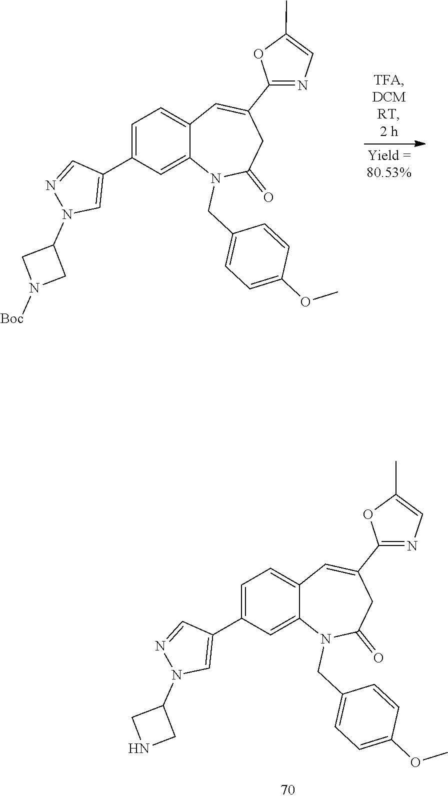 [SMILES: COc1ccc(CN2C(=O)CC(c3ncc(C)o3)=Cc3ccc(-c4cnn(C5CN(C(=O)OC(C)(C)C)C5)c4)cc32)cc1 ; COc1ccc(CN2C(=O)CC(c3ncc(C)o3)=Cc3ccc(-c4cnn(C5CNC5)c4)cc32)cc1]

### 8-(1-(Azetidin-3-yl)-1H-pyrazol-4-yl)-1-(4-methoxybenzyl)-4-(5-methyloxazol-2-yl)-1,3-dihydro-2H-benzo[b]azepin-2-one (70)

tert-butyl 3-(4-(1-(4-methoxybenzyl)-4-(5-methyloxazol-2-yl)-2-oxo-2,3-dihydro-1H-benzo[b]azepin-8-yl)-1H-pyrazol-1-yl)azetidine-1-carboxylate (210 mg, 0.361 mmol, 1 equiv) were placed in a round-bottom flask, followed by DCM (1.8 mL) and TFA (3.66 mL). The solution stirred at room temperature for 2 h. The solution was quenched with saturated ammonium chloride and extracted with DCM (3×10 mL). The organic solutions were combined, washed with H₂O (2×10 mL) and brine solution (1×20 mL), dried over anhydrous Na₂SO₄, filtered and concentrated by rotary evaporation to get the crude compound. Then, the resulting concentrate was purified on a silica column to get 140 mg (purity=89%) white solid compound with a yield of 80.53%.

TLC R_{f}=0.05 (DCM:EA=2:1);

LCMS (m/z)=482.40 (M+H)

### Example 71. Ethyl 2-(4-(1-(4-methoxybenzyl)-4-(5-methyloxazol-2-yl)-2-oxo-2,3-dihydro-1H-benzo[b]azepin-8-yl)-1H-pyrazol-1-yl)acetate (71)

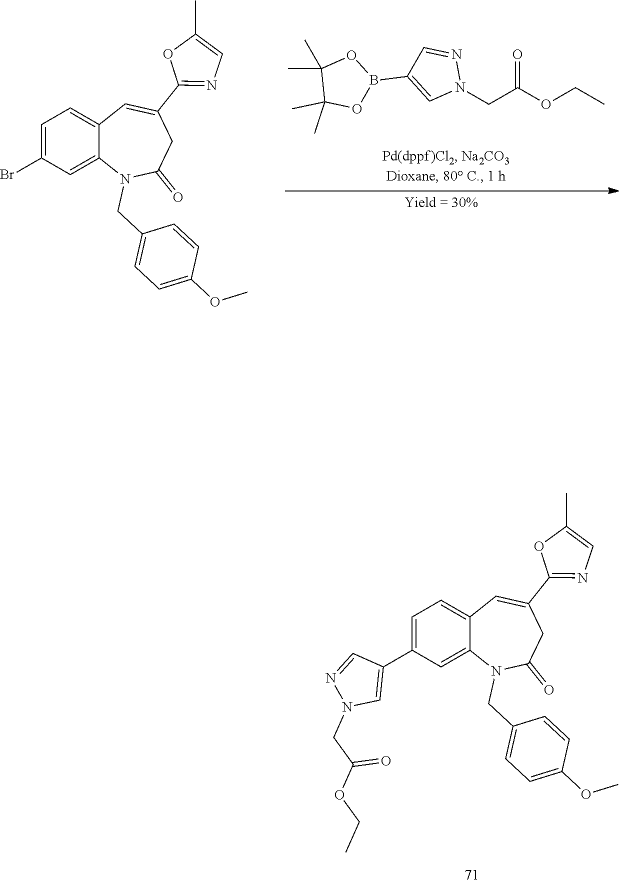 [SMILES: COc1ccc(CN2C(=O)CC(c3ncc(C)o3)=Cc3ccc(Br)cc32)cc1 ; CCOC(=O)Cn1cc(B2OC(C)(C)C(C)(C)O2)cn1 ; CCOC(=O)Cn1cc(-c2ccc3c(c2)N(Cc2ccc(OC)cc2)C(=O)CC(c2ncc(C)o2)=C3)cn1]

8-Bromo-1-(4-methoxybenzyl)-4-(5-methyloxazol-2-yl)-1,3-dihydro-2H-benzo[b]azepin-2-one (200 mg, 0.455 mmol, 1 equiv) and ethyl 2-(4-(4,4,5,5-tetramethyl-1,3,2-dioxaborolan-2-yl)-1H-pyrazol-1-yl)acetate (255 mg, 0.911 mmol, 2 equiv) and Pd(dppf)Cl₂ (83 mg, 0.114 mmol, 0.25 equiv) were placed in a round-bottom flask, followed by 1,4-dioxane (8.4 mL) and 2N Na₂CO₃ aqueous solution (4.2 mL). The solution was degassed and heated at 80° C. for 0.5 h. The solution was quenched with saturated ammonium chloride and extracted with EA (3×10 mL). The organic solutions were combined, washed with H₂O (2×10 mL) and brine solution (1×20 mL), dried over anhydrous Na₂SO₄, filtered and concentrated by rotary evaporation to get the crude compound. Then, the resulting concentrate was purified on a silica column to get 70 mg (purity=91%) yellow oil compound with a yield of 30%.

TLC R_{f}=0.5 (DCM:EA=2:1);

LCMS (m/z)=513.40 (M+H)

### Example 72. 2-(4-(1-(4-Methoxybenzyl)-4-(5-methyloxazol-2-yl)-2-oxo-2,3-dihydro-1H-benzo[b]azepin-8-yl)-1H-pyrazol-1-yl) propenamide (72)

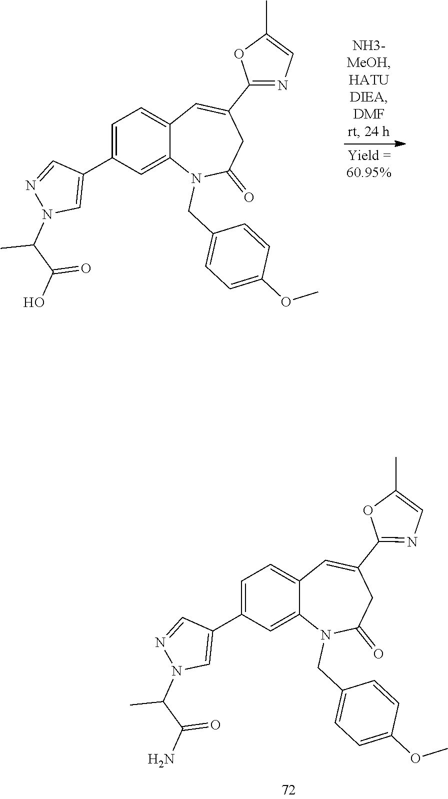

HATU (247 mg, 0.72 mmol, 2.7 equiv) and DIPEA (126 μL, 0.72 mmol, 3 equiv) were added to a solution of 2-(4-(1-(4-methoxybenzyl)-4-(5-methyloxazol-2-yl)-2-oxo-2,3-dihydro-1H-benzo[b]azepin-8-yl)-1H-pyrazol-1-yl) propanoic acid (120 mg, 0.24 mmol, 1 equiv) in DMF (1.2 mL) at 0° C. The mixture was stirred for 30 min followed by the addition of ammonia (2 M in MeOH) (913 μL, 1.83 mmol, 7.6 equiv). The reaction was then stirred at room temperature overnight. The reaction mixture was concentrated, adsorbed onto silica and purified by flash column chromatography with DCM/MeOH (10:1) to afford the title compound (73 mg, yield:60.95%, purity:100%) as a white solid.

TLC R_{f}=0.7 (DCM:MeOH=10:1);

LCMS (m/z)=498.30 (M+H)

¹H NMR (400 MHz, Chloroform-d) δ 7.75 (s, 1H), 7.71 (s, 1H), 7.49 (s, 1H), 7.46 (d, J=1.7 Hz, 1H), 7.32 (d, J=8.1 Hz, 1H), 7.27 (merged peak with CDCl3, d, J=8.1 Hz, 1H), 7.07 (d, J=8.7 Hz, 2H), 6.84 (d, J=1.3 Hz, 1H), 6.77 (d, J=8.7 Hz, 2H), 6.41 (s, 1H), 5.70 (s, 1H), 5.06 (s, 2H), 4.95 (q, J=7.3 Hz, 1H), 3.74 (s, 3H), 2.99 (s, 2H), 2.38 (d, J=1.2 Hz, 3H), 1.83 (d, J=7.3 Hz, 3H).

### Example 73. 2-(4-(1-(4-Methoxybenzyl)-4-(5-methyloxazol-2-yl)-2-oxo-2,3-dihydro-1H-benzo[b]azepin-8-yl)-1H-pyrazol-1-yl)-N,N-dimethylacetamide (73)

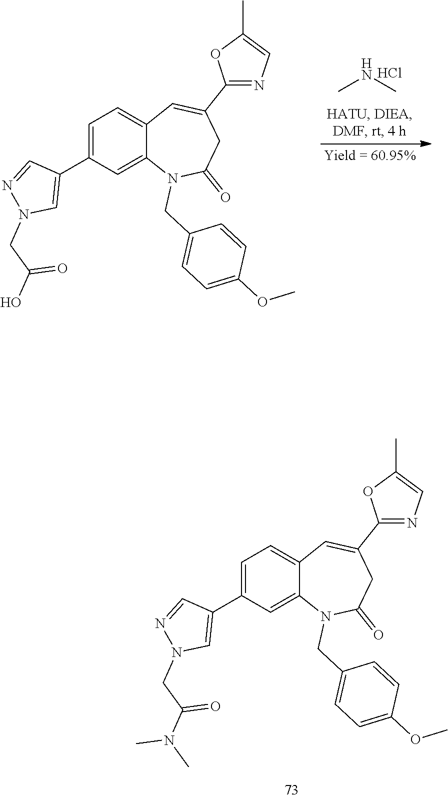

HATU(267 mg, 0.7 mmol, 2 equiv) and DIPEA (306 μL, 1.75 mmol, 5 equiv) were added to a solution of 2-(4-(1-(4-methoxybenzyl)-4-(5-methyloxazol-2-yl)-2-oxo-2,3-dihydro-1H-benzo[b]azepin-8-yl)-1H-pyrazol-1-yl)acetic acid (170 mg, 0.351 mmol, 1 equiv) in DMF (7.3 mL) and dimethylamine hydrochloride (34 mg, 0.421 mmol, 1.5 equiv) was added to the mixture at room temperature. The reaction was then stirred at room temperature for 4 h. The reaction mixture was concentrated, adsorbed onto silica and purified by flash column chromatography with DCM/MeOH (10:1) to afford the title compound (132 mg, yield:73.54%, purity:100%) as a brown oil.

TLC R_{f}=0.5 (DCM:MeOH=10:1);

LCMS (m/z)=512.40 (M+H)

¹H NMR (400 MHz, Chloroform-d) δ 7.73 (s, 1H), 7.69 (s, 1H), 7.48 (s, 1H), 7.46 (d, J=1.5 Hz, 1H), 7.30 (d, J=8.1 Hz, 1H), 7.26 (dd, J=8.0, 1.5 Hz, 1H), 7.06 (d, J=8.7 Hz, 2H), 6.84 (d, J=1.3 Hz, 1H), 6.77 (d, J=8.7 Hz, 2H), 5.06 (s, 2H), 5.00 (s, 2H), 4.21-3.25 (br., 2H), 3.74 (s, 3H), 3.10 (s, 3H), 3.00 (s, 3H), 2.37 (d, J=1.2 Hz, 3H).

### Example 74. Ethyl 2-(4-(1-(4-methoxybenzyl)-4-(5-methyloxazol-2-yl)-2-oxo-2,3-dihydro-1H-benzo[b]azepin-8-yl)-1H-pyrazol-1-yl)propanoate (74)

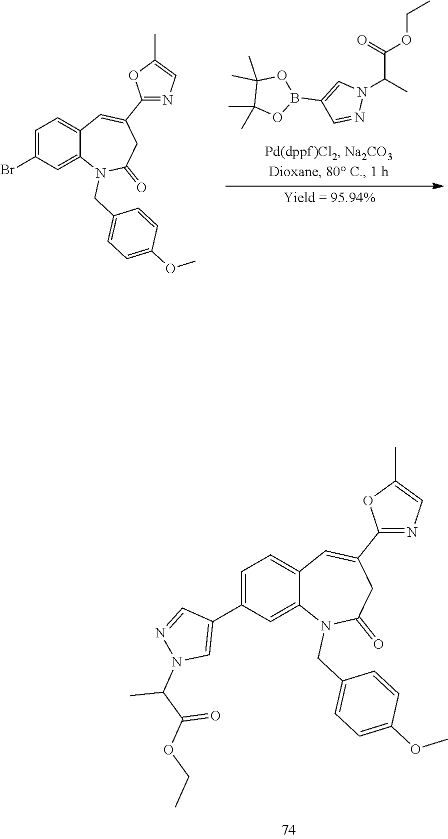 [SMILES: COc1ccc(CN2C(=O)CC(c3ncc(C)o3)=Cc3ccc(Br)cc32)cc1 ; CCOC(=O)C(C)n1cc(B2OC(C)(C)C(C)(C)O2)cn1 ; CCOC(=O)C(C)n1cc(-c2ccc3c(c2)N(Cc2ccc(OC)cc2)C(=O)CC(c2ncc(C)o2)=C3)cn1]

8-bromo-1-(4-methoxybenzyl)-4-(5-methyloxazol-2-yl)-1,3-dihydro-2H-benzo[b]azepin-2-one (200 mg, 0.455 mmol, 1 equiv) and ethyl 2-(4-(4,4,5,5-tetramethyl-1,3,2-dioxaborolan-2-yl)-1H-pyrazol-1-yl)propanoate (268 mg, 0.911 mmol, 2 equiv) and Pd(dppf)Cl₂ (83 mg, 0.114 mmol, 0.25 equiv) were placed in a round-bottom flask, followed by 1,4-dioxane (8.4 mL) and 2N Na₂CO₃ aqueous solution (4.2 mL). The solution was degassed and heated at 80° C. for 1 h. The solution was quenched with saturated ammonium chloride and extracted with EA (3×10 mL). The organic solutions were combined, washed with H₂O (2×10 mL) and brine solution (1×20 mL), dried over anhydrous Na₂SO₄, filtered and concentrated by rotary evaporation to get the crude compound. Then, the resulting concentrate was purified on a silica column to get 230 mg (purity=910%) yellow oil compound with a yield of 95.94%.

TLC R_{f}=0.6 (DCM:EA=2:1);

LCMS (m/z)=527.50 (M+H)

### Example 75. 2-(4-(1-(4-Methoxybenzyl)-4-(5-methyloxazol-2-yl)-2-oxo-2,3-dihydro-1H-benzo[b]azepin-8-yl)-1H-pyrazol-1-yl)propanoic acid (75)

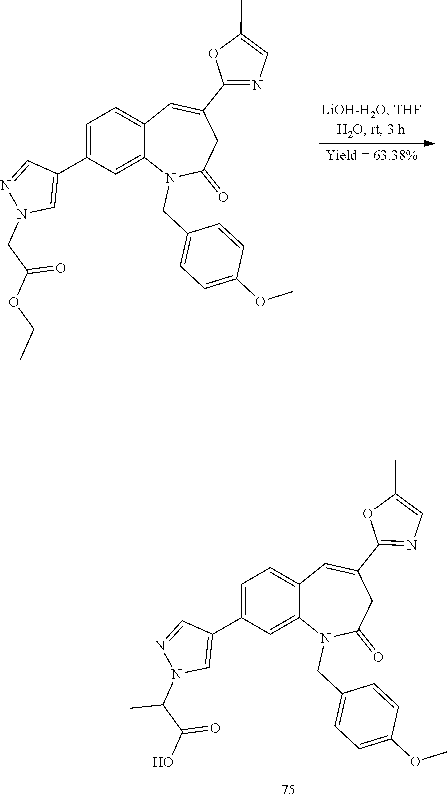

To a stirred solution ethyl 2-(4-(1-(4-methoxybenzyl)-4-(5-methyloxazol-2-yl)-2-oxo-2,3-dihydro-1H-benzo[b]azepin-8-yl)-1H-pyrazol-1-yl)propanoate (200 mg, 0.38 mmol, 1 equiv) in 1:1 mixture solvent-THF/H₂O (1.84 mL), was added LiOH·H₂O (80 mg, 1.9 mmol, 5 equiv) at ambient temperature. The reaction mixture was then stirred at rt for 3 h. The solution was quenched with 1N HCl and extracted with EA (3×10 mL). The organic solutions were combined, washed with H₂O (2×10 mL) and brine solution (1×20 mL), dried over anhydrous Na₂SO₄, filtered and concentrated by rotary evaporation to get the crude compound. Then, the resulting concentrate was purified on a silica column to get 120 mg (purity=100%) yellow solid compound with a yield of 63.38%.

TLC R_{f}=0.4 (DCM:MeOH=10:1);

LCMS (m/z)=499.10 (M+H)

### Example 76. 2-(4-(1-(4-Methoxybenzyl)-4-(5-methyloxazol-2-yl)-2-oxo-2,3-dihydro-1H-benzo[b]azepin-8-yl)-1H-pyrazol-1-yl)-2-methylpropanamide (76)

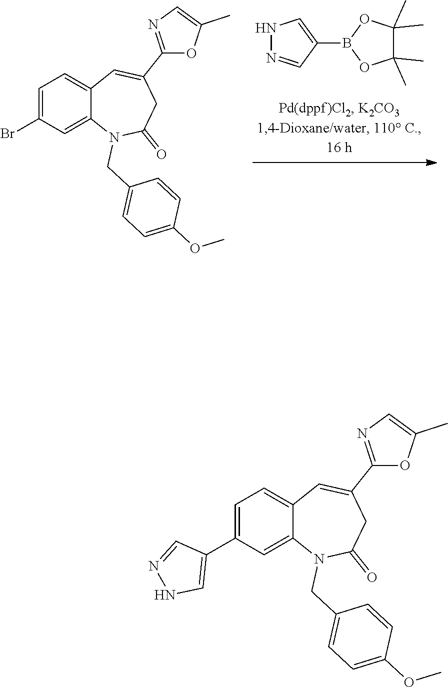 [SMILES: COc1ccc(CN2C(=O)CC(c3ncc(C)o3)=Cc3ccc(Br)cc32)cc1 ; CC1(C)OB(c2cn[nH]c2)OC1(C)C ; COc1ccc(CN2C(=O)CC(c3ncc(C)o3)=Cc3ccc(-c4cn[nH]c4)cc32)cc1]

### 1-(4-Methoxybenzyl)-4-(5-methyloxazol-2-yl)-8-(1H-pyrazol-4-yl)-1,3-dihydro-2H-benzo[b]azepin-2-one

To a stirred solution of 8-bromo-1-(4-methoxybenzyl)-4-(5-methyloxazol-2-yl)-1,3-dihydro-2H-benzo[b]azepin-2-one (1 g, 2.276 mmol, 1 equiv) in 1,4-dioxane/water (15 mL), to this add 4-(4,4,5,5-tetramethyl-1,3,2-dioxaborolan-2-yl)-1H-pyrazole (883 mg, 4.552 mmol, 2 equiv), K₂CO₃ (785 mg, 5.690 mmol). The solution was degassed and added Pd(dppf)Cl2 (166 mg, 0.227 mol, 0.1 equiv), then heated to 80° C. and stirred for 3 h. The reaction mass was quenched with saturated ammonium chloride and extracted with EA (3×50 mL). The organic solutions were combined, washed with H₂O (2×30 mL) and brine solution (1×30 mL), dried over anhydrous Na₂SO₄, filtered and concentrated by rotary evaporation to get the crude compound. The crude was purified by silica column to get pure compound (800 mg, purity=Matches with authentic, yield=80%) as a brown solid.

TLC R_{f}=0.4 (EA)

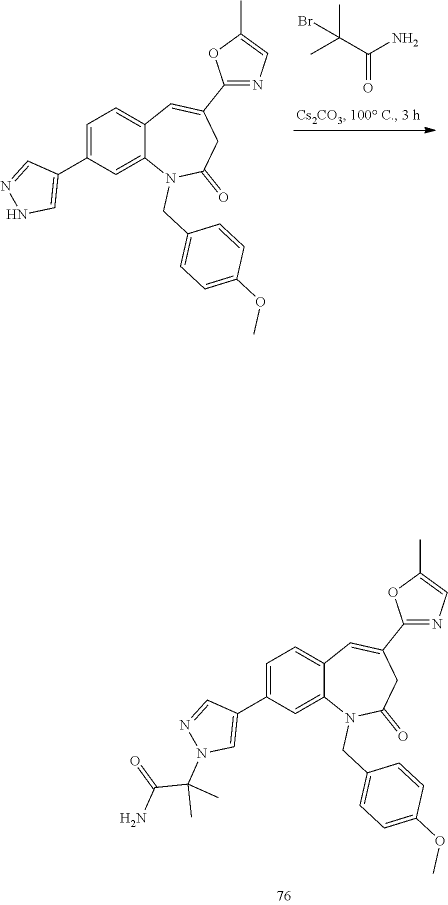

### 2-(4-(1-(4-Methoxybenzyl)-4-(5-methyloxazol-2-yl)-2-oxo-2,3-dihydro-1H-benzo[b]azepin-8-yl)-1H-pyrazol-1-yl)-2-methylpropanamide: (76)

To a stirred solution of 1-(4-methoxybenzyl)-4-(5-methyloxazol-2-yl)-8-(1H-pyrazol-4-yl)-1,3-dihydro-2H-benzo[b]azepin-2-one (300 mg, 0.704 mmol, 1 equiv) in DMF (10 mL) were added 2-bromo-2-methylpropanamide (233 mg, 1.408 mmol, 2 equiv) and Cs₂CO₃ (572 mg, 1.760 mmol). The reaction mixture was stirred at 100° C. for 3 h. Reaction mass was cooled to rt and poured into water (100 mL) and extracted with ethyl acetate (2×50 mL). The combined organic layers were washed with H₂O (3×50 mL) and brine solution (100 mL), dried over anhydrous Na₂SO₄, and concentrated by rotary evaporation. Then, the resulting concentrate was purified on a silica column to get 130 mg of an Off-white solid compound with a yield of 36%, purity=98.8%

TLC R_{f}=0.6 (DCM:MeOH=10:1);

LCMS (m/z)=512.40 (M+H)

¹H NMR (400 MHz, Chloroform-d) δ 7.78 (s, 1H), 7.76 (s, 1H), 7.50 (s, 1H), 7.46 (d, J=1.6 Hz, 1H), 7.33 (d, J=8.1 Hz, 1H), 7.28 (dd, J=8.1, 1.7 Hz, 1H), 7.09 (d, J=8.7 Hz, 2H), 6.84 (d, J=1.3 Hz, 1H), 6.78 (d, J=8.7 Hz, 2H), 6.21 (s, 1H), 5.56 (s, 1H), 5.05 (s, 2H), 4.08-3.27 (br., 2H), 3.75 (s, 3H), 2.37 (d, J=1.2 Hz, 3H), 1.88 (s, 6H).

### Example 77. 8-(1-(3-(Methoxy-d3)propyl)-1H-pyrazol-4-yl)-1-(4-methoxybenzyl)-4-(5-methyloxazol-2-yl)-1,3-dihydro-2H-benzo[b]azepin-2-one (77)

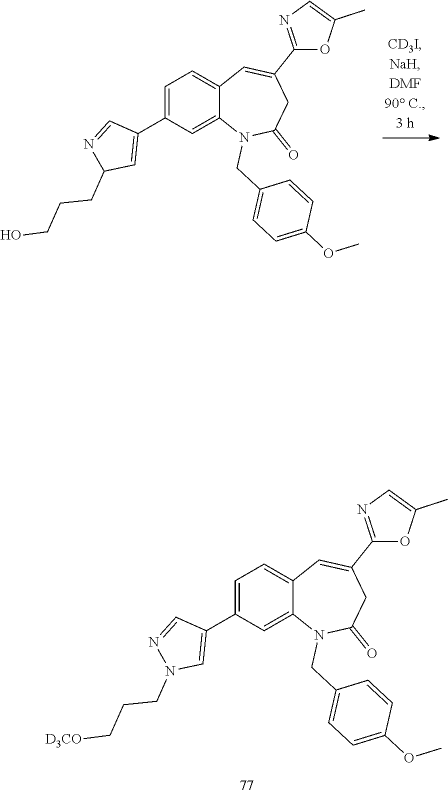

8-(1-(3-hydroxypropyl)-1H-pyrazol-4-yl)-1-(4-methoxybenzyl)-4-(5-methyloxazol-2-yl)-1,3-dihydro-2H-benzo[b]azepin-2-one (220 mg, 0.638 mmol, 1 equiv) and NaH (30 mg, 1.276 mmol, 2.0 equiv) in DMF (10 mL) was added CD₃I (149 mg, 0.957 mmol, 1.5 equiv) at rt, then reaction mass was stirred at 90° C. for 3 h. Reaction progress was monitored by TLC, after TLC complies reaction mass was cooled to rt, poured in to ice water (20 mL) and extracted with ethyl acetate (2×40 mL). The organic solutions were combined, washed with H₂O (30 mL) and brine solution (30 mL), dried over anhydrous Na₂SO₄, filtered and concentrated by rotary evaporation to get the crude compound. The crude was purified by silica column to get pure compound (90 mg, purity=92%, yield=39%) as a brown semi solid.

TLC R_{f}=0.4 (EA:PE 1:1)

LCMS (m/z)=519.40 (M+NH₄)

### Example 78. 1-(4-methoxybenzyl)-8-(1-(2-(2-methoxyethoxy)ethyl)-1H-pyrazol-4-yl)-4-(5-methyloxazol-2-yl)-1,3-dihydro-2H-benzo[b]azepin-2-one (78)

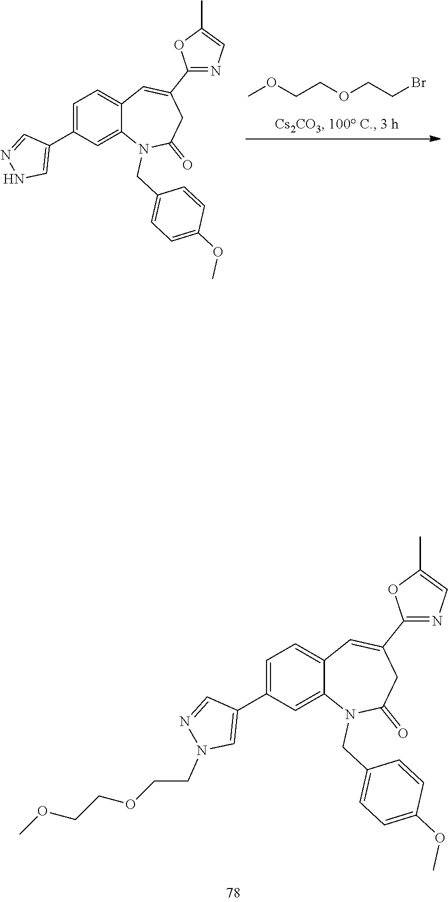

To a stirred solution of 1-(4-methoxybenzyl)-4-(5-methyloxazol-2-yl)-8-(1H-pyrazol-4-yl)-1,3-dihydro-2H-benzo[b]azepin-2-one (100 mg, 0.201 mmol, 1 equiv) in DMF (5 mL) were added 1-bromo-2-(2-methoxyethoxy)ethane (85.9 mg, 0.403 mmol, 2 equiv) and Cs₂CO₃ (185 mg, 0.504 mmol). The reaction mixture was stirred at 100° C. for 3 h. Reaction mass was cooled to rt and poured into water (20 mL) and extracted with ethyl acetate (2×20 mL). The combined organic layers were washed with H₂O (3×10 mL) and brine solution (20 mL), dried over anhydrous Na₂SO₄, and concentrated by rotary evaporation. Then, the resulting concentrate was purified on a silica column to get 55 mg of an Off-white solid compound with a yield of 44%, purity=97.9%

TLC R_{f}=0.5 (PE:EA=4:1);

LCMS (m/z)=529.30 (M+H)

¹H NMR (400 MHz, Chloroform-d) δ 7.72 (s, 1H), 7.66 (s, 1H), 7.49 (s, 1H), 7.46 (d, J=1.6 Hz, 1H), 7.31 (d, J=8.1 Hz, 1H), 7.26 (dd, J=8.1, 1.6 Hz, 1H), 7.07 (d, J=8.6 Hz, 2H), 6.84 (d, J=1.3 Hz, 1H), 6.77 (d, J=8.7 Hz, 2H), 5.07 (s, 2H), 4.33 (t, J=5.2 Hz, 2H), 3.87 (t, J=5.2 Hz, 2H), 3.74 (s, 3H), 3.63-3.55 (m, 2H), 3.55-3.47 (m, 2H), 3.35 (s, 3H), 2.38 (d, J=1.2 Hz, 3H).

### Example 79. 1-(4-methoxybenzyl)-8-(1-(4-methoxybutyl)-1H-pyrazol-4-yl)-4-(5-methyloxazol-2-yl)-1,3-dihydro-2H-benzo[b]azepin-2-one (79)

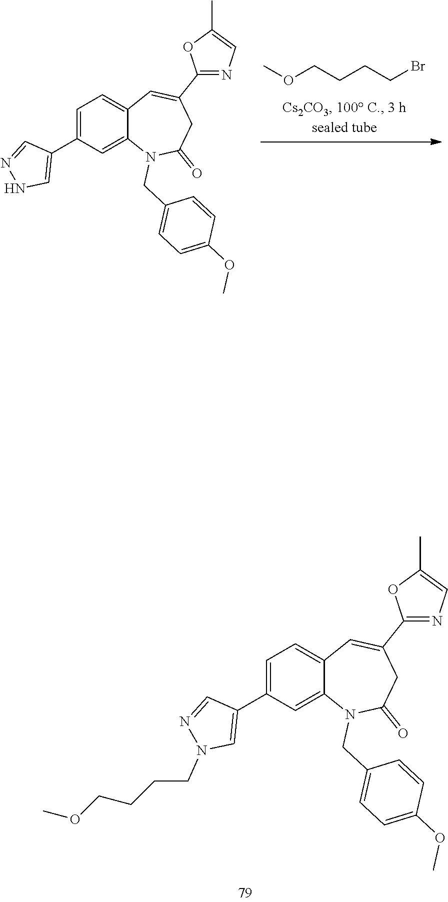

To a stirred solution of 1-(4-methoxybenzyl)-4-(5-methyloxazol-2-yl)-8-(1H-pyrazol-4-yl)-1,3-dihydro-2H-benzo[b]azepin-2-one (100 mg, 0.201 mmol, 1 equiv) in DMF (5 mL) were added 1-bromo-4-methoxybutane (76 mg, 0.403 mmol, 2 equiv) and Cs₂CO₃ (185 mg, 0.504 mmol). The reaction mixture was stirred at 100° C. for 3 h. Reaction mass was cooled to rt and poured into water (20 mL) and extracted with ethyl acetate (2×20 mL). The combined organic layers were washed with H₂O (3×10 mL) and brine solution (20 mL), dried over anhydrous Na₂SO₄, and concentrated by rotary evaporation. Then, the resulting concentrate was purified on a silica column to get 53 mg of an Off-white solid compound with a yield of 43%, purity=91.82%

TLC R_{f}=0.5 (PE:EA=4:1);

LCMS (m/z)=513.40 (M+H)

### Example 80. 8-(1-(2-ethoxyethyl)-1H-pyrazol-4-yl)-1-(4-methoxybenzyl)-4-(5-methyloxazol-2-yl)-1,3-dihydro-2H-benzo[b]azepin-2-one (80)

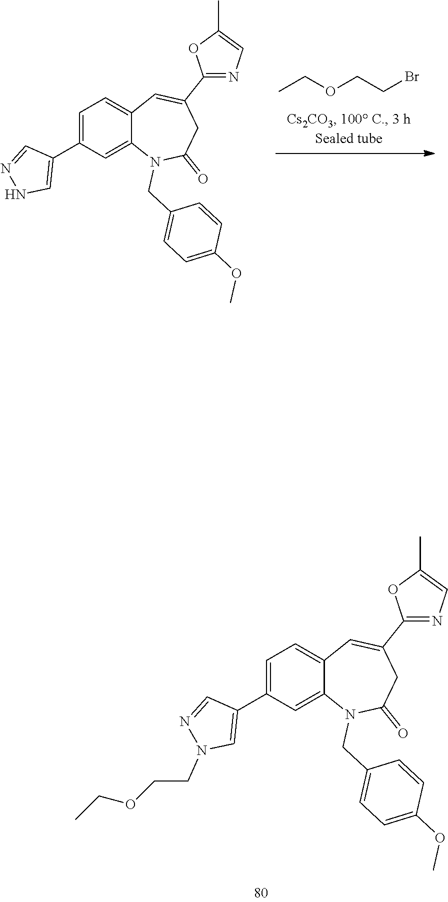

To a stirred solution of 1-(4-methoxybenzyl)-4-(5-methyloxazol-2-yl)-8-(1H-pyrazol-4-yl)-1,3-dihydro-2H-benzo[b]azepin-2-one (100 mg, 0.201 mmol, 1 equiv) in DMF (5 mL) were added 1-bromo-2-ethoxyethane (69.2 mg, 0.403 mmol, 2 equiv) and Cs₂CO₃ (185 mg, 0.504 mmol). The reaction mixture was stirred at 100° C. for 3 h. Reaction mass was cooled to rt and poured into water (20 mL) and extracted with ethyl acetate (2×20 mL). The combined organic layers were washed with H₂O (3×10 mL) and brine solution (20 mL), dried over anhydrous Na₂SO₄, and concentrated by rotary evaporation. Then, the resulting concentrate was purified on a silica column to get 121 mg of an Off-white solid compound with a yield of 97%, purity=91.6%

TLC R_{f}=0.5 (PE:EA=4:1);

LCMS (m/z)=499.30 (M+H)

### Example 81. 8-(1-Acetyl-1H-pyrazol-4-yl)-1-(4-methoxybenzyl)-4-(5-methyloxazol-2-yl)-1,3-dihydro-2H-benzo[b]azepin-2-one: (81)

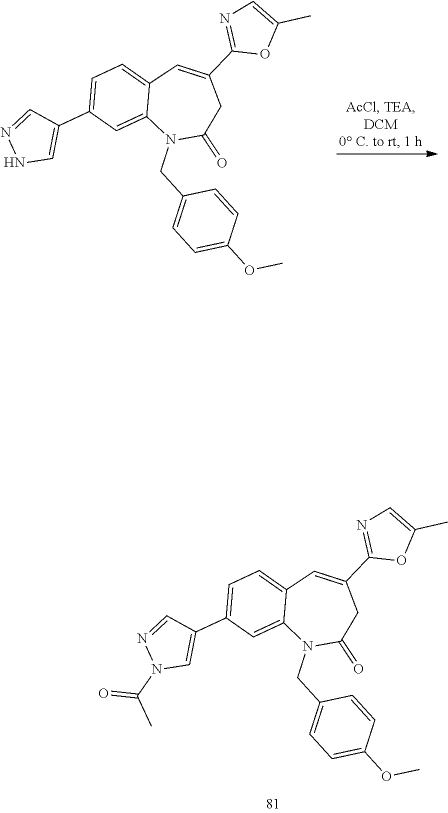 [SMILES: COc1ccc(CN2C(=O)CC(c3ncc(C)o3)=Cc3ccc(-c4cn[nH]c4)cc32)cc1 ; COc1ccc(CN2C(=O)CC(c3ncc(C)o3)=Cc3ccc(-c4cnn(C(C)=O)c4)cc32)cc1]

To a clear solution of 1-(4-methoxybenzyl)-4-(5-methyloxazol-2-yl)-8-(1H-pyrazol-4-yl)-1,3-dihydro-2H-benzo[b]azepin-2-one (100 mg, 0.234, 1 equiv.) was added TEA (0.1 mL, 1.1 mmol, 5 equiv.) and acetyl chloride (37 mg, 0.468 mmol, 2 equiv.) at 0° C., then the reaction mixture was allowed to stir at room temperature for 1h. after completion of the reaction shown by TLC, the reaction mixture was diluted with DCM, washed with water (2×10 mL) and brine solution (1×10 mL). The separated organic layer was dried over anhyd. Na₂SO₄ and concentrated under the rota evaporator. The separated solid was further purified using flash column chromatography to afford a pure compound (45 mg, purity=76.4%, yield=44.8%) as an off-white solid

TLC R_{f}=0.6 (1/4 PE/EA)

LCMS (m/z)=469.50 (M+1)

### Example 82. 8-(1-(3-Hydroxybutan-2-yl)-1H-pyrazol-4-yl)-1-(4-methoxybenzyl)-4-(5-methyloxazol-2-yl)-1,3-dihydro-2H-benzo[b]azepin-2-one (82)

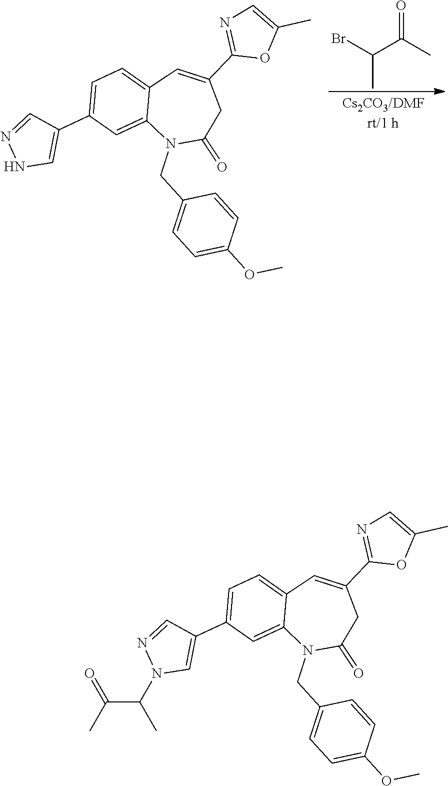

To a clear solution of 1-(4-methoxybenzyl)-4-(5-methyloxazol-2-yl)-8-(1H-pyrazol-4-yl)-1,3-dihydro-2H-benzo[b]azepin-2-one (200 mg, 0. 468 mmol, 1 equiv.) in dry DMF, Cs₂CO₃ (229 mg, 0.703 mmol, 3 equiv.) and 3-bromobutan-2-one (53 mg, 0.351 mmol, 1.5 equiv.) was added under nitrogen condition in a round-bottom flask. Then the reaction mixture was stirred at room temperature for 1 h. After completion of reaction, the mixture was diluted with ethyl acetate, washed with water (2×10 mL) and brine solution (1×10 mL), the separated organic layer was dried over anhydro Na₂SO₄, filtered, and concentrated under rotary evaporation. The crude was further purified through the flash column to get pure compound (192 mg, purity=88%, yield=82.4%) as a white solid.

TLC R_{f}=0.6 (1/4 PE/EA)

LCMS (m/z)=497.50 (M+1)

### Example 83. 8-(1-(3-Hydroxybutan-2-yl)-1H-pyrazol-4-yl)-1-(4-methoxybenzyl)-4-(5-methyloxazol-2-yl)-1,3-dihydro-2H-benzo[b]azepin-2-one (83)

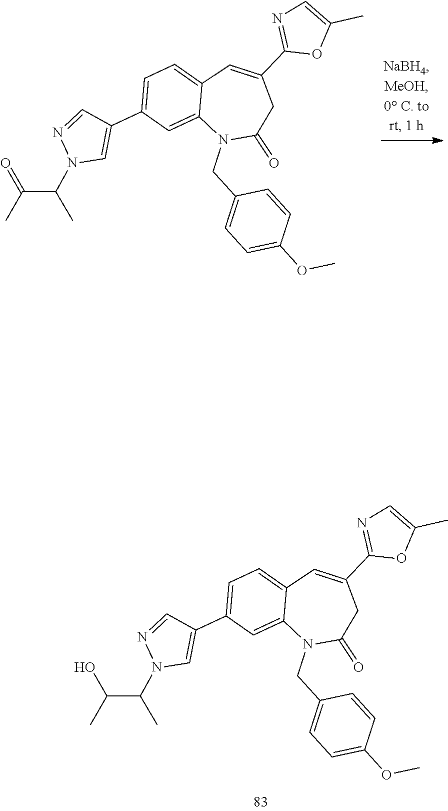 [SMILES: COc1ccc(CN2C(=O)CC(c3ncc(C)o3)=Cc3ccc(-c4cnn(C(C)C(C)=O)c4)cc32)cc1 ; COc1ccc(CN2C(=O)CC(c3ncc(C)o3)=Cc3ccc(-c4cnn(C(C)C(C)O)c4)cc32)cc1]

To a clear solution of 1-(4-methoxybenzyl)-4-(5-methyloxazol-2-yl)-8-(1-(3-oxobutan-2-yl)-1H-pyrazol-4-yl)-1,3-dihydro-2H-benzo[b]azepin-2-one (100 mg, 0.201 mmol, 1 equiv.) in methanol, NaBH₄ (4 mg, 0.1 mmol, 0.5 equiv.) was added at 0° C., after completion of addition the addition, the reaction mixture was slowly allowed to room temperature and continue the stirring at room for 1 h. After completion of reaction shown by TLC, the reaction mixture was cooled to 0° C., quenched with saturated NH₄Cl solution, and extracted into ethyl acetate, washed with water (2×5 mL) and brine solution (1×5 mL). The separated organic layer was dried over anhyd. Na₂SO₄, filtered, and concentrated by rotary evaporator. The crude was further purified through the flash column to get pure compound (45 mg, purity=76.4%, yield=44.8%) as a white solid.

TLC R_{f}=0.4 (1/4 PE/EA)

LCMS (m/z)=499.50 (M+1)

### Example 84. 2-(1-(Ethylsulfonyl)-3-(4-(1-(4-methoxybenzyl)-4-(5-methyloxazol-2-yl)-2-oxo-2,3-dihydro-1H-benzo[b]azepin-8-yl)-1H-pyrazol-1-yl)azetidin-3-yl)acetonitrile (84)

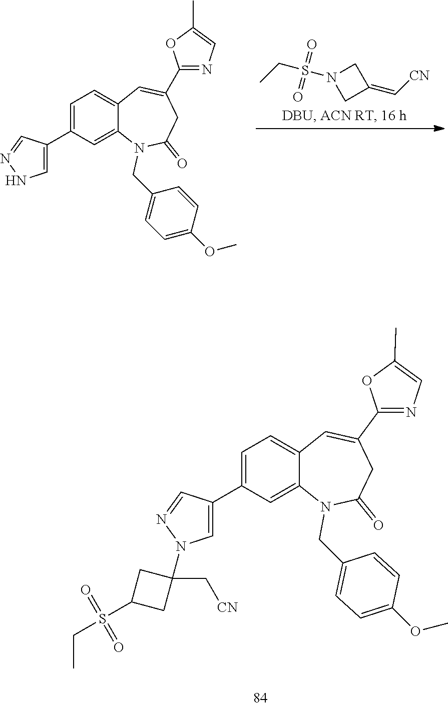 [SMILES: COc1ccc(CN2C(=O)CC(c3ncc(C)o3)=Cc3ccc(-c4cn[nH]c4)cc32)cc1 ; CCS(=O)(=O)N1CC(=CC#N)C1 ; CCS(=O)(=O)C1CC(CC#N)(n2cc(-c3ccc4c(c3)N(Cc3ccc(OC)cc3)C(=O)CC(c3ncc(C)o3)=C4)cn2)C1]

To a stirred solution of 1-(4-methoxybenzyl)-4-(5-methyloxazol-2-yl)-8-(1H-pyrazol-4-yl)-1,3-dihydro-2H-benzo[b]azepin-2-one (100 mg, 0.234 mmol, 1 equiv) in acetonitrile (5 mL), to this add 2-(1-(ethylsulfonyl)azetidin-3-ylidene)acetonitrile (68.3 mg, 0.234 mmol, 1 equiv), DBU (53.5 mg, 0.234 mmol, 1 equiv). The reaction mass was stirred at rt for 16 h. The reaction mass was diluted with water and extracted with EA (3×30 mL). The organic solutions were combined, washed with H₂O (2×20 mL) and brine solution (1×20 mL), dried over anhydrous Na₂SO₄, filtered and concentrated by rotary evaporation to get the crude. The crude was purified by silica column to get pure compound (70 mg, purity=100%, yield=47%) as an off white solid.

TLC R_{f}=0.4 (80%, EA:PE)

LCMS (m/z)=613.50 (M+H)

¹H NMR (400 MHz, Chloroform-d) δ 7.76 (s, 2H), 7.52 (s, 1H), 7.46 (s, 1H), 7.35 (d, J=7.4 Hz, 1H), 7.27 (d, J=9.6 Hz, 1H), 7.10 (d, J=7.8 Hz, 2H), 6.86 (s, 1H), 6.80 (d, J=7.5 Hz, 2H), 5.08 (s, 2H), 4.57 (d, J=8.1 Hz, 2H), 4.18 (d, J=8.0 Hz, 2H), 3.96-2.88 (br., 2H), 3.76 (s, 3H), 3.36 (s, 2H), 3.08 (q, J=7.2 Hz, 2H), 2.39 (s, 3H), 1.41 (t, J=6.9 Hz, 3H).

### Example 85. 2-(3-(4-(1-(4-Methoxybenzyl)-4-(5-methyloxazol-2-yl)-2-oxo-2,3-dihydro-1H-benzo[b]azepin-8-yl)-1H-pyrazol-1-yl)azetidin-3-yl)acetonitrile (85)

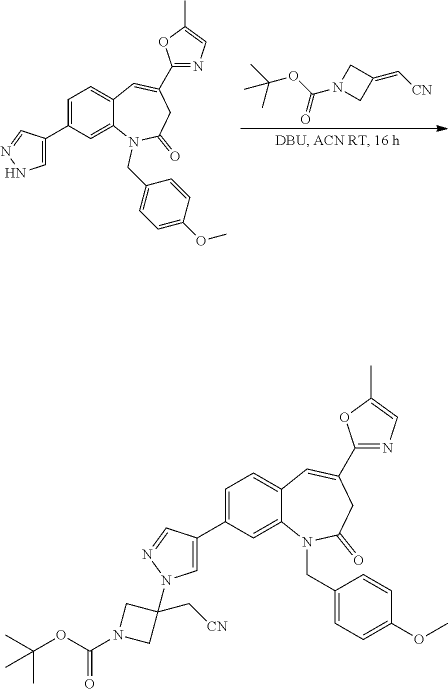 [SMILES: COc1ccc(CN2C(=O)CC(c3ncc(C)o3)=Cc3ccc(-c4cn[nH]c4)cc32)cc1 ; CC(C)(C)OC(=O)N1CC(=CC#N)C1 ; COc1ccc(CN2C(=O)CC(c3ncc(C)o3)=Cc3ccc(-c4cnn(C5(CC#N)CN(C(=O)OC(C)(C)C)C5)c4)cc32)cc1]

### Tert-butyl 3-(cyanomethyl)-3-(4-(1-(4-methoxybenzyl)-4-(5-methyloxazol-2-yl)-2-oxo-2,3-dihydro-1H-benzo[b]azepin-8-yl)-1H-pyrazol-1-yl)azetidine-1-carboxylate

To a stirred solution of 1-(4-methoxybenzyl)-4-(5-methyloxazol-2-yl)-8-(1H-pyrazol-4-yl)-1,3-dihydro-2H-benzo[b]azepin-2-one (150 mg, 0.352 mmol, 1 equiv) in acetonitrile (5 mL), to this add tert-butyl 3-(cyanomethylene)azetidine-1-carboxylate (68.3 mg, 0.352 mmol, 1 equiv), DBU (53.5 mg, 0.352 mmol, 1 equiv). The reaction mass was stirred at rt for 16 h. The reaction mass was diluted with water and extracted with EA (3×30 mL). The organic solutions were combined, washed with H₂O (2×20 mL) and brine solution (1×20 mL), dried over anhydrous Na₂SO₄, filtered and concentrated by rotary evaporation to get the crude compound (170 mg, purity=93.6%, yield=76%) as a brown solid.

TLC R_{f}=0.4 (1:1, EA:PE)

LCMS (m/z)=621.60 (M+H)

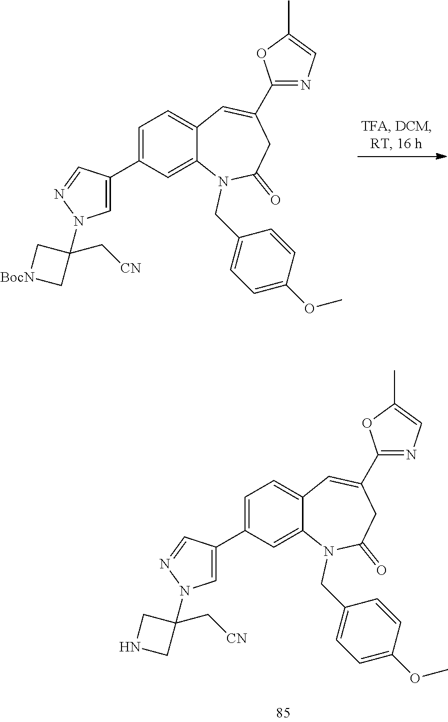 [SMILES: COc1ccc(CN2C(=O)CC(c3ncc(C)o3)=Cc3ccc(-c4cnn(C5(CC#N)CN(C(=O)OC(C)(C)C)C5)c4)cc32)cc1 ; COc1ccc(CN2C(=O)CC(c3ncc(C)o3)=Cc3ccc(-c4cnn(C5(CC#N)CNC5)c4)cc32)cc1]

### 2-(3-(4-(1-(4-Methoxybenzyl)-4-(5-methyloxazol-2-yl)-2-oxo-2,3-dihydro-1H-benzo[b]azepin-8-yl)-1H-pyrazol-1-yl)azetidin-3-yl)acetonitrile: 85

To a stirred solution of tert-butyl 3-(cyanomethyl)-3-(4-(1-(4-methoxybenzyl)-4-(5-methyloxazol-2-yl)-2-oxo-2,3-dihydro-1H-benzo[b]azepin-8-yl)-1H-pyrazol-1-yl)azetidine-1-carboxylate (150 mg, 0.241 mmol, 1 equiv) in DCM (10 mL), to this add TFA (0.5 mL). The reaction mass was stirred at rt for 16 h. The reaction mass was diluted with water and extracted with EA (3×30 mL). The organic solutions were combined, washed with H₂O (2×20 mL) and brine solution (1×20 mL), dried over anhydrous Na₂SO₄, filtered and concentrated by rotary evaporation to get the crude compound. The crude was purified by silica column to get pure compound (50 mg, purity=95%, yield=40%) as an off white solid.

TLC R_{f}=0.2 (10% MeOH:DCM)

LCMS (m/z)=521.40 (M+H)

### Example 86. 2-(4-(1-(4-Methoxybenzyl)-4-(5-methyloxazol-2-yl)-2-oxo-2,3-dihydro-1H-benzo[b]azepin-8-yl)-1H-pyrazol-1-yl)-2-methylpropanenitrile (86)

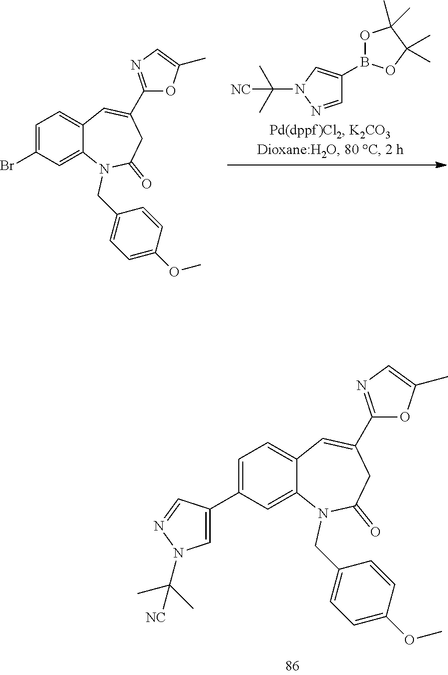 [SMILES: COc1ccc(CN2C(=O)CC(c3ncc(C)o3)=Cc3ccc(Br)cc32)cc1 ; CC(C)(C#N)n1cc(B2OC(C)(C)C(C)(C)O2)cn1 ; COc1ccc(CN2C(=O)CC(c3ncc(C)o3)=Cc3ccc(-c4cnn(C(C)(C)C#N)c4)cc32)cc1]

To a stirred solution of 8-bromo-1-(4-methoxybenzyl)-4-(5-methyloxazol-2-yl)-1,3-dihydro-2H-benzo[b]azepin-2-one (250 mg, 0.569 mmol, 1 equiv) in 1,4-dioxane/water (15 mL), to this add 2-methyl-2-(4-(4,4,5,5-tetramethyl-1,3,2-dioxaborolan-2-yl)-1H-pyrazol-1-yl)propanenitrile (222 mg, 0.854 mmol, 1.5 equiv), K₂CO₃ (196 mg, 1.423 mmol, 2.5 equiv). The solution was degassed and added Pd(dppf)Cl₂ (83 mg, 0.113 mmol, 0.2 equiv), then heated to 80° C. and stirred for 2 h. The reaction mass was quenched with saturated ammonium chloride and extracted with EA (3×20 mL). The organic solutions were combined, washed with H₂O (2×20 mL) and brine solution (1×20 mL), dried over anhydrous Na₂SO₄, filtered and concentrated by rotary evaporation to get the crude compound. The crude was purified by silica column to get pure compound (240 mg, purity=100%, yield=85%) as an off white solid.

TLC R_{f}=0.4 (1:1,EA/PE)

LCMS (m/z)=494.40 (M+H)

¹H NMR (400 MHz, Chloroform-d) δ 7.81 (s, 1H), 7.75 (s, 1H), 7.51 (s, 1H), 7.46 (s, 1H), 7.34 (d, J=8.0 Hz, 1H), 7.27 (d, J=9.0 Hz, 1H), 7.10 (d, J=8.6 Hz, 2H), 6.85 (d, J=1.4 Hz, 1H), 6.80 (d, J=8.6 Hz, 2H), 5.06 (s, 2H), 4.32-2.89 (br., 2H), 3.76 (s, 3H), 2.38 (s, 3H), 2.03 (s, 6H).

### Example 87. 2-(3-(4-(1-(4-Methoxybenzyl)-4-(5-methyloxazol-2-yl)-2-oxo-2,3-dihydro-1H-benzo[b]azepin-8-yl)-1H-pyrazol-1-yl)-1-(methylsulfonyl)azetidin-3-yl)acetonitrile (87)

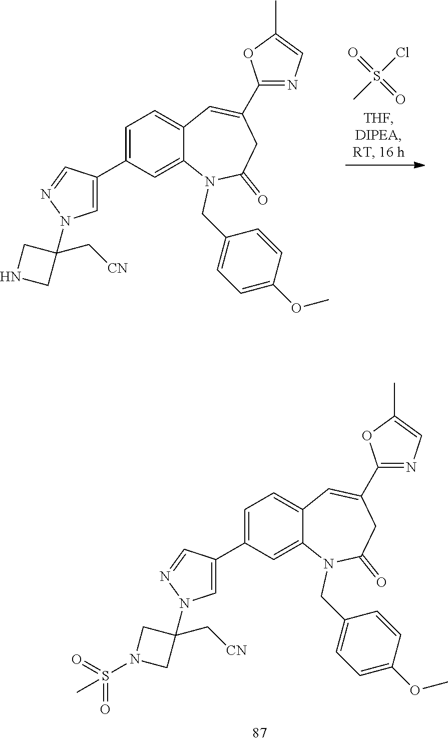

To a stirred solution of 2-(3-(4-(1-(4-methoxybenzyl)-4-(5-methyloxazol-2-yl)-2-oxo-2,3-dihydro-1H-benzo[b]azepin-8-yl)-1H-pyrazol-1-yl)azetidin-3-yl)acetonitrile (10 mg, 0.019 mmol, 1 equiv) in THF (1 mL), to this add methane sulfonyl chloride (0.002 mL, 0.0288 mmol, 1 equiv), DIPEA (0.008 mL, 0.0096 mmol, 2.5 equiv). The reaction mass was stirred at rt for 16 h. The reaction mass was concentrated by rotary evaporation to get the crude compound, The resulting crude compound was purified by column chromatography (7.9 mg, purity=100%, yield=68%) as a brown solid.

TLC R_{f}=0.6 (EA)

LCMS (m/z)=599.40 (M+H)

### Example 88. 2-(3-(4-(1-(4-Methoxybenzyl)-4-(5-methyloxazol-2-yl)-2-oxo-2,3-dihydro-1H-benzo[b]azepin-8-yl)-1H-pyrazol-1-yl)oxetan-3-yl)acetonitrile (88)

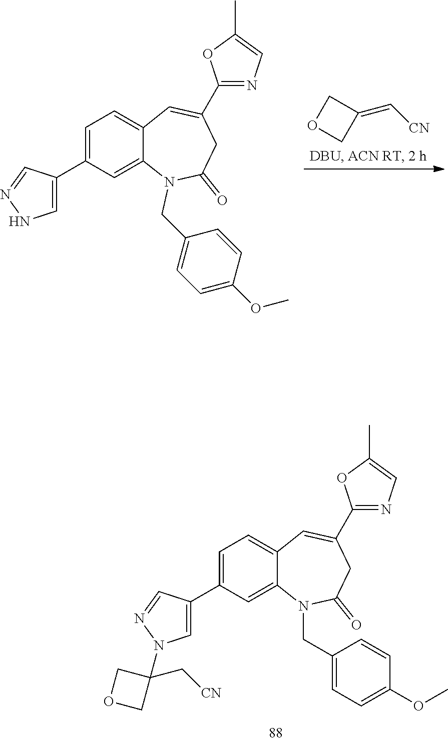 [SMILES: COc1ccc(CN2C(=O)CC(c3ncc(C)o3)=Cc3ccc(-c4cn[nH]c4)cc32)cc1 ; N#CC=C1COC1 ; COc1ccc(CN2C(=O)CC(c3ncc(C)o3)=Cc3ccc(-c4cnn(C5(CC#N)COC5)c4)cc32)cc1]

To a stirred solution of 1-(4-methoxybenzyl)-4-(5-methyloxazol-2-yl)-8-(1H-pyrazol-4-yl)-1,3-dihydro-2H-benzo[b]azepin-2-one (85 mg, 0.199 mmol, 1 equiv) in acetonitrile (5 mL), to this add 2-(oxetan-3-ylidene)acetonitrile (18.9 mg, 0.199 mmol, 1 equiv), DBU (30 mg, 0.199 mmol, 1 equiv). The reaction mass was stirred at rt for 2 h. The reaction mass was diluted with water and extracted with EA (3×30 mL). The organic solutions were combined, washed with H₂O (2×20 mL) and brine solution (1×20 mL), dried over anhydrous Na₂SO₄, filtered and concentrated by rotary evaporation to get the crude compound, The resulting crude was purified by column chromatography to get target compound (60 mg, purity=98%, yield=57%) as an off white solid.

TLC R_{f}=0.5 (EA)

LCMS (m/z)=522.40 (M+H)

¹H NMR (400 MHz, Chloroform-d) δ 7.78 (s, 1H), 7.76 (s, 1H), 7.52 (s, 1H), 7.46 (s, 1H), 7.35 (d, J=7.8 Hz, 1H), 7.28 (d, J=7.9 Hz, 1H), 7.10 (d, J=8.1 Hz, 2H), 6.86 (s, 1H), 6.80 (d, J=7.9 Hz, 2H), 5.12 (d, J=7.0 Hz, 2H), 5.07 (s, 2H), 4.84 (d, J=6.9 Hz, 2H), 4.06-2.82 (br., 2H), 3.76 (s, 3H), 3.40 (s, 2H), 2.39 (s, 3H).

### Example 89. 2-(1-(Cyclopropylsulfonyl)-3-(4-(1-(4-methoxybenzyl)-4-(5-methyloxazol-2-yl)-2-oxo-2,3-dihydro-1H-benzo[b]azepin-8-yl)-1H-pyrazol-1-yl)azetidin-3-yl)acetonitrile (89)

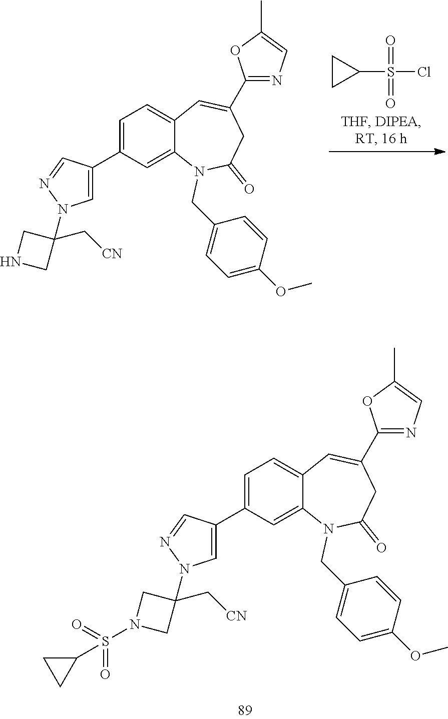 [SMILES: COc1ccc(CN2C(=O)CC(c3ncc(C)o3)=Cc3ccc(-c4cnn(C5(CC#N)CNC5)c4)cc32)cc1 ; O=S(=O)(Cl)C1CC1 ; COc1ccc(CN2C(=O)CC(c3ncc(C)o3)=Cc3ccc(-c4cnn(C5(CC#N)CN(S(=O)(=O)C6CC6)C5)c4)cc32)cc1]

To a stirred solution of 2-(3-(4-(1-(4-methoxybenzyl)-4-(5-methyloxazol-2-yl)-2-oxo-2,3-dihydro-1H-benzo[b]azepin-8-yl)-1H-pyrazol-1-yl)azetidin-3-yl)acetonitrile (10 mg, 0.019 mmol, 1 equiv) in THF (1 mL), to this add cyclopropane sulfonyl chloride (0.0013 mg, 0.0288 mmol, 1 equiv), DIPEA (0.008 mL, 0.0096 mmol, 2.5 equiv). The reaction mass was stirred at rt for 16 h. The reaction mass was concentrated by rotary evaporation to get the crude compound; The resulting crude compound was purified by column chromatography (5 mg, purity=100%, yield=45%) as an off-white semi-solid.

TLC R_{f}=0.6 (EA)

LCMS (m/z)=625.40 (M+H)

### Example 90. 2-(1-(Cyclopropanecarbonyl)-3-(4-(1-(4-methoxybenzyl)-4-(5-methyloxazol-2-yl)-2-oxo-2,3-dihydro-1H-benzo[b]azepin-8-yl)-1H-pyrazol-1-yl)azetidin-3-yl)acetonitrile (90)

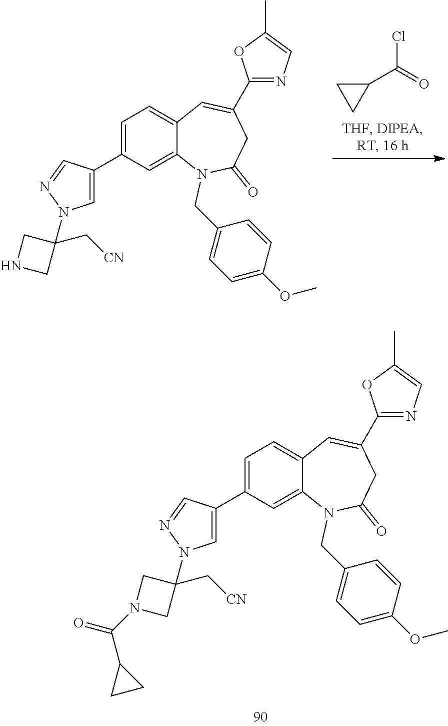 [SMILES: COc1ccc(CN2C(=O)CC(c3ncc(C)o3)=Cc3ccc(-c4cnn(C5(CC#N)CNC5)c4)cc32)cc1 ; O=C(Cl)C1CC1 ; COc1ccc(CN2C(=O)CC(c3ncc(C)o3)=Cc3ccc(-c4cnn(C5(CC#N)CN(C(=O)C6CC6)C5)c4)cc32)cc1]

To a stirred solution of 2-(3-(4-(1-(4-methoxybenzyl)-4-(5-methyloxazol-2-yl)-2-oxo-2,3-dihydro-1H-benzo[b]azepin-8-yl)-1H-pyrazol-1-yl)azetidin-3-yl)acetonitrile (10 mg, 0.019 mmol, 1 equiv) in THF (1 mL), to this add cyclopropane carbonyl chloride (0.003 mg, 0.0288 mmol, 1 equiv), DIPEA (0.008 mL, 0.0096 mmol, 2.5 equiv). The reaction mass was stirred at rt for 16 h. The reaction mass was extracted with ethyl acetate, the combined organic layers were dried over anhydrous Na₂SO₄, concentrated, and purified by column chromatography to afford the title compound (11 mg, purity=100%, yield=97%) as an off-white semi-solid.

TLC R_{f}=0.6 (EA)

LCMS (m/z)=589.50 (M+H)

### Example 91. 3-(Cyanomethyl)-3-(4-(1-(4-methoxybenzyl)-4-(5-methyloxazol-2-yl)-2-oxo-2,3-dihydro-1H-benzo[b]azepin-8-yl)-1H-pyrazol-1-yl)-N,N-dimethylazetidine-1-sulfonamide (91)

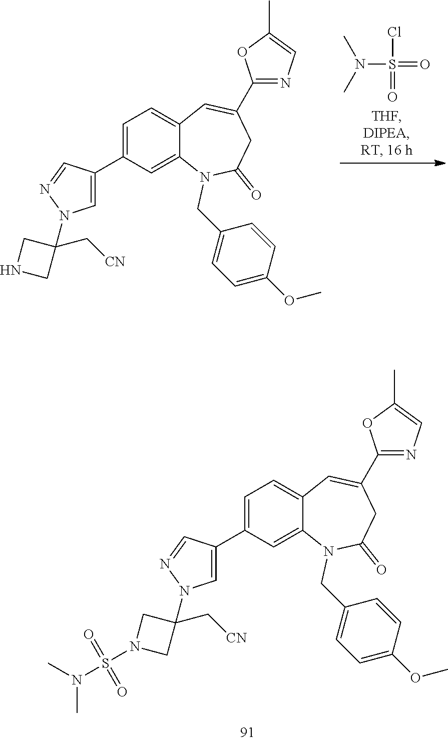

To a stirred solution of 2-(3-(4-(1-(4-methoxybenzyl)-4-(5-methyloxazol-2-yl)-2-oxo-2,3-dihydro-1H-benzo[b]azepin-8-yl)-1H-pyrazol-1-yl)azetidin-3-yl)acetonitrile (10 mg, 0.019 mmol, 1 equiv) in THF (1 mL), to this add N,N-dimethylsulfamoyl chloride (0.004 mg, 0.0288 mmol, 1 equiv), DIPEA (0.008 mL, 0.0096 mmol, 2.5 equiv). The reaction mass was stirred at rt for 16 h. The reaction mass was extracted with ethyl acetate; the combined organic layers were dried over anhydrous Na₂SO₄, filtered, concentrated, and purified by column chromatography to afford the title compound (4 mg, purity=64%, yield=33%) as an off-white semi-solid.

TLC R_{f}=0.6 (EA)

LCMS (m/z)=628.40 (M+H)

### Example 92. 1-(4-(1-(4-Methoxybenzyl)-4-(5-methyloxazol-2-yl)-2-oxo-2,3-dihydro-1H-benzo[b]azepin-8-yl)-1H-pyrazol-1-yl)cyclopropane-1-carboxamide (92)

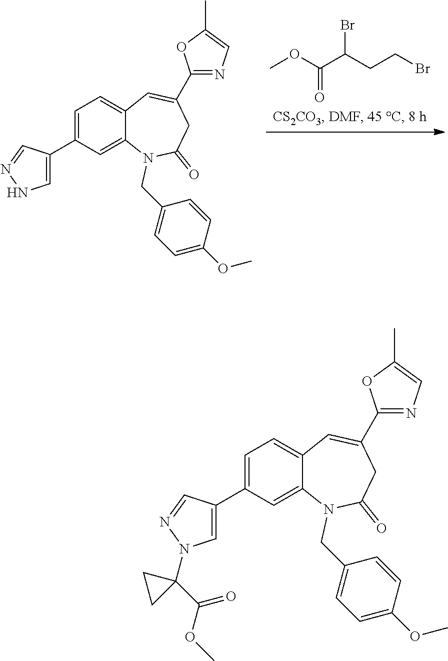

### Methyl 1-(4-(1-(4-methoxybenzyl)-4-(5-methyloxazol-2-yl)-2-oxo-2,3-dihydro-1H-benzo[b]azepin-8-yl)-1H-pyrazol-1-yl)cyclopropane-1-carboxylate

To a solution of a mixture of 1-(4-methoxybenzyl)-4-(5-methyloxazol-2-yl)-8-(1H-pyrazol-4-yl)-1,3-dihydro-2H-benzo[b]azepin-2-one (200 mg, 0.47 mmol, 1 equiv) and methyl 2,4-dibromobutanoate (145 mg, 0.56 mmol, 1.2 equiv) in DMF (3 mL) were added CS₂CO₃ (611 mg, 1.88 mmol, 4 equiv). The reaction was stirred at 50° C. for 8 h. The reaction was then diluted with saturated ammonium chloride and extracted with EtOAc. The organic extracts were washed with brine solution, dried over sodium sulfate, filtered, and concentrated. The crude product was purified by silica gel chromatography to give target product (110 mg, purity=100, yield=44%).

TLC R_{f}=0.4 (2:1, EA:PE)

LCMS (m/z)=525.40 (M+H)

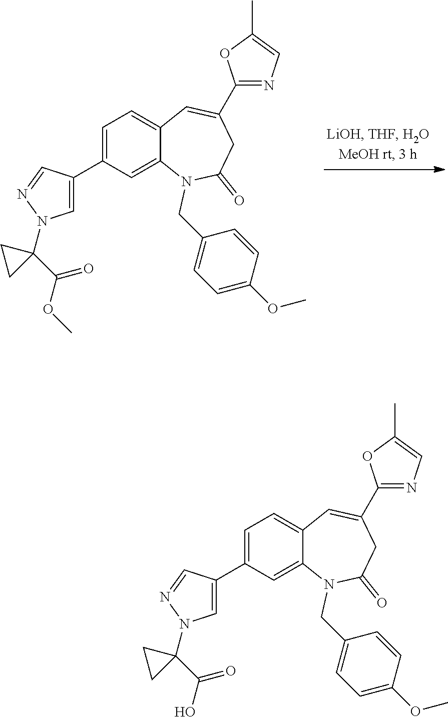 [SMILES: COC(=O)C1(n2cc(-c3ccc4c(c3)N(Cc3ccc(OC)cc3)C(=O)CC(c3ncc(C)o3)=C4)cn2)CC1 ; COc1ccc(CN2C(=O)CC(c3ncc(C)o3)=Cc3ccc(-c4cnn(C5(C(=O)O)CC5)c4)cc32)cc1]

### 1-(4-(1-(4-Methoxybenzyl)-4-(5-methyloxazol-2-yl)-2-oxo-2,3-dihydro-1H-benzo[b]azepin-8-yl)-1H-pyrazol-1-yl)cyclopropane-1-carboxylic acid

To a stirred solution methyl 1-(4-(1-(4-methoxybenzyl)-4-(5-methyloxazol-2-yl)-2-oxo-2,3-dihydro-1H-benzo[b]azepin-8-yl)-1H-pyrazol-1-yl)cyclopropane-1-carboxylate (30 mg, 0.05 mmol, 1 equiv) in 1:1:1 mixture solvent (3 mL), was added LiOH·H₂O (12 mg, 0.25 mmol, 5 equiv) at ambient temperature. The reaction mixture was then stirred at rt for 3 h. The solution was quenched with 1N HCl and extracted with EA (3×10 mL). The organic solutions were combined, washed with H₂O (2×10 mL) and brine solution solution (1×20 mL), dried over anhydrous Na₂SO₄, filtered and concentrated by rotary evaporation to get the crude compound. The resulting crude compound was purified by column chromatography to achieve the title compound (25 mg, purity=75%, yield=83%) as a white solid.

TLC R_{f}=0.25 (DCM:MeOH=10:1);

LCMS (m/z)=511.40 (M+H)

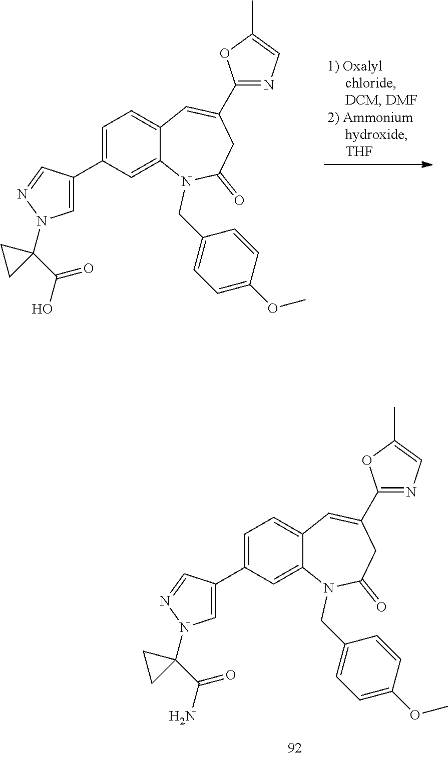 [SMILES: COc1ccc(CN2C(=O)CC(c3ncc(C)o3)=Cc3ccc(-c4cnn(C5(C(=O)O)CC5)c4)cc32)cc1 ; COc1ccc(CN2C(=O)CC(c3ncc(C)o3)=Cc3ccc(-c4cnn(C5(C(N)=O)CC5)c4)cc32)cc1]

### 1-(4-(1-(4-Methoxybenzyl)-4-(5-methyloxazol-2-yl)-2-oxo-2,3-dihydro-1H-benzo[b]azepin-8-yl)-1H-pyrazol-1-yl)cyclopropane-1-carboxamide (92)

To a solution of 1-(4-(1-(4-methoxybenzyl)-4-(5-methyloxazol-2-yl)-2-oxo-2,3-dihydro-1H-benzo[b]azepin-8-yl)-1H-pyrazol-1-yl)cyclopropane-1-carboxylic acid (25 mg, 0.05 mmol) in CH₂Cl₂ (1 mL) were added oxalyl chloride (0.15 mL) and 1 drop of DMF. The reaction was stirred for 30 min prior to being concentrated under vacuum. The residue was dissolved in THF (1 mL) and added to a stirred mixture of ammonium hydroxide (1 mL) in THF (1 mL). After 30 min of stirring, the mixture was diluted with water and extracted with EtOAc (3×10 mL). The combined extracts were washed with brine solution, dried over sodium sulfate, filtered, and concentrated. The crude product was purified by silica gel chromatography to give the target compound (20 mg, 80% over two steps, purity=94%).

TLC R_{f}=0.25 (DCM:MeOH=20:1);

LCMS (m/z)=510.30 (M+H)

¹H NMR (400 MHz, Chloroform-d) δ 7.77 (s, 1H), 7.71 (s, 1H), 7.54 (s, 1H), 7.44 (d, J=1.7 Hz, 1H), 7.35 (d, J=8.1 Hz, 1H), 7.26 (dd, J=8.1, 1.6 Hz, 1H), 7.08 (d, J=8.6 Hz, 2H), 6.86 (d, J=1.3 Hz, 1H), 6.79 (d, J=8.7 Hz, 2H), 5.67 (s, 1H), 5.59 (s, 1H), 5.06 (s, 2H), 4.21-2.88 (br., 2H), 3.75 (s, 3H), 2.38 (d, J=1.2 Hz, 3H), 1.88 (q, J=4.8 Hz, 2H), 1.53 (q, J=4.7 Hz, 2H).

### Example 93. 8-(1-(2-Hydroxy-2-methylpropyl)-1H-pyrazol-4-yl)-1-(4-methoxybenzyl)-4-(5-methyl-1,3,4-oxadiazol-2-yl)-1,3-dihydro-2H-benzo[b]azepin-2-one (93)

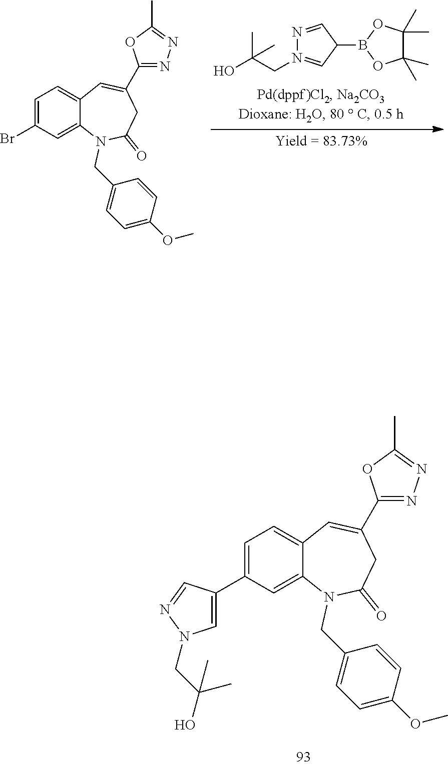

8-bromo-1-(4-methoxybenzyl)-4-(5-methyl-1,3,4-oxadiazol-2-yl)-1,3-dihydro-2H-benzo[b]azepin-2-one (200 mg, 0.454 mmol, 1 equiv) and 2-methyl-1-(4-(4,4,5,5-tetramethyl-1,3,2-dioxaborolan-2-yl)-1H-pyrazol-1-yl) propane-2-ol (242 mg, 0.908 mmol, 2 equiv) and Pd(dppf)Cl₂ (83 mg, 0.114 mmol, 0.25 equiv) were placed in a round-bottom flask, followed by 1,4-dioxane (8.4 mL) and 2N Na₂CO₃ aqueous solution (4.2 mL). The solution was degassed and heated at 80° C. for 0.5 h. The solution was quenched with saturated ammonium chloride and extracted with EA (3×10 mL). The organic solutions were combined, washed with H₂O (2×10 mL) and brine solution solution (1×20 mL), dried over anhydrous Na₂SO₄, and filtered, and concentrated by rotary evaporation to get the crude compound. Then, the resulting concentrate was purified on a silica column to get 190 mg (purity=100%) yellow solid compound with a yield of 83.73%.

TLC R_{f}=0.1 (DCM:EA=2:1);

LCMS (m/z)=500.70 (M+H)

¹H NMR (400 MHz, Chloroform-d) δ 7.72 (s, 1H), 7.66 (s, 1H), 7.58 (s, 1H), 7.48 (s, 1H), 7.33 (d, J=8.1 Hz, 1H), 7.30 (d, J=8.8 Hz, 1H), 7.07 (d, J=8.6 Hz, 2H), 6.78 (d, J=8.6 Hz, 2H), 5.06 (s, 2H), 4.09 (s, 2H), 3.75 (s, 3H), 2.59 (s, 3H), 1.21 (s, 7H).

### Example 94. 4-(5-Cyclopropyl-1,3,4-oxadiazol-2-yl)-8-(1-(2-hydroxy-2-methylpropyl)-1H-pyrazol-4-yl)-1-(4-methoxybenzyl)-1,3-dihydro-2H-benzo[b]azepin-2-one (94)

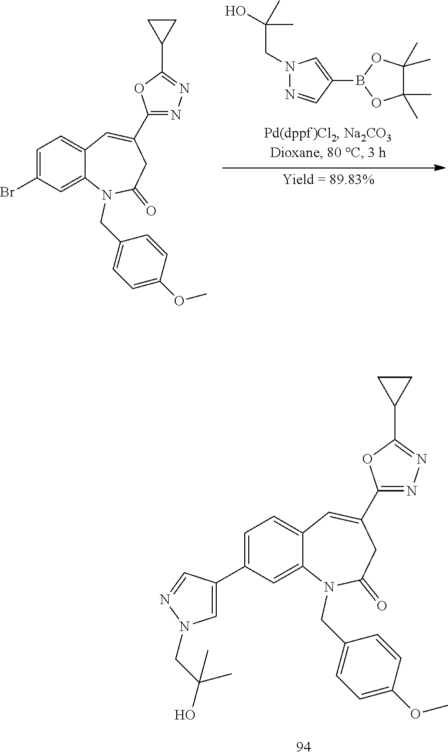 [SMILES: COc1ccc(CN2C(=O)CC(c3nnc(C4CC4)o3)=Cc3ccc(Br)cc32)cc1 ; CC(C)(O)Cn1cc(B2OC(C)(C)C(C)(C)O2)cn1 ; COc1ccc(CN2C(=O)CC(c3nnc(C4CC4)o3)=Cc3ccc(-c4cnn(CC(C)(C)O)c4)cc32)cc1]

8-bromo-4-(5-cyclopropyl-1,3,4-oxadiazol-2-yl)-1-(4-methoxybenzyl)-1,3-dihydro-2H-benzo[b]azepin-2-one (160 mg, 0.343 mmol, 1 equiv) and 2-methyl-1-(4-(4,4,5,5-tetramethyl-1,3,2-dioxaborolan-2-yl)-1H-pyrazol-1-yl)propan-2-ol (183 mg, 0.686 mmol, 2 equiv) and Pd(dppf)Cl₂ (86 mg, 0.628 mmol, 0.25 equiv) were placed in a round-bottom flask, followed by 1,4-dioxane (6.4 mL) and 2N Na₂CO₃ aqueous solution (3.2 mL) were added. The solution was degassed and heated at 80° C. for 3 h. The solution was quenched with saturated ammonium chloride and extracted with EA (3×10 mL). The organic layers were combined, washed with H₂O (2×10 mL) and brine solution (1×20 mL), dried over anhydrous Na₂SO₄, filtered and concentrated by rotary evaporation to get the crude compound. Then, the resulting concentrate was purified on a silica column to get desired compound (162 mg, purity=97%, yield=89.83%) as a yellow solid

TLC R_{f}=0.15 (DCM:EA=2:1);

LCMS (m/z)=526.2 (M+H)

¹H NMR (400 MHz, Chloroform-d) δ 7.71 (s, 1H), 7.65 (s, 1H), 7.51 (s, 1H), 7.48 (s, 1H), 7.36-7.27 (m, 2H), 7.08 (d, J=8.2 Hz, 2H), 6.78 (d, J=8.2 Hz, 2H), 5.06 (s, 2H), 4.10 (s, 2H), 3.75 (s, 3H), 3.53 (s, 2H), 2.99 (s, 1H), 2.19 (tt, J=7.9, 5.4 Hz, 1H), 1.23-1.14 (m, 10H).

### Example 95. 4-(3-cyclopropyl-1,2,4-oxadiazol-5-yl)-8-(1-(2-hydroxy-2-methylpropyl)-1H-pyrazol-4-yl)-1-(4-methoxybenzyl)-1,3-dihydro-2H-benzo[b]azepin-2-one (95)

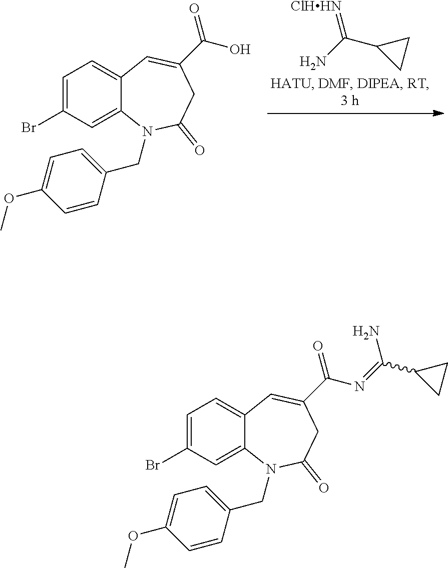

### N-(amino(cyclopropyl)methylene)-8-bromo-1-(4-methoxybenzyl)-2-oxo-2,3-dihydro-1H-benzo[b]azepine-4-carboxamide

A mixture of 8-bromo-1-(4-methoxybenzyl)-2-oxo-2,3-dihydro-1H-benzo[b]azepine-4-carboxylic acid (350 mg, 0.870 mmol, 1 equiv), HATU (397 mg, 1.044 mmol, 1.2 equiv), DIPEA (0.38 mL, 2.176 mmol, 2.5 equiv) in DMF at rt, then add cyclopropanecarboximidamide hydrochloride (157 mg, 1.305 mmol, 1.5 equiv). The reaction mass was stirred for 4 h. After completion of the reaction, the reaction mass was poured into the water (water quantity?), solid was precipitated, filtered, and dried to get 300 mg of an off-white solid. (purity=92.41%, yield=75%). This compound was used for next reaction without further purification.

TLC R_{f}=0.5 (EA)

LCMS (m/z)=470.40 (M+H)

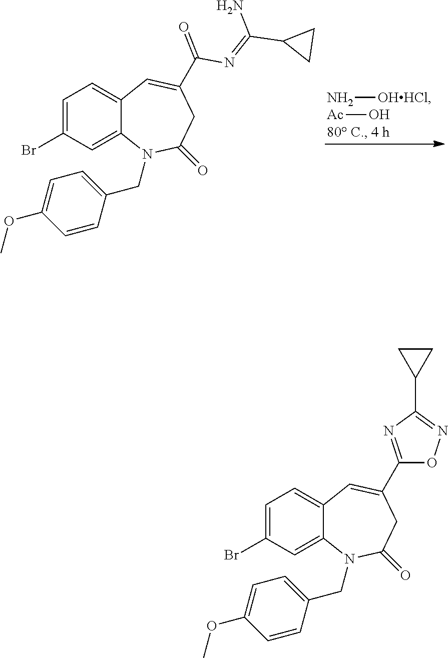

### 8-Bromo-4-(3-cyclopropyl-1,2,4-oxadiazol-5-yl)-1-(4-methoxybenzyl)-1,3-dihydro-2H-benzo[b]azepin-2-one

To a stirred solution of N-(amino(cyclopropyl)methylene)-8-bromo-1-(4-methoxybenzyl)-2-oxo-2,3-dihydro-1H-benzo[b]azepine-4-carboxamide (300 mg, 0.641 mmol, 1 equiv) in acetic acid (10 mL), was added hydroxylamine hydrochloride (89 mg, 1.282 mmol, 2.0 equiv). Then resulting solution was warmed to 80° C. and stirred for 4 h. The reaction mixture was poured into water (How many mL of water?), solid was precipitated, filtered and dried to get 300 mg of brown solid. (purity=86.4%, yield=100%). This compound was used for next reaction without further purification.

TLC R_{f}=0.5 (1:1, EA:PE)

LCMS (m/z)=468.10 (M+H)

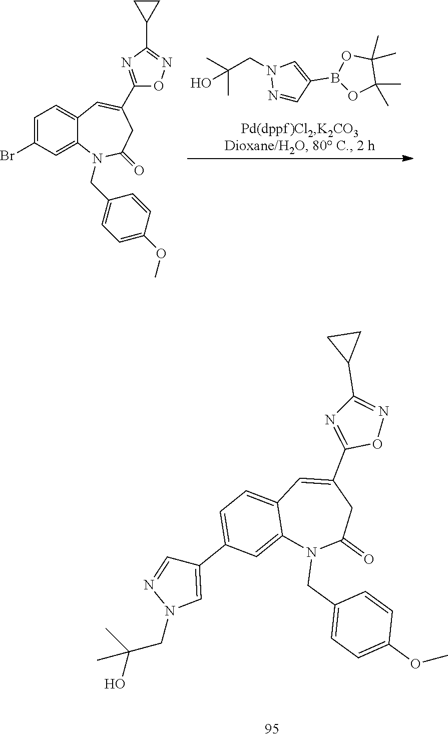 [SMILES: COc1ccc(CN2C(=O)CC(c3nc(C4CC4)no3)=Cc3ccc(Br)cc32)cc1 ; CC(C)(O)Cn1cc(B2OC(C)(C)C(C)(C)O2)cn1 ; COc1ccc(CN2C(=O)CC(c3nc(C4CC4)no3)=Cc3ccc(-c4cnn(CC(C)(C)O)c4)cc32)cc1]

### 4-(3-cyclopropyl-1,2,4-oxadiazol-5-yl)-8-(1-(2-hydroxy-2-methylpropyl)-1H-pyrazol-4-yl)-1-(4-methoxybenzyl)-1,3-dihydro-2H-benzo[b]azepin-2-one:95

To a stirred solution of 8-bromo-4-(3-cyclopropyl-1,2,4-oxadiazol-5-yl)-1-(4-methoxybenzyl)-1,3-dihydro-2H-benzo[b]azepin-2-one (300 mg, 0.608 mmol, 1 equiv) in 1,4-dioxane/water (15 mL), to this add 2-methyl-1-(4-(4,4,5,5-tetramethyl-1,3,2-dioxaborolan-2-yl)-1H-pyrazol-1-yl)propan-2-ol (242 mg, 0.912 mmol, 1.5 equiv), K₂CO₃ (210 mg, 1.521 mmol, 2.5 equiv). The solution was degassed and added Pd(dppf)Cl₂ (89 mg, 0.121 mmol, 0.2 equiv). The resulting solution was moved to 80° cand allowed to stir for 2 h. Subsequently, the reaction mass was quenched with saturated ammonium chloride and extracted with EA (3×20 mL). The organic layers were combined, washed with H₂O (2×20 mL) and brine solution (1×20 mL), dried over anhydrous Na₂SO₄, filtered and concentrated by rotary evaporation to get the crude compound. The crude was purified by silica column to get a pure compound (169 mg, purity=100%, yield=50%) as an off-white solid.

TLC R_{f}=0.3 (1:1,EA/PE)

LCMS (m/z)=526.40 (M+H)

¹H NMR (400 MHz, Chloroform-d) δ 7.76 (s, 1H), 7.72 (s, 1H), 7.63 (s, 1H), 7.47 (s, 1H), 7.35 (d, J=8.1 Hz, 1H), 7.30 (dd, J=8.1, 1.5 Hz, 1H), 7.07 (d, J=8.6 Hz, 2H), 6.79 (d, J=8.6 Hz, 2H), 5.06 (s, 2H), 4.10 (s, 2H), 3.98-3.18 (br., 2H), 3.76 (s, 3H), 3.04-2.53 (br., 1H), 2.12 (tt, J=8.2, 5.0 Hz, 1H), 1.21 (s, 6H), 1.16-1.00 (m, 4H).

### Example 96. 8-(1-(2-Hydroxy-2-methylpropyl)-1H-pyrazol-4-yl)-1-(4-methoxybenzyl)-4-(oxazol-5-yl)-1,3-dihydro-2H-benzo[b]azepin-2-one (96)

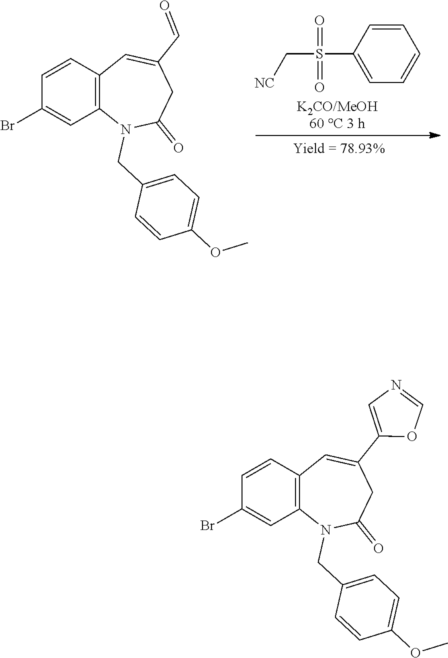 [SMILES: COc1ccc(CN2C(=O)CC(C=O)=Cc3ccc(Br)cc32)cc1 ; N#CCS(=O)(=O)c1ccccc1 ; COc1ccc(CN2C(=O)CC(c3cnco3)=Cc3ccc(Br)cc32)cc1]

### 8-Bromo-1-(4-methoxybenzyl)-4-(oxazol-5-yl)-1,3-dihydro-2H-benzo[b]azepin-2-one

To a solution of 2-tosyl acetonitrile (328 mg, 1.68 mmol, 1.2 equiv) in dry MeOH (40 mL) was added K₂CO₃ (580 mg, 4.2 mmol, 3 equiv) and the 8-bromo-1-(4-methoxybenzyl)-2-oxo-2,3-dihydro-1H-benzo[b]azepine-4-carbaldehyde (543 mg, 1.4 mmol, 1 equiv) under an argon atmosphere. to the resulting solution was reflux for 3 h. After completion of the reaction on analysis by TLC, which was indicated by the absence of aldehyde starting material, the mixture was quenched with cold water. After the mixture was quenched, all of the solvents were removed under reduced pressure, and the product was extracted with ethyl acetate (3×30 mL). The combined organic layers were washed with water and brine solution and dried (Na₂SO₄). The solvent was then removed under reduced pressure, and the residue was purified by flash chromatography to give the target compound (weight=470 mg, purity=98%, yield=78.93%) as a white solid.

TLC R_{f}=0.2 (PE:EA=2:1);

LCMS (m/z)=425.90 (M+H)

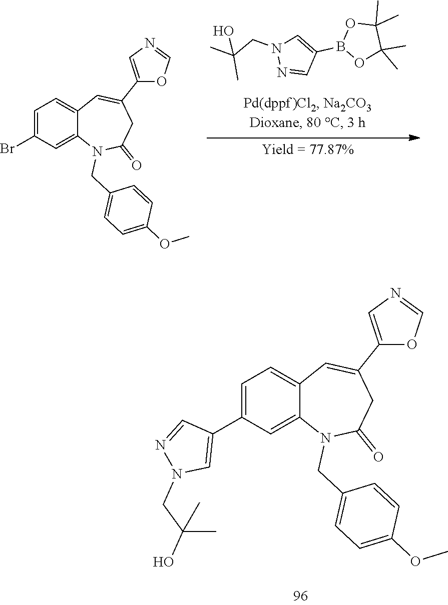 [SMILES: COc1ccc(CN2C(=O)CC(c3cnco3)=Cc3ccc(Br)cc32)cc1 ; CC(C)(O)Cn1cc(B2OC(C)(C)C(C)(C)O2)cn1 ; COc1ccc(CN2C(=O)CC(c3cnco3)=Cc3ccc(-c4cnn(CC(C)(C)O)c4)cc32)cc1]

### 8-(1-(2-Hydroxy-2-methylpropyl)-1H-pyrazol-4-yl)-1-(4-methoxybenzyl)-4-(oxazol-5-yl)-1,3-dihydro-2H-benzo[b]azepin-2-one: 96

8-bromo-1-(4-methoxybenzyl)-4-(oxazol-5-yl)-1,3-dihydro-2H-benzo[b]azepin-2-one (225 mg, 0.53 mmol, 1 equiv) and 2-methyl-1-(4-(4,4,5,5-tetramethyl-1,3,2-dioxaborolan-2-yl)-1H-pyrazol-1-yl)propan-2-ol (283 mg, 1.06 mmol, 2 equiv) and Pd(dppf)Cl2 (97 mg, 0.13 mmol, 0.25 equiv) were placed in a round-bottom flask, followed by 1,4-dioxane (9.6 mL) and 2N Na₂CO₃ aqueous solution (4.8 mL). The solution was degassed with organ, moved to 80° C. and allowed to stir for 0.5 h. The solution was quenched with saturated ammonium chloride and extracted with EA (3×30 mL). The combined organic layers were washed with H₂O (2×30 mL) and brine brine solution (20 mLmL), dried over anhydrous Na₂SO₄, filtered, and concentrated by rotary evaporation to get the crude compound. Then, the resulting concentrate was purified on a silica column to get desired compound (200 mg, purity=99%, yield=78%) as a yellow solid.

TLC R_{f}=0.1 (DCM:EA=2:1);

LCMS (m/z)=485.80 (M+H)

¹H NMR (400 MHz, DMSO-d₆) δ 8.46 (s, 1H), 8.16 (s, 1H), 7.91 (s, 1H), 7.79 (s, 1H), 7.63 (s, 1H), 7.45-7.35 (m, 2H), 7.21 (s, 1H), 6.91 (d, J=8.7 Hz, 2H), 6.74 (d, J=8.7 Hz, 2H), 5.18 (s, 2H), 4.75 (s, 1H), 4.03 (s, 2H), 3.81-2.84 (br., 2H), 3.64 (s, 3H), 1.09 (s, 6H).

### Example 97. 8-(1-(2-Hydroxy-2-methylpropyl)-1H-pyrazol-4-yl)-4-(isoxazol-3-yl)-1-(4-methoxybenzyl)-1,3-dihydro-2H-benzo[b]azepin-2-one (97)

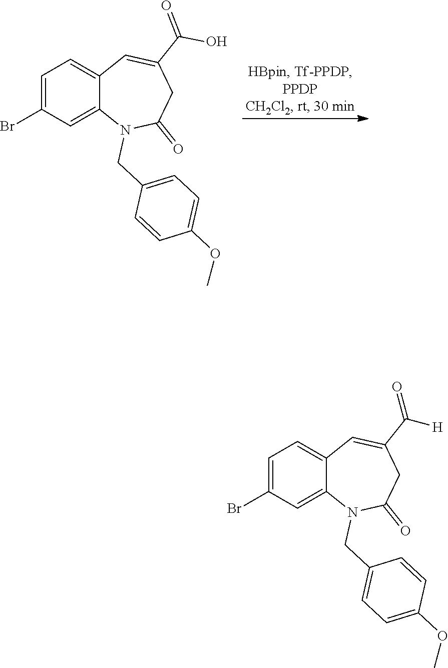 [SMILES: COc1ccc(CN2C(=O)CC(C(=O)O)=Cc3ccc(Br)cc32)cc1 ; COc1ccc(CN2C(=O)CC(C=O)=Cc3ccc(Br)cc32)cc1]

### 8-bromo-1-(4-methoxybenzyl)-2-oxo-2,3-dihydro-1H-benzo[b]azepine-4-carbaldehyde

To a solution of 8-bromo-l-(4-methoxybenzyl)-2-oxo-2,3-dihydro-1H-benzo[b]azepine-4-carboxylic acid (2 g, 4.975 mmol, 1.0 equiv), 4,4′-dihydroxybiphenyl (1.29 g, 7.960 mmol, 1.6 equiv) in DCM (40 mL) were added trifluoromethanesulfonyl pyridinium salt(3.74 g, 8.457 mmol, 1.7 equiv) and HBpin (700 mg, 5.472 mmol, 1.1 equiv). The resulting solution was allowed to stir for 10 minutes at room temperature, quenched with H₂O (volume of water?) and extracted by DCM (3×3.0 mm). The combined organic layers were dried over anhydrous Na₂SO₄, filtered, concentrated under reduced pressure. The resulting residue was purified by flash column chromatography on silica gel to get a pure compound (1.2 g, purity=96%, yield=62.5%)

TLC R_{f}=0.7 (EA);

LCMS (m/z)=388.00 (M+H)

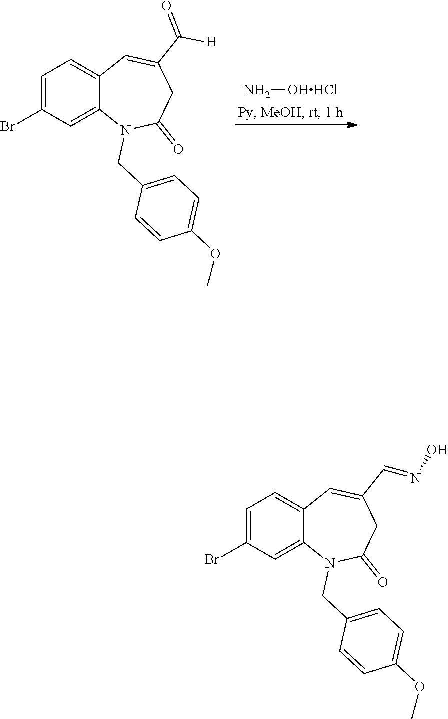

### 8-Bromo-1-(4-methoxybenzyl)-2-oxo-2,3-dihydro-1H-benzo[b]azepine-4-carbaldehyde oxime

To a stirred solution of 8-bromo-l-(4-methoxybenzyl)-2-oxo-2,3-dihydro-1H-benzo[b]azepine-4-carbaldehyde (1.2 g, 3.108 mmol, 1 equiv) and hydroxylamine hydrochloride (280 mg, 4.041 mmol, 1.3 equiv) in methanol (12 mL) at room temperature was added pyridine (0.3 mL, 3.730 mol, 1.2 equiv). The reaction mass was stirred at rt for 3 h, concentrated under reduced pressure. The resulting crude was purified by silica column to get a pure compound (1.0 g, purity=96.8%, yield=80.6%) as an off white solid.

TLC R_{f}=0.5 (20% EA:PE)

LCMS (m/z)=403.10 (M+H)

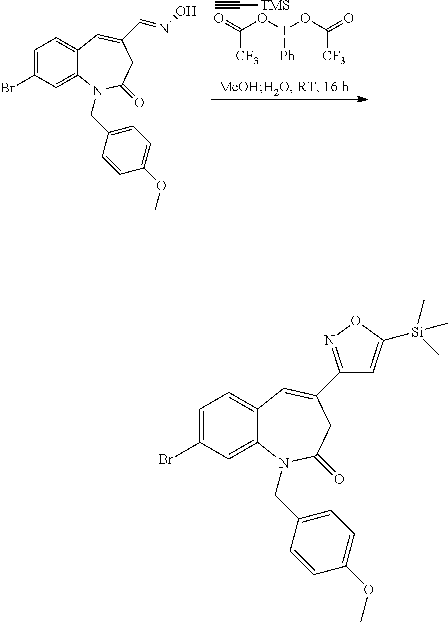

### 8-Bromo-1-(4-methoxybenzyl)-4-(5-(trimethylsilyl)isoxazol-3-yl)-1,3-dihydro-2H-benzo[b]azepin-2-one

To a stirred solution of (E)-8-bromo-1-(4-methoxybenzyl)-2-oxo-2,3-dihydro-1H-benzo[b]azepine-4-carbaldehyde oxime (1.0 g, 2.493 mmol, 1 equiv) in methanol:water (4:1, 150 mL) were added ethynyl trimethyl silane (1.7 mL, 4.987 mmol, 2.0 equiv) and phenyl-13-iodanediyl bis(2,2,2-trifluoroacetate) (1.17 mg, 2.743 mmol, 1.1 equiv). The resulting solution was allowed to stir for 16 h at room temperature and poured into methyl tert-butyl ether (100 mL). The organic layer was washed with aq. Na₂CO₃ solution, brine solution, and water. Finally, it was dried with anhydrous Na₂SO₄, filtered, and concentrated under reduced pressure. The crude compound was purified by silica column to get a title compound (0.43 g, purity=96.3%, yield=35%) as an off-white solid.

TLC R_{f}=0.7 (200% EA:PE)

LCMS (m/z)=499.20 (M+H)

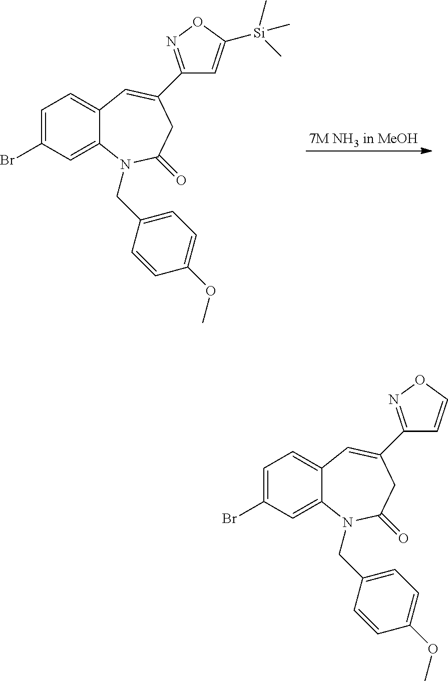 [SMILES: COc1ccc(CN2C(=O)CC(c3cc([Si](C)(C)C)on3)=Cc3ccc(Br)cc32)cc1 ; COc1ccc(CN2C(=O)CC(c3ccon3)=Cc3ccc(Br)cc32)cc1]

### 8-Bromo-4-(isoxazol-3-yl)-1-(4-methoxybenzyl)-1,3-dihydro-2H-benzo[b]azepin-2-one

7N NH₃ in methanol (50 mL) solution was added to the round bottom flask containing 8-bromo-1-(4-methoxybenzyl)-4-(5-(trimethylsilyl) isoxazole-3-yl)-1,3-dihydro-2H-benzo[b]azepin-2-one (0.42 g, 0.846 mmol, 1 equiv) and allowed to stir for 30 min. at room temperature. The resulting compound was concentrated under reduced pressure and diluted with n-hexane, and the supernatant was decanted to remove impurities. Finally, the solid was dried under a high vacuum to afford the product (350 mg, purity=93.58%, yield=97%) as an off-white solid.

TLC R_{f}=0.7 (EA:PE 1:1)

LCMS (m/z)=427.20 (M+H)

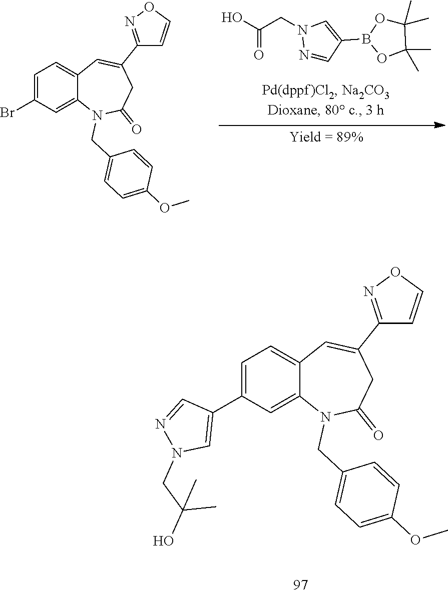 [SMILES: COc1ccc(CN2C(=O)CC(c3ccon3)=Cc3ccc(Br)cc32)cc1 ; CC1(C)OB(c2cnn(CC(=O)O)c2)OC1(C)C ; COc1ccc(CN2C(=O)CC(c3ccon3)=Cc3ccc(-c4cnn(CC(C)(C)O)c4)cc32)cc1]

### 8-(1-(2-Hydroxy-2-methylpropyl)-1H-pyrazol-4-yl)-4-(isoxazol-3-yl)-1-(4-methoxybenzyl)-1,3-dihydro-2H-benzo[b]azepin-2-one: 97

To a solution of 8-Bromo-4-(4,5-dihydroisoxazol-3-yl)-1-(4-methoxybenzyl)-1,3-dihydro-2H-benzo[b]azepin-2-one (250 mg, 0.58 mmol, 1 equiv), 2-methyl-1-(4-(4,4,5,5-tetramethyl-1,3,2-dioxaborolan-2-yl)-1H-pyrazol-1-yl)propan-2-ol (312 mg, 1.17 mmol, 2 equiv) and Pd(dppf)Cl₂ (86 mg, 0.11 mol, 0.25 equiv) in 1,4-dioxane (13 mL) were added 2N Na₂CO₃ aqueous solution (7 mL). The resulting solution was degassed with argon and allowed to stir for 3 h at 80° C. after completion of the reaction, quenched with saturated ammonium chloride and extracted with EA (3×50 mL). The organic solutions were combined, washed with H₂O (2×30 mL) and brine solution (1×30 mL), dried over anhydrous Na₂SO₄, filtered, and concentrated by rotary evaporation to get the crude compound. Finally, the crude was purified by a silica column to get the pure compound 97 (240 mg, purity=100%, yield=84%) as a white solid.

TLC R_{f}=0.4 (EA)

LCMS (m/z)=485.5 (M+H)

¹H NMR (400 MHz, DMSO-d₆) δ 8.96 (d, J=1.7 Hz, 1H), 8.17 (s, 1H), 7.92 (s, 1H), 7.80 (s, 1H), 7.57 (s, 1H), 7.48-7.35 (m, 2H), 7.13 (d, J=1.9 Hz, 1H), 6.93 (d, J=8.7 Hz, 2H), 6.74 (d, J=8.7 Hz, 2H), 5.19 (s, 2H), 4.75 (s, 1H), 4.03 (s, 2H), 3.97-2.90 (br., 2H), 3.64 (s, 3H), 1.09 (s, 6H).

### Example 98. 8-(1-(2-Hydroxy-2-methylpropyl)-1H-pyrazol-4-yl)-1-(4-methoxybenzyl)-4-(1,3,4-oxadiazol-2-yl)-1,3-dihydro-2H-benzo[b]azepin-2-one (98)

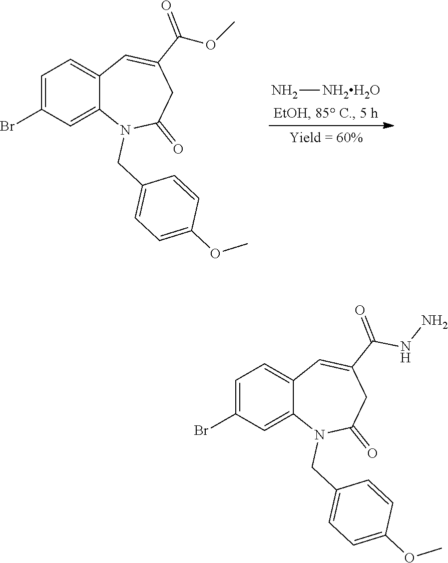

### 8-Bromo-1-(4-methoxybenzyl)-2-oxo-2,3-dihydro-1H-benzo[b]azepine-4-carbohydrazide

To a solution of methyl 8-bromo-1-(4-methoxybenzyl)-2-oxo-2,3-dihydro-1H-benzo[b]azepine-4-carboxylate (1.0 g, 153.846 mmol, 1 equiv) in EtOH (20 mL) was added NH₂—NH₂·H₂O (3 mL, 60.075 mmol, 25 equiv) and the reaction mixture was stirred for 5 h at 85° C. After completion of the reaction, reaction flask was moved to room temperature and observed precipitate. It was filtered and washed with water and pet ether to afford the desired compound (600 mg, yield=60%, purity=100%) as an Off-white solid.

TLC R_{f}=0.5 (PE:EA=4:1);

LCMS (m/z)=416.2 (M+H)

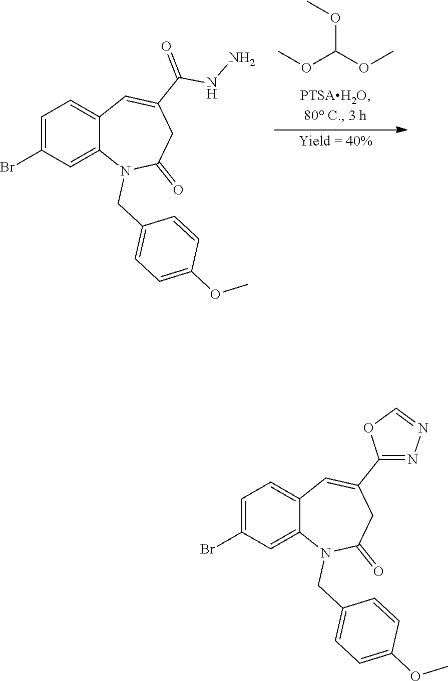

### 8-Bromo-1-(4-methoxybenzyl)-4-(1,3,4-oxadiazol-2-yl)-1,3-dihydro-2H-benzo[b]azepin-2-one

To a solution of 8-bromo-l-(4-methoxybenzyl)-2-oxo-2,3-dihydro-1H-benzo[b]azepine-4-carbohydrazide (500 mg, 153.846 mmol, 1 equiv) in Trimethyl orthoformate (40 mL) was added PTSA·H₂O (0.239 mL, 1.566 mmol, 1.3 equiv) and the reaction mixture was allowed to stir for 3 h at 80° C. After completion of the reaction, diluted with water (20 mL) and extracted with ethyl acetate (3×30 mL). The organic layers were combined, dried over anhydrous Na₂SO₄, filtered, and concentrated under reduced pressure to get a crude compound. The crude compound was as purified by flash column chromatography to afford the target compound (200 mg, Yield=39%, Purity=100%) as an off-white solid.

TLC R_{f}=0.1 (50% EA:PE)

LCMS (m/z)=427.80 (M+H)

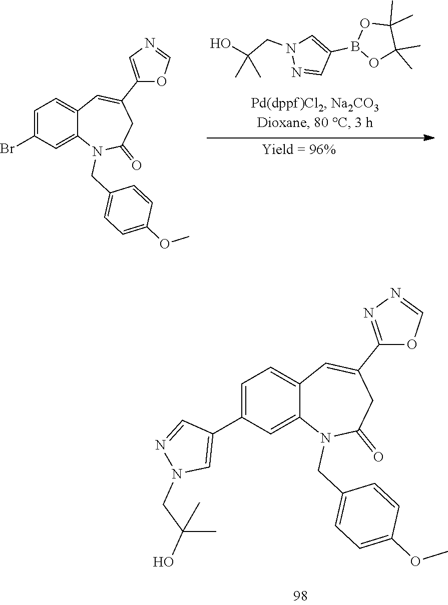 [SMILES: COc1ccc(CN2C(=O)CC(c3cnco3)=Cc3ccc(Br)cc32)cc1 ; CC(C)(O)Cn1cc(B2OC(C)(C)C(C)(C)O2)cn1 ; COc1ccc(CN2C(=O)CC(c3nnco3)=Cc3ccc(-c4cnn(CC(C)(C)O)c4)cc32)cc1]

### 8-(1-(2-Hydroxy-2-methylpropyl)-1H-pyrazol-4-yl)-1-(4-methoxybenzyl)-4-(1,3,4-oxadiazol-2-yl)-1,3-dihydro-2H-benzo[b]azepin-2-one:98

To a solution of 8-bromo-1-(4-methoxybenzyl)-4-(1,3,4-oxadiazol-2-yl)-1,3-dihydro-2H-benzo[b]azepin-2-one (200 mg, 0.469 mmol, 1 equiv) and 2-methyl-1-(4-(4,4,5,5-tetramethyl-1,3,2-dioxaborolan-2-yl)-1H-pyrazol-1-yl)propan-2-ol (250 mg, 0.938 mmol, 2 equiv) in 1,4-dioxane (13 mL) were added Pd(dppf)Cl2 (68 mg, 0.0938 mol, 0.2 equiv) and 2N Na₂CO₃ aqueous solution (7 mL). The solution was degassed with an argon gas and allowed to stir for 3 h at 80° C. The reaction mass was quenched with saturated ammonium chloride and extracted with EA (3×50 mL). The combined organic layers were washed with H₂O (2×30 mL) and brine solution (1×30 mL), dried over anhydrous Na₂SO₄, filtered, and concentrated by rotary evaporation to get the crude compound. The crude was purified by silica column to get a pure compound (220 mg, Purity=96.54%, Yield=96%) as an off-white solid.

TLC R_{f}=0.4 (EA)

LCMS (m/z)=486.40 (M+H)

¹H NMR (400 MHz, DMSO-d₆) δ 9.32 (s, 1H), 8.20 (s, 1H), 7.94 (s, OH), 7.83 (d, J=1.6 Hz, 1H), 7.72 (s, 1H), 7.50 (d, J=8.2 Hz, 1H), 7.44 (dd, J=8.1, 1.6 Hz, 1H), 6.93 (d, J=2.0 Hz, 1H), 6.92 (d, J=2.1 Hz, 1H), 6.75 (d, J=2.0 Hz, 1H), 6.74 (d, J=2.1 Hz, 1H), 5.20 (s, 2H), 4.75 (s, 1H), 4.04 (s, 2H), 3.81-3.27 (br., 2H), 3.64 (s, 3H), 1.09 (s, 6H).

### Example 99. 8-(1-(2-Hydroxy-2-methylpropyl)-1H-pyrazol-4-yl)-1-(4-methoxybenzyl)-4-(3-methyl-1,2,4-oxadiazol-5-yl)-1,3-dihydro-2H-benzo[b]azepin-2-one (99)

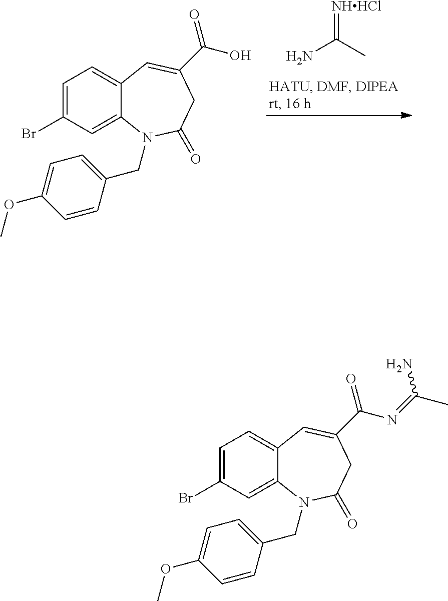

### N-(1-aminoethylidene)-8-bromo-1-(4-methoxybenzyl)-2-oxo-2,3-dihydro-1H-benzo[b]azepine-4-carboxamide

To a solution of 8-bromo-1-(4-methoxybenzyl)-2-oxo-2,3-dihydro-1H-benzo[b]azepine-4-carboxylic acid (50 mg, 0.124 mmol, 1 equiv), HATU (52 mg, 0.136, 1.1 equiv), DIPEA (0.06 mL, 0.373, 3 equiv) in DMF was added acetimidamide hydrochloride (17.6 mg, 0.186 mmol, 1.5 equiv) at room temperature. The reaction mass was allowed to stir for 16 h. After completion of the reaction, the reaction solution was poured into ice water and observed precipitate. The resulting precipitate was filtered and dried to get the desired compound (30 mg, Purity=86%, Yield=54%) as an off-white solid.

TLC R_{f}=0.2 (1:1, EA:PE)

LCMS (m/z)=443.90 (M+H)

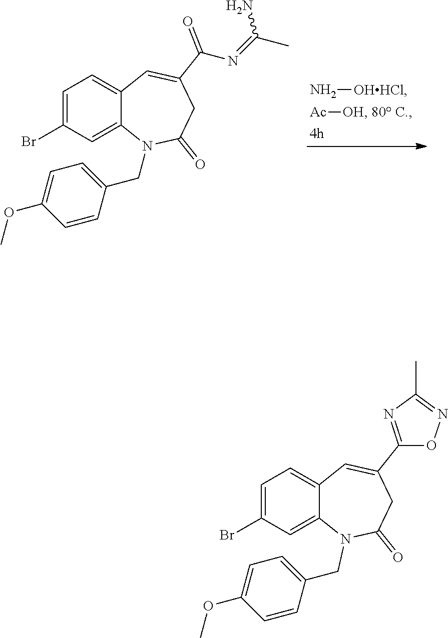

### 8-Bromo-1-(4-methoxybenzyl)-4-(3-methyl-1,2,4-oxadiazol-5-yl)-1,3-dihydro-2H-benzo[b]azepin-2-one

To a stirred solution of N-(1-aminoethylidene)-8-bromo-1-(4-methoxybenzyl)-2-oxo-2,3-dihydro-1H-benzo[b]azepine-4-carboxamide (30 mg, 0.0677 mmol, 1 equiv) in acetic acid (2 mL), was added hydroxyl amine hydrochloride (7 mg, 0.101 mmol, 1.5 equiv) and allowed to stir for 4 h at 80° C. After completion of the reaction, the reaction solution was poured into ice water and observed precipitate. The resulting precipitate was filtered and dried to get the desired compound (19 mg, Purity=89%, Yield=71%)

TLC R_{f}=0.5 (1:1, EA:PE)

LCMS (m/z)=440.40 (M+H)

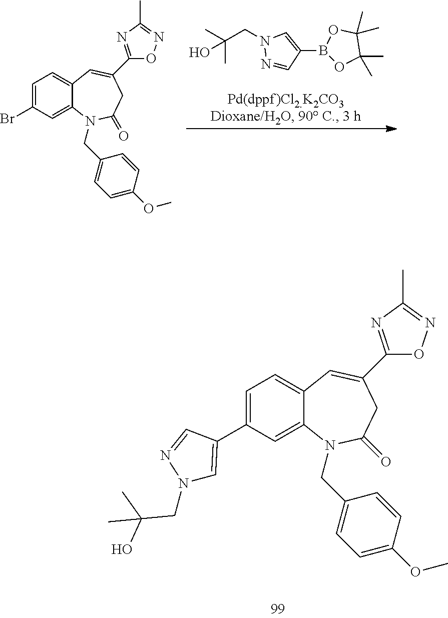 [SMILES: COc1ccc(CN2C(=O)CC(c3nc(C)no3)=Cc3ccc(Br)cc32)cc1 ; CC(C)(O)Cn1cc(B2OC(C)(C)C(C)(C)O2)cn1 ; COc1ccc(CN2C(=O)CC(c3nc(C)no3)=Cc3ccc(-c4cnn(CC(C)(C)O)c4)cc32)cc1]

### 8-(1-(2-Hydroxy-2-methylpropyl)-1H-pyrazol-4-yl)-1-(4-methoxybenzyl)-4-(3-methyl-1,2,4-oxadiazol-5-yl)-1,3-dihydro-2H-benzo[b]azepin-2-one: 99

To a stirred solution of 8-bromo-1-(4-methoxybenzyl)-4-(3-methyl-1,2,4-oxadiazol-5-yl)-1,3-dihydro-2H-benzo[b]azepin-2-one (180 mg, 0.409 mmol, 1 equiv) in 1,4-dioxane/water (10 mL) was added 2-methyl-1-(4-(4,4,5,5-tetramethyl-1,3,2-dioxaborolan-2-yl)-1H-pyrazol-1-yl)propan-2-ol (163 mg, 0.613 mmol, 1.5 equiv), K₂CO₃ (141 mg, 1.022 mmol, 2.5 equiv) and Pd(dppf)Cl₂ (45 mg, 0.0818 mmol, 0.2 equiv). The resulting solution was degassed with argon gas and stirred for 3 h at 90° C. After completion of the reaction, quenched with saturated ammonium chloride and extracted with EA (3×30 mL). The combined organic layers were washed with H₂O (2×20 mL) and brine solution (1×20 mL), dried over anhydrous Na₂SO₄, filtered, and concentrated by rotary evaporation to get the crude compound. The crude was purified by silica column to get a pure compound (55 mg, purity=97.7%, yield=27%) as an off-white solid.

TLC R_{f}=0.2 (EA)

LCMS (m/z)=485.40 (M+H)

¹H NMR (400 MHz, Chloroform-d) δ 7.79 (s, 1H), 7.72 (s, 1H), 7.64 (s, 1H), 7.49 (d, J=1.6 Hz, 1H), 7.36 (d, J=8.1 Hz, 1H), 7.31 (dd, J=8.1, 1.6 Hz, 1H), 7.07 (d, J=8.6 Hz, 2H), 6.78 (d, J=8.7 Hz, 2H), 5.07 (s, 2H), 4.10 (s, 2H), 3.88-3.23 (br., 2H), 3.75 (s, 3H), 2.44 (s, 3H), 2.22-1.75 (br., 1H), 1.21 (s, 6H).

### Example 100. 8-(1-(2-Hydroxy-2-methylpropyl)-1H-pyrazol-4-yl)-1-(4-(methoxy-d₃)benzyl)-4-(5-methyloxazol-2-yl)-1,3-dihydro-2H-benzo[b]azepin-2-one (100)

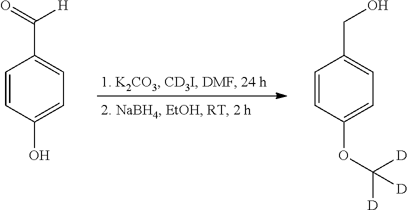

### (4-(Methoxy-d3)phenyl)methanol

To a stirred solution of 4-hydroxybenzaldehyde (1 g, 0.619 mmol, 1 equiv), K₂CO₃ (1.9 gm, 13.93, 1.7 equiv), CD₃I (0.7 mL, 10.65 mmol, 1.3 equiv) in DMF. The reaction mixture was stirred at rt for 24 h. Then add EtOH and NaBH₄ and stirred for 2 h at rt. Reaction mass was poured into water (40 mL) and extracted with ethyl acetate (2×40 mL). The combined organic layers were washed with H₂O (20 mL) and brine solution (20 mL), dried over anhydrous Na₂SO₄, and concentrated by rotary evaporation to get crude compound 900 mg as a yellow liquid. with a yield of 81%, purity=98%

TLC R_{f}=0.3 (PE:EA=1:1);

LCMS (m/z)=125.00 (M+H)

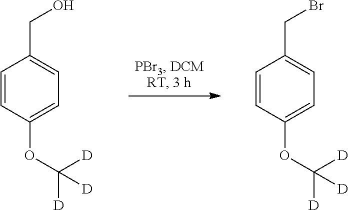 [SMILES: [2H]C([2H])([2H])Oc1ccc(CO)cc1 ; "PBr3, DCMRT, 3 h" ; [2H]C([2H])([2H])Oc1ccc(CBr)cc1]

### 1-(Bromomethyl)-4-(methoxy-d3)benzene

To a stirred solution of (4-(methoxy-d3)phenyl)methanol (50 mg, 0.354 mmol, 1 equiv), PBr₃ (0.1 mL, 0.708 mmol, 3 equiv) in DCM (5 mL) and the reaction mass was stirred at rt for 3 h. The reaction mass was poured into NaHCO₃ solution, extracted with DCM (3×10 mL). The organic solutions were combined, washed with H₂O (2×10 mL) and brine solution (1×10 mL), dried over anhydrous Na₂SO₄, filtered and concentrated by rotary evaporation to get the crude compound (50 mg, purity=TLC, yield=69%) as a color less liquid. Go next step without purification.

TLC R_{f}=0.7 (1:3, EA/PE)

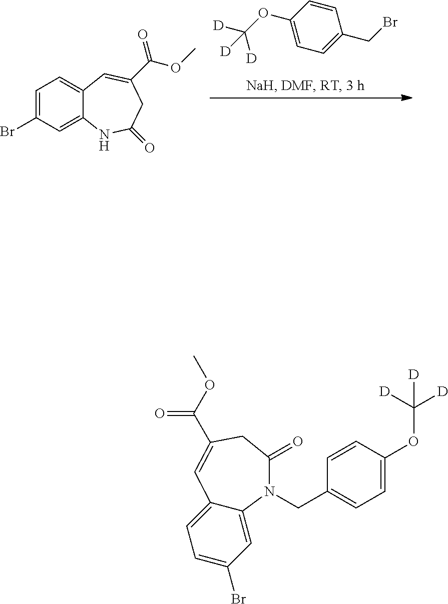

### Methyl 8-bromo-1-(4-(methoxy-d3)benzyl)-2-oxo-2,3-dihydro-1H-benzo[b]azepine-4-carboxylate

To a stirred solution of methyl 8-bromo-2-oxo-2,3-dihydro-1H-benzo[b]azepine-4-carboxylate (500 mg, 0.680 mmol, 1 equiv), NaH (150 mg, 6.25 mmol, 3.7 equiv) in DMF (20 mL) was added 1-(bromomethyl)-4-(methoxy-d3)benzene (516 mg, 2.533 mmol, 1.5 equiv) at rt. The reaction mixture was stirred at rt for 3 h. Reaction mass was cooled to rt and poured into water (20 mL) and extracted with ethyl acetate (2×30 mL). The combined organic layers were washed with H₂O (3×50 mL) and brine solution (30 mL), dried over anhydrous Na₂SO₄, and concentrated by rotary evaporation. Then, the resulting concentrate was purified on a silica column to get 280 mg brown solid. yield of 40%, purity=TLC.

TLC R_{f}=0.5 (PE:EA=4:1)

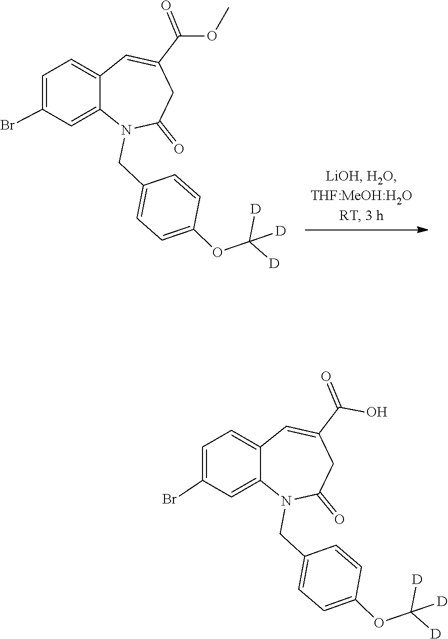 [SMILES: [2H]C([2H])([2H])Oc1ccc(CN2C(=O)CC(C(=O)OC)=Cc3ccc(Br)cc32)cc1 ; [2H]C([2H])([2H])Oc1ccc(CN2C(=O)CC(C(=O)O)=Cc3ccc(Br)cc32)cc1]

### 8-Bromo-1-(4-(methoxy-d3)benzyl)-2-oxo-2,3-dihydro-1H-benzo[b]azepine-4-carboxylic acid

To a mixture of methyl 8-bromo-1-(4-(methoxy-d3)benzyl)-2-oxo-2,3-dihydro-1H-benzo[b]azepine-4-carboxylate (50 mg, 0.119 mmol, 1 equiv) in THF:MeOH:H₂O (10 mL) was added LiOH·H₂O (25 mg, 0.595 mmol, 5 equiv) at rt. The reaction mixture was stirred at rt for 3 h; the reaction mixture solvent was evaporated, obtained crude was adjusted pH=3-4 with 1N HCl, and then extracted with EtOAc (2×20 mL). The organic layer was washed with water and brine solution, dried over Na₂SO₄, and concentrated in vacuo to get a crude 40 mg brown solid.

TLC R_{f}=0.2 (DCM:MeOH=9:1)

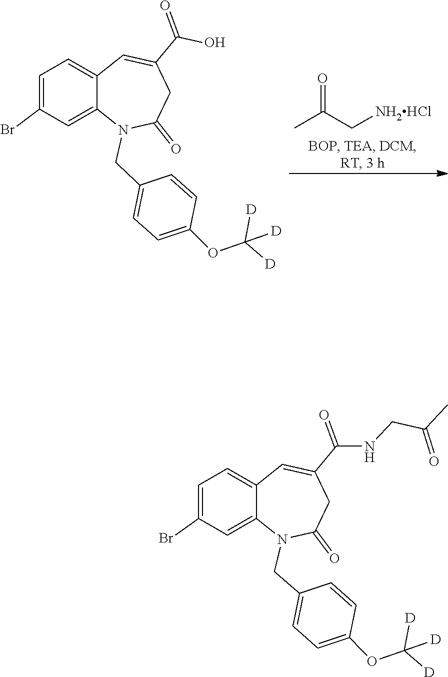

### 8-Bromo-1-(4-(methoxy-d3)benzyl)-2-oxo-N-(2-oxopropyl)-2,3-dihydro-1H-benzo[b]azepine-4-carboxamide

To a stirred solution of 8-bromo-1-(4-(methoxy-d3)benzyl)-2-oxo-2,3-dihydro-1H-benzo[b]azepine-4-carboxylic acid (40 mg, 0.0981 mmol, 1 equiv), 1-aminoacetone HCl salt (16 mg, 0.148 mmol, 1.5 equiv), BOP reagent (65 mg, 0.148 mmol, 1.5 equiv), and triethylamine (0.03 mL, 0.246 mmol, 2.5 equiv) in DCM (5 mL) was stirred for 2-3 h. The solvent was removed by rotary evaporation to get the crude, which was purified on a silica column to get 40 mg of product as a brown liquid. Yield=88%, purity=88%.

TLC R_{f}=0.5 (EA)

LCMS (m/z)=462.10 (M+H)

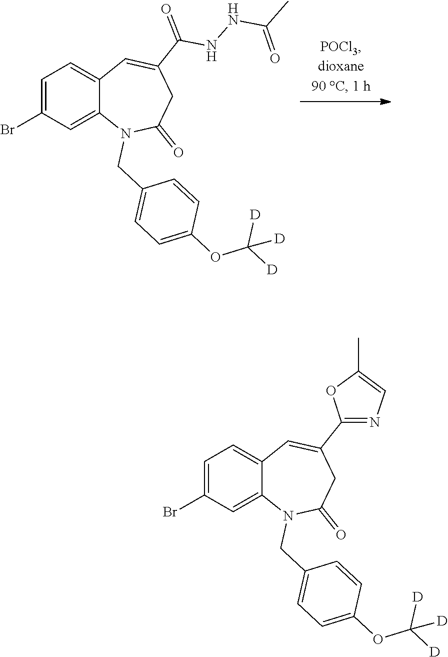 [SMILES: [2H]C([2H])([2H])Oc1ccc(CN2C(=O)CC(C(=O)NNC(C)=O)=Cc3ccc(Br)cc32)cc1 ; [2H]C([2H])([2H])Oc1ccc(CN2C(=O)CC(c3ncc(C)o3)=Cc3ccc(Br)cc32)cc1]

### 8-Bromo-1-(4-(methoxy-d3)benzyl)-4-(5-methyloxazol-2-yl)-1,3-dihydro-2H-benzo[b]azepin-2-one

8-bromo-1-(4-(methoxy-d3)benzyl)-2-oxo-N-(2-oxopropyl)-2,3-dihydro-1H-benzo[b]azepine-4-carboxamide (90 mg, 0.195 mmol, 1 equiv) was dissolved in 1,4-dioxane (5 mL) and phosphoryl chloride (0.4 mL, 1.271 mmol, 6.5 equiv) was added and the mixture was heated at 90° C. for 1 h, then cooled to room temperature and diluted with ethyl acetate. The mixture was washed with saturated NaHCO₃ and brine solution, dried over sodium sulfate, filtered and concentrated in vacuum. The residue was purified by silica gel column chromatography to get the product (60 mg) as a brown semi solid. Yield: 100%; Purity: 73.1%.

TLC R_{f}=0.8 (PE:EA=1:1);

LCMS (m/z)=442.2 (M+H)

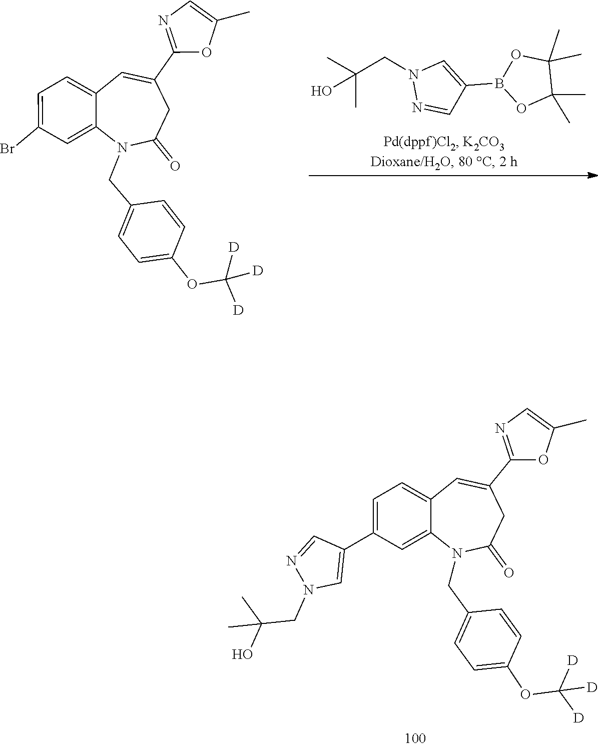 [SMILES: [2H]C([2H])([2H])Oc1ccc(CN2C(=O)CC(c3ncc(C)o3)=Cc3ccc(Br)cc32)cc1 ; CC(C)(O)Cn1cc(B2OC(C)(C)C(C)(C)O2)cn1 ; [2H]C([2H])([2H])Oc1ccc(CN2C(=O)CC(c3ncc(C)o3)=Cc3ccc(-c4cnn(CC(C)(C)O)c4)cc32)cc1]

### 8-(1-(2-Hydroxy-2-methylpropyl)-1H-pyrazol-4-yl)-1-(4-(methoxy-d3)benzyl)-4-(5-methyloxazol-2-yl)-1,3-dihydro-2H-benzo[b]azepin-2-one: 100

To a stirred solution of 8-bromo-1-(4-(methoxy-d3)benzyl)-4-(5-methyloxazol-2-yl)-1,3-dihydro-2H-benzo[b]azepin-2-one (60 mg, 0.136 mmol, 1 equiv) in 1,4-dioxane/water (5 mL) was added 2-methyl-1-(4-(4,4,5,5-tetramethyl-1,3,2-dioxaborolan-2-yl)-1H-pyrazol-1-yl)propan-2-ol (90 mg, 0.204 mmol, 1.5 equiv), K₂CO₃ (47 mg, 0.34 mmol, 2.5 equiv). The solution was degassed and added Pd(dppf)Cl2 (20 mg, 0.0272 mmol, 0.2 equiv), then heated to 80° C. and stirred for 2 h. The reaction mass was quenched with saturated ammonium chloride and extracted with EA (3×10 mL). The organic solutions were combined, washed with H₂O (2×10 mL) and brine solution (1×10 mL), dried over anhydrous Na₂SO₄, filtered and concentrated by rotary evaporation to get the crude compound. The crude was purified by silica column to get pure compound (20 mg, purity=93.7%, yield=30%) as a brown semi solid.

TLC R_{f}=0.3 (1:1,EA/PE)

LCMS (m/z)=502.50 (M+H)

### Example 101. 1-(3-fluoro-4-methylbenzyl)-8-(1-(2-hydroxy-2-methylpropyl)-1H-pyrazol-4-yl)-4-(5-methyloxazol-2-yl)-1,3-dihydro-2H-benzo[b]azepin-2-one (101)

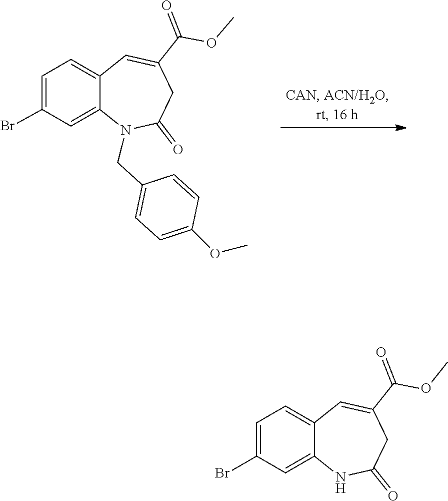 [SMILES: COC(=O)C1=Cc2ccc(Br)cc2N(Cc2ccc(OC)cc2)C(=O)C1 ; COC(=O)C1=Cc2ccc(Br)cc2NC(=O)C1]

### Methyl 8-bromo-2-oxo-2,3-dihydro-1H-benzo[b]azepine-4-carboxylate

To a stirred solution methyl 8-bromo-1-(4-methoxybenzyl)-2-oxo-2,3-dihydro-1H-benzo[b]azepine-4-carboxylate (1.248 g, 3 mmol, 1 equiv) in a 9:1 mixture of solvent ACN/H₂O (16 mL), was added CAN (4.934 g, 9 mmol, 3 equiv) at ambient temperature. The reaction mixture was stirred at rt for 16 h. After TLC complies reaction mass was poured in to water (40 mL), solid was precipitated, filtered and dried to 1.03 mg dark brown solid. yield of >99% (solvent contain), Purity=>58%

TLC R_{f}=0.2 (50% EA:PE)

LCMS (m/z)=295.90 (M+H)

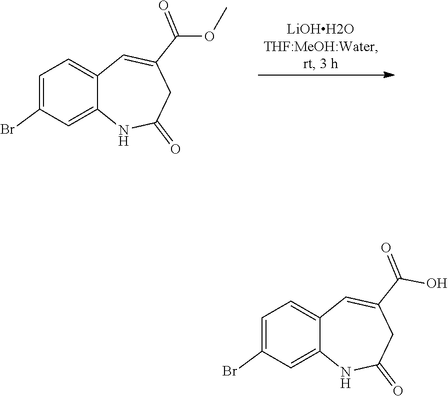 [SMILES: COC(=O)C1=Cc2ccc(Br)cc2NC(=O)C1 ; O=C1CC(C(=O)O)=Cc2ccc(Br)cc2N1]

### 8-Bromo-2-oxo-2,3-dihydro-1H-benzo[b]azepine-4-carboxylic acid

To a solution of methyl 8-bromo-2-oxo-2,3-dihydro-1H-benzo[b]azepine-4-carboxylate (1.03 g, 3.47 mmol, 1 equiv) in 1:1:1 mixture solvent MeOH/THF/H₂O (21 mL), was added LiOH·H₂O (730 mg, 17.39 mmol, 5 equiv) at ambient temperature. The reaction mixture was stirred at r.t for 2 h. Solvent removed completely, added water (5 mL), adjust pH=3-4 with 1N HCl and filtered. The solid was washed with H₂O (2×20 mL) and dried to get the 717 mg white solid. purity=97.77%, yield of 73%.

TLC R_{f}=0.1 (EA)

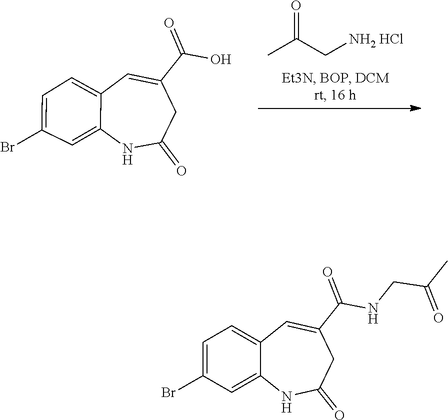

### 8-Bromo-2-oxo-N-(2-oxopropyl)-2,3-dihydro-1H-benzo[b]azepine-4-carboxamide

A mixture of 8-bromo-2-oxo-2,3-dihydro-1H-benzo[b]azepine-4-carboxylic acid (1.4 g, 4.96 mmol, 1 equiv), 1-aminoacetone HCl salt (811 mg, 7.44 mmol, 1.5 equiv), BOP reagent (3.28 g, 7.44 mmol, 1.5 equiv), and triethylamine (2.16 mL, 12.4 mmol, 2.5 equiv) in DCM (24 mL) was stirred for 16 h. The solvent was removed by rotary evaporation, to get the crude, added water (50 mL), filtered and washed with water (30 mL), dried to get crude product, which was purified by stirring with EA (10 mL) to get 1.06 g (purity=100%) brown solid compound with a yield of 63%.

TLC R_{f}=0.5 (EA)

LCMS (m/z)=337.00 (M+H)

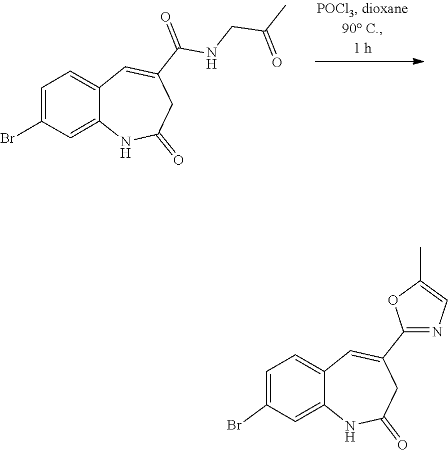 [SMILES: CC(=O)CNC(=O)C1=Cc2ccc(Br)cc2NC(=O)C1 ; Cc1cnc(C2=Cc3ccc(Br)cc3NC(=O)C2)o1]

### 8-Bromo-4-(5-methyloxazol-2-yl)-1,3-dihydro-2H-benzo[b]azepin-2-one

8-bromo-2-oxo-N-(2-oxopropyl)-2,3-dihydro-1H-benzo[b]azepine-4-carboxamide (1.06 g, 3.14 mmol, 1 equiv) was dissolved in 1,4-dioxane (46 mL) and phosphoryl chloride (1.92 mL, 20.66 mmol, 6.57 equiv) was added and the mixture was heated at 90° C. for 1 h, then cooled to room temperature and diluted with ethyl acetate. The mixture was washed with sated. NaHCO₃ and brine solution, dried over sodium sulfate, filtered and concentrated in vacuum. The residue was purified by silica column to get the product (800 mg) as a white solid. Yield: 80%; Purity: 97.02%.

TLC R_{f}=0.8 (PE:EA=1:1)

LCMS (m/z)=319.30 (M+H)

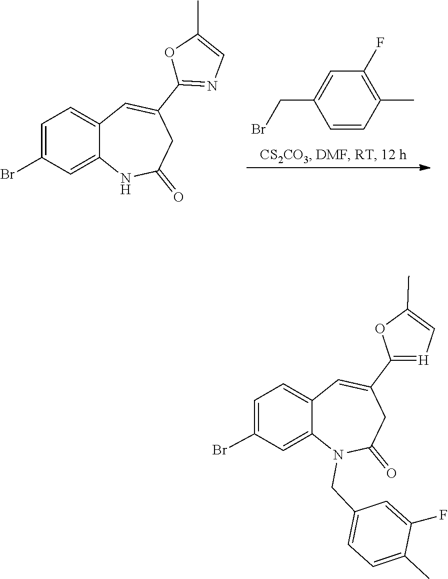

### 8-Bromo-1-(3-fluoro-4-methylbenzyl)-4-(5-methyloxazol-2-yl)-1,3-dihydro-2H-benzo[b]azepin-2-one

To a stirred solution of 8-bromo-4-(5-methyloxazol-2-yl)-1,3-dihydro-2H-benzo[b]azepin-2-one (60 mg, 0.188 mmol, 1 equiv), CS₂CO₃ (184 mg, 0.564 mmol, 3 equiv) in DMF (20 mL) was added 4-(bromomethyl)-2-fluoro-1-methylbenzene (56 mg, 0.225 mmol, 1.2 equiv) at rt. The reaction mixture was stirred at rt for 12 h. Reaction mass was cooled to rt and poured into water (20 mL) and extracted with ethyl acetate (2×20 mL). The combined organic layers were washed with H₂O (3×20 mL) and brine solution (20 mL), dried over anhydrous Na₂SO₄, and concentrated by rotary evaporation. Then, the resulting concentrate was purified on a silica column to get 40 mg of compound as an off white solid. yield of 48%, purity=100%

TLC R_{f}=0.4 (PE:EA=1:1)

LCMS (m/z)=441.10 (M+H)

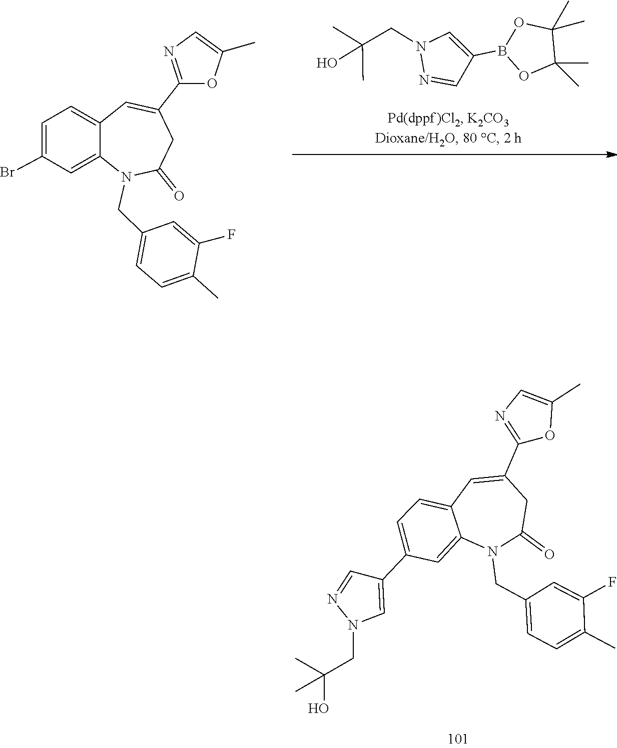 [SMILES: Cc1cnc(C2=Cc3ccc(Br)cc3N(Cc3ccc(C)c(F)c3)C(=O)C2)o1 ; CC(C)(O)Cn1cc(B2OC(C)(C)C(C)(C)O2)cn1 ; Cc1cnc(C2=Cc3ccc(-c4cnn(CC(C)(C)O)c4)cc3N(Cc3ccc(C)c(F)c3)C(=O)C2)o1]

### 1-(3-fluoro-4-methylbenzyl)-8-(1-(2-hydroxy-2-methylpropyl)-1H-pyrazol-4-yl)-4-(5-methyloxazol-2-yl)-1,3-dihydro-2H-benzo[b]azepin-2-one: 101

To a stirred solution of 8-bromo-1-(3-fluoro-4-methylbenzyl)-4-(5-methyloxazol-2-yl)-1,3-dihydro-2H-benzo[b]azepin-2-one (40 mg, 0.090 mmol, 1 equiv) in 1,4-dioxane/water (5 mL), to this add 2-methyl-1-(4-(4,4,5,5-tetramethyl-1,3,2-dioxaborolan-2-yl)-1H-pyrazol-1-yl)propan-2-ol (90 mg, 0.136 mmol, 1.5 equiv), K₂CO₃ (47 mg, 0.227 mmol, 2.5 equiv). The solution was degassed and added Pd(dppf)Cl₂ (20 mg, 0.018 mmol, 0.2 equiv), then heated to 80° C. and stirred for 2 h. The reaction mass was quenched with saturated ammonium chloride and extracted with EA (3×10 mL). The organic solutions were combined, washed with H₂O (2×10 mL) and brine solution (1×10 mL), dried over anhydrous Na₂SO₄, filtered and concentrated by rotary evaporation to get the crude compound. The crude was purified by silica column to get pure compound (22 mg, purity=92%, yield=48%) as a brown semi solid.

TLC R_{f}=0.3 (1:1,EA/PE)

LCMS (m/z)=501.40 (M+H)

### Example 102. 1-(3-fluoro-4-methoxybenzyl)-8-(1-(2-hydroxy-2-methylpropyl)-1H-pyrazol-4-yl)-4-(5-methyloxazol-2-yl)-1,3-dihydro-2H-benzo[b]azepin-2-one (102)

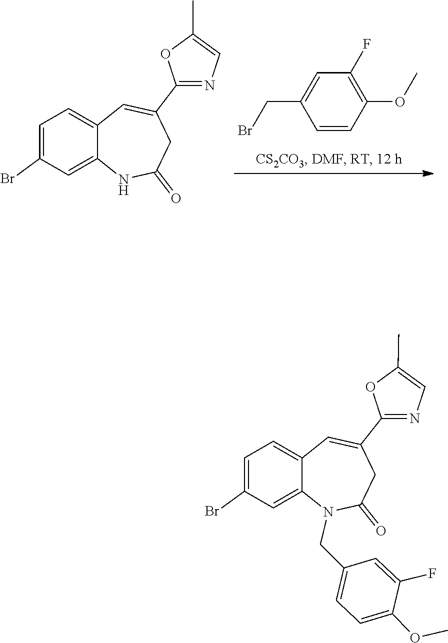 [SMILES: Cc1cnc(C2=Cc3ccc(Br)cc3NC(=O)C2)o1 ; COc1ccc(CBr)cc1F ; COc1ccc(CN2C(=O)CC(c3ncc(C)o3)=Cc3ccc(Br)cc32)cc1F]

### 8-Bromo-1-(3-fluoro-4-methoxybenzyl)-4-(5-methyloxazol-2-yl)-1,3-dihydro-2H-benzo[b]azepin-2-one

To a stirred solution of 8-bromo-4-(5-methyloxazol-2-yl)-1,3-dihydro-2H-benzo[b]azepin-2-one (100 mg, 0.313 mmol, 1 equiv), CS₂CO₃ (305 mg, 0.705 mmol, 3 equiv) in DMF (10 mL) was added 4-(bromomethyl)-2-fluoro-1-methoxybenzene (107 mg, 0.470 mmol, 1.5 equiv) at rt. The reaction mixture was stirred at rt for 12 h. Reaction mass was cooled to rt and poured into water (20 mL) and extracted with ethyl acetate (2×20 mL). The combined organic layers were washed with H₂O (3×20 mL) and brine solution (20 mL), dried over anhydrous Na₂SO₄, and concentrated by rotary evaporation. Then, the resulting concentrate was purified on a silica column to get 90 mg of compound (yield of 63%, purity=69.2%) as a brown solid.

TLC R_{f}=0.4 (PE:EA=1:1)

LCMS (m/z)=457.30 (M+H)

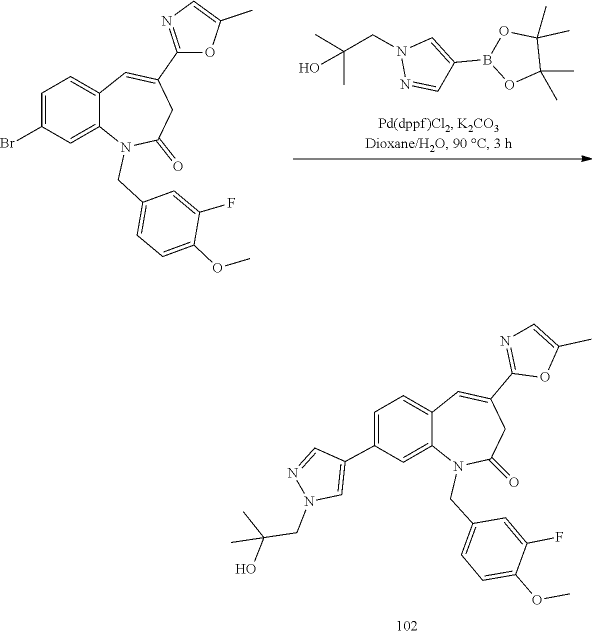 [SMILES: COc1ccc(CN2C(=O)CC(c3ncc(C)o3)=Cc3ccc(Br)cc32)cc1F ; CC(C)(O)Cn1cc(B2OC(C)(C)C(C)(C)O2)cn1 ; COc1ccc(CN2C(=O)CC(c3ncc(C)o3)=Cc3ccc(-c4cnn(CC(C)(C)O)c4)cc32)cc1F]

### 1-(3-fluoro-4-methoxybenzyl)-8-(1-(2-hydroxy-2-methylpropyl)-1H-pyrazol-4-yl)-4-(5-methyloxazol-2-yl)-1,3-dihydro-2H-benzo[b]azepin-2-one: 102

To a stirred solution of 8-bromo-1-(3-fluoro-4-methoxybenzyl)-4-(5-methyloxazol-2-yl)-1,3-dihydro-2H-benzo[b]azepin-2-one (90 mg, 0.196 mmol, 1 equiv) in 1,4-dioxane/water (10 mL), to this add 2-methyl-1-(4-(4,4,5,5-tetramethyl-1,3,2-dioxaborolan-2-yl)-1H-pyrazol-1-yl)propan-2-ol (62.8 mg, 0.236 mmol, 1.2 equiv), K₂CO₃ (68 mg, 0.492 mmol, 2.5 equiv). The solution was degassed and added Pd(dppf)Cl₂ (28.7 mg, 0.039 mmol, 0.2 equiv), then heated to 90° C. and stirred for 3 h. The reaction mass was quenched with saturated ammonium chloride and extracted with EA (3×30 mL). The organic solutions were combined, washed with H₂O (2×20 mL) and brine solution (1×20 mL), dried over anhydrous Na₂SO₄, filtered and concentrated by rotary evaporation to get the crude compound. The crude was purified by silica column to get pure compound (44 mg, purity=94%, yield 44%) as an off white solid.

TLC R_{f}=0.2 (1:1,EA/PE)

LCMS (m/z)=517.50 (M+H)

¹H NMR (400 MHz, Chloroform-d) δ 7.72 (s, 1H), 7.62 (s, 1H), 7.52 (s, 1H), 7.42 (s, 1H), 7.35 (d, J=8.0 Hz, 1H), 7.29 (d, J=8.1 Hz, 1H), 6.89 (s, 1H), 6.85 (m, 3H), 5.05 (s, 2H), 4.26-2.89 (br., 2H), 4.10 (s, 2H), 3.83 (s, 3H), 2.38 (s, 3H), 2.02-1.85 (br., 1H), 1.21 (s, 6H).

### Example 103. 8-(1-(2-Hydroxy-2-methylpropyl)-1H-pyrazol-4-yl)-1-((6-methoxypyridin-3-yl)methyl)-4-(5-methyloxazol-2-yl)-1,3-dihydro-2H-benzo[b]azepin-2-one (103)

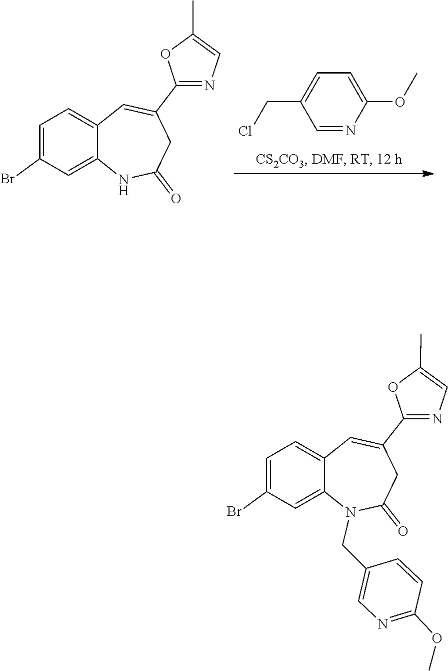

8-Bromo-I-((6-methoxypyridin-3-yl)methyl)-4-(5-methyloxazol-2-yl)-1,3-dihydro-2H-benzo[b]azepin-2-one:

To a stirred solution of 8-bromo-4-(5-methyloxazol-2-yl)-1,3-dihydro-2H-benzo[b]azepin-2-one (93 mg, 0.29 mmol, 1 equiv), CS₂C₀₃ (282 mg, 0.87 mmol, 3 equiv) in DMF (8 mL) was added 5-(chloromethyl)-2-methoxypyridine (67 mg, 0.43 mmol, 1.5 equiv) at rt. The reaction mixture was stirred at rt for 12 h. Reaction mass was cooled to rt and poured into water (20 mL) and extracted with ethyl acetate (2×20 mL). The combined organic layers were washed with H₂O (3×20 mL) and brine solution (20 mL), dried over anhydrous Na₂SO₄, and concentrated by rotary evaporation. Then, the resulting concentrate was purified on a silica column to get 92 mg of compound as an oil. yield of 72%, purity=97%

TLC R_{f}=0.4 (DCM:EA=2:1)

LCMS (m/z)=441.80 (M+H)

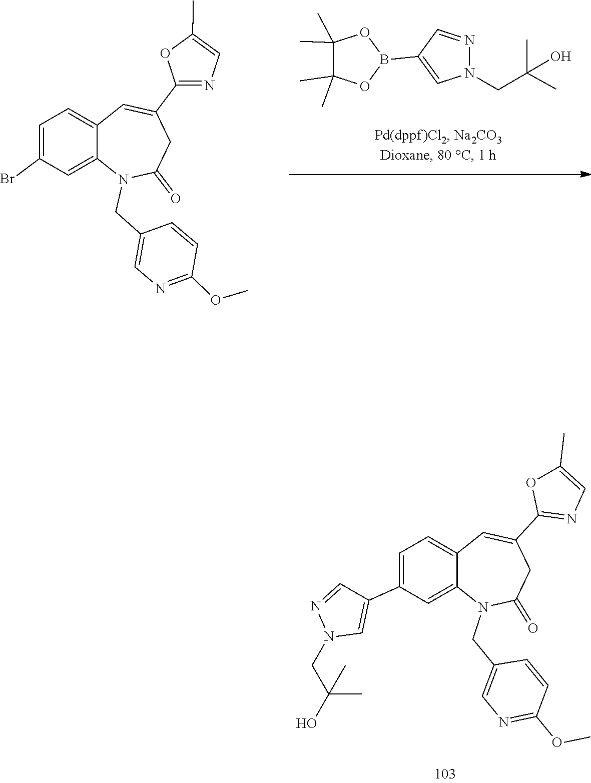 [SMILES: COc1ccc(CN2C(=O)CC(c3ncc(C)o3)=Cc3ccc(Br)cc32)cn1 ; CC(C)(O)Cn1cc(B2OC(C)(C)C(C)(C)O2)cn1 ; COc1ccc(CN2C(=O)CC(c3ncc(C)o3)=Cc3ccc(-c4cnn(CC(C)(C)O)c4)cc32)cn1]

### 8-(1-(2-Hydroxy-2-methylpropyl)-1H-pyrazol-4-yl)-1-((6-methoxypyridin-3-yl)methyl)-4-(5-methyloxazol-2-yl)-1,3-dihydro-2H-benzo[b]azepin-2-one (103)

8-bromo-1-((6-methoxypyridin-3-yl)methyl)-4-(5-methyloxazol-2-yl)-1,3-dihydro-2H-benzo[b]azepin-2-one (92 mg, 0.209 mmol, 1 equiv) and 2-methyl-2-(4-(4,4,5,5-tetramethyl-1,3,2-dioxaborolan-2-yl)-1H-pyrazol-1-yl)propan-1-ol (111 mg, 0.418 mmol, 2 equiv) and Pd(dppf)Cl₂ (38 mg, 0.05 mmol, 0.25 equiv) were placed in a round-bottom flask, followed by 1,4-dioxane (4.8 mL) and 2N Na₂CO₃ aqueous solution (2.4 mL). The solution was degassed and heated at 80° C. for 1 h. The solution was quenched with saturated ammonium chloride and extracted with EA (3×10 mL). The organic solutions were combined, washed with H₂O (2×10 mL) and brine solution (1×20 mL), dried over anhydrous Na₂SO₄, filtered and concentrated by rotary evaporation to get the crude compound. Then, the resulting concentrate was purified on a silica column to get 71 mg (purity=96%) yellow oil compound with a yield of 68%.

TLC R_{f}=0.2 (DCM:MeOH=40:1);

LCMS (m/z)=500.30 (M+H)

### Example 104. (8-(1-(2-Hydroxy-2-methylpropyl)-1H-pyrazol-4-yl)-4-(5-methyloxazol-2-yl)-2,3-dihydro-1H-benzo[b]azepin-1-yl) (4-methoxyphenyl)methanone (104)

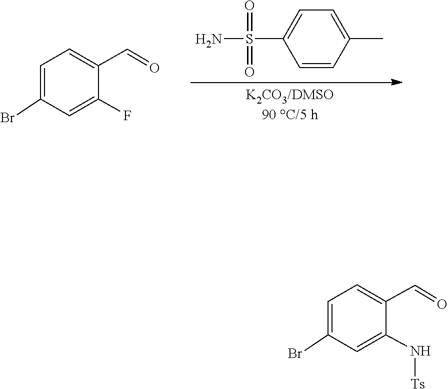 [SMILES: O=Cc1ccc(Br)cc1F ; Cc1ccc(S(N)(=O)=O)cc1 ; Cc1ccc(S(=O)(=O)Nc2cc(Br)ccc2C=O)cc1]

### N-(5-bromo-2-formylphenyl)-4-nethylbenzenesulfonamide

To a clear solution of 4-methylbenzenesulfonamide (1.27 g, 7.39 mmol, 1.5 equiv) in dry DMSO in the round-bottom flask was added K₂CO₃ (2.04 g, 14.78 mmol, 3 equiv) and 4-bromo-2-fluorobenzaldehyde (1 g, 4.93 mmol, 1 equiv) at room temperature. The reaction flask was placed in a pre-heated oil bath at 90° C. and allowed to stir for 5 h. After completion of the reaction monitored by TLC, the reaction mixture cooled to room temperature, poured into ice-cold water, and extracted with ethyl acetate. The combined organic layers were washed with brine solution, dried over anhydrous Na₂SO₄, and concentrated under a vacuum. The crude was further purified by flash column chromatography to yield a pure compound (1.5 g, Purity=84.7%, Yield=86%) as an off-yellow solid.

TLC R_{f}=0.4 (1/4 EA/PE)

LCMS (m/z)=355.80 (M+1)

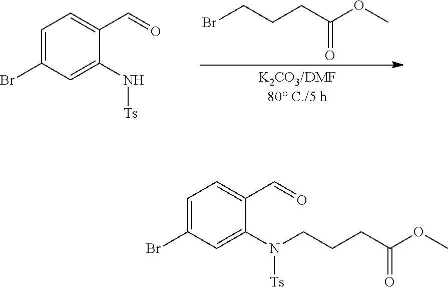

### Methyl 4-((N-(5-bromo-2-formylphenyl)-4-methylphenyl)sulfonamido)butanoate

To a solution of N-(5-bromo-2-formylphenyl)-4-methylbenzenesulfonamide (500 mg, 1.41 mmol, 1 equiv) in dry DMF was added K₂CO₃ (390 mg, 2.82 mmol, 2 equiv.) and methyl 4-bromobutanoate (307 mg, 1.69 mmol, 1.2 equiv.) at room temperature. The reaction flask was placed in a preheated oil bath with 80° C. and allowed to stir for 5 h. After completion of the reaction shown by TLC, the reaction mixture was cooled to room temperature, poured into ice-cold water, extracted with ethyl acetate, washed the organic layer with brine solution, dried the organic layer over NaSO4, and concentrated under a vacuum. The crude was further purified by flash column chromatography to yield a pure compound (520 mg, purity=77%, yield=81%) as an off-white solid.

TLC R_{f}=0.35 (1/4 EA/PE)

LCMS (m/z)=454.0 (M+H)

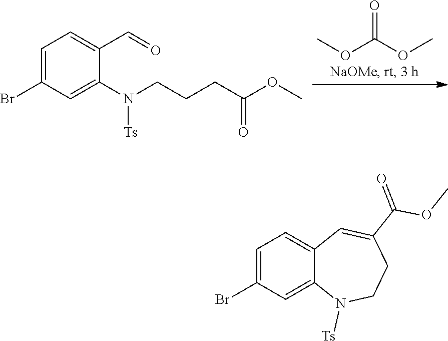

### Methyl 8-bromo-1-tosyl-2,3-dihydro-1H-benzo[b]azepine-4-carboxylate

To a solution of methyl 4-((N-(5-bromo-2-formylphenyl)-4-methylphenyl)sulfonamido)butanoate (500 mg, 1.10 mmol, 1 equiv.) and dry dimethyl carbonate (15 mL) in the round bottom flask was added NaOMe (1.5 mL, 5.50 mmol 3 equiv.) dropwise over 10 minutes at room temperature and allowed to stir for 3 h. After completion of the reaction, monitored by TLC, the reaction mixture was poured into ice-cold water, extracted with ethyl acetate, washed the organic layer with brine solution, dried the organic layer over NaSO4, and concentrated under vacuum. The crude was further purified by flash column chromatography to yield a pure compound (430 mg, purity=97.54%, yield=89%) as an off-white solid.

TLC R_{f}=0.4 (1/4 EA/PE)

LCMS (m/z)=437.40 (M+H)

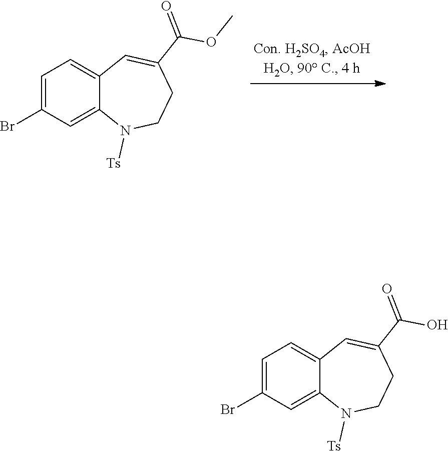 [SMILES: COC(=O)C1=Cc2ccc(Br)cc2N(S(=O)(=O)c2ccc(C)cc2)CC1 ; Cc1ccc(S(=O)(=O)N2CCC(C(=O)O)=Cc3ccc(Br)cc32)cc1]

### 8-Bromo-1-tosyl-2,3-dihydro-1H-benzo[b]azepine-4-carboxylic acid

To a solution of methyl 8-bromo-l-tosyl-2,3-dihydro-1H-benzo[b]azepine-4-carboxylate (400 mg, 0.917 mmol, 1 equiv.) and AcOH (4 mL) in round bottom flask was added con. H₂SO₄ (0.5 mL) and water (0.4 mL) at room temperature. The reaction flask was moved to a pre-heated oil bath with 90° C. and allowed to stir for 4 h. After completion of the reaction monitored by TLC, the reaction mixture was cooled to room temperature, poured into ice-cold water, and the separated solid was filtered. The crude was further used without purification (310 mg, purity=96%, yield=80%) as an off-white solid.

TLC R_{f}=0.2 (1/1 EA/PE)

LCMS (m/z)=423.29 (M+H)

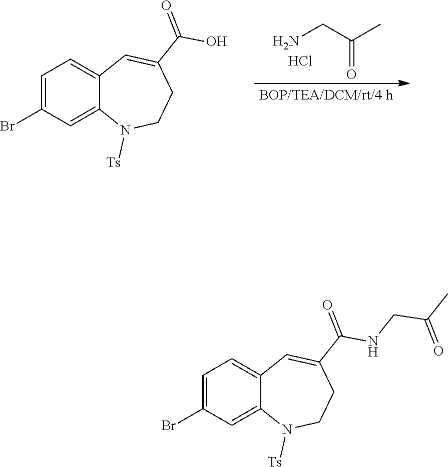

### 8-bromo-N-(2-oxopropyl)-l-tosyl-2, 3-dihydro-1H-benzo[b]azepine-4-carboxamide

To a solution of 8-bromo-1-tosyl-2,3-dihydro-1H-benzo[b]azepine-4-carboxylic acid (900 mg, 2.13 mmol, 1 equiv.) in dry CH₂Cl₂ at room temperature was added BOP (1.4 g, 3.20 mmol, 1.5 equiv), Et₃N (0.9 mL, 6.39 mmol, 3 equiv.) and 1-aminopropan-2-one hydrochloride (280 mg, 2.56 mmol, 1.2 equiv.). The reaction mixture was allowed to stir for 4h. Subsequently, CH₂Cl₂ (volume?) added to the reaction mixture and washed with water (volume of water). The organic layer was dried over anhydrous sodium sulphate, filtered and concentrated in vacuo to give crude compound. Purification using flash column chromatography afforded the desired compound (840 mg, purity=93.8%, yield=82%) as an off-white solid.

TLC R_{f}=0.4 (1/1 EA/PE)

LCMS (m/z)=478.8 (M+H)

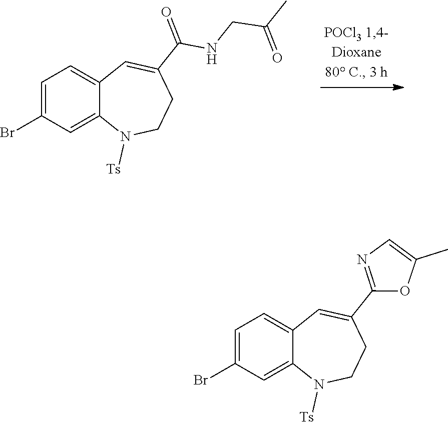 [SMILES: CC(=O)CNC(=O)C1=Cc2ccc(Br)cc2N(S(=O)(=O)c2ccc(C)cc2)CC1 ; Cc1ccc(S(=O)(=O)N2CCC(c3ncc(C)o3)=Cc3ccc(Br)cc32)cc1]

### 2-(8-Bromo-1-tosyl-2,3-dihydro-1H-benzo[b]azepin-4-yl)-5-methyloxazole

To a solution of 8-bromo-N-(2-oxopropyl)-1-tosyl-2,3-dihydro-1H-benzo[b]azepine-4-carboxamide (1.2 g, 2.09 mmol, 1 equiv.) in 1,4-Dioxane (10 mL) was added POCl₃ (2 mL, 20.95 mmol, 10 equiv.) at room temperature. The reaction mixture was placed in a pre-heated oil bath and stirred at 80° C. for 3 h. After completion of the reaction, monitored by TLC, the reaction mixture was cooled to room temperature and poured into ice-cold water. The reaction mixture pH was adjusted to 7-8 with saturated NaHCO₃ and extracted with ethyl acetate (how many times and volume). The combined organic layer was washed with a brine solution, and the organic layer was dried over anhydrous Na₂SO₄ and concentrated under a vacuum. The crude was further purified by flash column chromatography to yield a pure compound (950 mg, purity=82.3%, yield=82%) as an off-white solid.

TLC R_{f}=0.4 (2/3 EA/PE)

LCMS (m/z)=460.80 (M+H)

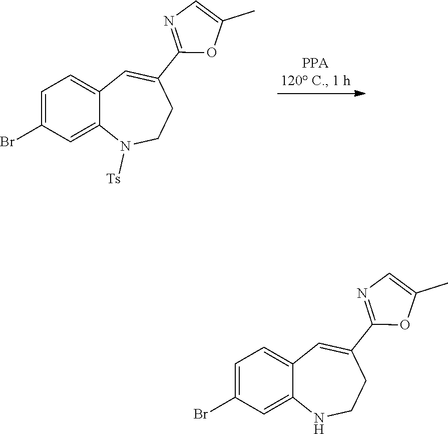 [SMILES: Cc1ccc(S(=O)(=O)N2CCC(c3ncc(C)o3)=Cc3ccc(Br)cc32)cc1 ; Cc1cnc(C2=Cc3ccc(Br)cc3NCC2)o1]

### 2-(8-Bromo-2,3-dihydro-1H-benzo[b]azepin-4-yl)-5-methyloxazole

The round bottom flask with 2-(8-bromo-1-tosyl-2,3-dihydro-1H-benzo[b]azepin-4-yl)-5-methyl oxazole (200 mg, 0.435 mmol, 1 equiv.) and polyphosphoric acid (200 mg) was placed on a pre-heated oil bath with 120° C. and allowed to stir for 1 h. After completion of the reaction, monitored by TLC, the reaction mixture was cooled to room temperature, diluted with water, and extracted with ethyl acetate (2×10 mL). The separated organic layer was further washed with brine solution. The combined organic layer was dried over Na₂SO₄, filtered, and concentrated under a vacuum. The crude was further purified through flash column chromatography to afford pure compound (92 mg, purity=92.02%, yield=69.2) as yellow solid

TLC R_{f}=0.2 (2/3 EA/PE)

LCMS (m/z)=305.10 (M+H)

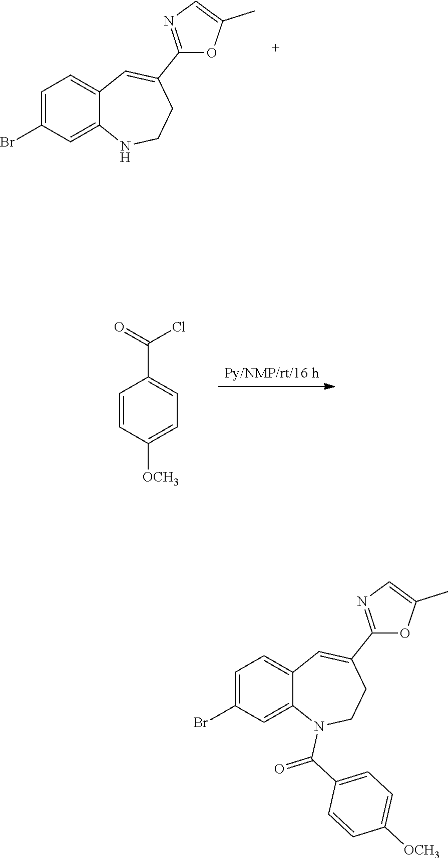 [SMILES: Cc1cnc(C2=Cc3ccc(Br)cc3NCC2)o1 ; COc1ccc(C(=O)Cl)cc1 ; COc1ccc(C(=O)N2CCC(c3ncc(C)o3)=Cc3ccc(Br)cc32)cc1]

### (8-Bromo-4-(5-methyloxazol-2-yl)-2,3-dihydro-1H-benzo[b]azepin-1-yl)(4-methoxyphenyl)methanone

To a solution of 2-(8-bromo-2,3-dihydro-1H-benzo[b]azepin-4-yl)-5-methyloxazole (100 mg, 0.327 mmol, 1 equiv.) in N-methylpyrrolidone (10 mL) was added pyridine (0.1 mL, 1.64 mmol, 5 equiv.), and 4-methoxybenzoyl chloride (112 mg, 0.655 mmol, 2 equiv.) at room temperature under N₂ atmosphere and allowed to stir 16 h. After completion of the reaction, monitored by TLC, the reaction mixture was poured into water, and the solution was adjusted to pH 2 with 5M HCl and extracted with ethyl acetate (15 mL). The separated organic layer was washed with brine solution. The combined organic layer was dried over anhydrous Na₂SO₄, filtered, and concentrated under a vacuum to get the desired compound (120 mg, yield=83.3) as a dark brown solid. The resulting compound was used for the next reaction without further purification.

TLC R_{f}=0.4 (1/1 PE/EA)

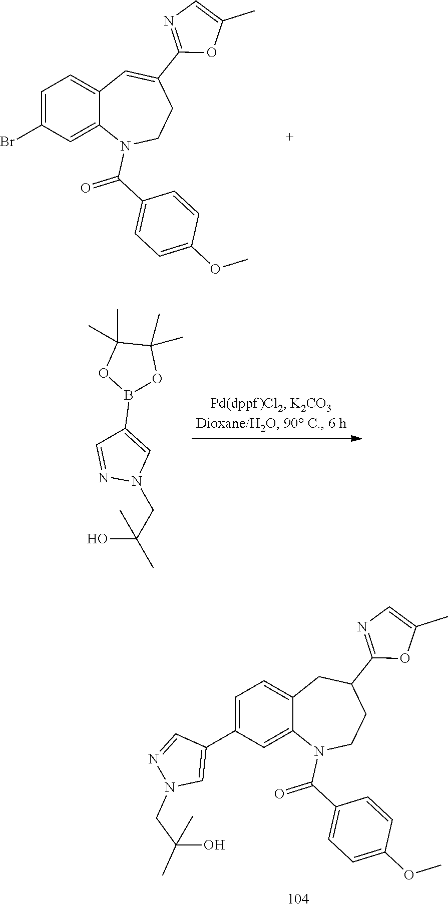

### (8-(1-(2-Hydroxy-2-methylpropyl)-1H-pyrazol-4-yl)-4-(5-methyloxazol-2-yl)-2,3-dihydro-1H-benzo[b]azepin-1-yl) (4-methoxyphenyl)methanone: 104

To a solution of (8-bromo-4-(5-methyloxazol-2-yl)-2,3-dihydro-1H-benzo[b]azepin-1-yl)(4-methoxyphenyl)methanone (100 mg, 0.227 mmol, 1 equiv.) in 1,4-dioxane:water=8:2 (2 mL) was added 2-methyl-I-(4-(4,4,5,5-tetramethyl-1,3,2-dioxaborolan-2-yl)-1H-pyrazol-1-yl)propan-2-ol (73 mg, 0.273 mmol, 1.2 equiv.), K₂CO₃ (94 mg, 0.682 mmol, 3 equiv.) and Pd(dppf)Cl₂ (10 mg, 0.013 mmol, 0.06 equiv.) at room temperature. The reaction flask was degassed with nitrogen and, placed in a preheated oil bath at 90° C. and allowed to stir for 6 h. After completion of the reaction, quenched with saturated ammonium chloride and extracted with EA (3×30 mL). The combined organic layers were washed with H₂O (2×10 mL) and brine solution (1×10 mL), dried over anhydrous Na₂SO₄, filtered, and concentrated by rotary evaporation to get the crude compound. The crude was purified by silica column to get a pure compound (82 mg, purity=88%, yield=72.2%) as pale yellow solid.

TLC R_{f}=0.2 (1/1 PE/EA)

LCMS (m/z)=499.40 (M+1)

### Example 105. (8-(1-(2-Hydroxy-2-methylpropyl)-1H-pyrazol-4-yl)-4-(5-methyloxazol-2-yl)-2,3-dihydro-1H-benzo[b]azepin-1-yl)(6-methoxypyridin-3-yl)methanone (105)

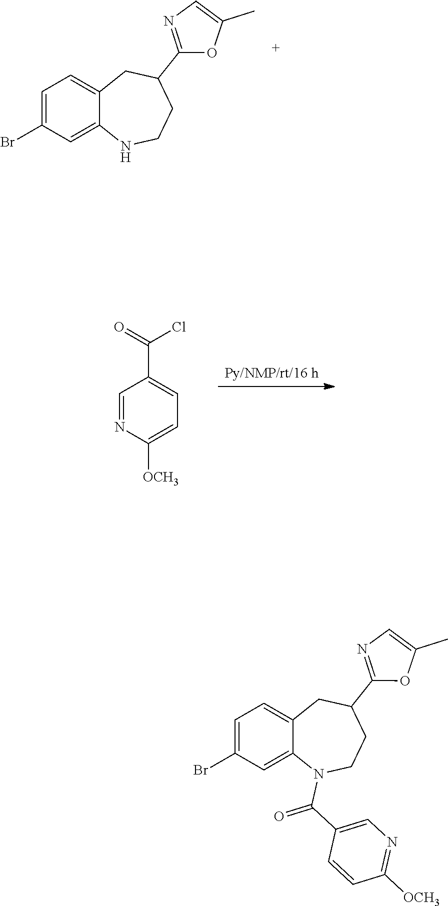 [SMILES: Cc1cnc(C2CCNc3cc(Br)ccc3C2)o1 ; COc1ccc(C(=O)Cl)cn1 ; COc1ccc(C(=O)N2CCC(c3ncc(C)o3)Cc3ccc(Br)cc32)cn1]

### (8-Bromo-4-(5-methyloxazol-2-yl)-2,3-dihydro-1H-benzo[b]azepin-1-yl)(6-methoxypyridin-3-yl)methanone

To a solution of 2-(8-bromo-2,3-dihydro-1H-benzo[b]azepin-4-yl)-5-methyloxazole (100 mg, 0.327 mmol, 1 equiv.) in N-methylpyrrolidone (5 mL) was added pyridine (1 mL, 0.327 mmol, 5 equiv.), 6-methoxynicotinoyl chloride (100 mg, 0.327 mmol, 2 equiv.) at room temperature under N₂ atmosphere and allowed to stir for 16h.

After the reaction was completed and monitored by TLC, the reaction mixture was poured into water; the solution was adjusted to pH 2 with 5M HCl and extracted with ethyl acetate (1×15 mL). The separated organic layer was dried over anhydrous Na₂SO₄, filtered, and concentrated to obtain a crude product. The resulting compound was purified using flash column chromatography to afford a pure product (135 mg, Yield=93.5%) as a brown solid.

TLC R_{f}=0.4 (1/4 PE/EA)

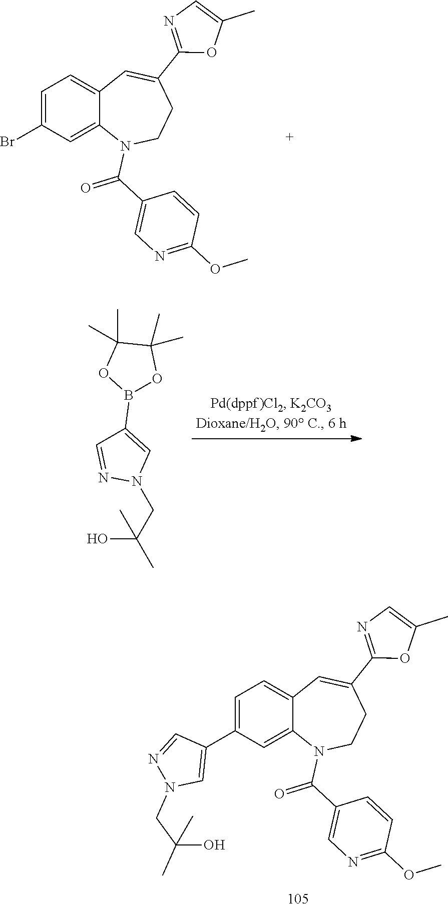

### (8-(1-(2-Hydroxy-2-methylpropyl)-1H-pyrazol-4-yl)-4-(5-methyloxazol-2-yl)-2,3-dihydro-1H-benzo[b]azepin-1-yl)(6-methoxypyridin-3-yl)methanone: 105

To a solution of (8-bromo-4-(5-methyloxazol-2-yl)-2,3-dihydro-1H-benzo[b]azepin-1-yl)(6-methoxypyridin-3-yl)methanone (100 mg, 0.227 mmol, 1 equiv.) in 1,4-dioxane:water (8:2) (2 mL), was added 2-methyl-1-(4-(4,4,5,5-tetramethyl-1,3,2-dioxaborolan-2-yl)-1H-pyrazol-1-yl)propan-2-ol (72 mg, 0.272 mmol, 1.2 equiv.), K₂CO₃ (94 mg, 0.681 mmol, 3 equiv.) and Pd(dppf)Cl₂ (10 mg, 0.013 mmol, 0.06 equiv.) at room temperature. The reaction flask was degassed with nitrogen and, placed in a preheated oil bath at 90° C. and allowed to stir for 6 h. After completion of the reaction, quenched with saturated ammonium chloride and extracted with EA (3×30 mL). The combined organic layers were washed with H₂O (2×10 mL) and brine solution (1×10 mL), dried over anhydrous Na₂SO₄, filtered, and concentrated by rotary evaporation to get the crude compound. The crude was purified by flash column chromatography to get a pure compound (76 mg, purity=81.9%, yield=66.9%) as yellow solid.

TLC R_{f}=0.2 (1/4 PE/EA)

LCMS (m/z)=500.30 (M+1)

### Example 106. (3-Fluoro-4-methoxyphenyl)(8-(1-(2-hydroxy-2-methylpropyl)-1H-pyrazol-4-yl)-4-(5-methyloxazol-2-yl)-2,3-dihydro-1H-benzo[b]azepin-1-yl)methanone (106)

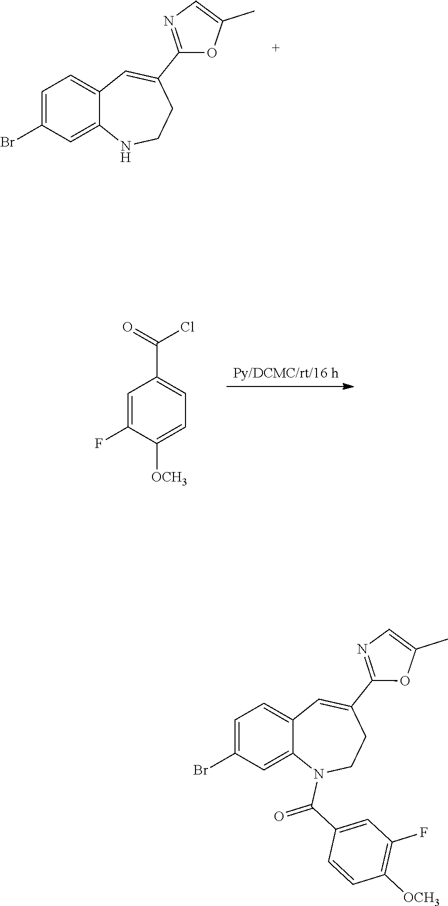 [SMILES: Cc1cnc(C2=Cc3ccc(Br)cc3NCC2)o1 ; COc1ccc(C(=O)Cl)cc1F ; COc1ccc(C(=O)N2CCC(c3ncc(C)o3)=Cc3ccc(Br)cc32)cc1F]

### (8-Bromo-4-(5-methyloxazol-2-yl)-2,3-dihydro-1H-benzo[b]azepin-1-yl) (3-fluoro-4-methoxyphenyl)methanone

To a solution of 2-(8-bromo-2,3-dihydro-1H-benzo[b]azepin-4-yl)-5-methyloxazole (100 mg, 0.327 mmol, 1 equiv.) in N-methylpyrrolidone (5 mL) was added pyridine (0.1 mL, 1.64 mmol, 5 equiv.), 3-fluoro-4-methoxybenzoyl chloride (123 mg, 0.655 mmol, 2 equiv.) at room temperature under N₂ atmosphere and allowed to stir for 16 h. After completion of the reaction monitored by TLC, the reaction mixture was poured into water; the solution was adjusted to pH 2 with 5M HCl and extracted with ethyl acetate (1×15 mL). The separated organic layer was dried over anhydrous Na₂SO₄, filtered, and concentrated to obtain a crude product (135 mg, Yield=90%) as a brown solid. The resulting compound was used for the next reaction without further purification.

TLC R_{f}=0.4 (1/1 PE/EA)

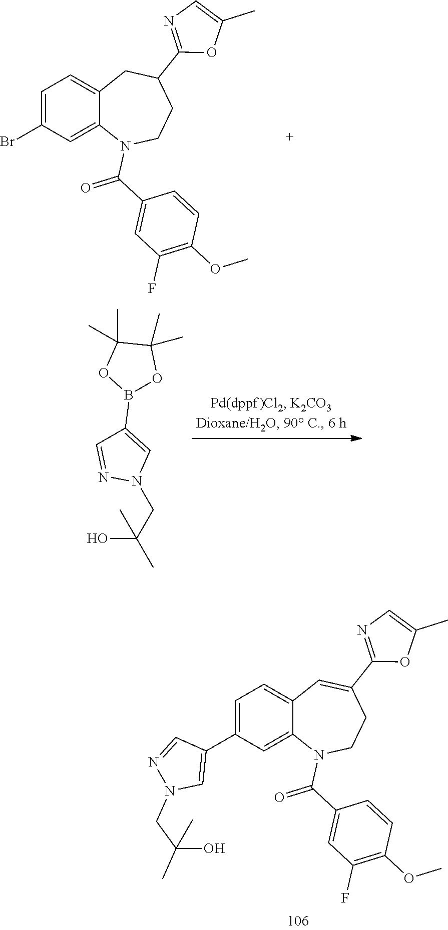

### (3-Fluoro-4-methoxyphenyl)(8-(1-(2-hydroxy-2-methylpropyl)-1H-pyrazol-4-yl)-4-(5-methyloxazol-2-yl)-2,3-dihydro-1H-benzo[b]azepin-1-yl)methanone: 106

To a solution of (8-bromo-4-(5-methyloxazol-2-yl)-2,3-dihydro-1H-benzo[b]azepin-1-yl)(3-fluoro-4-methoxyphenyl)methanone (100 mg, 0.218 mmol, 1 equiv.) in 1,4-dioxane:water (8:2) (2 mL), was added 2-methyl-1-(4-(4,4,5,5-tetramethyl-1,3,2-dioxaborolan-2-yl)-1H-pyrazol-1-yl)propan-2-ol (70 mg, 0.262 mmol, 1.2 equiv.), K₂CO₃ (91 mg, 0.656 mmol, 3 equiv.) and Pd(dppf)Cl₂ (10 mg, 0.013 mmol, 0.06 equiv.) at room temperature. The reaction flask was degassed with nitrogen and, placed in a preheated oil bath at 90° C. and allowed to stir for 6 h. After completion of the reaction, quenched with saturated ammonium chloride and extracted with EA (3×30 mL). The combined organic layers were washed with H₂O (2×10 mL) and brine solution (1×10 mL), dried over anhydrous Na₂SO₄, filtered, and concentrated by rotary evaporation to get the crude compound. The crude was purified by silica column to get a pure compound (92 mg, purity=91.8%, yield=81.4%) as brown solid.

TLC R_{f}=0.2 (1/1 PE/EA)

LCMS (m/z)=517.50 (M+1)

### Example 107. 2-(4-(1-(3-fluoro-4-methoxybenzyl)-4-(5-methyloxazol-2-yl)-2-oxo-2,3-dihydro-1H-benzo[b]azepin-8-yl)-1H-pyrazol-1-yl)-2-methylpropanamide (107)

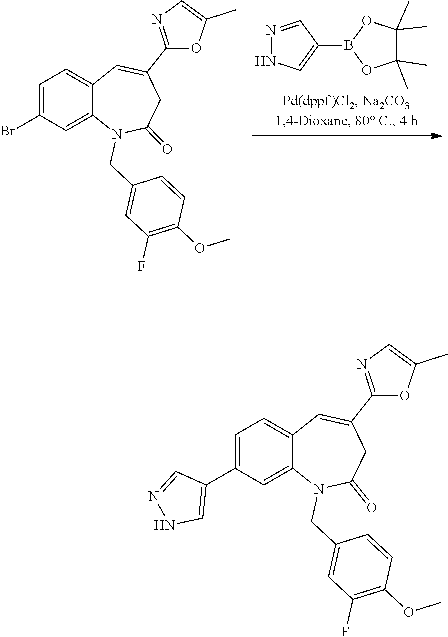

### 1-(3-Fluoro-4-methoxybenzyl)-4-(5-methyloxazol-2-yl)-8-(1H-pyrazol-4-yl)-1,3-dihydro-2H-benzo[b]azepin-2-one

8-bromo-1-(3-fluoro-4-methoxybenzyl)-4-(5-methyloxazol-2-yl)-1,3-dihydro-2H-benzo[b]azepin-2-one (220 mg, 0.48 mmol, 1 equiv) and 4-(4,4,5,5-tetramethyl-1,3,2-dioxaborolan-2-yl)-1H-pyrazole (157 mg, 0.96 mmol, 1.5 equiv) and Pd(dppf)Cl₂ (87 mg, 0.12 mol, 0.25 equiv.) were placed in a round-bottom flask, followed by 1,4-dioxane (9.6 mL) and 2 N Na₂CO₃ (4.8 mL). The solution was degassed and heated at 80° C. for 4 h. After completion of reaction, the reaction mixture was diluted with ethyl acetate, washed with water (2×30 mL) and brine solution (1×30 mL), the separated organic layer was dried over anhydrous Na₂SO₄, filtered, and concentrated by rotary evaporation to get the crude compound. The crude was further purified by flash column to get pure compound (70 mg, purity=99%, yield=32%) as an off white solid.

TLC R_{f}=0.15 (DCM:EA=2:1)

LCMS (m/z)=445.10 (M+H)

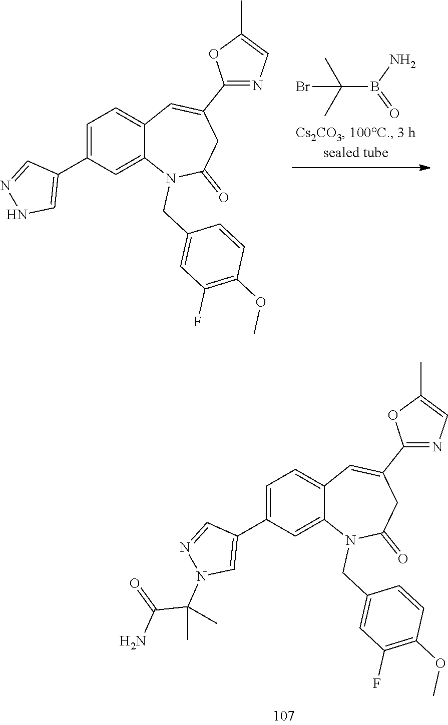

### 2-(4-(1-(3-fluoro-4-methoxybenzyl)-4-(5-methyloxazol-2-yl)-2-oxo-2,3-dihydro-1H-benzo[b]azepin-8-yl)-1H-pyrazol-1-yl)-2-methylpropanamide (107)

To a stirred solution of 1-(3-fluoro-4-methoxybenzyl)-4-(5-methyloxazol-2-yl)-8-(1H-pyrazol-4-yl)-1,3-dihydro-2H-benzo[b]azepin-2-one (70 mg, 0.15 mmol, 1 equiv) in DMF (2 mL) were added 2-bromo-2-methylpropanamide (51 mg, 0.31 mmol, 2 equiv) and Cs₂CO₃ (121 mg, 1.760 mmol, 2.5 equiv). The reaction mixture was stirred at 100° C. for 3 h. Reaction mass was cooled to rt and poured into water (100 mL) and extracted with ethyl acetate (2×50 mL). The combined organic layers were washed with H₂O (3×50 mL) and brine solution (100 mL), dried over anhydrous Na₂SO₄, and concentrated by rotary evaporation. Then, the resulting concentrate was purified on a silica column to get 45 mg of an Off-white solid compound with a yield of 54%, purity=99%

TLC R_{f}=0.4 (EA);

LCMS (m/z)=530.40 (M+H)

### Example 108. In Vitro Assays

### Cell Culture and Treatment

Human cancer cell lines were obtained from ATCC and cultured in DMEM (Gibco, Cleveland, TN, USA) supplemented with 5% fetal bovine serum (Gibco), penicillin (100 U/mL)-streptomycin (100 μg/mL) (Gibco, Cat. No. 15140-122), 2 mM L-glutamine (Gibco, 200 mM solution, Cat. No. 25030081), and 1 mM sodium pyruvate (Gibco, 100 mM solution, Cat. No. 11360070) at 37° C. in a humidified incubator that was maintained at 5% CO₂.

### Phenotypic Screening Assays and MTT Assays

Screening was conducted as described before (Li et al. 2019). Briefly, RPE-Neo or RPE-MYC or RPE-MYC expressing an EGFP-Histone 2B fusion protein were passaged as batches of 96-well plates, 18-24 hours before exposure to the chemical compounds of the present disclosure at concentrations from 20 nM to 20 μM. At 24, 48 or 72 hours after initiation of treatment, cells were analyzed for either an arrest in mitosis or a change in DNA content by GE IN-Cell Analyzer 2000. MTT assays of cellular proliferation were performed as previously described (Li et al. 2019).

Antibodies Used in this Study

### Primary Antibodies:

Histone H3(phospho-Ser10), Mouse Monoclonal Antibody, Upstate, Cat #05-806, 1:1000
Phospho-Aurora A (Thr288)(C39D8), Rabbit mAb, Cell Signalling, #3079, 1:1000 Aurora B Antibody, Rabbit, Absin, abs1460-100ug, lot #AH22, 1:50 dilution
CREST antibody, Thermofisher, Cat. #PA5-30500, 1:200 dilution
Mouse Anti-β-tubulin, Sigma, Cat. #F 2043, clone TUB 2.1 1:50 dilution

### Secondary Antibodies:

Rhodamine (TRITC) AffiniPure Goat Anti-mouse IgG (H+L), Proteintech, SA00007-1, 1:100
Rhodamine (TRITC) AffiniPure Goat Anti-Rabbit IgG (H+L), Jackson, 111-025-003, 1:100
Fluorescein (FITC)-conjugated Affinipure Goat Anti-Rabbit IgG(H+L), Jackson, 111-095-144, 1:100
Rhodamine Red™-X-conjugated AffiniPure Donkey Anti-Human IgG (H+L), Jackson, 709-295-149, 1:100

### Crystal Violet Assay

Cells were fixed in 4% PFA, stained with 0.1% crystal violet (Aladdin, 548-62-9). Bound crystal violet was eluted with 95% ethanol and the absorbance of eluate was measured at 570 nm using a Microplate reader (BioTek ELX808iu).

The results of these assays are summarized in Table 2. As set forth in table 2 below, an values of greater than or equal to 1 nM and less than or equal to 1.0 μM is marked “A”; a value greater than 1.00 μM and less than or equal to 10.0 μM is marked “B”; a value greater than 10.0 μM and less than or equal to 30.0 μM is marked “C”; and a value greater than 30.0 μM and less than 1000 μM is marked “D.”

TABLE 2

| | | Min. Effective | | |
|---|---|---|---|---|
| | Mitotic | conc. for | Along with | Proliferative |
| Compound | Arrest | Polyploidy | Cell Death | arrest |
| No. | (≥5%) (μM) | (≥5%) (μM) | (μM) | (>50%) (μM) |
| 1 | A | A | A | A |
| 2 | B | B | B | B |
| 3 | B | B | B | B |
| 4 | A | A | A | A |
| 5 | A | A | A | A |
| 6 | A | A | A | A |
| 7 | A | A | A | A |
| 8 | A | A | A | A |
| 9 | A | A | A | A |
| 10 | A | A | A | A |
| 11 | A | A | A | A |
| 12 | A | A | A | A |
| 13 | A | A | A | A |
| 14 | A | A | A | A |
| 15 | B | B | B | B |
| 16 | A | A | A | A |
| 17 | B | B | B | B |
| 18 | A | A | A | A |
| 19 | A | A | A | A |
| 20 | A | A | A | A |
| 21 | A | A | A | A |
| 22 | A | A | A | A |
| 23 | A | A | A | A |
| 24 | A | A | A | A |
| 25 | A | A | A | A |
| 26 | A | A | A | A |
| 27 | A | A | A | A |
| 28 | A | A | A | A |
| 29 | A | A | A | A |
| 30 | A | A | A | A |
| 31 | A | A | A | A |
| 32 | A | A | A | A |
| 33 | A | A | A | A |
| 34 | A | A | A | A |
| 35 | A | A | A | A |
| 36 | A | A | A | A |
| 37 | A | A | A | A |
| 38 | A | A | A | A |
| 39 | A | A | A | A |
| 40 | A | A | A | A |
| 41 | A | A | A | A |
| 42 | A | A | A | A |
| 43 | A | A | A | A |
| 44 | A | A | A | A |
| 45 | A | A | A | A |
| 46 | A | A | A | A |
| 47 | A | A | A | A |
| 48 | A | A | A | A |
| 49 | A | A | A | A |
| 50 | A | A | A | A |
| 51 | A | A | A | A |
| 52 | A | A | A | A |
| 53 | A | A | A | A |
| 54 | A | A | A | A |
| 55 | A | A | A | A |
| 56 | A | A | A | A |
| 57 | A | A | A | A |
| 58 | A | A | A | A |
| 59 | A | A | A | A |
| 60 | A | A | A | A |
| 61 | A | A | A | A |
| 62 | A | A | A | A |
| 63 | A | A | A | A |
| 64 | A | A | A | A |
| 65 | A | A | A | A |
| 66 | A | A | A | A |
| 67 | A | A | A | A |
| 68 | A | A | A | A |
| 69 | B | B | B | B |
| 70 | A | A | A | A |
| 71 | A | A | A | A |
| 72 | A | A | A | A |
| 73 | A | A | A | A |
| 74 | A | A | A | A |
| 75 | B | B | B | B |
| 76 | A | A | A | A |
| 77 | A | A | A | A |
| 78 | A | A | A | A |
| 79 | A | A | A | A |
| 80 | A | A | A | A |
| 81 | A | A | A | A |
| 82 | A | A | A | A |
| 83 | A | A | A | A |
| 84 | A | A | A | A |
| 85 | A | A | A | A |
| 86 | A | A | A | A |
| 87 | A | A | A | A |
| 88 | A | A | A | A |
| 89 | A | A | A | A |
| 90 | A | A | A | A |
| 91 | A | A | A | A |
| 92 | A | A | A | A |
| 93 | A | A | A | A |
| 94 | A | A | A | A |
| 95 | A | A | A | A |
| 96 | A | A | A | A |
| 97 | A | A | A | A |
| 98 | A | A | A | A |
| 99 | A | A | A | A |
| 100 | A | A | A | A |
| 101 | A | A | A | A |
| 102 | A | A | A | A |
| 103 | A | A | A | A |
| 104 | A | A | A | A |
| 105 | A | A | A | A |
| 106 | A | A | A | A |
| 107 | A | A | A | A |

### Example 109. Tumor Growth Inhibition

Six- to seven-week-old female BAL B/c nude mice were used in the human cancer cell line NCI-H460, which was derived from a male lung cancer patient. Six- to seven-week-old female BAL B/c mice were used in the mouse renal cancer cell line CT26 allograft models. 100 μL cell suspensions containing one million cells with 5000 matrigel (Corning, Cat. No. 354254) were implanted subcutaneously into the right flank of mice. Mice were monitored daily for well-being and weighed twice weekly. Caliper measurements of tumor sizes began when tumors became visible. Tumor volume (TV) was calculated by measuring two perpendicular diameters using the following formula: (L×W2)/2 in which L and W refer to the length and width of a tumor, respectively. When tumors reached an average size of 100 mm3, mice were first ranked according to tumor size and divided into “n” cohorts with X mice in each cohort. Each cohort was then sorted into 1 to X groups according to the random numbers size order generated by the RAND 0)function in Excel. In this way, the mice were randomized into X groups (n=4-6). Then the mice were orally administrated with vehicle (30% PEG 300) or indicated compounds at a dose volume of 5 mL/kg in a double-blinded manner. Oral administration was chosen because this is the intended route of use in the clinic. In all cases, no tumor measurement data or animals were excluded from the data analysis. Percentage of tumor growth inhibition (TGI) was calculated using the following formula: % TGI=average 100×[1−(final TV−initial TV of treatment group)/(final TV−initial TV of control group)]. The results of these assays are summarized in Table 2. As set forth in table 3 below, an values of greater than or equal to 35% and less than or equal to 45% μM is marked “C”; a value greater than 45% and less than or equal to 55% is marked “B”; a value greater than 55% and less than or equal to 70% is marked “A”; and a value greater than 30.0 μM and less than 1000 μM is marked “D.”

TABLE 3

| Compound | | Dose | Dosing | Days of | | | Number of | |
|---|---|---|---|---|---|---|---|---|
| No. | Formulation | (mg/kg) | frequency | treatment | Animal | Tumor | animals | TGI % |
| 42 | 30% PEG300 | 36 | 4 days-on, | 13 | BAL B/c | NCI- | 4 | C |
| | | | 3 days-off | | nude mice | H460 | | |
| 45 | 30% PEG300 | 36 | 4 days-on, | 20 | BAL B/c | NCI- | 4 | A |
| | | | 3 days-off | | nude mice | H460 | | |
| 36 | 30% PEG300 | 15 | 4 days-on, | 20 | BAL B/c | NCI- | 4 | A |
| | | | 3 days-off | | nude mice | H460 | | |
| 38 | 30% PEG300 | 36 | 4 days-on, | 20 | BAL B/c | NCI- | 4 | B |
| | | | 3 days-off | | nude mice | H460 | | |
| 37 | 30% PEG300 | 8 | 4 days-on, | 20 | BAL B/c | NCI- | 4 | A |
| | | | 3 days-off | | nude mice | H460 | | |
| 37 | 30% PEG300 | 8 | 3 days-on, | 20 | BAL B/c | CT26 | 5 | B |
| | | | 4 days-off | | mice | | | |
| 51 | 30% PEG300 | 15 | 4 days-on, | 29 | BAL B/c | NCI- | 6 | A |
| | | | 3 days-off | | nude mice | H460 | | |
| 53 | 30% PEG300 | 40 | 4 days-on, | 20 | BAL B/c | NCI- | 4 | C |
| | | | 3 days-off | | nude mice | H460 | | |
| 54 | 30% PEG300 | 50 | 4 days-on, | 20 | BAL B/c | NCI- | 4 | C |
| | | | 3 days-off | | nude mice | H460 | | |
| 57 | 30% PEG300 | 80 | 4 days-on, | 20 | BAL B/c | NCI- | 4 | A |
| | | | 3 days-off | | nude mice | H460 | | |
| 94 | 30% PEG300 | 8 | 4 days-on, | 20 | BAL B/c | CT26 | 5 | B |
| | | | 3 days-off | | mice | | | |
| 61 | 30% PEG300 | 10 | 4 days-on, | 20 | BAL B/c | CT26 | 5 | A |
| | | | 3 days-off | | mice | | | |
| 96 | 30% PEG300 | 20 | 4 days-on, | 20 | BAL B/c | CT26 | 5 | A |
| | | | 3 days-off | | mice | | | |

Note:
1. 30% PEG300 formulation: 5% DMSO, 30% PEG300, 1% Tween 80 in ddH2O, pH 2.2;
2. TGI %: tumor growth inhibition.

### REFERENCES

Li et al, 2019
Purification, identification, and characterization of two benzophenanthridine alkaloids from Corydalis longicalcarata rhizomes with anti-mitotic and polyploidy-inducing activities
Jinhua Li, Ziqi Yan, Hongmei Li, Qiong Shi, Linfang Huang, Thaddeus D. Allen, Dun Yang, Jing Zhang, bioRxiv 821124; doi: https://doi.org/10.1101/821124
Yang et al. 2010
Therapeutic potential of a synthetic lethal interaction between the MYC proto-oncogene and inhibition of aurora-B kinase
Dun Yang, Hong Liu, Andrei Goga, Suwon Kim, Maniia Yuneva, and J. Michael Bishop PNAS Aug. 3, 2010 107 (31) 13836-13841; https://doi.org/10.1073/pnas.1008366107

1. A compound of formula (I): 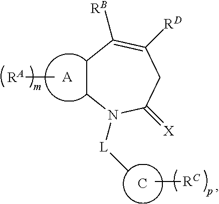
or a pharmaceutically acceptable salt thereof
wherein
X is O═ or S═;
Ring A is selected from the group consisting of optionally substituted phenyl, optionally substituted 5-membered heteroaryl, and optionally substituted 6-membered heteroaryl;
L is selected from the group consisting of a bond, optionally substituted C₁-C₃ alkylene, —CH(D)-, —C(D)₂-, —C(O)—, —C(O)O—, —C(O)NH—, and —S(O)₂—;
Group C is selected from the group consisting of C₁-C₆ aliphatic, —C≡CR₃, optionally substituted phenyl, optionally substituted 5-membered heteroaryl, optionally substituted 6-membered heteroaryl, optionally substituted 6-membered carbocyclyl, and optionally substituted 6-membered heterocyclyl;
each R^{A} is independently selected from the group consisting of halogen, —CN, —C≡CR³, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR¹, —SR¹, —N(R¹)₂, —C(O)OR¹, C(O)N(R¹)₂, —N(H)C(O)R¹, and —N(H)C(O)N(R¹)₂, wherein R^{A} is substituted with 0-4 instances of Ri; or
R^{B} is independently selected from the group consisting of hydrogen, halogen, —C≡CR³, optionally substituted 5-6-membered heteroaryl, optionally substituted C₁-C₃ aliphatic, and optionally substituted C₁-C₃ alkoxy, and optionally substituted 3-6-membered carbocyclyl;
each R^{C} is independently selected from the group consisting of halogen, —CN, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR³, —SR³, —N(R³)₂, —C(O)OR³, —C(O)N(R³)₂, —N(H)C(O)R³, and —N(H)C(O)N(R³)₂;
wherein optionally two instances of R^{C} are taken together with the atoms on which they are attached to form an optionally substituted 6-membered aryl or optionally substituted 6-membered heteroaryl;
R^{D} is selected from the group consisting of optionally substituted 5-membered heteroaryl, —C(O)OR³, C(O)N(R³)₂, —(CH₂)₁₋₃N(R²)₂, —(CH₂)₁₋₃OR², —(CH₂)₁₋₃O(CH₂)₁₋₃R², and —CHO;
each R¹ is independently selected from the group consisting of hydrogen, —C(O)R³, —(CH₂)₁₋₃OR³, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
each R² is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
wherein optionally two instances of R² are taken together to form an optionally substituted 3-7 membered heterocyclyl ring;
each R³ is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
m is 0, 1, 2, 3, or 4;
p is 0, 1, 2, 3, 4, or 5.

2. The compound of claim 1, wherein the compound is of Formula (I-a), (I-b), or (I-c): 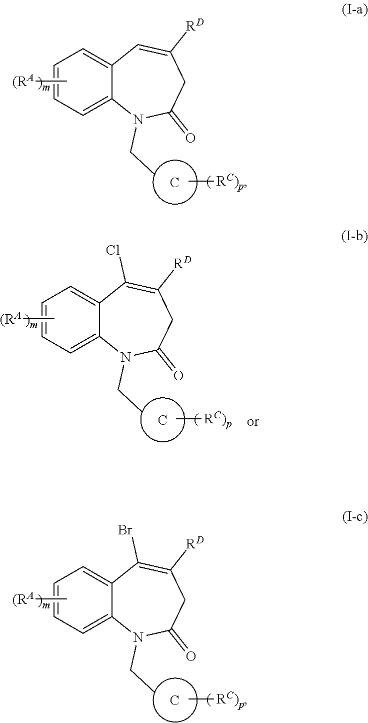
or a pharmaceutically acceptable salt thereof.

3. The compound of claim 2, wherein Group C is optionally substituted phenyl.

4. The compound of claim 3, wherein Group C is an optionally substituted pyridinyl.

5. The compound of any of claims 1-4, wherein R^{C} is selected from halogen, optionally substituted C₁-C₆ aliphatic, —COOR³, —CN, and —OR³.

6. The compound of any of claim 1-5, wherein p is 2.

7. The compound of claim 6, wherein one instance of R^{C} is halogen and the other is optionally substituted C₁-C₆ aliphatic or —OR³.

8. The compound of any of claim 1-7, wherein R^{C} is selected from —F, -Me, and —OMe.

9. The compound of any of claims 1-9, wherein p is 2 and one instance of R^{C} is fluoro and the other is -Me or —OMe.

10. The compound of any of claims 1-9, wherein L and Group C are taken together to form

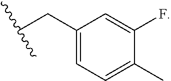 [SMILES: *Cc1ccc(C)c(F)c1]

11. The compound of any of claims 1-9, wherein L and Group C are taken together to form a group selected from:

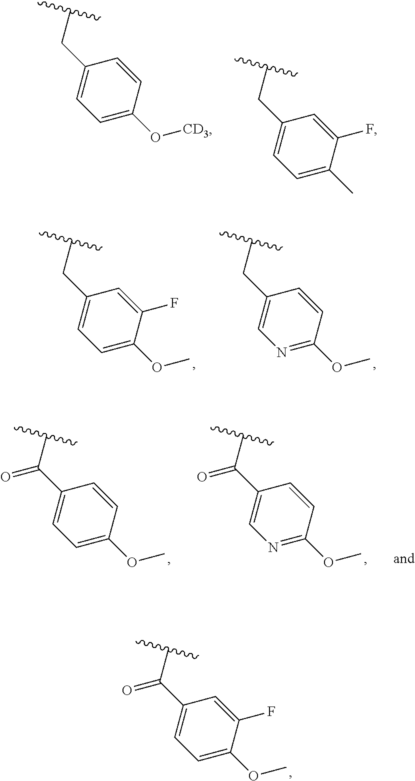

12. The compound of claim 1, wherein the compound is of Formula (I-al), (1-b1) or (I-c1): 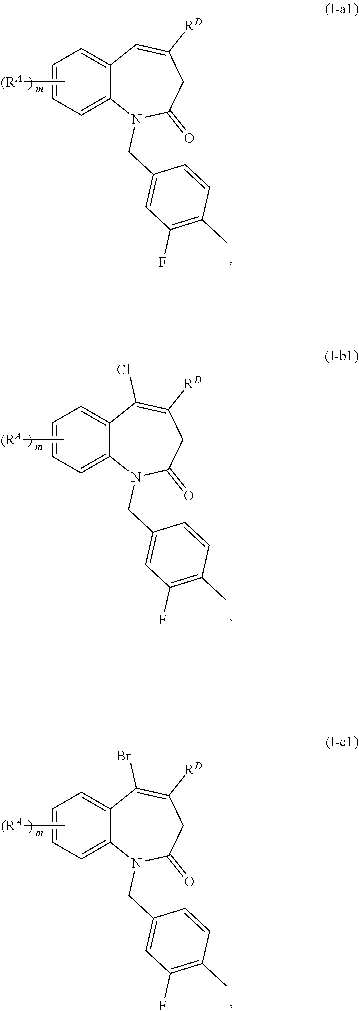
or a pharmaceutically acceptable salt thereof.

13. The compound of any of claims 1-12, wherein R^{D} is an optionally substituted 5-membered heteroaryl.

14. The compound of any of claims 1-12, wherein R^{D} is selected from the group consisting of

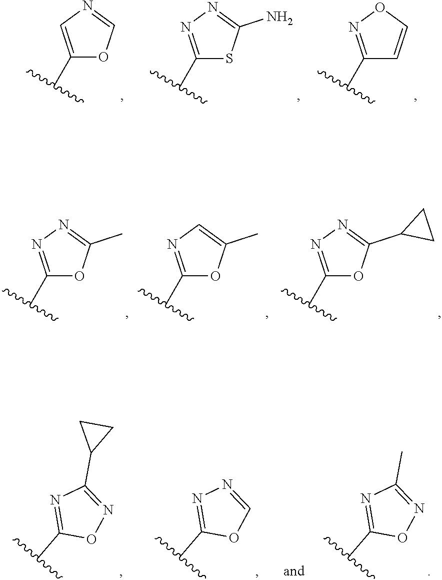

15. The compound of claim 2, wherein the compound is of formula (I-b2): 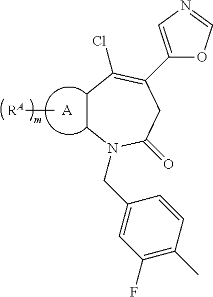
or a pharmaceutically acceptable salt thereof.

16. The compound of claim 2, wherein the compound is of formula (I-c2), (I-c3) or (I-c4) 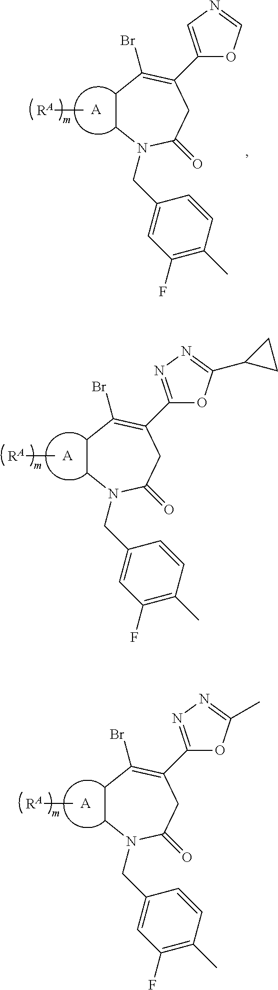
or a pharmaceutically acceptable salt thereof.

17. The compound of claim 2, wherein the compound is of formula (T-e) 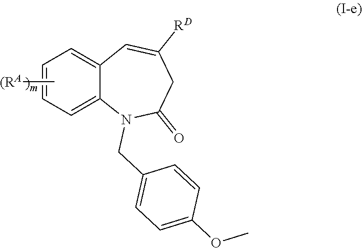
or a pharmaceutically acceptable salt thereof.

18. The compound of claim 2, wherein the compound is of formula (I-e1) 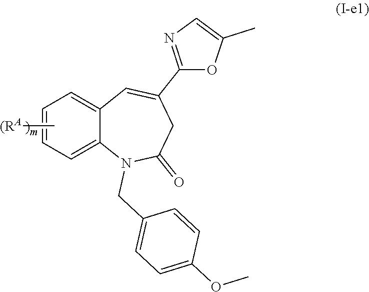
or a pharmaceutically acceptable salt thereof.

19. The compound of claim 2, wherein the compound is of formula (I-e2) 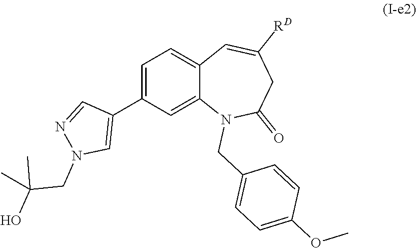
or a pharmaceutically acceptable salt thereof.

20. The compound of claim 2, wherein the compound is of formula (I-f) 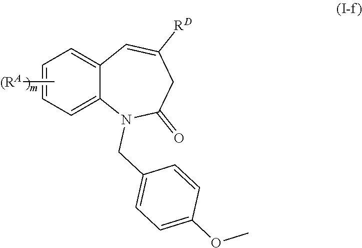
or a pharmaceutically acceptable salt thereof.

21. The compound of any of claims 1-20, wherein R^{A} is optionally substituted phenyl.

22. The compound of any of claims 1-20, wherein R^{A} is 5-6 membered heteroaryl substituted with one instance of R¹.

23. The compound of claim 21, wherein R^{A} is 5-membered heteroaryl substituted with one instance of R¹.

24. The compound of claim 18, wherein R^{A} is pyrazolyl substituted with one instance of R¹.

25. The compound of claim 18, wherein R^{A} is imidozyl substituted with one instance of R¹.

26. The compound of claim 21, wherein R^{A} is 6-membered heteroaryl substituted with one instance of R¹.

27. The compound of claim 26, wherein R^{A} is pyridinyl substituted with one instance of R¹.

28. The compound of claim 26, wherein R^{A} is pyrimidinyl substituted with one instance of R¹.

29. The compound of any of claims 1-20, wherein A and R^{A} together is selected from the group consisting of

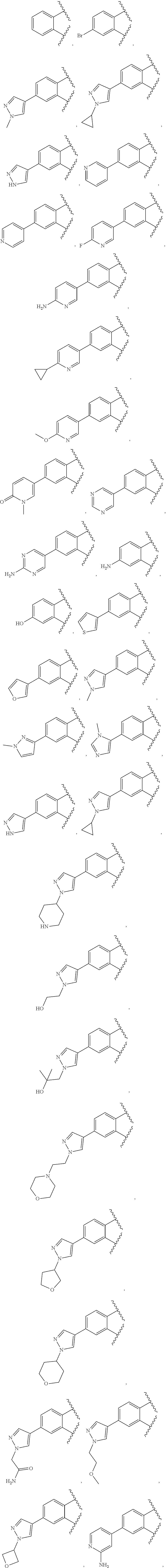

[chemical structure]

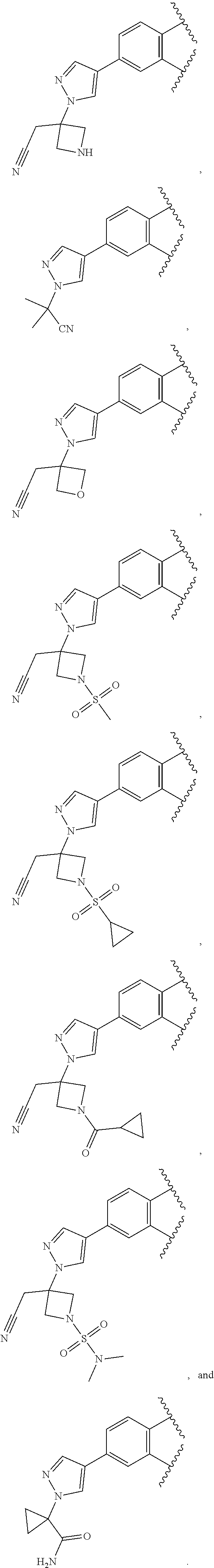

30. The compound of any of the previous claims, wherein the compound is described in Table 1.

31. A pharmaceutical composition comprising a compound of any of the previous claims and a pharmaceutically acceptable excipient.

32. A method of treat cancer comprising administering to a patient in need thereof the compound of any of claims 1-30 or the pharmaceutical composition of claim 31.
